(19) United States
(12) Patent Application Publication
(10) Pub. No.: US 2012/0029190 A1
(43) Pub. Date: Feb. 2, 2012

(54) COMPOUNDS FOR TREATING DISORDERS MEDIATED BY METABOTROPIC GLUTAMATE RECEPTOR 5, AND METHODS OF USE THEREOF

(76) Inventors: Douglas Burdi, Arlington, MA (US); Kerry L. Spear, Concord, MA (US); Larry Wendell Hardy, Sturbridge, MA (US)

(21) Appl. No.: 13/262,608

(22) PCT Filed: Apr. 1, 2010
(86) PCT No.: PCT/US10/29575

§ 371 Date: Oct. 18, 2011

Related U.S. Application Data
(60) Provisional application No. 61/255,790, filed on Oct. 28, 2009.
(60) Provisional application No. 61/166,661, filed on Apr. 3, 2009.

(51) Int. Cl. C07D 498/04; C07D 487/04; C07D 513/04

(52) U.S. Cl. 540/593; 546/115; 544/127; 544/405; 546/271.7; 546/256; 546/114; 544/350

(57) ABSTRACT

Provided herein are compounds and methods of synthesis thereof. The compounds provided herein are useful for the treatment, prevention, and/or management of various disorders, such as neurological disorders, psychiatric disorders, neuro-muscular disorders, gastrointestinal disorders, lower urinary tract disorder, and cancer. Compounds provided herein modulate the activity of metabotropic glutamate receptor 5 (mGluR5) in the central nervous system or the periphery. Pharmaceutical formulations containing the compounds and their methods of use are also provided herein.

This application claims priority to U.S. Provisional Patent Application No. 61/166,661, filed on Apr. 3, 2009, and U.S. Provisional Patent Application No. 61/255,790, filed on Oct. 28, 2009, both of which are hereby incorporated by reference herein in their entireties.

### I. FIELD

Provided herein are compounds useful for treating disorders mediated by metabotropic glutamate receptor 5 (mGluR5), compositions comprising the compounds, and methods of use thereof.

### II. BACKGROUND

L-Glutamate is a major excitatory neurotransmitter in the central nervous system, which binds to neurons and activates cell surface receptors. L-Glutamate acts through two heterogeneous families of receptors: ionotropic and metatropic glutamate receptors (mGluR). mGluRs are G protein-coupled receptors that activate intracellular second messengers when bound to glutamate. Eight subtypes of mGluRs have been cloned and classified into three groups on the basis of sequence similarities and pharmacological properties. mGluR1 and mGluR5 belong to Group I, which initiate cellular responses through a G-protein mediated mechanism and activate phospholipase C, leading to phosphoinositide hydrolysis and the mobilization of intracellular calcium (Schoepp, D. D., et al., Neuropharmacology 1999, 38, 1431).

mGluR5 is expressed in both the central nervous system and the periphery (Chizh, B. A., et al., Amino Acids 2002, 23, 169). Therefore, modulation of mGluR5 activity is useful in the treatment of both peripheral and CNS disorders. With respect to peripheral disorders, mGluR5 negative modulators have shown efficacy in the treatment of gastrointestinal (GI) tract disorders, such as gastroesophageal reflux disease (GERD).

In the CNS, excessive activation of mGluR5 has been implicated in a number of diseases, such as various pain states, psychiatric disorders such as anxiety and depression, and other neurological impairments such as drug addiction and drug withdrawal. For example, mGluR5 negative modulators are efficacious in the treatment of anxiety in a variety of animal models including stress-induced hyperthermia and fear-potentiated startle.

Migraine is another CNS disorder relevant to mGluR5 modulation. Migraine is a chronic debilitating condition characterized by recurrent severe headaches that are often accompanied by a variety of other symptoms, such as nausea and fatigue. Pharmacologic therapies for the treatment of migraine may be divided into two classes, acute therapies for the treatment of symptoms when they arise, and chronic therapies designed to prevent the onset of migraine (prophylactics) (Goadsby, P. J., et al., N. Engl. J. Med. 2002, 346, 257). The best known therapeutics for the treatment of acute migraine are triptans, dual 5-HT_{1b}/5-HT_{1d} agonists that exert their therapeutic effects through cranial vasoconstriction. Although generally well-tolerated, their use is restricted in the presence of cardiovascular disease due to their 5-HT_{1b} agonism.

In contrast to the treatment for acute attacks, the current therapies for migraine prophylaxis may be subdivided into three classes: β-blockers, anticonvulsants, and antidepressants. All are moderately effective and carry substantial side-effects. Most prominent among the β-blockers is propranolol, whose side-effects include lethargy and hypotension. Valproate and topiramate are the most commonly used anticonvulsants, but, like the antidepressants, they cause side-effects such as fatigue. There is a clear medical need for a novel prophylactic therapy that is effective and free from the side-effects. Recently, an mGluR5 antagonist demonstrated efficacy in treating acute migraine in human clinical trials. The robust anxiolytic and antidepressant activities of mGluR5 antagonists should be beneficial to migraine patients, who often suffer anxiety and depression.

Other peripheral and CNS disorders relevant to mGluR5 modulation include schizophrenia, neurodegenerative diseases, levodopa-induced dyskinesia, fragile X syndrome, substance abuse/addiction, epilepsy, inflammatory, visceral and neuropathic pain, and post-traumatic stress disorder. Therefore, there is a great need for effective mGluR5 modulators as therapeutics for the treatment of various disorders, such as neurological disorders.

### III. SUMMARY

Provided herein are compounds of formula (I), or pharmaceutically acceptable salts, solvates, or stereoisomers thereof:

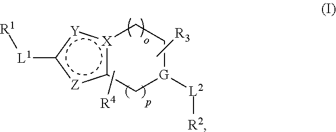

wherein X, Y, Z, L¹, L², R¹, R², R³, R⁴, G, o, and p are defined herein elsewhere. The compounds are useful in modulating the activity of mGluR5.

Also provided herein are compositions and dosage forms comprising compounds provided herein. Compositions and dosage forms provided herein may comprise one or more additional active ingredients.

Also provided herein are methods for the treatment, prevention, and/or management of various disorders mediated by mGluR5 using the compounds and compositions provided herein. Also provided herein are uses of the compounds and compositions provided herein in the manufacture of a medicament for the treatment, prevention, and/or management of one or more disorders provided herein. Also provided herein are compounds and compositions for use in the treatment, prevention, and/or management of one or more disorders provided herein. Disorders that may be treated, prevented, and/or managed include, but are not limited to, migraine, anxiety, dental phobia, depression, pain, inflammatory pain, neuropathic pain, postoperative pain, acute thermal hyperalgesia, mechanical allodynia, visceral pain, chronic pain, neurodegenerative disease, Alzheimer's disease, Parkinson's disease, levodopa-induced dyskinesia, Huntington's disease, amyotropic lateral sclerosis, epilepsy, seizure, psychosis, schizophrenia, substance abuse/addiction such as cocaine, nicotine, morphine, opioid, or alcohol abuse/addiction, bulimia, anorexia, smoking, obsessive compulsive disorder, aggression, post-traumatic stress disorder, autism, fragile X syndrome, excessive tactile sensitivity, sensory hyper-excitability, attention deficit hyperactivity disorder, bipolar disorder, mood disorder, cognitive disorder, mental retardation, Down syndrome, memory deficit, dementia, GERD, acid reflux, irritable bowel syndrome, lower urinary tract disorder, overactive bladder, urinary incontinence, oral cancer, glioneuronal cancer, asthma, chronic pharyngitis, lung disease, dyspepsia, stroke, head trauma, anoxic and ischemic injuries, and any other psychiatric, neurological, or neuromuscular disorders described herein elsewhere.

In one embodiment, provided herein is a method of modulating the activity of mGluR5. The method comprises contacting mGluR5 with a compound provided herein. In some embodiments, provided herein is a method of inhibiting or reducing the activity of mGluR5. In some embodiments, the method comprises contacting mGluR5 with an antagonist or a negative allosteric modulator, such as a compound provided herein. In one embodiment, the method comprises contacting the cell with a compound provided herein. In an exemplary embodiment, the cell is a brain cell, such as, for example, a neuronal cell or a glial cell.

### IV. DETAILED DESCRIPTION

Unless defined otherwise, all technical and scientific terms used herein have the same meaning as those commonly understood by one of ordinary skill in the art. All publications and patents referred to herein are incorporated by reference herein in their entireties.

### A. Definitions

As used herein, and unless otherwise indicated, the term “alkyl” refers to a linear or branched saturated monovalent hydrocarbon radical, wherein the alkyl may optionally be substituted with one or more substituents. In some embodiment, the alkyl may be optionally substituted with one or more halogen. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

As used herein, and unless otherwise specified, the term “alkenyl” refers to a linear or branched monovalent hydrocarbon radical, which contains one or more, in one embodiment, one to five, carbon-carbon double bonds. The alkenyl may be optionally substituted one or more substituents. In some embodiments, the alkenyl may be optionally substituted with one or more halogen. The term “alkenyl” also encompasses radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

As used herein, and unless otherwise specified, the term “alkynyl” refers to a linear or branched monovalent hydrocarbon radical, which contains one or more, in one embodiment, one to five, carbon-carbon triple bonds. The alkynyl may be optionally substituted one or more substituents. In some embodiments, the alkynyl may be optionally substituted with one or more halogen. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

As used herein, and unless otherwise specified, the term “cycloalkyl” refers to a cyclic saturated bridged and/or non-bridged monovalent hydrocarbon radical, which may be optionally substituted one or more substituents as described herein elsewhere. In certain embodiments, the cycloalkyl has from 3 to 20 (C₃₋₂₀), from 3 to 15 (C₃₋₁₅), from 3 to 12 (C₃₋₁₂), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents as described herein elsewhere.

As used herein, and unless otherwise specified, the term “arylalkyl” or “aralkyl” refers to a monovalent alkyl group substituted with aryl. In certain embodiments, both alkyl and aryl may be optionally substituted with one or more substituents as described herein elsewhere.

As used herein, and unless otherwise specified, the term “heteroaryl” refers to a monocyclic aromatic group and/or multicyclic aromatic group that contain at least one aromatic ring, wherein at least one ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may be optionally substituted with one or more substituents as described herein elsewhere.

As used herein, and unless otherwise specified, the term “heterocyclyl” or “heterocyclic” refers to a monocyclic non-aromatic ring system and/or multicyclic ring system that contains at least one non-aromatic ring, wherein at least one ring contains one or more heteroatoms independently selected from O, S, or N. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrofuryl, dihydrobenzisoxazinyl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl or heterocyclic may be optionally substituted with one or more substituents as described herein elsewhere.

As used herein, and unless otherwise specified, the term “halogen”, “halide” or “halo” refers to fluorine, chlorine, bromine, and/or iodine.

As used herein, and unless otherwise specified, the atoms of the compounds provided herein are meant to represent any stable isotope of that atom. For example, as used herein, and unless otherwise specified, hydrogen encompasses proton (¹H), deuterium (²H), tritium (³H), and/or mixtures thereof. In one embodiment, when a position is designated as “H” or “hydrogen”, the position is understood to have hydrogen at its natural isotopic composition. In one embodiment, when a position is designated as “H” or “hydrogen”, the position is understood to have hydrogen at an isotopically enriched composition, i.e., an isotopic composition other than the natural isotopic composition of that atom. In one embodiment, the compounds provided herein optionally comprise deuterium at one or more positions where hydrogen atoms are present, and wherein the deuterium composition of the atom or atoms is other than the natural isotopic composition. In one embodiment, the compounds provided herein optionally comprise isotopes for other elements at one or more positions, including but not limited to, ¹³C, ¹⁴C, ³³S, ³⁴S, ³⁶S, ¹⁵N, ¹⁷O and/or ¹⁸O, and wherein the isotopic composition of the atom or atoms is other than the natural isotopic composition.

As used herein, and unless otherwise specified, the term “optionally substituted” refers to a group, such as an alkyl, alkenyl, alkynyl, cycloalkyl, aryl, aralkyl, heteroaryl, or heterocyclyl, which may be substituted with one or more substituents independently selected from, e.g., (a) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (b) halo, cyano (—CN), nitro (—NO₂), —C(O)R^{a}, —C(O)OR^{a}, —C(O)NR^{b}R^{c}, —C(NR^{a})NR^{b}R^{c}, —OR^{a}, —OC(O)R^{a}, —OC(O)ORₐ, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heteroaryl or heterocyclyl, optionally substituted with one or more, in one embodiment, one, two, three, or four, substituents Q¹. As used herein, and unless otherwise specified, all groups that can be substituted are “optionally substituted.”

In one embodiment, each Q¹ is independently selected from the group consisting of (a) cyano, halo, and nitro; and (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heteroaryl or heterocyclyl.

As used herein, and unless otherwise specified, the term “stereoisomer” encompasses all enantiomerically/stereomerically pure and enantiomerically/stereomerically enriched compounds provided herein.

As used herein and unless otherwise specified, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

As used herein and unless otherwise specified, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise specified, the term “enantiomerically pure” means a stereomerically pure composition of a compound having one chiral center. Similarly, the term “enantiomerically enriched” means a stereomerically enriched composition of a compound having one chiral center.

As used herein, and unless otherwise specified, the term “optically active” or “enantiomerically active” refers to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

As used herein, and unless otherwise specified, the term “pharmaceutically acceptable salts” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. In some embodiments, the salt is formed from hydrochloric, hydrobromic, phosphoric, or sulfuric acid. In one embodiment, the salt is formed from hydrochloride salt.

As used herein, and unless otherwise specified, the term “solvate” refers to a compound provided herein or a salt thereof, which further includes a stoichiometric or non-stoichiometric amount of solvent bound by non-covalent intermolecular forces. Where the solvent is water, the solvate is a hydrate.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

As used herein, and unless otherwise specified, the terms “drug” and “therapeutic agent” refer to a compound, or a pharmaceutical composition thereof, which is administered to a subject for treating, preventing, or ameliorating one or more symptoms of a condition, disorder, or disease.

As used herein, and unless otherwise indicated, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

As used herein, and unless otherwise indicated, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice and the like. In specific embodiments, the subject is a human.

As used herein, and unless otherwise specified, the term “metabotropic glutamate receptor ligand” or “mGluR ligand” refers to any compound, which binds to an mGluR receptor. Unless otherwise specified, the mGluR receptor includes, but is not limited to mGluR5. Ligands include endogenous ligands for a given metabotropic glutamate receptor as well as drug molecules and other compounds, such as synthetic molecules known to bind to a particular metabotropic glutamate receptor. In some embodiments, the ligand is an allosteric modulator. In one embodiment, the ligands include those labeled with one or more radioisotopes, such as tritium or ¹¹C, or otherwise (e.g., fluorescently) labeled. In some embodiments, the ligand is a positron-emission tomography (PET) ligand. It is within the abilities of the skilled person to select an appropriate ligand, for example, an agonist or an antagonist, for a given metabotropic glutamate receptor.

As used herein, and unless otherwise specified, the term “neurological disorder” refers to any condition of the central or peripheral nervous system of a mammal. The term “neurological disorder” includes, but is not limited to, neurodegenerative diseases (e.g., Alzheimer's disease, Parkinson's disease, levodopa-induced dyskinesia, and amyotrophic lateral sclerosis), neuropsychiatric diseases (e.g., schizophrenia and anxiety, such as general anxiety disorder), and affective disorders (e.g., depression, anxiety, and attention deficit disorder). Exemplary neurological disorders include, but are not limited to, MLS (cerebellar ataxia), Huntington's disease, Down syndrome, multi-infarct dementia, status epilecticus, contusive injuries (e.g., spinal cord injury and head injury), viral infection induced neurodegeneration, (e.g., AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. For instance, a method of treating a neurodegenerative disorder includes methods of treating loss of memory and/or loss of cognition associated with a neurodegenerative disorder. An exemplary method would also include treating or preventing loss of neuronal function characteristic of neurodegenerative disorder. “Neurological disorder” also includes any disease or condition that is implicated, at least in part, in monoamine (e.g., norepinephrine) signaling pathways (e.g., cardiovascular disease).

As used herein, and unless otherwise specified, the term “affective disorder” includes depression, anxiety (e.g., generalized anxiety disorder (GAD)), attention deficit disorder, attention deficit disorder with hyperactivity, bipolar and manic conditions, obsessive-compulsive disorder, and the like. The terms “attention deficit disorder” (ADD) and “attention deficit disorder with hyperactivity” (ADDH), or attention deficit/hyperactivity disorder (AD/HD), are used herein in accordance with the accepted meanings as found in the Diagnostic and Statistical Manual of Mental Disorders, 4^{th} Ed., American Psychiatric Association (1997) (DSM-IV™).

As used herein, and unless otherwise specified, the term “depression” includes all forms of depression including, but not limited to, major depressive disorder (MDD), bipolar disorder, seasonal affective disorder (SAD) and dysthymia. “Major depressive disorder” is used herein interchangeably with “unipolar depression” and “major depression.” “Depression” may also includes any condition commonly associated with depression, such as all forms of fatigue (e.g., chronic fatigue syndrome) and cognitive deficits.

As used herein, and unless otherwise specified, the terms “substance abuse” and “eating disorders” are used herein in a manner consistent with their accepted meanings in the art. See, e.g., DSM-IV™. For example, the term “eating disorder,” as used herein, refers to abnormal compulsions to avoid eating or uncontrollable impulses to consume abnormally large amounts of food. These disorders may affect not only the social well-being, but also the physical well-being of sufferers. Examples of eating disorders include, but are not limited to, anorexia nervosa, bulimia, and binge eating.

As used herein, and unless otherwise specified, the term “pain” refers to an unpleasant sensory and emotional experience. The term “pain,” as used herein, refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (see, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia. In addition, The term “pain” includes pain resulting from dysfunction of the nervous system: organic pain states that share clinical features of neuropathic pain and possible common pathophysiology mechanisms, but are not initiated by an identifiable lesion in any part of the nervous system.

The term “somatic pain,” as used herein, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

The term “neuropathic pain,” as used herein, refers to a heterogeneous group of neurological conditions that result from damage to the nervous system. The term also refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Common types of peripheral neuropathic pain include diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis, and is also encompassed by the term. Other types of pain that are meant to be included in the definition of neuropathic pain include, but are not limited to, pain from neuropathic cancer pain, HIV/AIDS induced pain, phantom limb pain, and complex regional pain syndrome.

The term also encompasses the common clinical features of neuropathic pain including, but not limited to, sensory loss, allodynia (non-noxious stimuli produce pain), hyperalgesia and hyperpathia (delayed perception, summation, and painful after sensation). Pain is often a combination of nociceptive and neuropathic types, for example, mechanical spinal pain and radiculopathy or myelopathy.

As used herein, and unless otherwise specified, the term “acute pain” refers to the normal, predicted physiological response to a noxious chemical, thermal or mechanical stimulus typically associated with invasive procedures, trauma and disease. It is generally time-limited, and may be viewed as an appropriate response to a stimulus that threatens and/or produces tissue injury. The term also refers to pain which is marked by short duration or sudden onset.

As used herein, and unless otherwise specified, the term “chronic pain” encompasses the pain occurring in a wide range of disorders, for example, trauma, malignancies and chronic inflammatory diseases such as rheumatoid arthritis. Chronic pain may last more than about six months. In addition, the intensity of chronic pain may be disproportionate to the intensity of the noxious stimulus or underlying process. The term also refers to pain associated with a chronic disorder, or pain that persists beyond resolution of an underlying disorder or healing of an injury, and that is often more intense than the underlying process would predict. It may be subject to frequent recurrence.

As used herein, and unless otherwise specified, the term “inflammatory pain” is pain in response to tissue injury and the resulting inflammatory process. Inflammatory pain is adaptive in that it elicits physiologic responses that promote healing. However, inflammation may also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. The term also refers to pain which is produced as a symptom or a result of inflammation or an immune system disorder.

As used herein, and unless otherwise specified, the term “visceral pain” refers to pain which is located in an internal organ.

As used herein, and unless otherwise specified, the term “mixed etiology pain” refers to pain that contains both inflammatory and neuropathic components.

As used herein, and unless otherwise specified, the term “dual mechanism pain” refers to pain that is amplified and maintained by both peripheral and central sensitization.

As used herein, and unless otherwise specified, the term “causalgia” refers to a syndrome of sustained burning, allodynia, and hyperpathia after a traumatic nerve lesion, often combined with vasomotor and sudomotor dysfunction and later trophic changes. As used herein, and unless otherwise specified, the term “central pain” refers to pain initiated by a primary lesion or dysfunction in the central nervous system.

As used herein, and unless otherwise specified, the term “hyperesthesia” refers to increased sensitivity to stimulation, excluding the special senses.

As used herein, and unless otherwise specified, the term “hyperpathia” refers to a painful syndrome characterized by an abnormally painful reaction to a stimulus, especially a repetitive stimulus, as well as an increased threshold. It may occur with allodynia, hyperesthesia, hyperalgesia, or dysesthesia.

As used herein, and unless otherwise specified, the term “dysesthesia” refers to an unpleasant abnormal sensation, whether spontaneous or evoked. In certain embodiments, dysesthesia include hyperalgesia and allodynia.

As used herein, and unless otherwise specified, the term “hyperalgesia” refers to an increased response to a stimulus that is normally painful. It reflects increased pain on suprathreshold stimulation.

As used herein, and unless otherwise specified, the term “allodynia” refers to pain due to a stimulus that does not normally provoke pain.

As used herein, and unless otherwise specified, the term “Diabetic Peripheral Neuropathic Pain” (DPNP), also called diabetic neuropathy, DN or diabetic peripheral neuropathy), refers to chronic pain caused by neuropathy associated with diabetes mellitus. The classic presentation of DPNP is pain or tingling in the feet that can be described not only as “burning” or “shooting” but also as severe aching pain. Less commonly, patients may describe the pain as itching, tearing, or like a toothache. The pain may be accompanied by allodynia and hyperalgesia and an absence of symptoms, such as numbness.

As used herein, and unless otherwise specified, the term “Post-Herpetic Neuralgia”, also called “Postherpetic Neuralgia (PHN)”, refers to a painful condition affecting nerve fibers and skin. Without being limited by a particular theory, it is a complication of shingles, a second outbreak of the varicella zoster virus (VZV), which initially causes chickenpox.

As used herein, and unless otherwise specified, the term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

As used herein, and unless otherwise specified, the term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

As used herein, and unless otherwise specified, the term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

As used herein, and unless otherwise specified, the term “Trigeminal Neuralgia (TN)” refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

As used herein, and unless otherwise specified, the term “Complex Regional Pain Syndrome (CRPS),” formerly known as Reflex Sympathetic Dystrophy (RSD), refers to a chronic pain condition whose key symptom is continuous, intense pain out of proportion to the severity of the injury, which gets worse rather than better over time. The term encompasses type 1 CRPS, which includes conditions caused by tissue injury other than peripheral nerve, and type 2 CRPS, in which the syndrome is provoked by major nerve injury, and is sometimes called causalgia.

As used herein, and unless otherwise specified, the term “fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

As used herein, and unless otherwise specified, the term “convulsion” refers to a neurological disorder and is used interchangeably with “seizure,” although there are many types of seizure, some of which have subtle or mild symptoms instead of convulsions. Seizures of all types may be caused by disorganized and sudden electrical activity in the brain. In some embodiments, convulsions are a rapid and uncontrollable shaking during which the muscles contract and relax repeatedly.

### B. Compounds

In one embodiment, provided herein is a compound of formula (I):

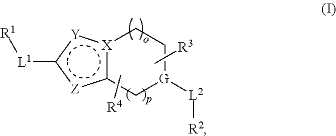

or a pharmaceutically acceptable salt, solvate, or stereoisomer thereof, wherein

R¹ is cycloalkyl, heterocyclyl, aryl, or heteroaryl, each of which is optionally substituted;

R² is cycloalkyl, heterocyclyl, aryl, or heteroaryl, each of which is optionally substituted;

R³ and R⁴ are each independently hydrogen, halogen, or lower alkyl; or

when R³ and R⁴ are attached to the same carbon atom, CR³R⁴ is C═O, or R³ and R⁴ may be combined with the carbon atom to which they are attached to form a 3- to 7-membered spiro cycloalkyl; or

when R³ and R⁴ are attached to different carbon atoms, R³ and R⁴ may be combined with the carbon atoms to which they are attached to form a 3- to 7-membered bridged or fused cycloalkyl;

L¹ is a bond, —S—, —SO—, —SO₂—, —O—, —NR⁹—, —CR⁵R⁶—, —CR⁵R⁶—CR⁷R⁸—, optionally substituted cycloalkyl, optionally substituted heterocyclyl; optionally substituted aryl, or optionally substituted heteroaryl;

L² is a bond, —O—, —NR⁹—, —CR⁵R⁶— or —CR⁵R⁶—CR⁷R⁸—;

X is C or N;

Y is O, S, N, NR¹⁰, or CR¹⁰;

Z is O, S, N, NR¹⁰, or CR¹⁰; wherein Y and Z are not both O or both S;

R⁵ and R⁶ are each independently hydrogen, halogen, or lower alkyl, or CR⁵R⁶ is C═O; or R⁵ and R⁶ may be combined with the carbon atom to which they are attached to form a 3- to 7-membered cycloalkyl;

R⁷ and R⁸ are each independently hydrogen, halogen, or lower alkyl, or CR⁷R⁸ is C═O; or R⁷ and R⁸ may be combined with the carbon atom to which they are attached to form a 3- to 7-membered cycloalkyl;

R⁹ and R¹⁰ are each independently hydrogen or lower alkyl;

G is N, CH, CR′, COR′, or CNR′R″;

R′ is lower alkyl;

R″ is lower alkyl;

L² and R′ or L² and R″ may be combined to form a 3- to 10-membered ring;

o is 0, 1, or 2; and

p is 1 or 2.

In one embodiment, provided herein is a compound of formula (I):

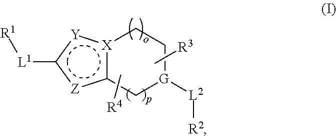

or a pharmaceutically acceptable salt, solvate, or stereoisomer thereof, wherein

R¹ is cycloalkyl, heterocyclyl, aryl, or heteroaryl, each of which is optionally substituted;

R² is cycloalkyl, heterocyclyl, aryl, or heteroaryl, each of which is optionally substituted;

R³ and R⁴ are each independently hydrogen, halogen, or lower alkyl; or

when R³ and R⁴ are attached to the same carbon atom, CR³R⁴ is C═O, or R³ and R⁴ may be combined with the carbon atom to which they are attached to form a 3- to 7-membered spiro cycloalkyl; or

when R³ and R⁴ are attached to different carbon atoms, R³ and R⁴ may be combined with the carbon atoms to which they are attached to form a 3- to 7-membered bridged or fused cycloalkyl;

L¹ is a bond, —S—, —SO—, —SO₂—, —O—, —NR⁹—, —CR⁵R⁶—, —CR⁵R⁶—CR⁷R⁸—, optionally substituted cycloalkyl, optionally substituted heterocyclyl; optionally substituted aryl, or optionally substituted heteroaryl;

L² is a bond, —O—, —NR⁹—, —CR⁵R⁶— or —CR⁵R⁶—CR⁷R⁸—;

X is C or N;

Y is O, S, N, NR¹⁰, or CR¹⁰;

Z is O, S, N, NR¹⁰, or CR¹⁰; wherein Y and Z are not both 0 or both S;

R⁵ and R⁶ are each independently hydrogen, halogen, or lower alkyl, or CR⁵R⁶ is C═O; or R⁵ and R⁶ may be combined with the carbon atom to which they are attached to form a 3- to 7-membered cycloalkyl;

R⁷ and R⁸ are each independently hydrogen, halogen, or lower alkyl, or CR⁷R⁸ is C═O; or R⁷ and R⁸ may be combined with the carbon atom to which they are attached to form a 3- to 7-membered cycloalkyl;

R⁹ and R¹⁰ are each independently hydrogen or lower alkyl;

G is N or CH;

o is 0, 1, or 2; and

p is 1 or 2.

In one embodiment, provided herein is a compound of formula (I):

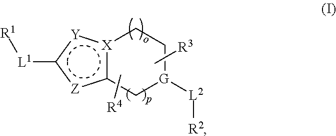

or a pharmaceutically acceptable salt, solvate, or stereoisomer thereof, wherein:

R¹ is cycloalkyl, heterocyclyl, aryl, or heteroaryl, each of which is optionally substituted;

R² is cycloalkyl, heterocyclyl, aryl, or heteroaryl, each of which is optionally substituted;

R³ and R⁴ are each independently hydrogen, halogen, or lower alkyl; or

when R³ and R⁴ are attached to the same carbon atom, CR³R⁴ is C═O, or R³ and R⁴ may be combined with the carbon atom to which they are attached to form a 3- to 7-membered spiro cycloalkyl; or

when R³ and R⁴ are attached to different carbon atoms, R³ and R⁴ may be combined with the carbon atoms to which they are attached to form a 3- to 7-membered bridged or fused cycloalkyl;

L¹ is a bond, —S—, —SO—, —SO₂—, —O—, —NR⁹—, —CR⁵R⁶—, —CR⁵R⁶—CR⁷R⁸—, optionally substituted cycloalkyl, optionally substituted heterocyclyl; optionally substituted aryl, or optionally substituted heteroaryl;

L² is a bond, —O—, —NR⁹—, or —CR⁵R⁶—;

X is C or N;

Y is O, S, N, NR¹⁰, or CR¹⁰;

Z is O, S, N, NR¹⁰, or CR¹⁰; wherein Y and Z are not both 0 or both S;

R⁵ and R⁶ are each independently hydrogen, halogen, or lower alkyl, or CR⁵R⁶ is C═O; or R⁵ and R⁶ may be combined with the carbon atom to which they are attached to form a 3- to 7-membered cycloalkyl;

R⁷ and R⁸ are each independently hydrogen, halogen, or lower alkyl, or CR⁷R⁸ is C═O; or R⁷ and R⁸ may be combined with the carbon atom to which they are attached to form a 3- to 7-membered cycloalkyl;

R⁹ and R¹⁰ are each independently hydrogen or lower alkyl;

G is N or CH;

o is 0, 1, or 2; and

p is 1 or 2.

In one embodiment, R¹ is optionally substituted cycloalkyl. In another embodiment, R¹ is optionally substituted monocyclic cycloalkyl. In another embodiment, R¹ is optionally substituted heterocyclyl. In another embodiment, R¹ is optionally substituted monocyclic heterocyclyl. In another embodiment, R¹ is optionally substituted aryl. In another embodiment, R¹ is optionally substituted monocyclic aryl. In another embodiment, R¹ is optionally substituted heteroaryl. In another embodiment, R¹ is optionally substituted monocyclic heteroaryl.

In one embodiment, R² is optionally substituted cycloalkyl. In another embodiment, R² is optionally substituted monocyclic cycloalkyl. In another embodiment, R² is optionally substituted heterocyclyl. In another embodiment, R² is optionally substituted monocyclic heterocyclyl. In another embodiment, R² is optionally substituted aryl. In another embodiment, R² is optionally substituted monocyclic aryl. In another embodiment, R² is optionally substituted heteroaryl. In another embodiment, R² is optionally substituted monocyclic heteroaryl.

In one embodiment, R³ is hydrogen. In another embodiment, R³ is halogen. In another embodiment, R³ is lower alkyl.

In one embodiment, R⁴ is hydrogen. In another embodiment, R⁴ is halogen. In another embodiment, R⁴ is lower alkyl.

In one embodiment, when R³ and R⁴ are attached to the same carbon atom, CR³R⁴ is C═O. In another embodiment, when R³ and R⁴ are attached to the same carbon atom, R³ and R⁴ are combined with the carbon atom to which they are attached to form a 3- to 7-membered spiro cycloalkyl. In another embodiment, when R³ and R⁴ are attached to different carbon atoms, R³ and R⁴ are combined with the carbon atoms to which they are attached to form a 3- to 7-membered bridged or fused cycloalkyl;

In one embodiment, L¹ is a bond. In another embodiment, L¹ is —S—. In another embodiment, L¹ is —SO—. In another embodiment, L¹ is —SO₂—. In another embodiment, L¹ is —O—. In another embodiment, L¹ is —NR⁹—. In another embodiment, L¹ is —CR⁵R⁶—. In another embodiment, L¹ is —CR⁵R⁶—CR⁷R⁸—. In another embodiment, L¹ is optionally substituted cycloalkyl. In another embodiment, L¹ is optionally substituted heterocyclyl. In another embodiment, L¹ is optionally substituted aryl. In another embodiment, L¹ is optionally substituted heteroaryl. R⁵, R⁶, R⁷, R⁸, and R⁹ are defined herein elsewhere.

In one embodiment, L² is a bond. In another embodiment, L² is —O—. In another embodiment, L² is —NR⁹—. In another embodiment, L² is —CR⁵R⁶—. In another embodiment, L¹ is —CR⁵R⁶—CR⁷R⁸—. R⁵, R⁶, R⁷, R⁸, and R⁹ are defined herein elsewhere.

In one embodiment, R⁵ is hydrogen. In another embodiment, R⁵ is halogen. In another embodiment, R⁵ is lower alkyl.

In one embodiment, R⁶ is hydrogen. In another embodiment, R⁶ is halogen. In another embodiment, R⁶ is lower alkyl.

In one embodiment, CR⁵R⁶ is C═O. In another embodiment, R⁵ and R⁶ are combined with the carbon atom to which they are attached to form a 3- to 7-membered cycloalkyl.

In one embodiment, R⁷ is hydrogen. In another embodiment, R⁷ is halogen. In another embodiment, R⁷ is lower alkyl.

In one embodiment, R⁸ is hydrogen. In another embodiment, R⁸ is halogen. In another embodiment, R⁸ is lower alkyl.

In one embodiment, CR⁷R⁸ is C═O. In another embodiment, R⁷ and R⁸ are combined with the carbon atom to which they are attached to form a 3- to 7-membered cycloalkyl.

In one embodiment, R⁹ is hydrogen. In another embodiment, R⁹ is lower alkyl.

In one embodiment, X is C. In another embodiment, X is N.

In one embodiment, Y is O. In another embodiment, Y is S. In another embodiment, Y is N. In another embodiment, Y is NR¹⁰. In another embodiment, Y is CR¹⁰. R¹⁰ is defined herein elsewhere.

In one embodiment, Z is O. In another embodiment, Z is S. In another embodiment, Z is N. In another embodiment, Z is NR¹⁰. In another embodiment, Z is CR¹⁰. R¹⁰ is defined herein elsewhere.

In one embodiment, R¹⁰ is hydrogen. In another embodiment, R¹⁰ is lower alkyl.

In one embodiment, G is N. In another embodiment, G is CH.

In one embodiment, G is CR′. In one embodiment, G is COR′. In one embodiment, G is CNR′R″. In one embodiment, R′ is lower alkyl. In one embodiment, R″ is lower alkyl.

In one embodiment, L² and R′ may be combined to form a 3- to 10-membered ring. In one embodiment, when L² is —NR⁹— and R⁹ is lower alkyl, R⁹ and R′ may be combined to form a 3- to 10-membered ring. In one embodiment, when L² is —CR⁵R⁶— and R⁵ is lower alkyl, R⁵ and R′ may be combined to form a 3- to 10 membered ring. In one embodiment, when L² is —CR⁵R⁶— and R⁶ is lower alkyl, R⁶ and R′ may be combined to form a 3- to 10 membered ring. In one embodiment, when L² is —CR⁵R⁶—CR⁷R⁸ and R⁵ is lower alkyl, R⁵ and R′ may be combined to form a 3- to 10 membered ring. In one embodiment, when L² is —CR⁵R⁶—CR⁷R⁸ and R⁶ is lower alkyl, R⁶ and R′ may be combined to form a 3- to 10 membered ring. In one embodiment, when L² is —CR⁵R⁶—CR⁷R⁸ and R⁷ is lower alkyl, R⁷ and R′ may be combined to form a 3- to 10 membered ring. In one embodiment, when L² is —CR⁵R⁶—CR⁷R⁸ and R⁸ is lower alkyl, R⁸ and R′ may be combined to form a 3- to 10 membered ring.

In one embodiment, L² and R″ may be combined to form a 3- to 10-membered ring. In one embodiment, when L² is —NR⁹— and R⁹ is lower alkyl, R⁹ and R″may be combined to form a 3- to 10-membered ring. In one embodiment, when L² is —CR⁵R⁶— and R⁵ is lower alkyl, R⁵ and R″ may be combined to form a 3- to 10 membered ring. In one embodiment, when L² is —CR⁵R⁶— and R⁶ is lower alkyl, R⁶ and R″ may be combined to form a 3- to 10 membered ring. In one embodiment, when L² is —CR⁵R⁶—CR⁷R⁸ and R⁵ is lower alkyl, R⁵ and R″ may be combined to form a 3- to 10 membered ring. In one embodiment, when L² is —CR⁵R⁶—CR⁷R⁸ and R⁶ is lower alkyl, R⁶ and R″ may be combined to form a 3- to 10 membered ring. In one embodiment, when L² is —CR⁵R⁶—CR⁷R⁸ and R⁷ is lower alkyl, R⁷ and R″ may be combined to form a 3- to 10 membered ring. In one embodiment, when L² is —CR⁵R⁶—CR⁷R⁸ and R⁸ is lower alkyl, R⁸ and R″ may be combined to form a 3- to 10 membered ring.

In one embodiment, o is 0. In another embodiment, o is 1. In another embodiment, o is 2.

In one embodiment, p is 1. In another embodiment p is 2.

Any of the combinations of R¹, R², R³, R⁴, R⁵, R⁶, R⁷, R⁸, R⁹, R¹⁰, L¹, L², X, Y, Z, G, o, and p are encompassed by this disclosure and specifically provided herein.

In one embodiment, provided herein is a compound of formula (Ia):

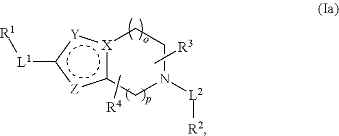

or a pharmaceutically acceptable salt, solvate, or stereoisomer thereof, wherein R¹, R², R³, R⁴, L¹, L², X, Y, Z, o, and p are as defined herein elsewhere.

In one embodiment, provided herein is a compound of formula (Ib):

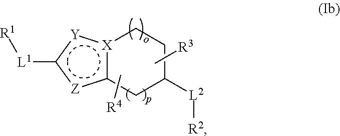

or a pharmaceutically acceptable salt, solvate, or stereoisomer thereof, wherein R¹, R², R³, R⁴, L¹, L², X, Y, Z, o, and p are as defined herein elsewhere.

In one embodiment, L¹ is a bond or —CR⁵R⁶—, wherein R⁵ and R⁶ are each independently hydrogen or lower alkyl, or R⁵ and R⁶ may be combined with the carbon atom to which they are attached to form a 3- to 7-membered cycloalkyl. In one embodiment, L² is a bond or —CR⁵R⁶—, wherein R⁵ and R⁶ are each independently hydrogen or lower alkyl, or CR⁵R⁶ is C═O, or R⁵ and R⁶ may be combined with the carbon atom to which they are attached to form a 3- to 7-membered cycloalkyl. In one embodiment, R¹ is aryl or heteroaryl, each of which is optionally substituted. In one embodiment, R² is aryl or heteroaryl, each of which is optionally substituted. In one embodiment, R¹ and R² are as defined herein elsewhere. In one embodiment, L¹ is a bond. In another embodiment, L¹ is —CH₂—. In another embodiment, L¹ is —C(CH₃)₂—. In one embodiment, L² is a bond. In another embodiment, L² is —CH₂—. In another embodiment, L² is —C(O)—. In another embodiment, L² is —CH₂—C(O)—. In one embodiment, L¹ is a bond, —CH₂—, or —C(CH₃)₂—. In one embodiment, L² is a bond, —CH₂—, —C(O)—, or —CH₂—C(O)—.

In one embodiment, R³ is hydrogen or methyl. In one embodiment, R⁴ is hydrogen or methyl. In one embodiment, R³ and R⁴ are both hydrogen. In one embodiment, R³ and R⁴ are both methyl. In one embodiment, one of R³ and R⁴ is methyl and the other is hydrogen. In one embodiment, R³ is methyl and R⁴ is hydrogen.

In one embodiment, specific examples include, but are not limited to, the following compounds:

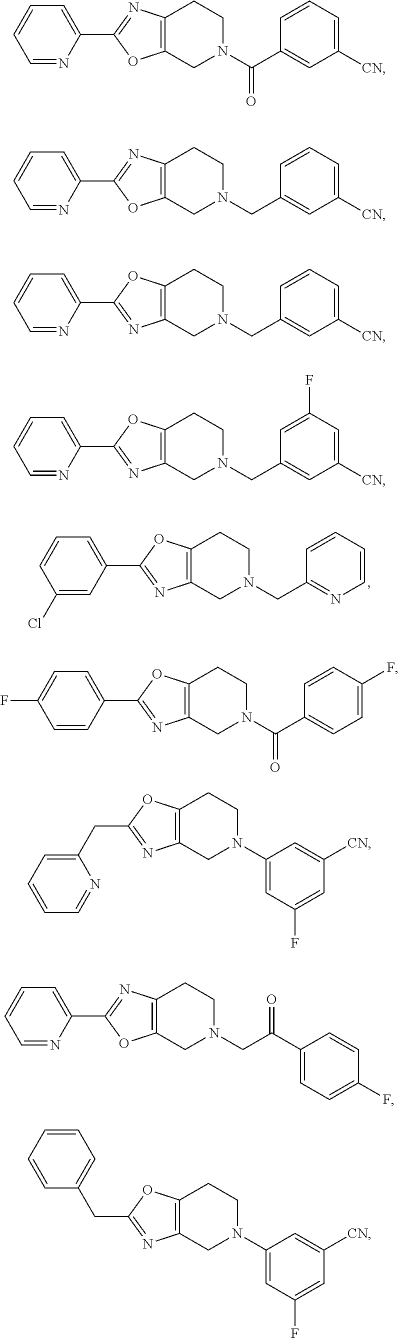

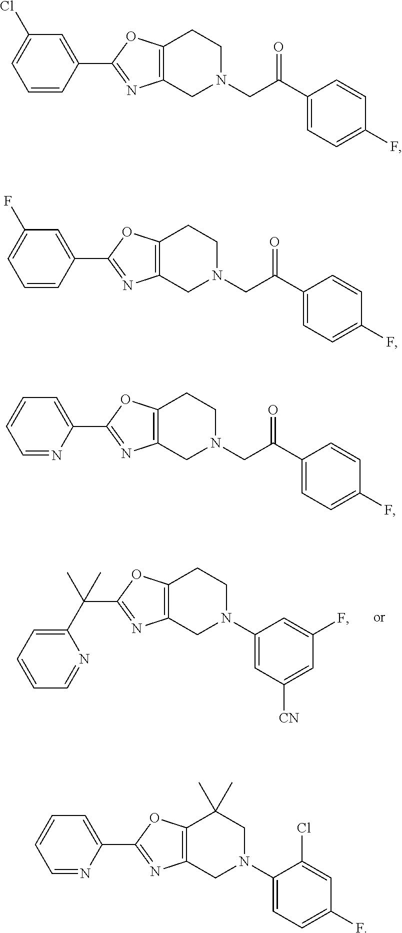

In one embodiment, specific examples include, but are not limited to, the following compounds:

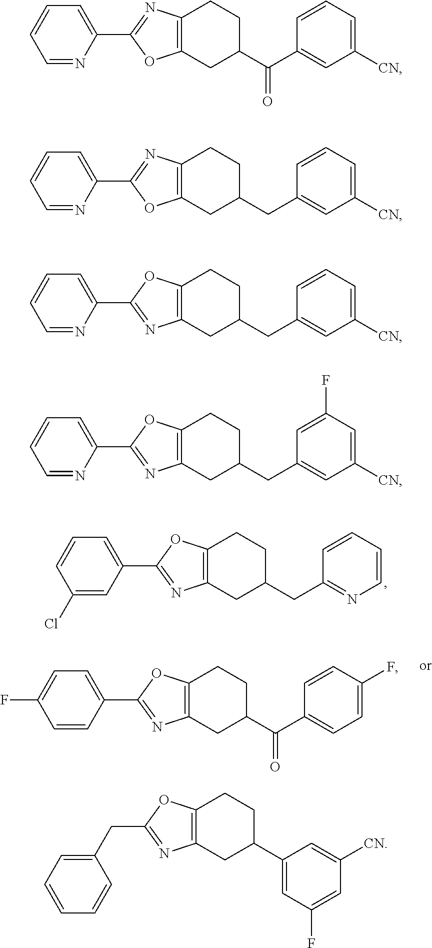

In one embodiment, provided herein is a compound of formula (II):

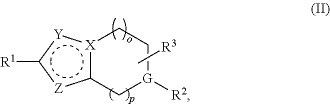

or a pharmaceutically acceptable salt, solvate, or stereoisomer thereof, wherein

R¹ is 5- or 6-membered aryl, or 5- or 6-membered heteroaryl, each of which is optionally substituted;

R² is 5- or 6-membered aryl, or 5- or 6-membered heteroaryl, each of which is optionally substituted;

R³ is hydrogen or lower alkyl;

X is C or N;

Y is O, S, N, NR¹⁰, or CR¹⁰;

Z is O, S, N, NR¹⁰, or CR¹⁰; wherein Y and Z are not both 0 or both S;

R¹⁰ is hydrogen or lower alkyl;

G is N or CH;

o is 0, 1, or 2; and

p is 1 or 2.

In one embodiment, R¹ is optionally substituted 5- or 6-membered aryl. In another embodiment, R¹ is optionally substituted 5- or 6-membered heteroaryl. In one embodiment, R¹ is optionally substituted phenyl. In another embodiment, R¹ is optionally substituted pyridyl. In another embodiment, R¹ is optionally substituted pyrimidinyl. In another embodiment, R¹ is optionally substituted pyrazinyl. In another embodiment, R¹ is optionally substituted pyridazinyl. In another embodiment, R¹ is optionally substituted triazinyl. In another embodiment, R¹ is optionally substituted furanyl. In another embodiment, R¹ is optionally substituted thienyl. In another embodiment, R¹ is optionally substituted pyrrolyl. In another embodiment, R¹ is optionally substituted pyrazolyl. In another embodiment, R¹ is optionally substituted imidazolyl. In another embodiment, R¹ is optionally substituted thiazolyl. In another embodiment, R¹ is optionally substituted oxazolyl. In another embodiment, R¹ is optionally substituted isothiazolyl. In another embodiment, R¹ is optionally substituted isoxazolyl. In another embodiment, R¹ is optionally substituted oxadiazolyl. In another embodiment, R¹ is optionally substituted thiadiazolyl. In another embodiment, R¹ is optionally substituted triazolyl. In another embodiment, R¹ is optionally substituted tetrazolyl. In another embodiment, R¹ is optionally substituted pyridine oxide.

In one embodiment, R² is optionally substituted 5- or 6-membered aryl. In another embodiment R² is optionally substituted 5- or 6-membered heteroaryl. In one embodiment, R² is optionally substituted phenyl. In another embodiment, R² is optionally substituted pyridyl. In another embodiment, R² is optionally substituted pyrimidinyl. In another embodiment, R² is optionally substituted pyrazinyl. In another embodiment, R² is optionally substituted pyridazinyl. In another embodiment, R² is optionally substituted triazinyl. In another embodiment, R² is optionally substituted furanyl. In another embodiment, R² is optionally substituted thienyl. In another embodiment, R² is optionally substituted pyrrolyl. In another embodiment, R² is optionally substituted pyrazolyl. In another embodiment, R² is optionally substituted imidazolyl. In another embodiment, R² is optionally substituted thiazolyl. In another embodiment, R² is optionally substituted oxazolyl. In another embodiment, R² is optionally substituted isothiazolyl. In another embodiment, R² is optionally substituted isoxazolyl. In another embodiment, R² is optionally substituted oxadiazolyl. In another embodiment, R² is optionally substituted thiadiazolyl. In another embodiment, R² is optionally substituted triazolyl. In another embodiment, R² is optionally substituted tetrazolyl. In another embodiment, R² is optionally substituted pyridine oxide.

In one embodiment, R¹ is substituted with one or more halogen. In another embodiment, R¹ is substituted with one or more CN. In another embodiment, R¹ is substituted with one or more lower alkyl. In another embodiment, R¹ is substituted with one or more methyl. In another embodiment, R¹ is substituted with one or more trifluoromethyl. In another embodiment, R¹ is substituted with one or more —O(lower alkyl). In another embodiment, R¹ is substituted with one or more —OMe. In another embodiment, R¹ is substituted with one or more heterocyclyl. In another embodiment, R¹ is substituted with one or more morpholinyl. In another embodiment, R¹ is substituted with one or more aryl. In another embodiment, R¹ is substituted with one or more heteroaryl. In another embodiment, R¹ is substituted with one or more pyridine.

In one embodiment, R² is substituted with one or more halogen. In another embodiment, R² is substituted with one or more CN. In another embodiment, R² is substituted with one or more lower alkyl. In another embodiment, R² is substituted with one or more methyl. In another embodiment, R² is substituted with one or more trifluoromethyl. In another embodiment, R² is substituted with one or more —O(lower alkyl). In another embodiment, R² is substituted with one or more —OMe. In another embodiment, R² is substituted with one or more heterocyclyl. In another embodiment, R² is substituted with one or more morpholine. In another embodiment, R² is substituted with one or more aryl. In another embodiment, R² is substituted with one or more heteroaryl. In another embodiment, R² is substituted with one or more pyridine.

In one embodiment, R³ is hydrogen. In another embodiment, R³ is lower alkyl. In one embodiment, R³ is methyl. In another embodiment, R³ is ethyl. In another embodiment, R³ is propyl. In another embodiment, R³ is isopropyl.

In one embodiment, X is C. In another embodiment, X is N.

In one embodiment, Y is O. In another embodiment, Y is S. In another embodiment, Y is N. In another embodiment, Y is NR¹⁰. In another embodiment, Y is CR¹⁰. R¹⁰ is defined herein elsewhere.

In one embodiment, Z is O. In another embodiment, Z is S. In another embodiment, Z is N. In another embodiment, Z is NR¹⁰. In another embodiment, Z is CR¹⁰. R¹⁰ is defined herein elsewhere.

In one embodiment, R¹⁰ is hydrogen. In another embodiment, R¹⁰ is lower alkyl. In one embodiment, R¹⁰ is methyl. In another embodiment, R¹⁰ is ethyl. In another embodiment, R¹⁰ is propyl. In another embodiment, R¹⁰ is isopropyl. In another embodiment, R¹⁰ is butyl. In another embodiment, R¹⁰ is isobutyl. In another embodiment, R¹⁰ is t-butyl.

In one embodiment, G is N. In another embodiment, G is CH.

In one embodiment, o is 0. In another embodiment, o is 1. In another embodiment, o is 2.

In one embodiment, p is 1. In another embodiment p is 2.

Any of the combinations of R¹, R², R³, R¹⁰, X, Y, Z, G, o, and p are encompassed by this disclosure and specifically provided herein.

In one embodiment, provided herein is a compound of formula (IIa):

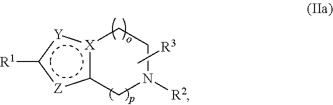

or a pharmaceutically acceptable salt, solvate, or stereoisomer thereof, wherein R¹, R², R³, X, Y, Z, o, and p are as defined herein elsewhere.

In one embodiment, provided herein is a compound of formula (IIb):

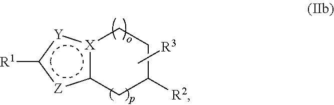

or a pharmaceutically acceptable salt, solvate, or stereoisomer thereof, wherein R¹, R², R³, X, Y, Z, o, and p are as defined herein elsewhere.

In one embodiment, X is C, Y is N, and Z is S. In another embodiment, X is C, Y is N, and Z is NH. In another embodiment, X is N, Y is CH, and Z is N. In another embodiment, X is N, Y is N, and Z is N.

In one embodiment, R¹ is optionally substituted pyridyl or optionally substituted phenyl. In one embodiment, R¹ is pyridyl substituted with one or more halo or —CN. In another embodiment, R¹ is phenyl substituted with one or more halo or —CN. In one embodiment, R² is optionally substituted pyridyl or optionally substituted phenyl. In one embodiment, R² is pyridyl, substituted with one or more halo or —CN. In another embodiment, R² is phenyl, substituted with one or more halo or —CN.

In one embodiment, specific examples include, but are not limited to, the following compounds:

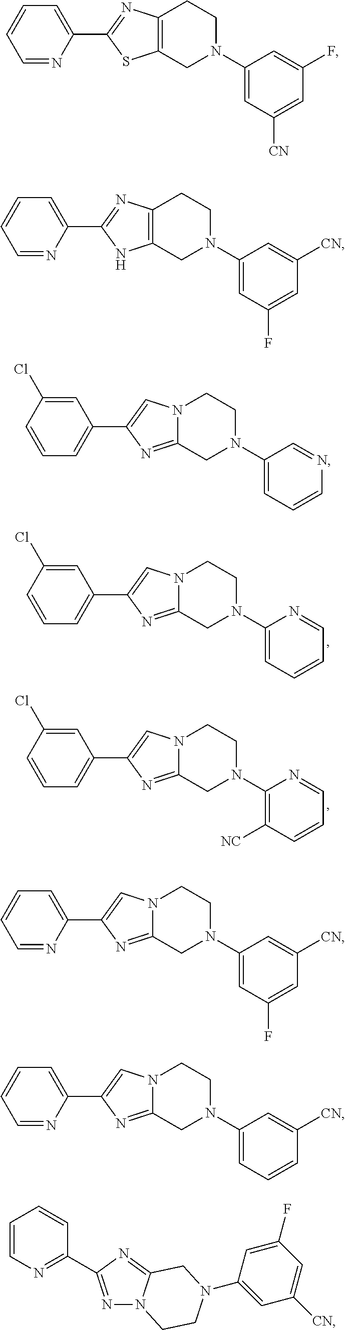

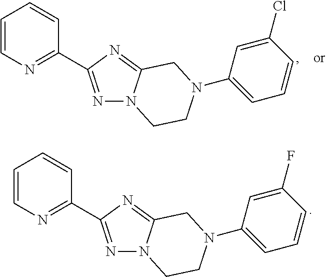

In one embodiment, specific examples include, but are not limited to, the following compounds:

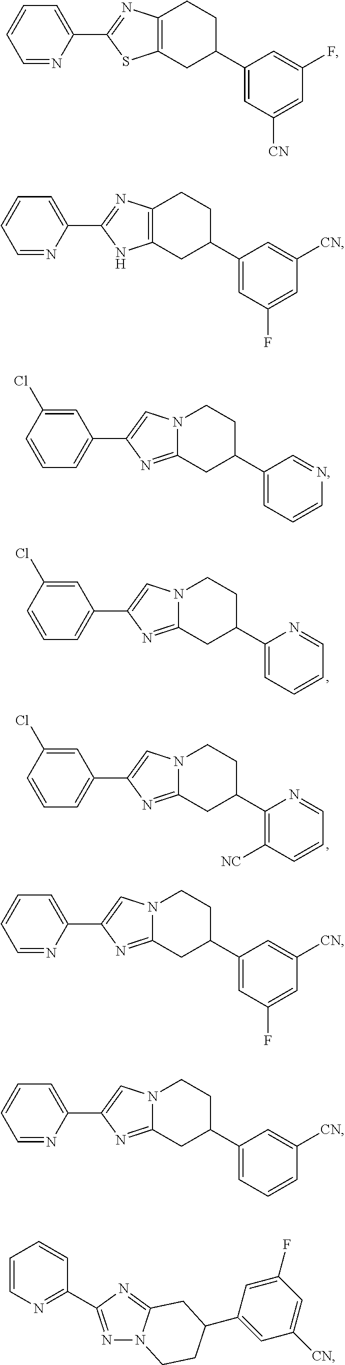

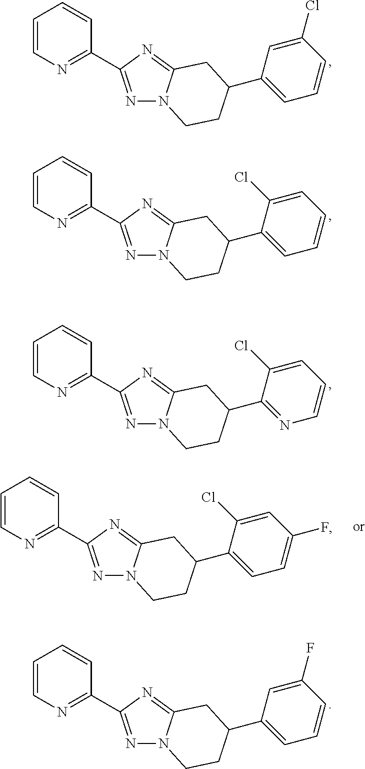

In one embodiment, provided herein is a compound of formula (III):

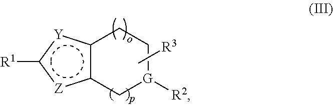

or a pharmaceutically acceptable salt, solvate, or stereoisomer thereof, wherein

R¹ is 5- or 6-membered aryl, or 5- or 6-membered heteroaryl, each of which is optionally substituted;

R² is 5- or 6-membered aryl, or 5- or 6-membered heteroaryl, each of which is optionally substituted;

R³ is hydrogen or lower alkyl;

Y is O and Z is N; or Y is N and Z is O;

G is N or CH;

o is 0, 1, or 2; and

p is 1 or 2.

In one embodiment, R¹ is optionally substituted 5- or 6-membered aryl. In another embodiment, R¹ is optionally substituted 5- or 6-membered heteroaryl. In one embodiment, R¹ is optionally substituted phenyl. In another embodiment, R¹ is optionally substituted pyridyl. In another embodiment, R¹ is optionally substituted pyrimidinyl. In another embodiment, R¹ is optionally substituted pyrazinyl. In another embodiment, R¹ is optionally substituted pyridazinyl. In another embodiment, R¹ is optionally substituted triazinyl. In another embodiment, R¹ is optionally substituted furanyl. In another embodiment, R¹ is optionally substituted thienyl. In another embodiment, R¹ is optionally substituted pyrrolyl. In another embodiment, R¹ is optionally substituted pyrazolyl. In another embodiment, R¹ is optionally substituted imidazolyl. In another embodiment, R¹ is optionally substituted thiazolyl. In another embodiment, R¹ is optionally substituted oxazolyl. In another embodiment, R¹ is optionally substituted isothiazolyl. In another embodiment, R¹ is optionally substituted isoxazolyl. In another embodiment, R¹ is optionally substituted oxadiazolyl. In another embodiment, R¹ is optionally substituted thiadiazolyl. In another embodiment, R¹ is optionally substituted triazolyl. In another embodiment, R¹ is optionally substituted tetrazolyl. In another embodiment, R¹ is optionally substituted pyridine oxide.

In one embodiment, R² is optionally substituted 5- or 6-membered aryl. In another embodiment R² is optionally substituted 5- or 6-membered heteroaryl. In one embodiment, R² is optionally substituted phenyl. In another embodiment, R² is optionally substituted pyridyl. In another embodiment, R² is optionally substituted pyrimidinyl. In another embodiment, R² is optionally substituted pyrazinyl. In another embodiment, R² is optionally substituted pyridazinyl. In another embodiment, R² is optionally substituted triazinyl. In another embodiment, R² is optionally substituted furanyl. In another embodiment, R² is optionally substituted thienyl. In another embodiment, R² is optionally substituted pyrrolyl. In another embodiment, R² is optionally substituted pyrazolyl. In another embodiment, R² is optionally substituted imidazolyl. In another embodiment, R² is optionally substituted thiazolyl. In another embodiment, R² is optionally substituted oxazolyl. In another embodiment, R² is optionally substituted isothiazolyl. In another embodiment, R² is optionally substituted isoxazolyl. In another embodiment, R² is optionally substituted oxadiazolyl. In another embodiment, R² is optionally substituted thiadiazolyl. In another embodiment, R² is optionally substituted triazolyl. In another embodiment, R² is optionally substituted tetrazolyl. In another embodiment, R² is optionally substituted pyridine oxide.

In one embodiment, R¹ is substituted with one or more halogen. In another embodiment, R¹ is substituted with one or more CN. In another embodiment, R¹ is substituted with one or more lower alkyl. In another embodiment, R¹ is substituted with one or more methyl. In another embodiment, R¹ is substituted with one or more trifluoromethyl. In another embodiment, R¹ is substituted with one or more —O(lower alkyl). In another embodiment, R¹ is substituted with one or more —OMe. In another embodiment, R¹ is substituted with one or more heterocyclyl. In another embodiment, R¹ is substituted with one or more morpholine. In another embodiment, R¹ is substituted with one or more aryl. In another embodiment, R¹ is substituted with one or more heteroaryl. In another embodiment, R¹ is substituted with one or more pyridine.

In one embodiment, R² is substituted with one or more halogen. In another embodiment, R² is substituted with one or more CN. In another embodiment, R² is substituted with one or more lower alkyl. In another embodiment, R² is substituted with one or more methyl. In another embodiment, R² is substituted with one or more trifluoromethyl. In another embodiment, R² is substituted with one or more —O(lower alkyl). In another embodiment, R² is substituted with one or more —OMe. In another embodiment, R² is substituted with one or more heterocyclyl. In another embodiment, R² is substituted with one or more morpholine. In another embodiment, R² is substituted with one or more aryl. In another embodiment, R² is substituted with one or more heteroaryl. In another embodiment, R² is substituted with one or more pyridine.

In one embodiment, R³ is hydrogen. In another embodiment, R³ is lower alkyl. In one embodiment, R³ is methyl. In another embodiment, R³ is ethyl. In another embodiment, R³ is propyl. In another embodiment, R³ is isopropyl.

In one embodiment, Y is O and Z is N. In another embodiment, Y is N and Z is O.

In one embodiment, G is N. In another embodiment, G is CH.

In one embodiment, o is 0. In another embodiment, o is 1. In another embodiment, o is 2.

In one embodiment, p is 1. In another embodiment p is 2.

Any of the combinations of R¹, R², R³, Y, Z, G, o, and p are encompassed by this disclosure and specifically provided herein.

In one embodiment, provided herein is a compound of formula (IIIa):

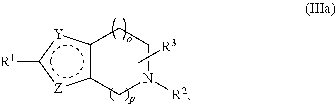

or a pharmaceutically acceptable salt, solvate, or stereoisomer thereof, wherein R¹, R², R³, Y, Z, o, and p are as defined herein elsewhere.

In one embodiment, provided herein is a compound of formula (IIIb):

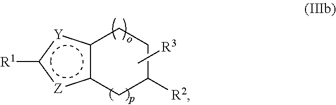

or a pharmaceutically acceptable salt, solvate, or stereoisomer thereof, wherein R¹, R², R³, Y, Z, o, and p are as defined herein elsewhere.

In one embodiment, provided herein is a compound of formula (IVa):

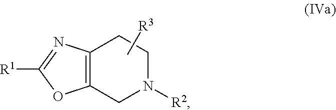

or a pharmaceutically acceptable salt, solvate, or stereoisomer thereof, wherein R¹, R², and R³ are defined herein elsewhere.

In one embodiment, R¹ is optionally substituted pyridyl or optionally substituted phenyl. In another embodiment, R¹ is pyridyl-oxide. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe. In one embodiment, R¹ is optionally substituted cyclohexyl. In one embodiment, R¹ is unsubstituted cyclohexyl. In another embodiment, R¹ is cyclohexyl substituted with one or more halo, CN, lower alkyl, or —O(lower alkyl). In another embodiment, R¹ is cyclohexyl substituted with one or more halo, CN, Me, CF₃, or OMe. In one embodiment, R¹ is pyridyl, phenyl, or cyclohexyl, each of which is optionally substituted with one or more halo, CN, lower alkyl, or —O(lower alkyl).

In one embodiment, R² is optionally substituted pyridyl, optionally substituted phenyl, optionally substituted pyrazinyl, or optionally substituted thiazolyl. In one embodiment, R² is pyridyl substituted with one or more halo, CN, heterocyclyl, or heteroaryl. In another embodiment, R² is pyridyl substituted with one or more halo, CN, morpholinyl, or pyridyl. In another embodiment, R² is phenyl substituted with one or more halo, CN, heterocyclyl, or heteroaryl. In another embodiment, R² is phenyl substituted with one or more halo, CN, morpholinyl, or pyridyl. In another embodiment, R² is pyrazinyl substituted with one or more halo, CN, heterocyclyl, or heteroaryl. In another embodiment, R² is pyrazinyl substituted with one or more halo, CN, morpholinyl, or pyridyl. In another embodiment, R² is thiazolyl substituted with one or more halo, CN, heterocyclyl, or heteroaryl. In another embodiment, R² is thiazolyl substituted with one or more halo, CN, morpholinyl, or pyridyl.

In one embodiment, R³ is hydrogen. In another embodiment, R³ is methyl.

Specific examples include, but are not limited to, the following compounds:

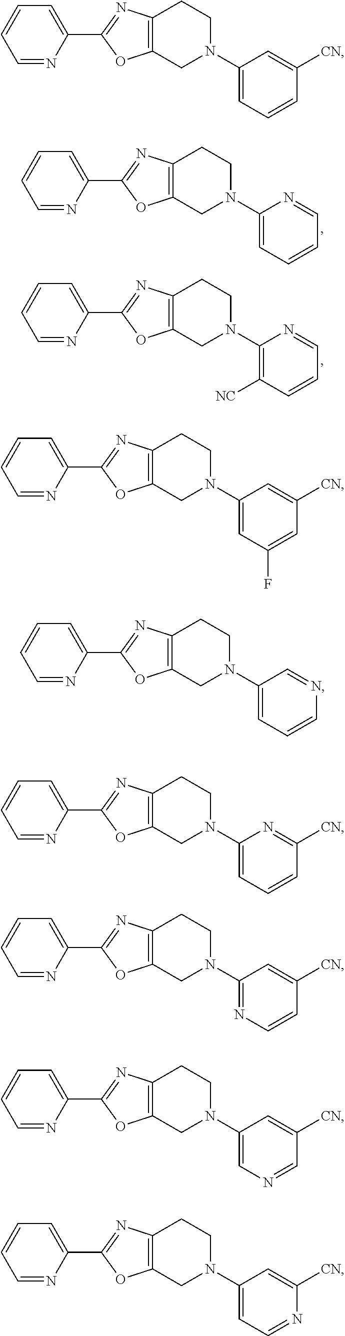

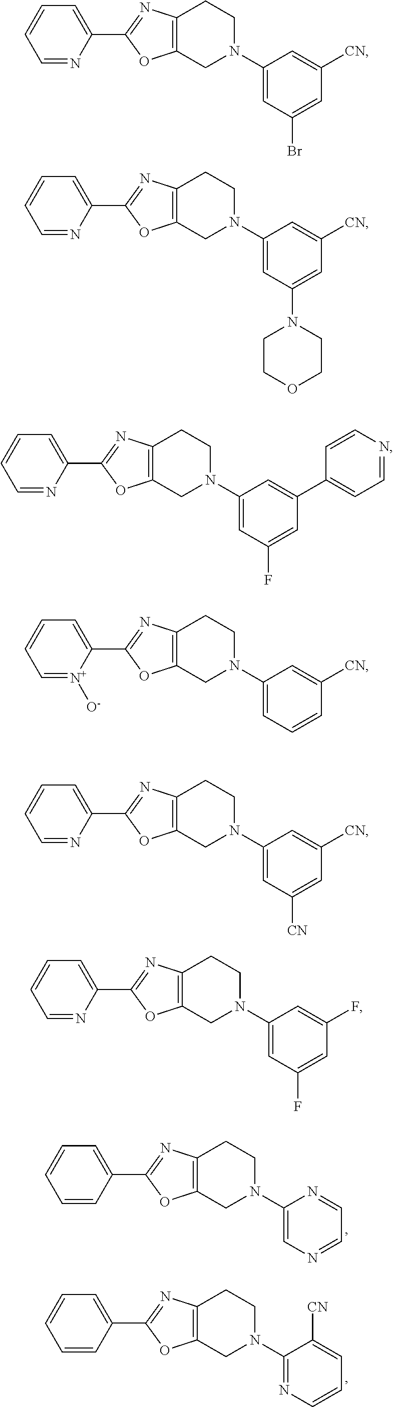

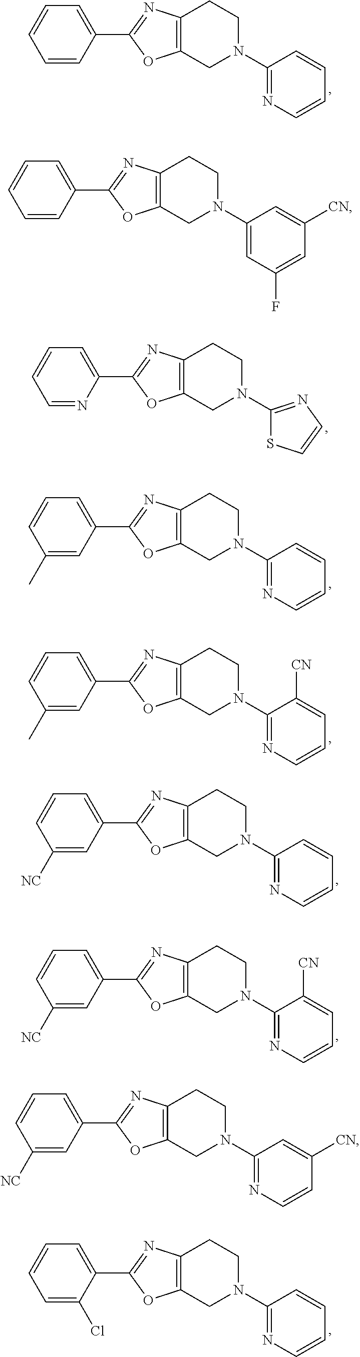

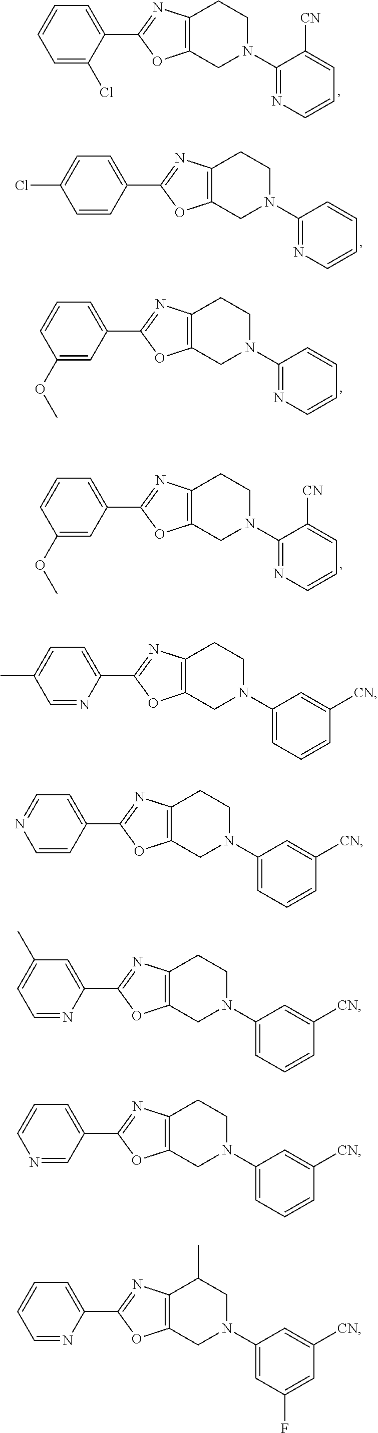

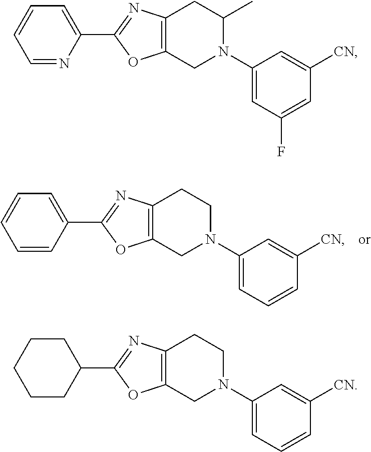

In one embodiment, provided herein is a compound of formula (IVb):

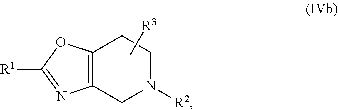

or a pharmaceutically acceptable salt, solvate, or stereoisomer thereof, wherein R¹, R², and R³ are defined herein elsewhere.

In one embodiment, R¹ is optionally substituted pyridyl, optionally substituted phenyl, optionally substituted pyrimidinyl, optionally substituted pyrazinyl, optionally substituted isoxazolyl, optionally substituted oxazolyl, or optionally substituted thiazolyl. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe. In another embodiment, R¹ is pyrimidinyl substituted with one or more halo, CN, lower alkyl, or —O(lower alkyl). In another embodiment, R¹ is pyrimidinyl substituted with one or more halo, CN, Me, CF₃, or OMe. In another embodiment, R¹ is pyrazinyl substituted with one or more halo, CN, lower alkyl, or —O(lower alkyl). In another embodiment, R¹ is pyrazinyl substituted with one or more halo, CN, Me, CF₃, or OMe. In another embodiment, R¹ is isoxazolyl substituted with one or more halo, CN, lower alkyl, or —O(lower alkyl). In another embodiment, R¹ is isoxazolyl substituted with one or more halo, CN, Me, CF₃, or OMe. In another embodiment, R¹ is oxazolyl substituted with one or more halo, CN, lower alkyl, or —O(lower alkyl). In another embodiment, R¹ is oxazolyl substituted with one or more halo, CN, Me, CF₃, or OMe. In another embodiment, R¹ is thiazolyl substituted with one or more halo, CN, lower alkyl, or —O(lower alkyl). In another embodiment, R¹ is thiazolyl substituted with one or more halo, CN, Me, CF₃, or OMe.

In one embodiment, R² is optionally substituted pyridyl, optionally substituted phenyl, optionally substituted pyrimidinyl, optionally substituted pyrazinyl, or optionally substituted thiazolyl. In one embodiment, R² is pyridyl substituted with one or more halo, CN, lower alkyl, or —O(lower alkyl). In another embodiment, R² is pyridyl substituted with one or more halo, CN, Me, CF₃, or OMe. In another embodiment, R² is phenyl substituted with one or more halo, CN, lower alkyl, or —O(lower alkyl). In another embodiment, R² is phenyl substituted with one or more halo, CN, Me, CF₃, or OMe. In another embodiment, R² is pyrimidinyl substituted with one or more halo, CN, lower alkyl, or —O(lower alkyl). In another embodiment, R² is pyrimidinyl substituted with one or more halo, CN, Me, CF₃, or OMe. In another embodiment, R² is pyrazinyl substituted with one or more halo, CN, lower alkyl, or —O(lower alkyl). In another embodiment, R² is pyrazinyl substituted with one or more halo, CN, Me, CF₃, or OMe. In another embodiment, R² is thiazolyl substituted with one or more halo, CN, lower alkyl, or —O(lower alkyl). In another embodiment, R² is thiazolyl substituted with one or more halo, CN, Me, CF₃, or OMe. In one embodiment, R² is optionally substituted naphthyl or optionally substituted indolyl. In one embodiment, R² is unsubstituted naphthyl or unsubstituted indolyl. In another embodiment, R² is naphthyl substituted with one or more halo, CN, lower alkyl, or —O(lower alkyl). In another embodiment, R² is naphthyl substituted with one or more halo, CN, Me, CF₃, or OMe. In another embodiment, R² is indolyl substituted with one or more halo, CN, lower alkyl, or —O(lower alkyl). In another embodiment, R² is indolyl substituted with one or more halo, CN, Me, CF₃, or OMe. In one embodiment, R² is phenyl optionally substituted with one or more halo, CN, OH, lower alkyl, —O(lower alkyl), or heterocyclyl. In one embodiment, R² is phenyl substituted with one or more halo, CN, OH, lower alkyl, —O(lower alkyl), or heterocyclyl. In one embodiment, R² is phenyl substituted with one or more halo, CN, OH, Me, CF₃, OMe, or morpholinyl. In one embodiment, R² is pyridyl, phenyl, pyrimidinyl, pyrazinyl, thiazolyl, naphthyl, or indolyl, each of which is optionally substituted with one or more halo, CN, OH, lower alkyl, —O(lower alkyl), or heterocyclyl.

In one embodiment, R³ is hydrogen. In another embodiment, R³ is methyl.

Specific examples include, but are not limited to, the following compounds:

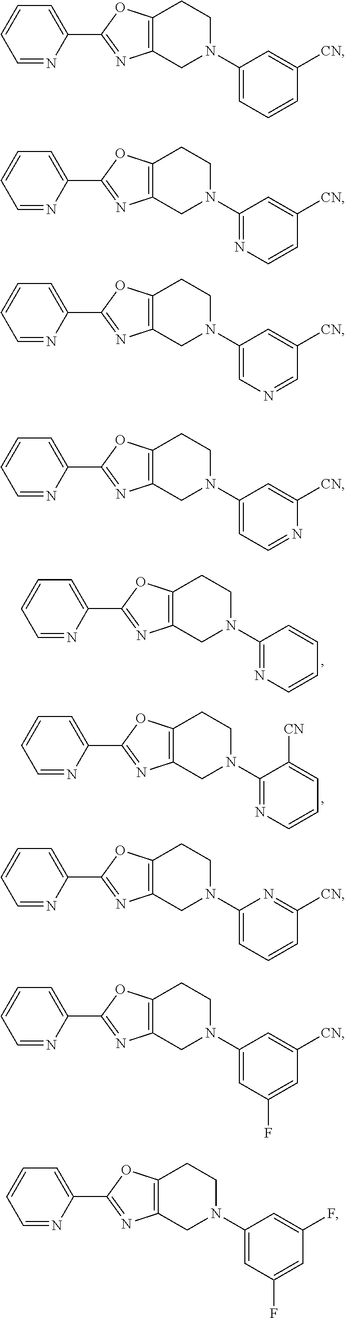

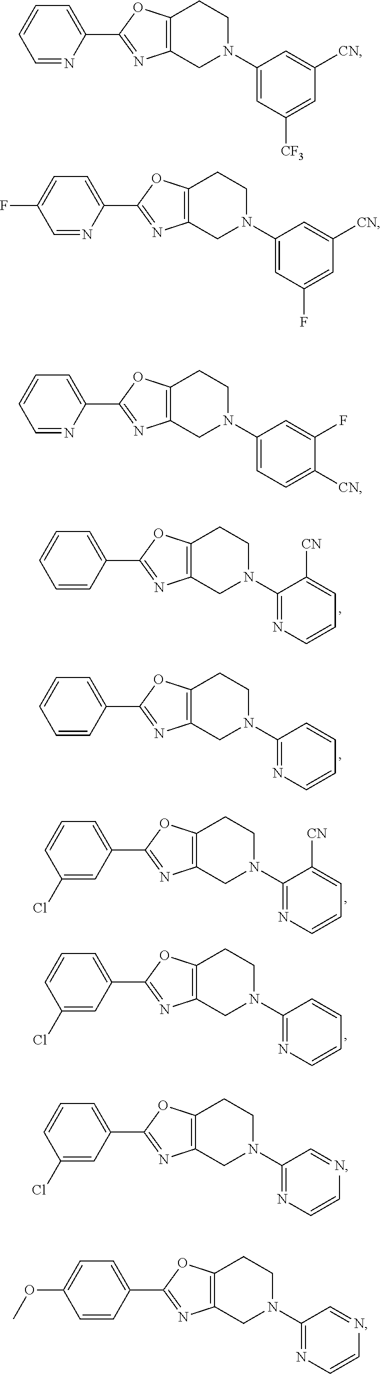

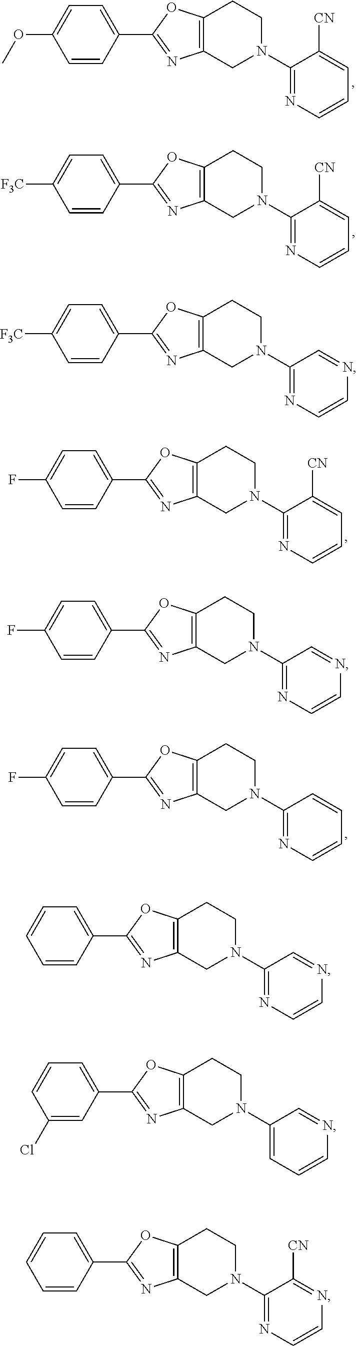

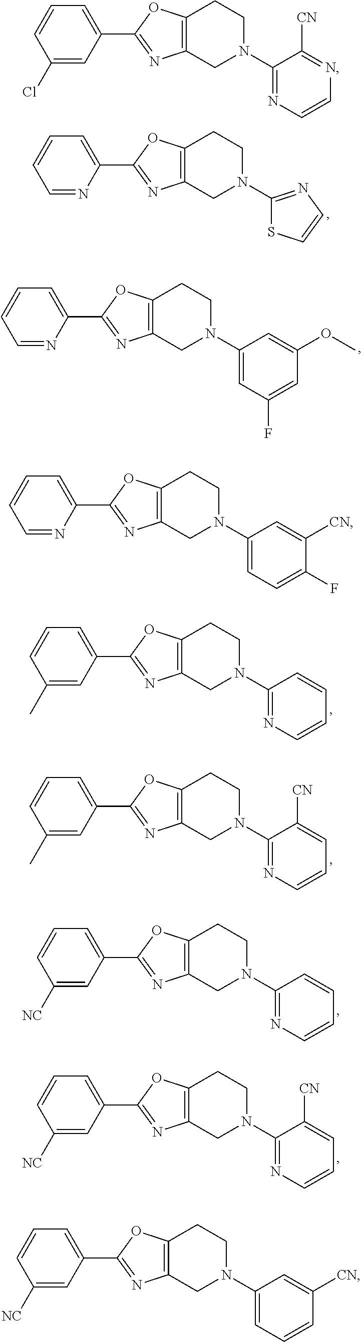

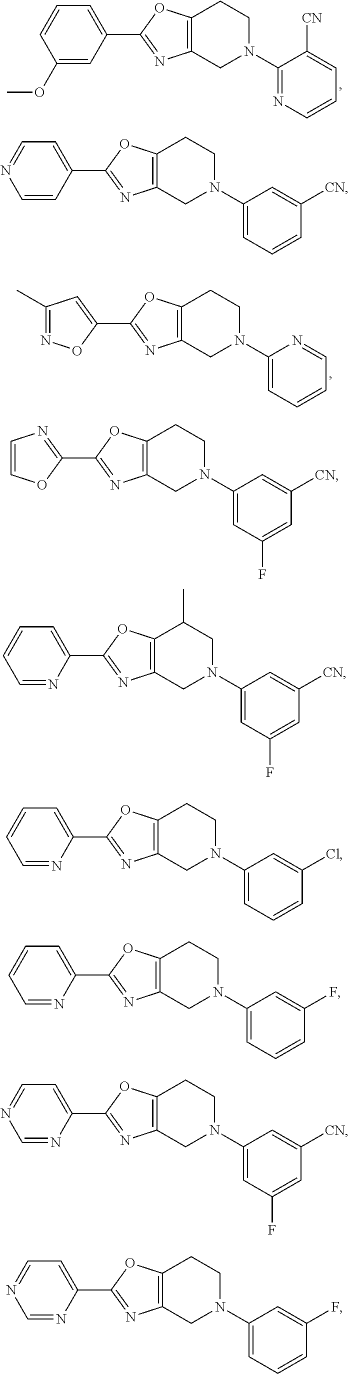

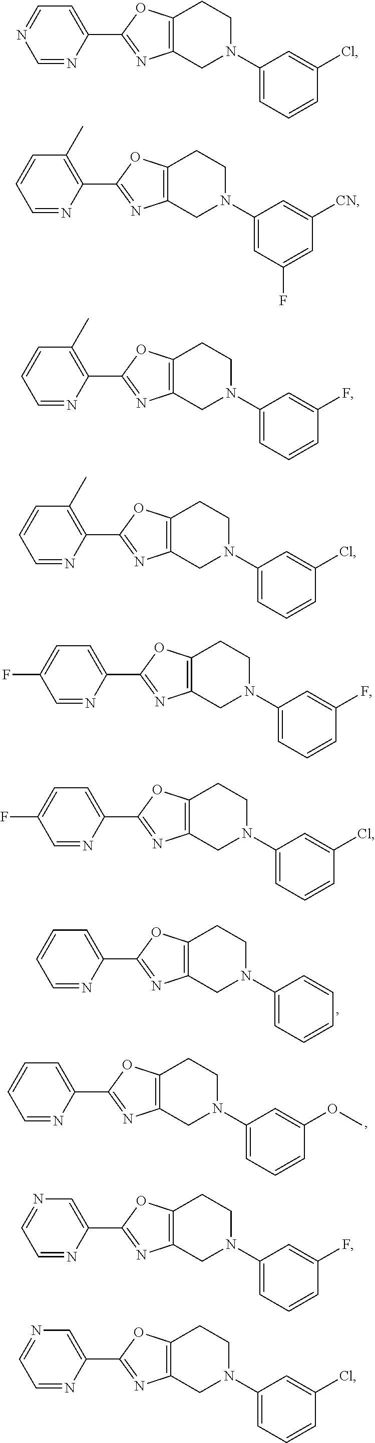

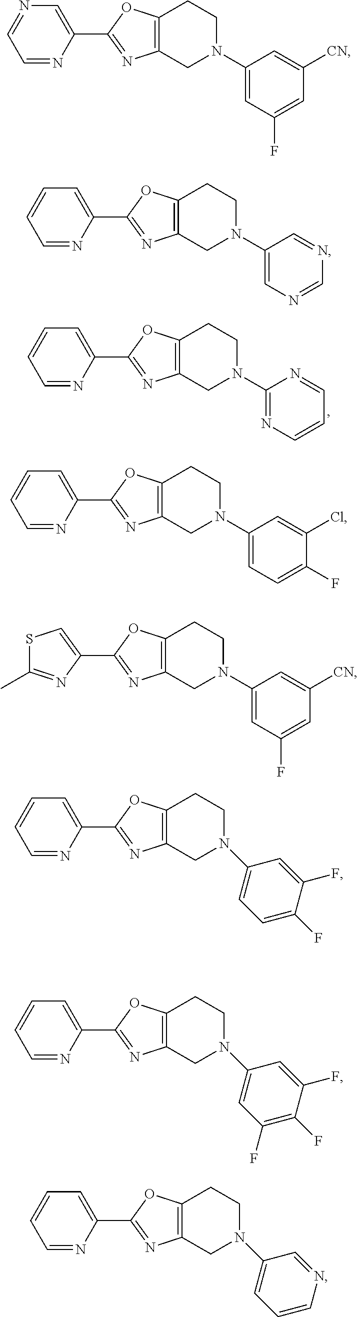

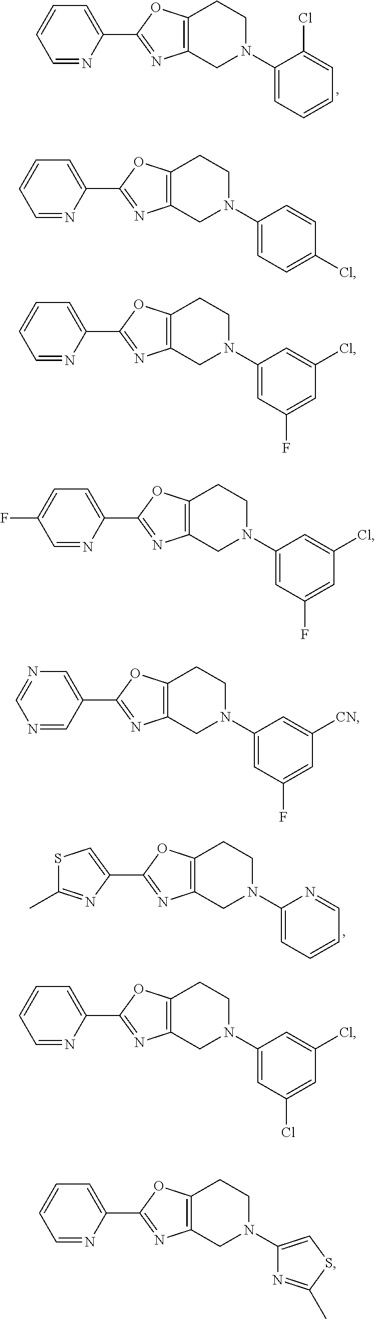

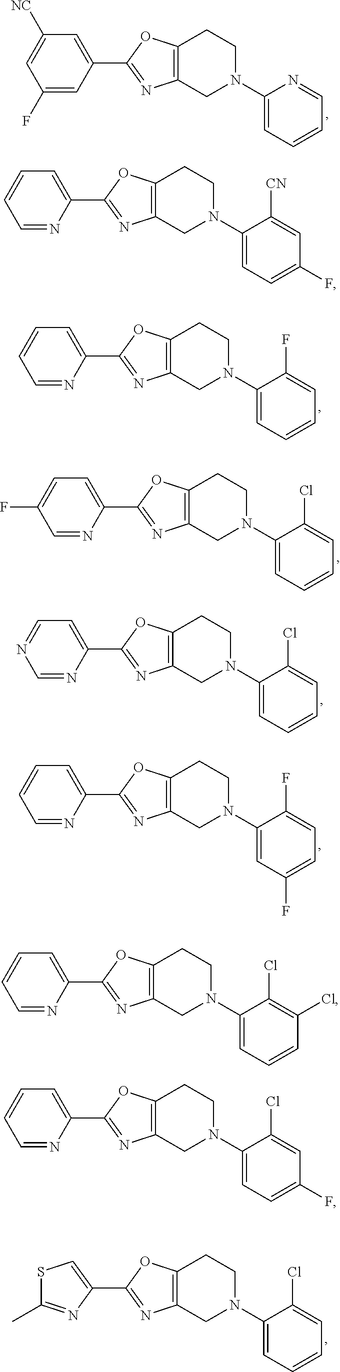

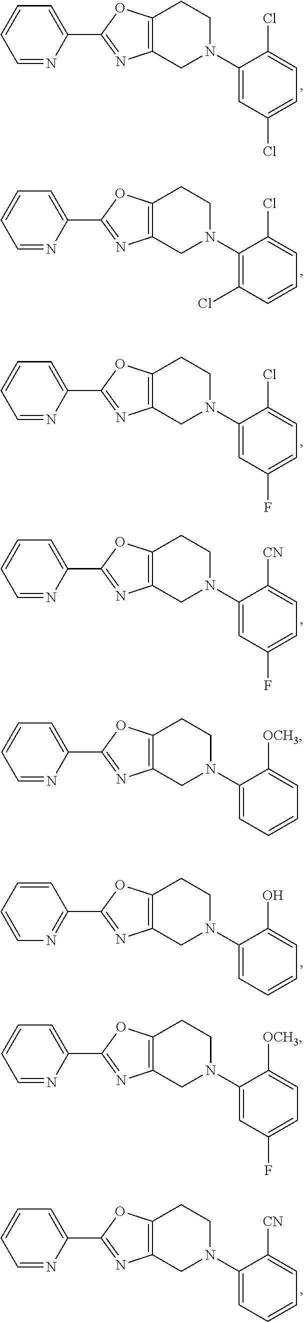

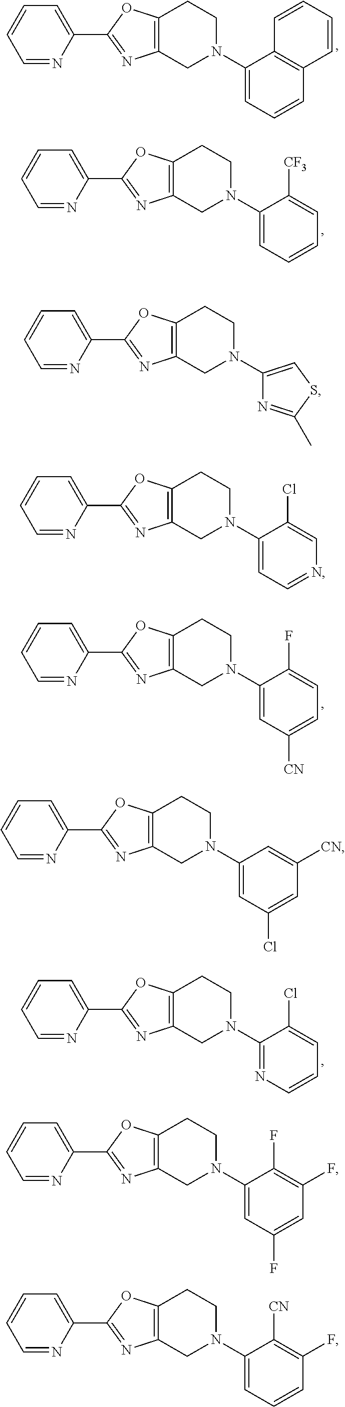

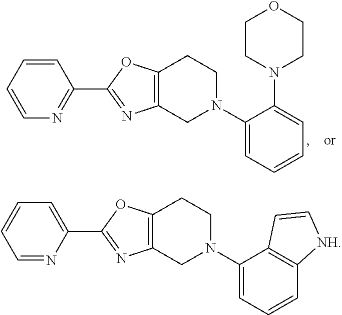

In one embodiment, provided herein is a compound of formula (IVc):

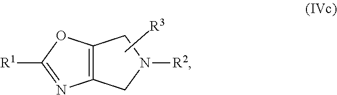

or a pharmaceutically acceptable salt, solvate, or stereoisomer thereof, wherein R¹, R², and R³ are defined herein elsewhere. In one embodiment, R¹ is optionally substituted pyridyl or optionally substituted phenyl. In another embodiment, R¹ is pyridyl substituted with one or more halo or CN. In another embodiment, R¹ is phenyl substituted with one or more halo or CN. In one embodiment, R² is optionally substituted pyridyl or optionally substituted phenyl. In another embodiment, R² is pyridyl substituted with one or more halo or CN. In another embodiment, R² is phenyl substituted with one or more halo or CN. In one embodiment, R³ is hydrogen.

Specific examples include, but are not limited to, the following compounds:

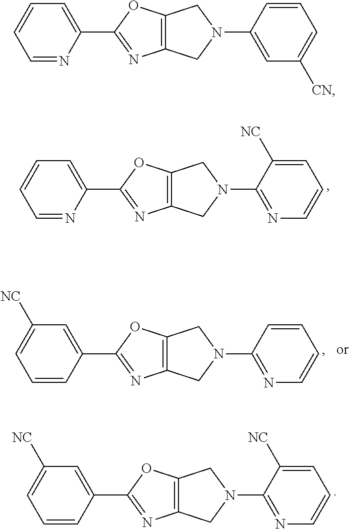

In one embodiment, provided herein is a compound of formula (IVd):

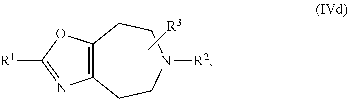

or a pharmaceutically acceptable salt, solvate, or stereoisomer thereof, wherein R′, R², and R³ are defined herein elsewhere. In one embodiment, R¹ is optionally substituted pyridyl or optionally substituted phenyl. In another embodiment, R¹ is pyridyl substituted with one or more halo or CN. In another embodiment, R¹ is phenyl substituted with one or more halo or CN. In one embodiment, R² is optionally substituted pyridyl or optionally substituted phenyl. In another embodiment, R² is pyridyl substituted with one or more halo or CN. In another embodiment, R² is phenyl substituted with one or more halo or CN. In one embodiment, R³ is hydrogen.

Specific examples include, but are not limited to, the following compounds:

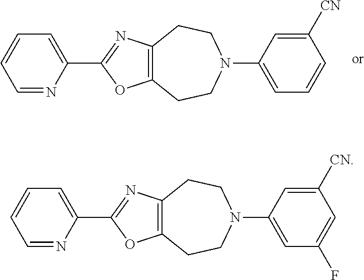

In one embodiment, provided herein is a compound of formula (IVe):

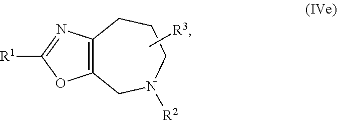

or a pharmaceutically acceptable salt, solvate, or stereoisomer thereof, wherein R¹, R², and R³ are defined herein elsewhere. In one embodiment, R¹ is optionally substituted pyridyl, optionally substituted phenyl, or optionally substituted pyrimidinyl. In another embodiment, R¹ is pyridyl substituted with one or more halo or CN. In another embodiment, R¹ is phenyl substituted with one or more halo or CN. In another embodiment, R¹ is pyrimidinyl substituted with one or more halo or CN. In one embodiment, R² is optionally substituted pyridyl or optionally substituted phenyl. In another embodiment, R² is pyridyl substituted with one or more halo, CN, lower alkyl, or —O(lower alkyl). In another embodiment, R² is pyridyl substituted with one or more halo, CN, Me, CF₃ or OMe. In another embodiment, R² is phenyl substituted with one or more halo, CN, lower alkyl, or —O(lower alkyl). In another embodiment, R² is phenyl substituted with one or more halo, CN, Me, CF₃ or OMe. In one embodiment, R³ is hydrogen.

Specific examples include, but are not limited to, the following compounds:

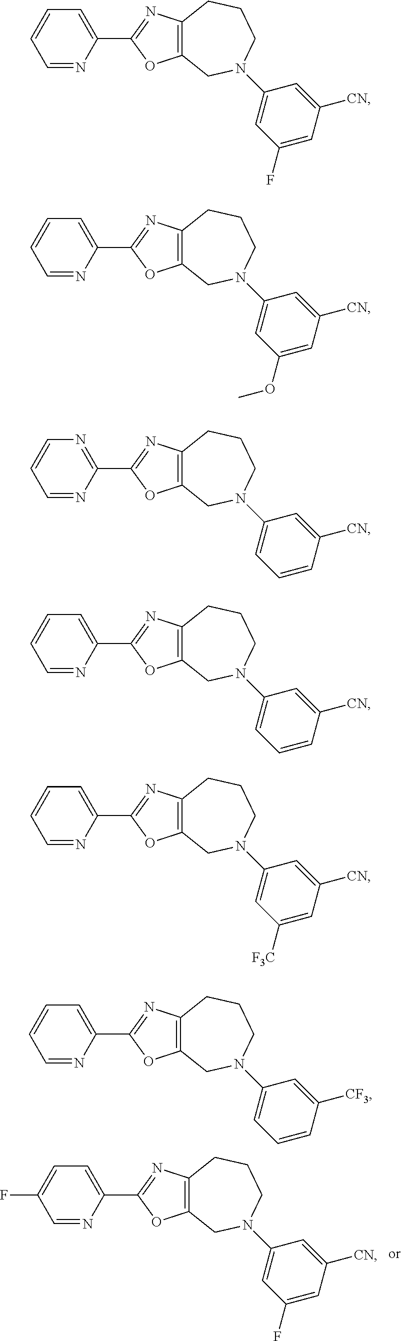

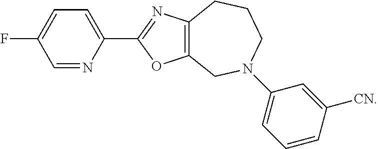

In one embodiment, provided herein is a compound of formula (IVf):

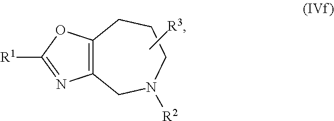

or a pharmaceutically acceptable salt, solvate, or stereoisomer thereof, wherein R¹, R², and R³ are defined herein elsewhere.

In one embodiment, R¹ is optionally substituted pyridyl, optionally substituted phenyl, optionally substituted pyrimidinyl, optionally substituted pyrazinyl, or optionally substituted thiazolyl. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe. In another embodiment, R¹ is pyrimidinyl substituted with one or more halo, CN, lower alkyl, or —O(lower alkyl). In another embodiment, R¹ is pyrimidinyl substituted with one or more halo, CN, Me, CF₃, or OMe. In another embodiment, R¹ is pyrazinyl substituted with one or more halo, CN, lower alkyl, or —O(lower alkyl). In another embodiment, R¹ is pyrazinyl substituted with one or more halo, CN, Me, CF₃, or OMe. In another embodiment, R¹ is thiazolyl substituted with one or more halo, CN, lower alkyl, or —O(lower alkyl). In another embodiment, R¹ is thiazolyl substituted with one or more halo, CN, Me, CF₃, or OMe.

In one embodiment, R² is optionally substituted pyridyl, optionally substituted phenyl, or optionally substituted pyrimidinyl. In one embodiment, R² is pyridyl substituted with one or more halo, CN, lower alkyl, or —O(lower alkyl). In another embodiment, R² is pyridyl substituted with one or more halo, CN, Me, CF₃ or OMe. In another embodiment, R² is phenyl substituted with one or more halo, CN, lower alkyl, or —O(lower alkyl). In another embodiment, R² is phenyl substituted with one or more halo, CN, Me, CF₃ or OMe. In another embodiment, R² is pyrimidinyl substituted with one or more halo, CN, lower alkyl, or —O(lower alkyl). In another embodiment, R² is pyrimidinyl substituted with one or more halo, CN, Me, CF₃ or OMe. In one embodiment, R² is thiazolyl optionally substituted with one or more halo, CN, lower alkyl, or —O(lower alkyl). In another embodiment, R² is thiazolyl substituted with one or more halo, CN, lower alkyl, or —O(lower alkyl). In another embodiment, R² is thiazolyl substituted with one or more halo, CN, Me, CF₃, or —OMe. In one embodiment, R² is phenyl optionally substituted with one or more halo, CN, OH, lower alkyl, or —O(lower alkyl). In another embodiment, R² is phenyl substituted with one or more halo, CN, OH, lower alkyl, or —O(lower alkyl). In another embodiment, R² is phenyl substituted with one or more halo, CN, OH, Me, CF₃, or OMe. In one embodiment, R² is pyridyl, phenyl, pyrimidinyl, or thiazolyl, each of which is optionally substituted with one or more halo, CN, OH, lower alkyl, or —O(lower alkyl).

In one embodiment, R³ is hydrogen. In another embodiment, R³ is methyl.

Specific examples include, but are not limited to, the following compounds:

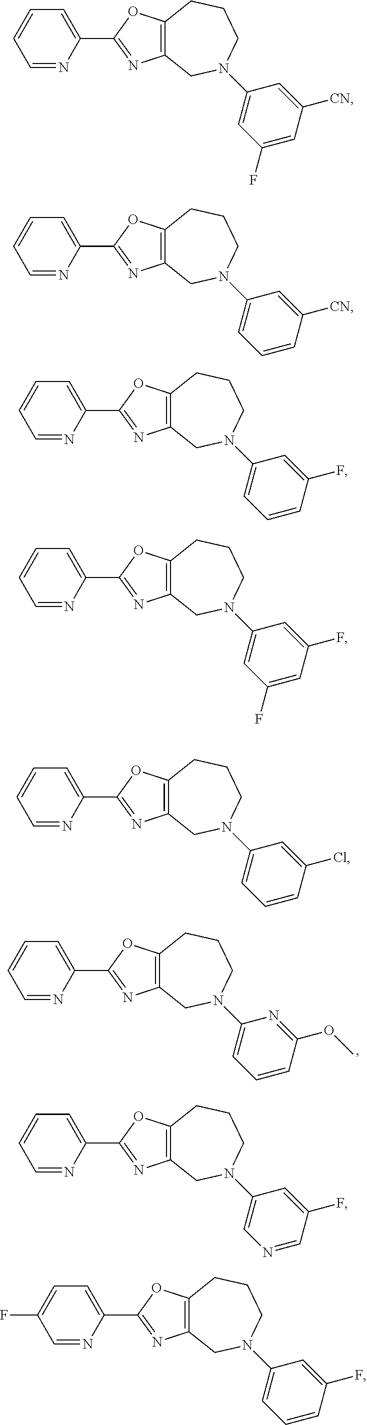

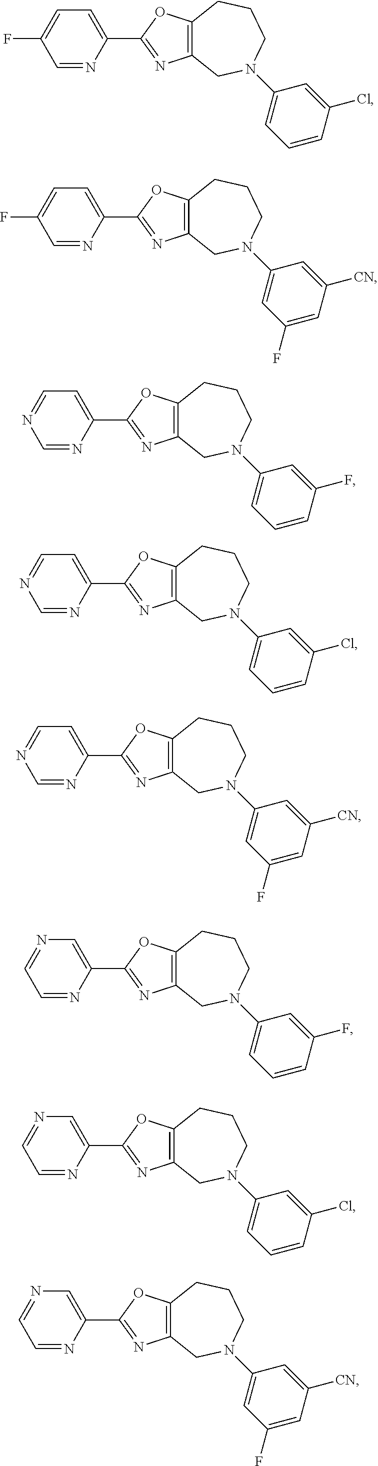

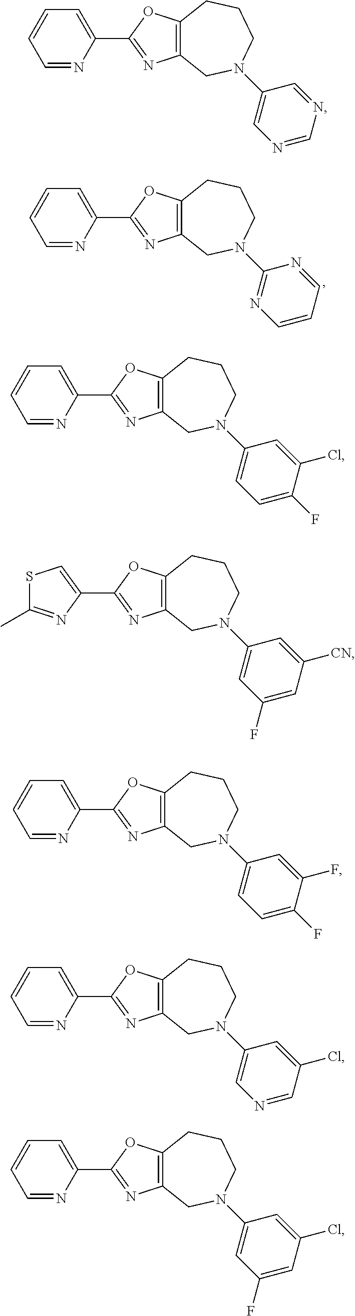

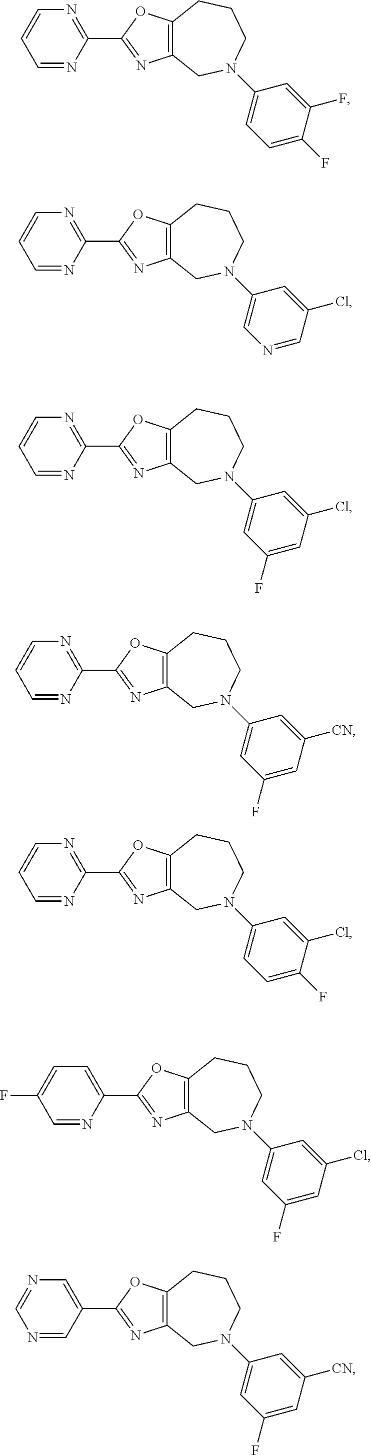

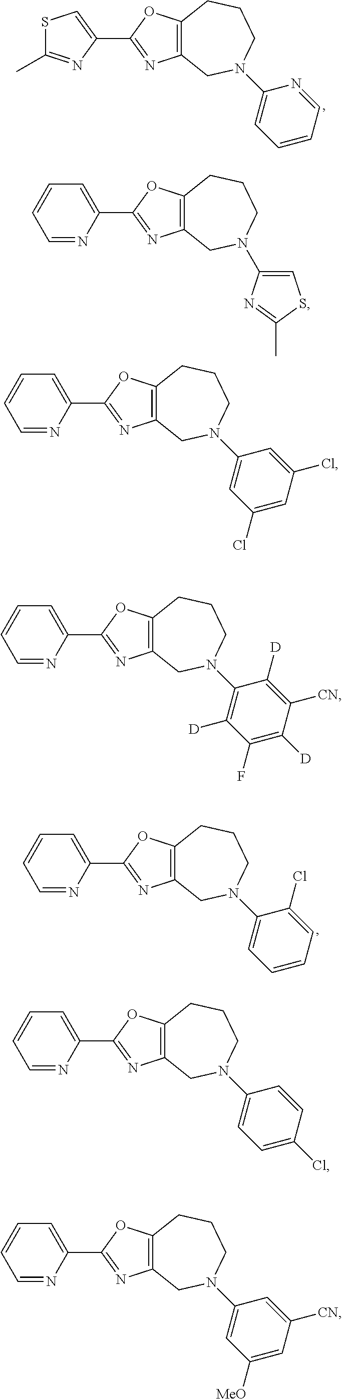

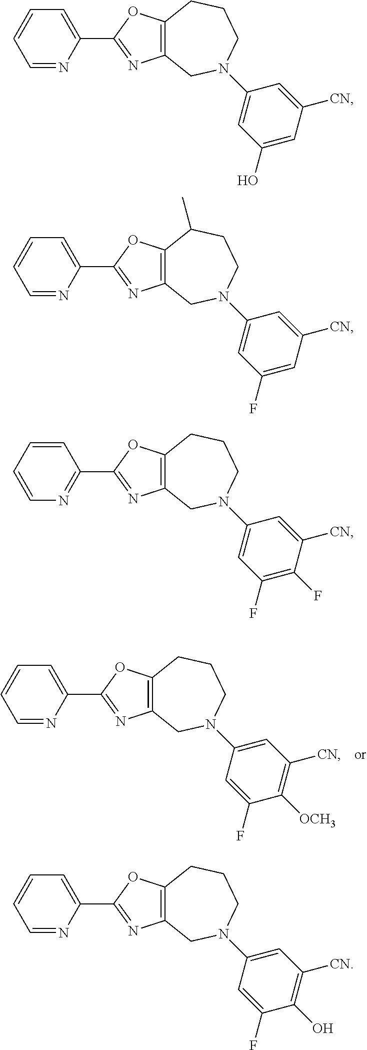

In one embodiment, provided herein is a compound of formula (Va):

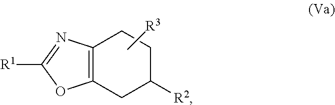

or a pharmaceutically acceptable salt, solvate, or stereoisomer thereof, wherein R¹, R², and R³ are defined herein elsewhere.

In one embodiment, R¹ is optionally substituted pyridyl or optionally substituted phenyl. In another embodiment, R¹ is pyridyl-oxide. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe.

In one embodiment, R² is optionally substituted pyridyl, optionally substituted phenyl, optionally substituted pyrazinyl, or optionally substituted thiazolyl. In one embodiment, R² is pyridyl substituted with one or more halo, CN, heterocyclyl, or heteroaryl. In another embodiment, R² is pyridyl substituted with one or more halo, CN, morpholinyl, or pyridyl. In another embodiment, R² is phenyl substituted with one or more halo, CN, heterocyclyl, or heteroaryl. In another embodiment, R² is phenyl substituted with one or more halo, CN, morpholinyl, or pyridyl. In another embodiment, R² is pyrazinyl substituted with one or more halo, CN, heterocyclyl, or heteroaryl. In another embodiment, R² is pyrazinyl substituted with one or more halo, CN, morpholinyl, or pyridyl. In another embodiment, R² is thiazolyl substituted with one or more halo, CN, heterocyclyl, or heteroaryl. In another embodiment, R² is thiazolyl substituted with one or more halo, CN, morpholinyl, or pyridyl.

In one embodiment, R³ is hydrogen. In another embodiment, R³ is methyl.

Specific examples include, but are not limited to, the following compounds:

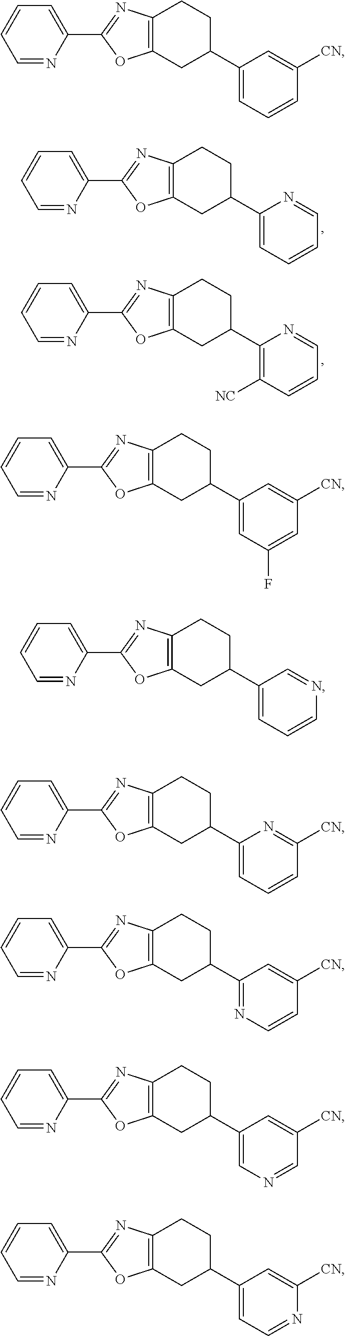

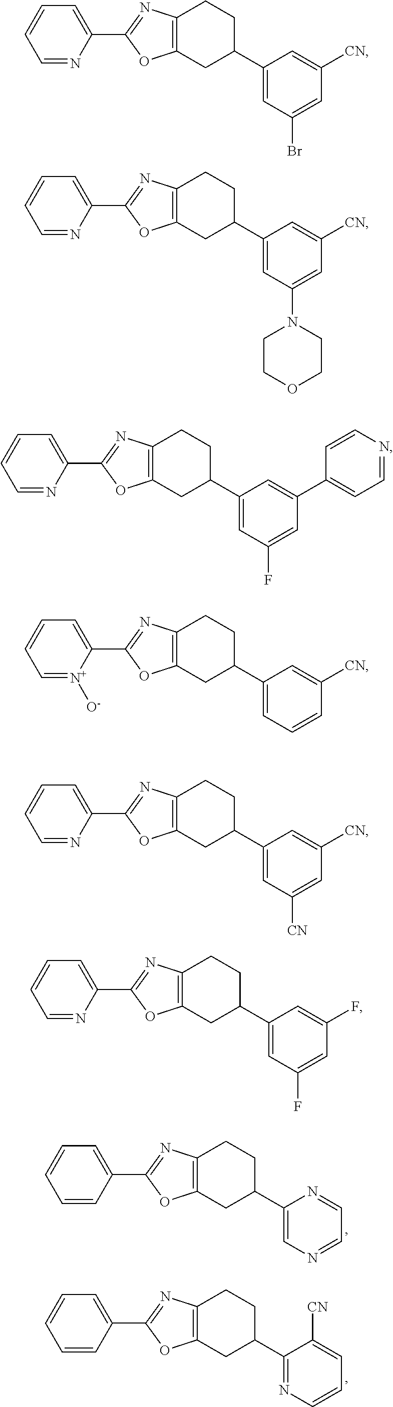

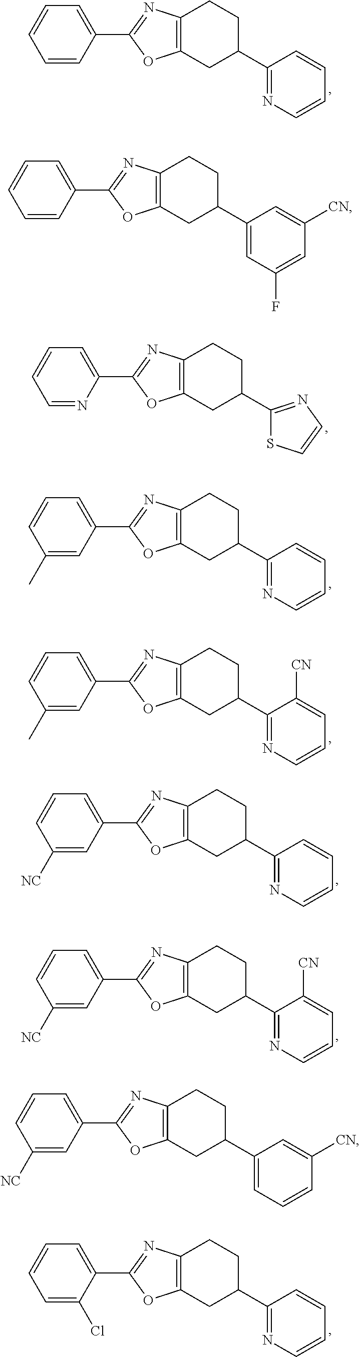

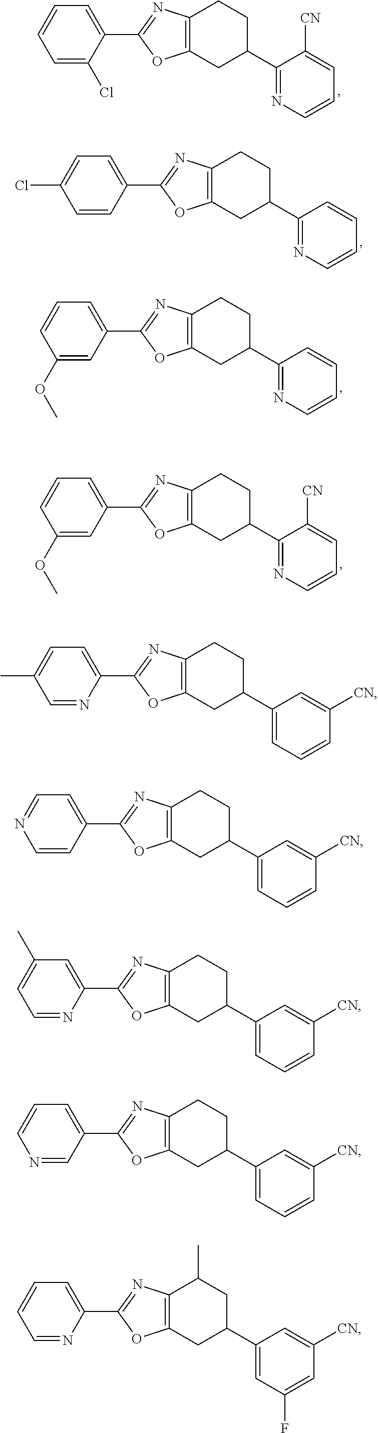

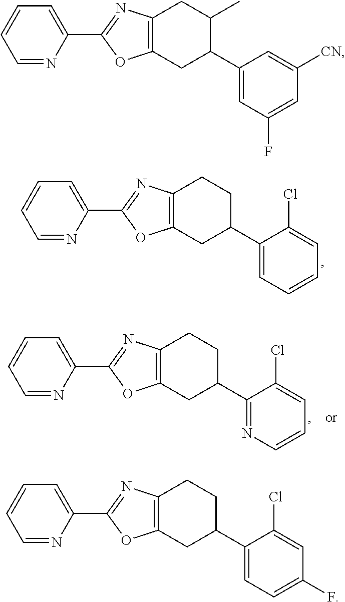

In one embodiment, provided herein is a compound of formula (Vb):

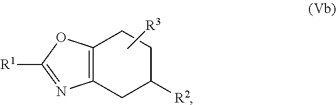

or a pharmaceutically acceptable salt, solvate, or stereoisomer thereof, wherein R¹, R², and R³ are defined herein elsewhere.

In one embodiment, R¹ is optionally substituted pyridyl, optionally substituted phenyl, optionally substituted pyrimidinyl, optionally substituted pyrazinyl, optionally substituted isoxazolyl, optionally substituted oxazolyl, or optionally substituted thiazolyl. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe. In another embodiment, R¹ is pyrimidinyl substituted with one or more halo, CN, lower alkyl, or —O(lower alkyl). In another embodiment, R¹ is pyrimidinyl substituted with one or more halo, CN, Me, CF₃, or OMe. In another embodiment, R¹ is pyrazinyl substituted with one or more halo, CN, lower alkyl, or —O(lower alkyl). In another embodiment, R¹ is pyrazinyl substituted with one or more halo, CN, Me, CF₃, or OMe. In another embodiment, R¹ is isoxazolyl substituted with one or more halo, CN, lower alkyl, or —O(lower alkyl). In another embodiment, R¹ is isoxazolyl substituted with one or more halo, CN, Me, CF₃, or OMe. In another embodiment, R¹ is oxazolyl substituted with one or more halo, CN, lower alkyl, or —O(lower alkyl). In another embodiment, R¹ is oxazolyl substituted with one or more halo, CN, Me, CF₃, or OMe. In another embodiment, R¹ is thiazolyl substituted with one or more halo, CN, lower alkyl, or —O(lower alkyl). In another embodiment, R¹ is thiazolyl substituted with one or more halo, CN, Me, CF₃, or OMe.

In one embodiment, R² is optionally substituted pyridyl, optionally substituted phenyl, optionally substituted pyrimidinyl, optionally substituted pyrazinyl, or optionally substituted thiazolyl. In one embodiment, R² is pyridyl substituted with one or more halo, CN, lower alkyl, or —O(lower alkyl). In another embodiment, R² is pyridyl substituted with one or more halo, CN, Me, CF₃, or OMe. In another embodiment, R² is phenyl substituted with one or more halo, CN, lower alkyl, or —O(lower alkyl). In another embodiment, R² is phenyl substituted with one or more halo, CN, Me, CF₃, or OMe. In another embodiment, R² is pyrimidinyl substituted with one or more halo, CN, lower alkyl, or —O(lower alkyl). In another embodiment, R² is pyrimidinyl substituted with one or more halo, CN, Me, CF₃, or OMe. In another embodiment, R² is pyrazinyl substituted with one or more halo, CN, lower alkyl, or —O(lower alkyl). In another embodiment, R² is pyrazinyl substituted with one or more halo, CN, Me, CF₃, or OMe. In another embodiment, R² is thiazolyl substituted with one or more halo, CN, lower alkyl, or —O(lower alkyl). In another embodiment, R² is thiazolyl substituted with one or more halo, CN, Me, CF₃, or OMe.

In one embodiment, R³ is hydrogen. In another embodiment, R³ is methyl.

Specific examples include, but are not limited to, the following compounds:

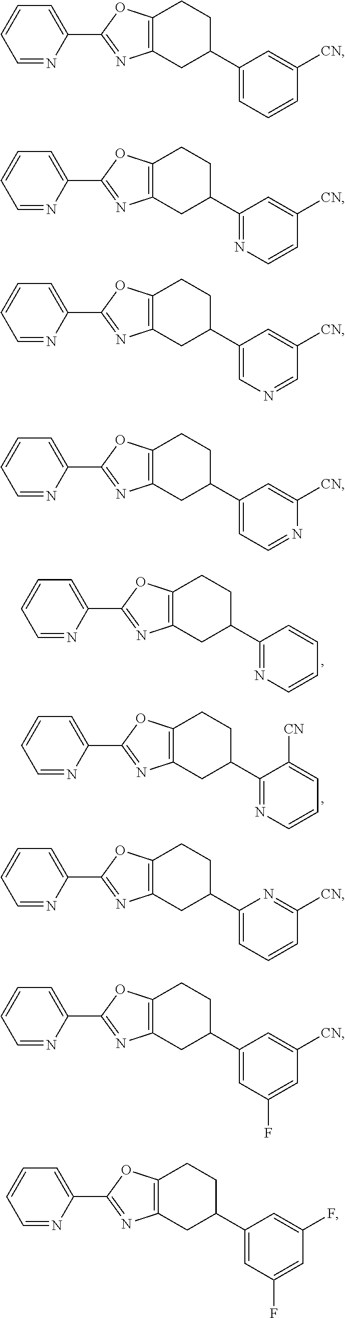

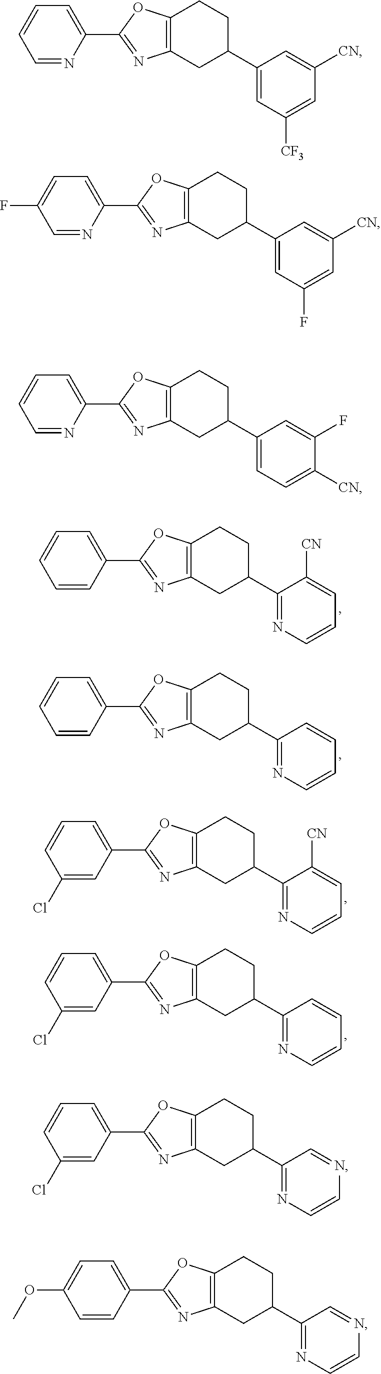

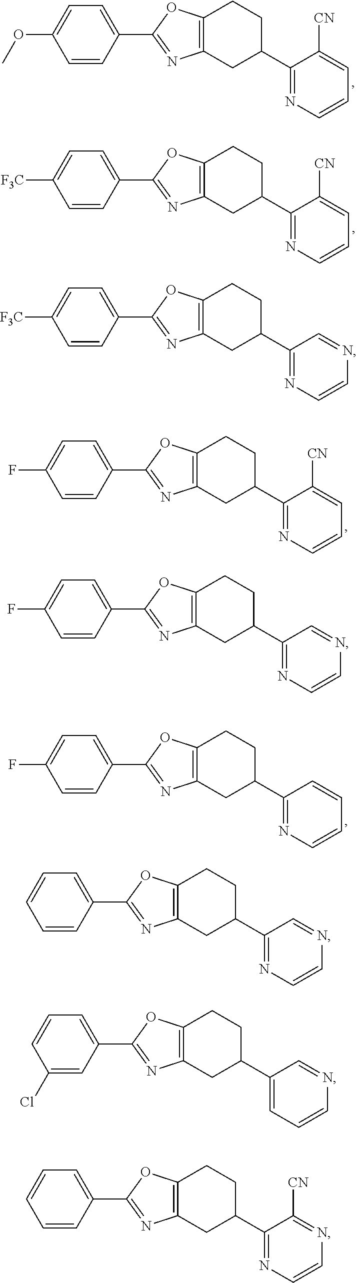

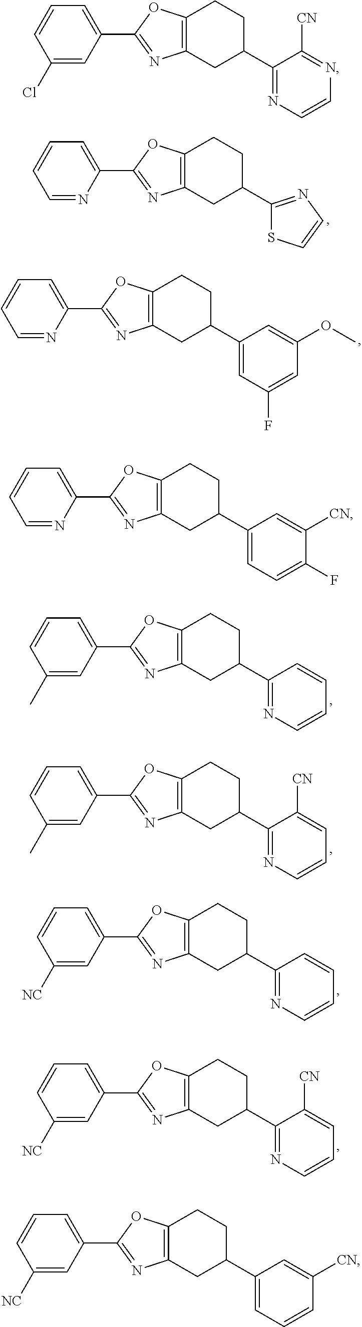

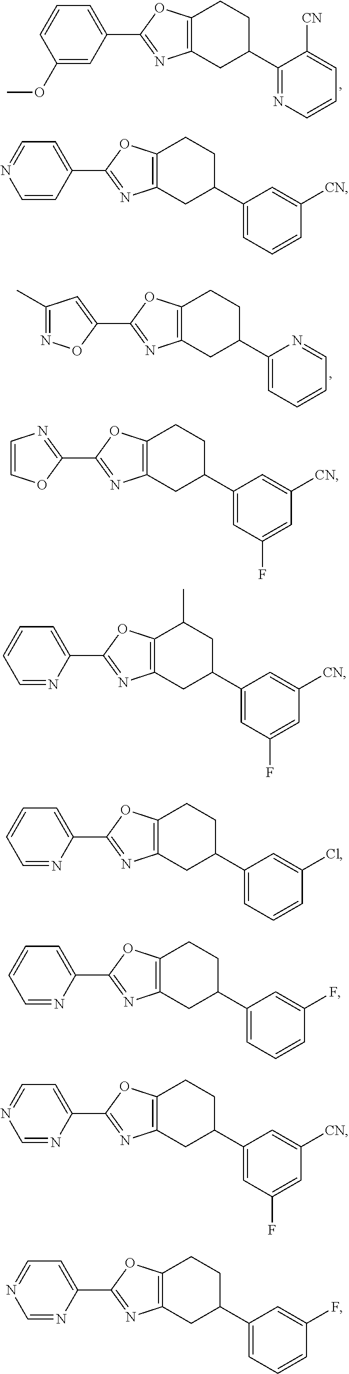

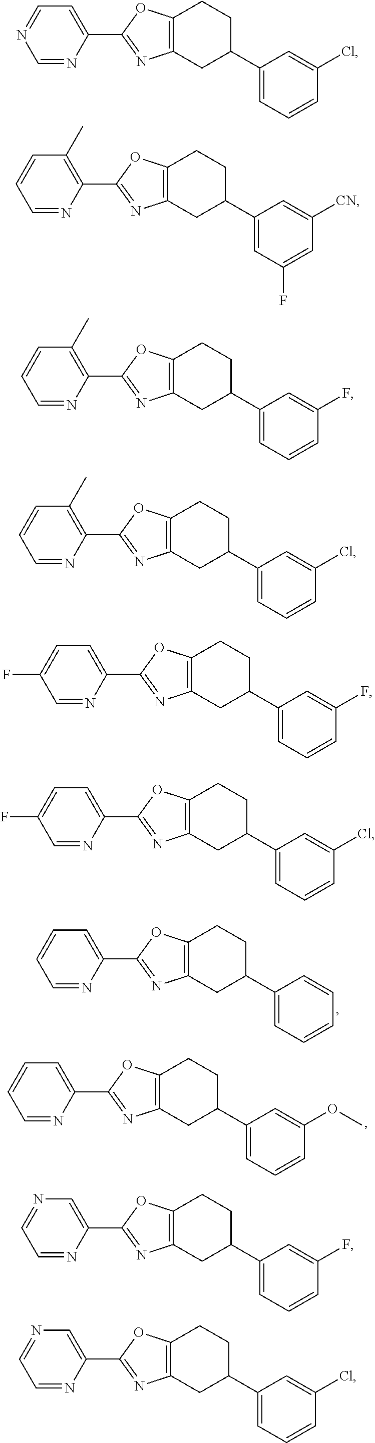

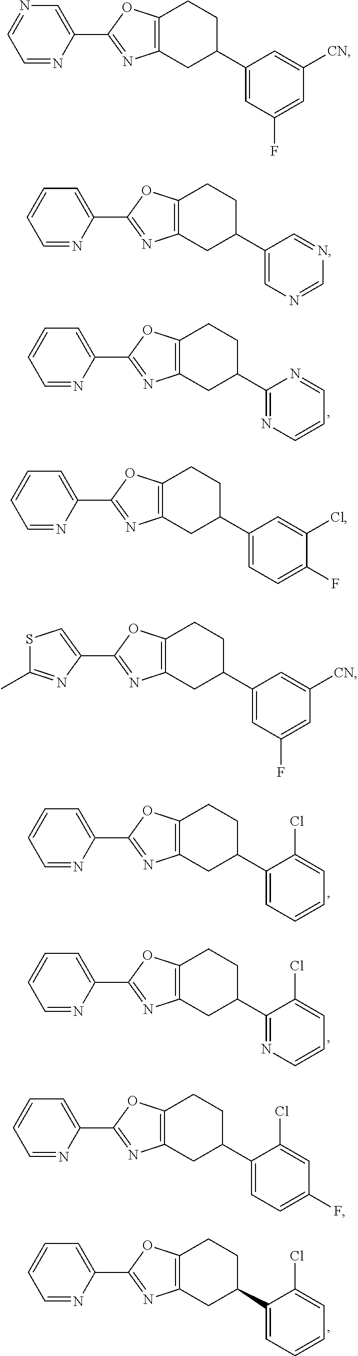

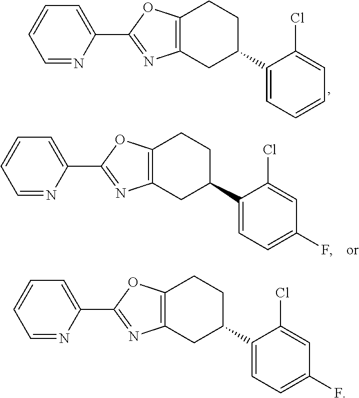

In one embodiment, provided herein is a compound of formula (Vc):

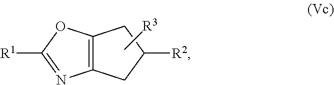

or a pharmaceutically acceptable salt, solvate, or stereoisomer thereof, wherein R¹, R², and R³ are defined herein elsewhere. In one embodiment, R¹ is optionally substituted pyridyl or optionally substituted phenyl. In another embodiment, R¹ is pyridyl substituted with one or more halo or CN. In another embodiment, R¹ is phenyl substituted with one or more halo or CN. In one embodiment, R² is optionally substituted pyridyl or optionally substituted phenyl. In another embodiment, R² is pyridyl substituted with one or more halo or CN. In another embodiment, R² is phenyl substituted with one or more halo or CN. In one embodiment, R³ is hydrogen.

Specific examples include, but are not limited to, the following compounds:

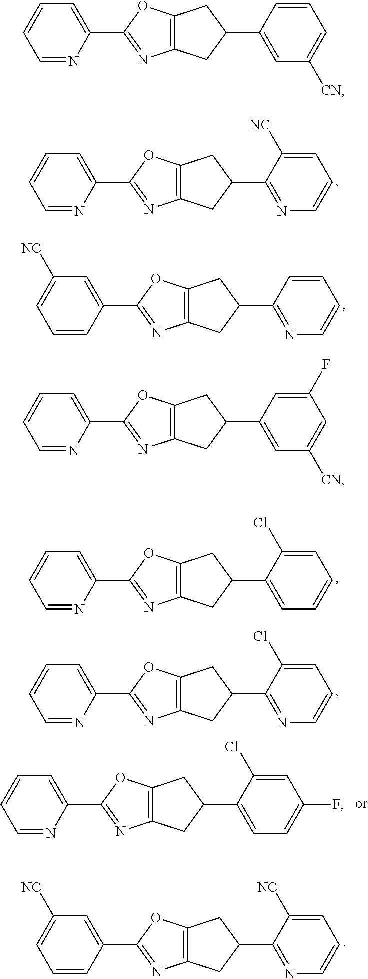

In one embodiment, provided herein is a compound of formula (Vd):

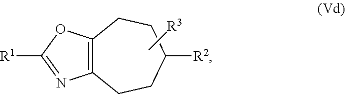

or a pharmaceutically acceptable salt, solvate, or stereoisomer thereof, wherein R¹, R², and R³ are defined herein elsewhere. In one embodiment, R¹ is optionally substituted pyridyl or optionally substituted phenyl. In another embodiment, R¹ is pyridyl substituted with one or more halo or CN. In another embodiment, R¹ is phenyl substituted with one or more halo or CN. In one embodiment, R² is optionally substituted pyridyl or optionally substituted phenyl. In another embodiment, R² is pyridyl substituted with one or more halo or CN. In another embodiment, R² is phenyl substituted with one or more halo or CN. In one embodiment, R³ is hydrogen.

Specific examples include, but are not limited to, the following compounds:

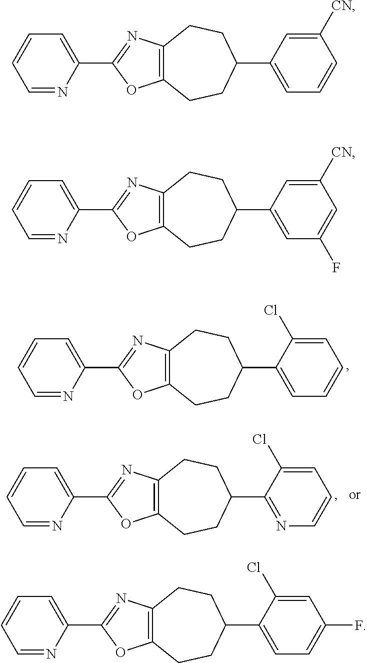

In one embodiment, provided herein is a compound of formula (Ve):

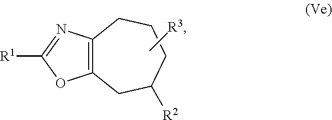

or a pharmaceutically acceptable salt, solvate, or stereoisomer thereof, wherein R¹, R², and R³ are defined herein elsewhere. In one embodiment, R¹ is optionally substituted pyridyl, optionally substituted phenyl, or optionally substituted pyrimidinyl. In another embodiment, R¹ is pyridyl substituted with one or more halo or CN. In another embodiment, R¹ is phenyl substituted with one or more halo or CN. In another embodiment, R¹ is pyrimidinyl substituted with one or more halo or CN. In one embodiment, R² is optionally substituted pyridyl or optionally substituted phenyl. In another embodiment, R² is pyridyl substituted with one or more halo, CN, lower alkyl, or —O(lower alkyl). In another embodiment, R² is pyridyl substituted with one or more halo, CN, Me, CF₃ or OMe. In another embodiment, R² is phenyl substituted with one or more halo, CN, lower alkyl, or —O(lower alkyl). In another embodiment, R² is phenyl substituted with one or more halo, CN, Me, CF₃ or OMe. In one embodiment, R³ is hydrogen.

Specific examples include, but are not limited to, the following compounds:

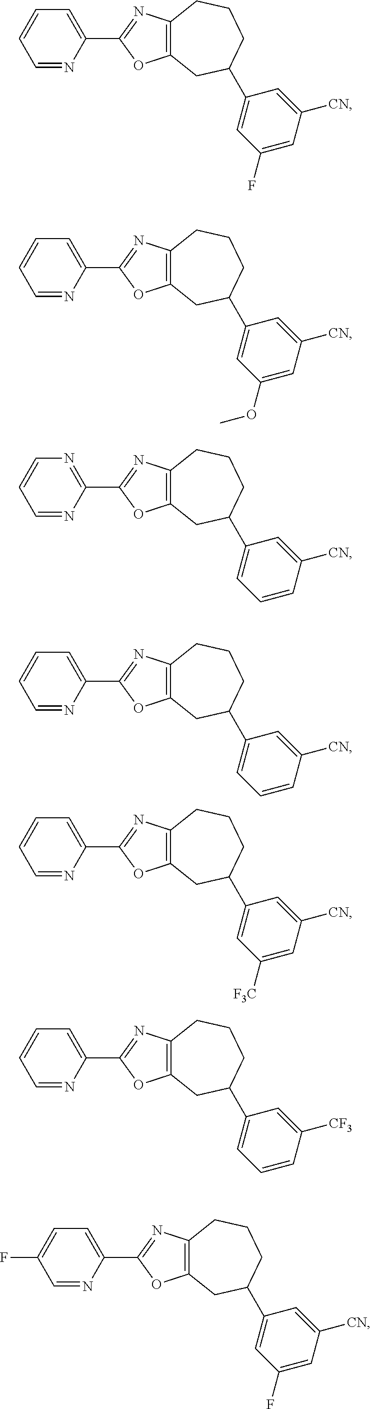

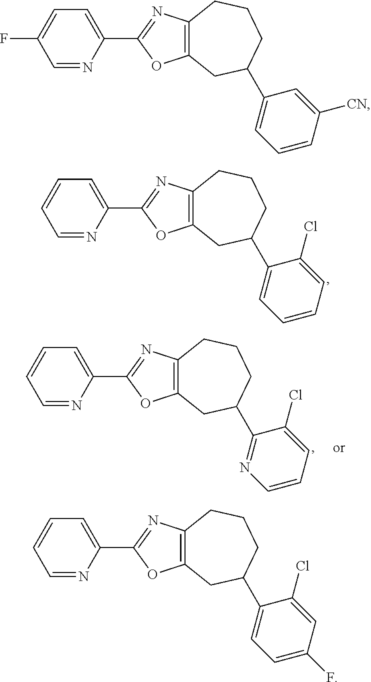

In one embodiment, provided herein is a compound of formula (Vf):

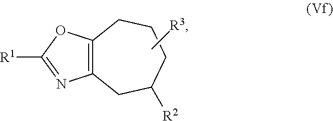

or a pharmaceutically acceptable salt, solvate, or stereoisomer thereof, wherein R¹, R², and R³ are defined herein elsewhere.

In one embodiment, R¹ is optionally substituted pyridyl, optionally substituted phenyl, optionally substituted pyrimidinyl, optionally substituted pyrazinyl, or optionally substituted thiazolyl. In one embodiment, R¹ is pyridyl substituted with one or more halo, CN, lower alkyl, or —O(lower alkyl). In another embodiment, R¹ is pyridyl substituted with one or more halo, CN, Me, CF₃, or OMe. In another embodiment, R¹ is phenyl substituted with one or more halo, CN, lower alkyl, or —O(lower alkyl). In another embodiment, R¹ is phenyl substituted with one or more halo, CN, Me, CF₃, or OMe. In another embodiment, R¹ is pyrimidinyl substituted with one or more halo, CN, lower alkyl, or —O(lower alkyl). In another embodiment, R¹ is pyrimidinyl substituted with one or more halo, CN, Me, CF₃, or OMe. In another embodiment, R¹ is pyrazinyl substituted with one or more halo, CN, lower alkyl, or —O(lower alkyl). In another embodiment, R¹ is pyrazinyl substituted with one or more halo, CN, Me, CF₃, or OMe. In another embodiment, R¹ is thiazolyl substituted with one or more halo, CN, lower alkyl, or —O(lower alkyl). In another embodiment, R¹ is thiazolyl substituted with one or more halo, CN, Me, CF₃, or OMe.

In one embodiment, R² is optionally substituted pyridyl, optionally substituted phenyl, or optionally substituted pyrimidinyl. In one embodiment, R² is pyridyl substituted with one or more halo, CN, lower alkyl, or —O(lower alkyl). In another embodiment, R² is pyridyl substituted with one or more halo, CN, Me, CF₃ or OMe. In another embodiment, R² is phenyl substituted with one or more halo, CN, lower alkyl, or —O(lower alkyl). In another embodiment, R² is phenyl substituted with one or more halo, CN, Me, CF₃ or OMe. In another embodiment, R² is pyrimidinyl substituted with one or more halo, CN, lower alkyl, or —O(lower alkyl). In another embodiment, R² is pyrimidinyl substituted with one or more halo, CN, Me, CF₃ or OMe.

In one embodiment, R³ is hydrogen. In another embodiment, R³ is methyl.

Specific examples include, but are not limited to, the following compounds:

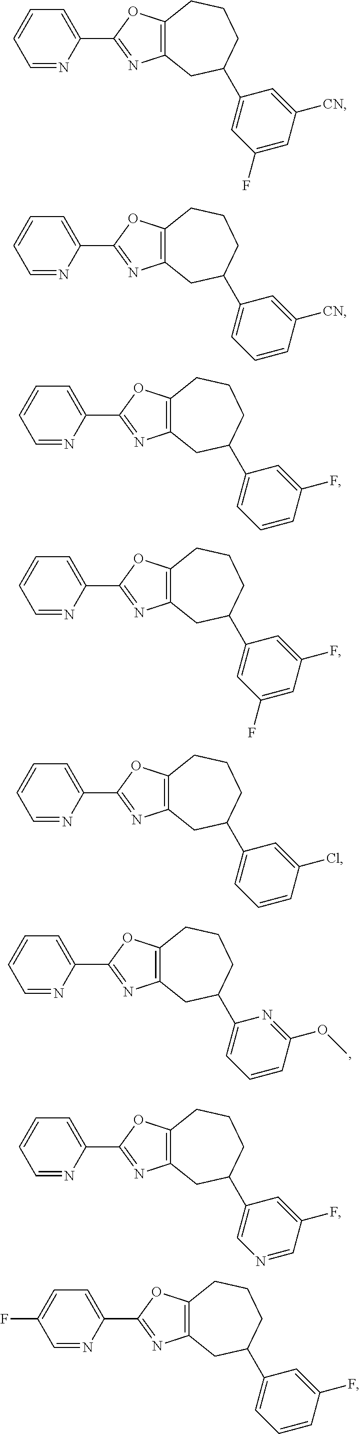

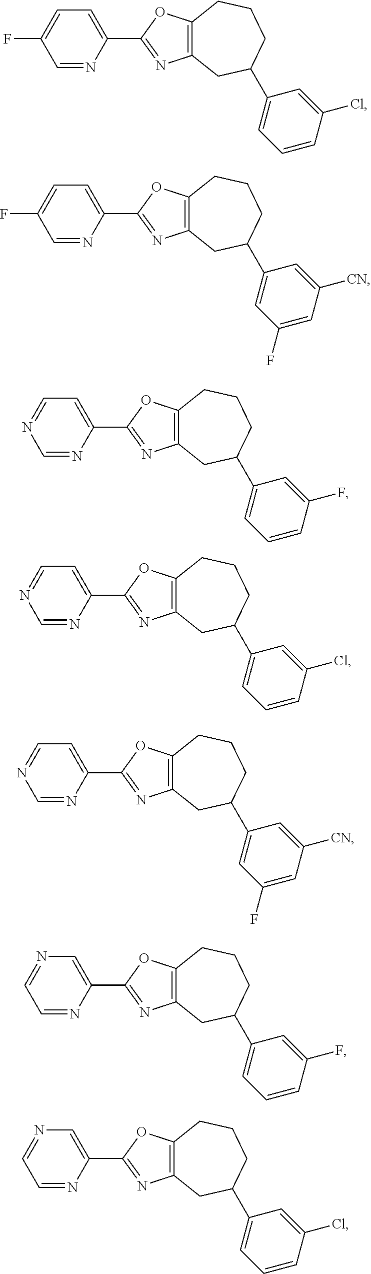

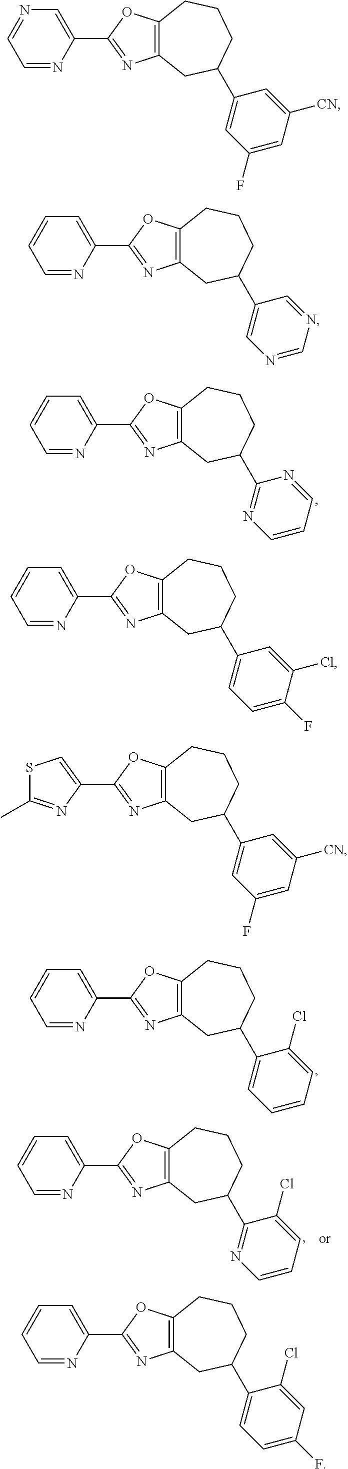

In one embodiment, provided herein is a compound or a pharmaceutically acceptable salt, solvate, or stereoisomer thereof, wherein the compound is:

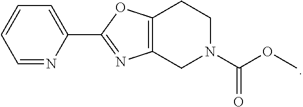

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it. Where the compound provided herein contains an alkenyl or alkenylene group, the compound may exist as one or mixture of geometric cis/trans (or Z/E) isomers. Where structural isomers are inter-convertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. In some instances, for compounds that undergo epimerization in vivo, one of skill in the art will recognize that administration of a compound in its (R) form is equivalent to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, by chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt (See, e.g., Berge et al., J. Pharm. Sci. 1977, 66, 1-19; and Handbook of Pharmaceutical Salts, Properties, and Use, Stahl and Wermuth, ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound provided herein may also be provided as a prodrug, which is a functional derivative of the compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See, e.g., Harper, Progress in Drug Research 1962, 4, 221-294; Morozowich et al. in Design of Biopharmaceutical Properties through Prodrugs and Analogs, Roche ed., APHA Acad. Pharm. Sci. 1977; Bioreversible Carriers in Drug in Drug Design, Theory and Application, Roche ed., APHA Acad. Pharm. Sci. 1987; Design of Prodrugs, Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in Transport Processes in Pharmaceutical Systems, Amidon et al., ed., Marcell Dekker, 185-218, 2000; Balant et al., Eur. J. Drug Metab. Pharmacokinet. 1990, 15, 143-53; Balimane & Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

In one embodiment, also provided herein are an isotopically enriched compound of formula (I), (Ia), (Ib), (II), (IIa), (IIb), (III), (IIIa), (IIIb), (IVa), (IVb), (IVc), (IVd), (IVe), (IVf), (Va), (Vb), (Vc), (Vd), (Ve), or (Vf).

Isotopic enrichment (for example, deuteration) of pharmaceuticals to improve pharmacokinetics (“PK”), pharmacodynamics (“PD”), and toxicity profiles, has been demonstrated previously with some classes of drugs. See, e.g., Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a deuterium for that hydrogen will cause a decrease in the reaction rate and the process will slow down. This phenomenon is known as the Deuterium Kinetic Isotope Effect (“DKIE”). See, e.g., Foster et al., Adv. Drug Res., vol. 14, pp. 1-36 (1985); Kushner et al., Can. J. Physiol. Pharmacol., vol. 77, pp. 79-88 (1999).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has two neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### C. Synthetic Schemes

Schemes below provide exemplary synthetic methods for the preparation of the compounds provided herein. One of ordinary skills in the art will understand that similar methods may be employed to prepare the compounds provided herein. In other words, one of ordinary skills in the art will recognize that suitable adjustments to reagents, protecting groups, reaction conditions, and reaction sequences may be employed to prepare a desired embodiment. The reactions may be scaled upwards or downwards to suit the amount of material to be prepared.

In one embodiment, a compound of formula (Ia) may be prepared by coupling a cyclic amine Ia-1 with R²-L²R¹¹ wherein R¹¹ is halogen, such as Cl, Br, or I, or —OH, or other suitable leaving groups, such as -OMesylate, or -OTosylate (See Scheme 1). Optionally, further organic transformations may convert a certain set of L¹, L², R¹ and R² to a new set of L¹, L², R¹ and R² groups to prepare a specific compound of formula (Ia).

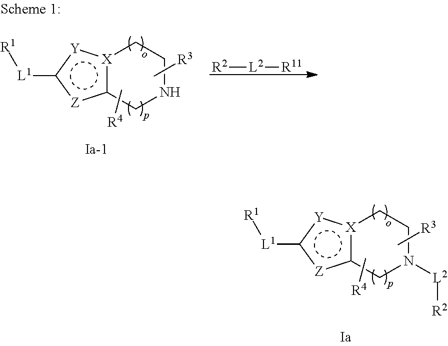

In one embodiment, the cyclic amine Ia-1 may be coupled with an alkyl halide, such as a substituted benzyl bromide, under basic conditions, such as K₂CO₃ or Na₂CO₃, in a solvent such as DMF or acetonitrile, to render a compound of formula (Ia) where L² is —CH₂— (See Scheme 2).

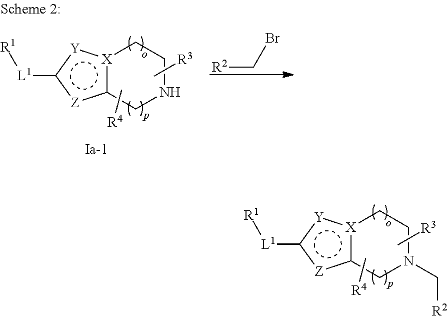

In another embodiment, the cyclic amine Ia-1 may also be coupled with an acid chloride, or an acid in the presence of amide coupling reagents, such as EDCI and HOBt, with base such as TEA, in a solvent such as DCM, to render an amide (See Scheme 3).

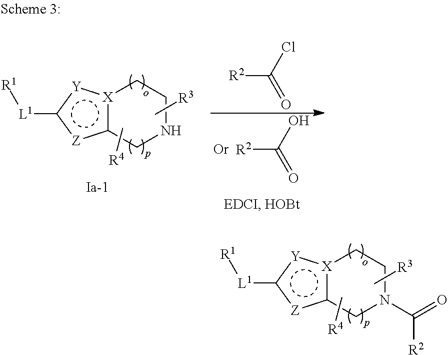

In another embodiment, when L² is a bond, and R² is aryl or heteroaryl, a compound of formula (Ia) may be prepared according to Scheme 4 by coupling a cyclic amine Ia-1 with aryl halide or heteroaryl halide, such as aryl or heteroaryl chlorides, bromides, or iodides, in the presence of suitable coupling reagents and under heating. Exemplary coupling reagents include, but are not limited to the following: (1) Pd(OAc)₂, Xantphos, and Cs₂CO₃ in toluene; (2) Pd(OAc)₂, Xantphos, and K₂CO₃ in toluene; (3) Pd(OAc)₂, Xantphos, and NaOtBu in toluene; (4) Pd₂(dba)₃, BINAP, and NaOtBu in t-BuOH or toluene; (5) Pd₂(dba)₃, (tBu)₃P.BF₄, and NaOtBu in toluene; or (6) Pd₂(dba)₃, Xantphos, and Cs₂CO₃ in xylene. When the heteroaryl halide is an electrophile, such as 2-chloropyridine, the coupling reaction could be carried out in the presence of a base, such as DIEA, in a solvent such as DMF, under thermal or microwave heating.

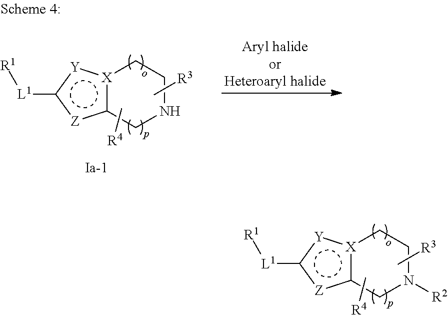

In some embodiments, the cyclic amine Ia-1 may be prepared using the following specific schemes. Detailed reaction conditions are provided herein below for various specific examples. These specific schemes and specific examples of Ia-1 are not limiting on the scope of this disclosure. One of ordinary skills of the art will understand that the following schemes may be modified with appropriate reagents, protecting groups, conditions, starting materials, or reaction sequences to suit the preparation of various other embodiments provided herein.

In one embodiment, the cyclic amine Ia-1 is a 2-position substituted 4,5,6,7-tetrahydrooxazolo[5,4-c]pyridine, which may be prepared using procedures exemplified in Scheme 5. trans-Benzyl 4-amino-3-hydroxypiperidine-1-carboxylate is prepared according to procedures in WO1994/20062, and coupled to an acid, such as picolinic acid, under standard amid coupling conditions, such as EDCI, HOBt, and TEA in DCM. The resulting hydroxy-amide compound is oxidized and subsequently cyclized to form the Cbz-protected 4,5,6,7-tetrahydrooxazolo[5,4-c]pyridine. The Cbz protecting group may be removed by catalytic hydrogenation or by treatment with TMSI to render the desired cyclic amine Ia-1.

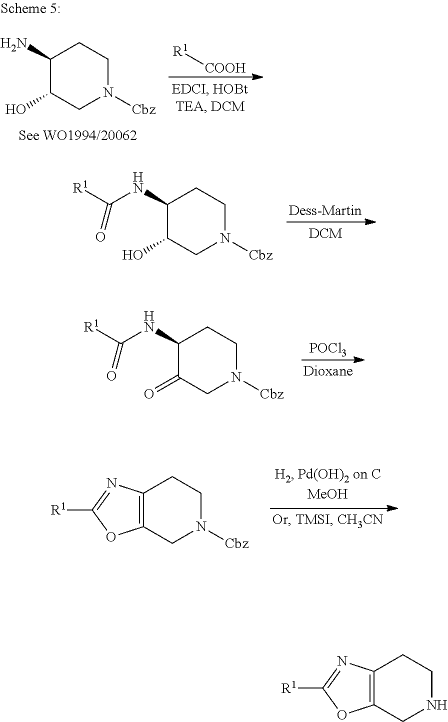

In another embodiment, the cyclic amine Ia-1 is a 2-position substituted 4,5,6,7-tetrahydrooxazolo[4,5-c]pyridine, which may be prepared using procedures exemplified in Scheme 6. trans-Benzyl 3-amino-4-hydroxypiperidine-1-carboxylate is prepared according to procedures in WO1994/20062, and coupled to an acid, such as picolinic acid, under standard amid coupling conditions, such as EDCI, HOBt, and TEA in DCM. The resulting hydroxy-amide compound is oxidized and subsequently cyclized to form the Cbz-protected 4,5,6,7-tetrahydrooxazolo[4,5-c]pyridine. The Cbz protecting group may be removed by catalytic hydrogenation or by treatment with TMSI to render the desired cyclic amine Ia-1.

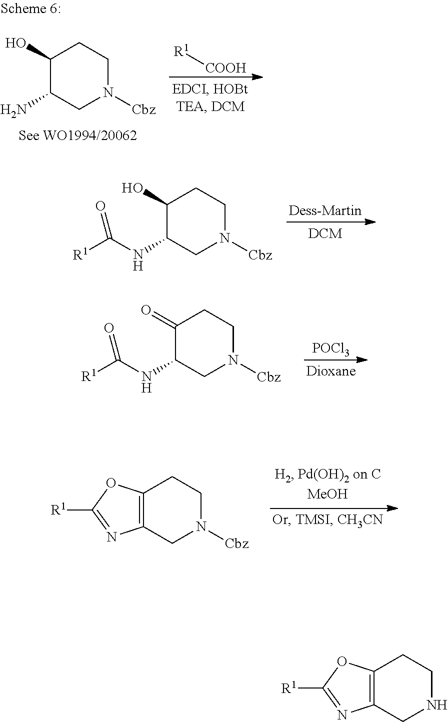

In one embodiment, the cyclic amine Ia-1 is 2-(oxazol-2-yl)-4,5,6,7-tetra-hydrooxazolo[4,5-c]pyridine, which may be prepared using procedures exemplified in Scheme 7. trans-Benzyl 3-amino-4-hydroxypiperidine-1-carboxylate is prepared according to procedures in WO1994/20062, and treated with oxalic acid diethyl ester, followed by oxidation to render a keto-amide. The keto-amide is cyclized to render 5-benzyl 2-ethyl 6,7-dihydrooxazolo[4,5-c]pyridine-2,5(4H)-dicarboxylate, which is coupled to 2,2-dimethoxy-ethylamine to yield an amide. The acetal amide is treated with TFA to render the corresponding aldehyde, which may be cyclized to form the oxazole. The Cbz protecting group is removed to provide the desired cyclic amine Ia-1.

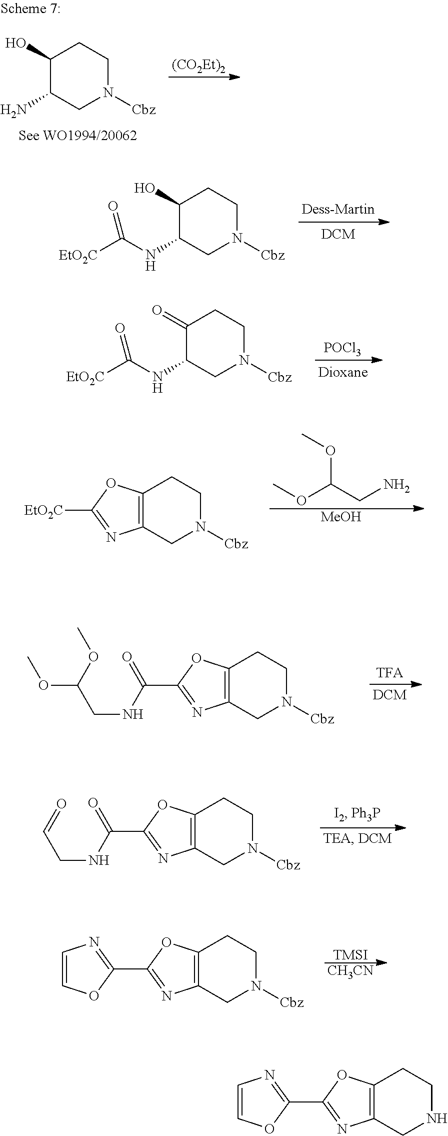

In another embodiment, the cyclic amine Ia-1 is a 2-position substituted 7-methyl-4,5,6,7-tetrahydrooxazolo[5,4-c]pyridine, a 2-position substituted 7-methyl-4,5,6,7-tetrahydrooxazolo[4,5-c]pyridine or a 2-position substituted 6-methyl-4,5,6,7-tetrahydrooxazolo[5,4-c]pyridine, which may be prepared using procedures exemplified in Schemes 8-11. One skilled in the art will recognize that suitable substituted piperidine or pyridine analogs may be prepared using known literature procedures or purchased from commercial sources to substitute various piperidine and pyridine intermediates exemplified in Schemes 8 and 11, and prepare suitable other embodiments of a compound of formula (Ia).

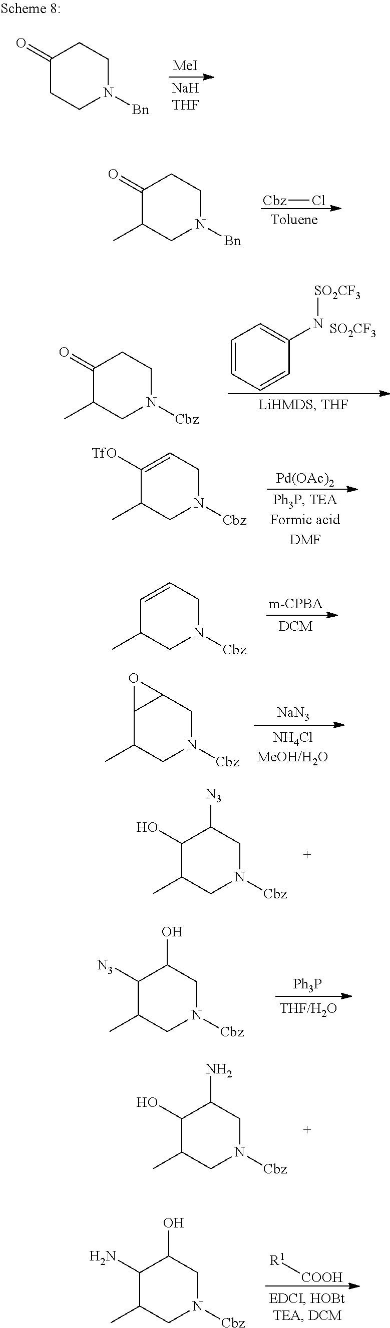

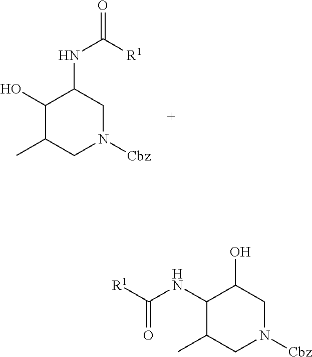

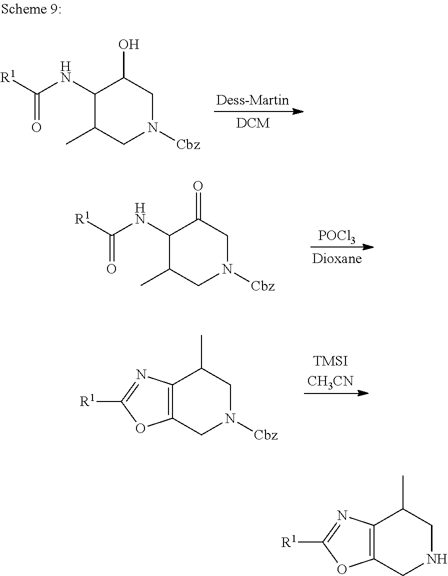

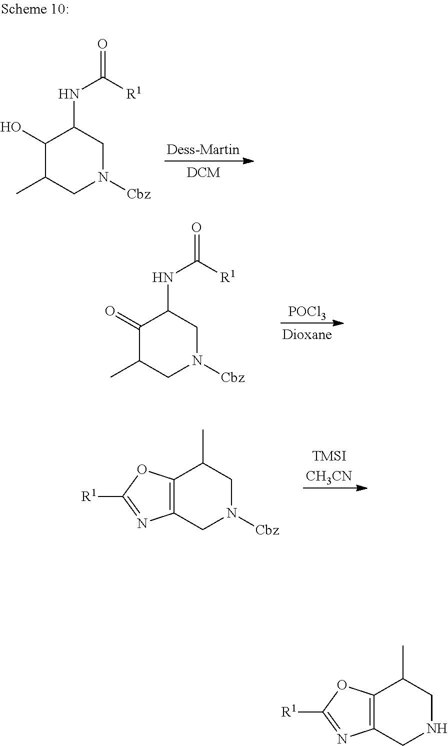

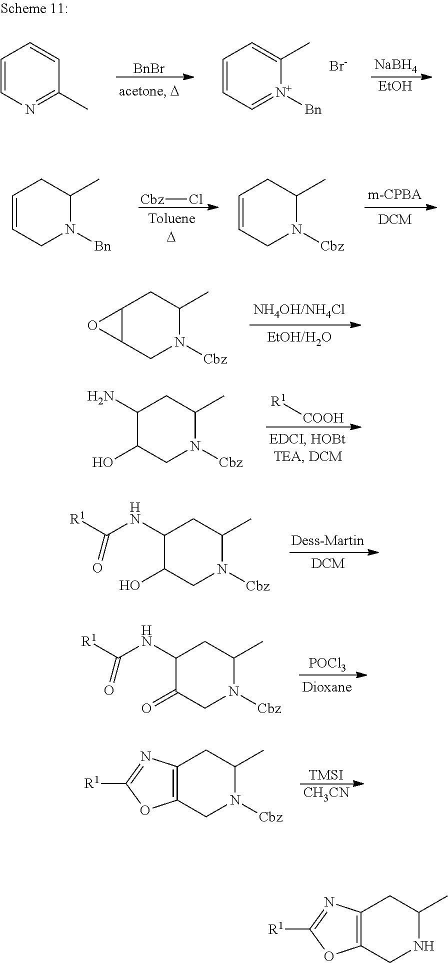

In another embodiment, the cyclic amine Ia-1 is a 2-position substituted 5,6-dihydro-4H-pyrrolo[3,4-d]oxazole, which may be prepared using procedures exemplified in Scheme 12. Pyrrole is reduced by Zn/HCl and protected with Cbz to yield benzyl 2,5-dihydro-1H-pyrrole-1-carboxylate, which is oxidized with m-CPBA to render the corresponding epoxide. The epoxide is treated with aqueous ammonia to yield benzyl 3-amino-4-hydroxypyrrolidine-1-carboxylate, which is coupled to an acid, such as picolinic acid, under standard amid coupling conditions, such as EDCI, HOBt, and TEA in DCM. The resulting hydroxy-amide compound is oxidized and subsequently cyclized to form the Cbz-protected 5,6-dihydro-4H-pyrrolo[3,4-d]oxazole. The Cbz protecting group may be removed by treatment with TMSI to render the desired cyclic amine Ia-1.

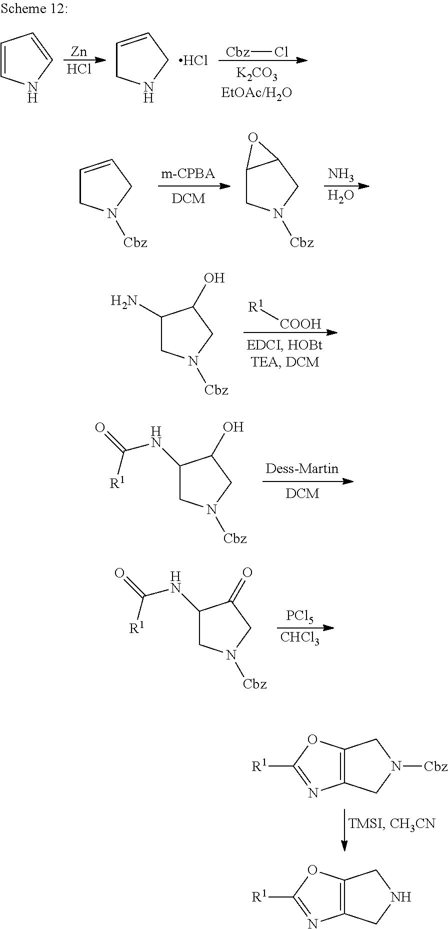

Alternatively, when the cyclic amine Ia-1 is a 2-position substituted 5,6-dihydro-4H-pyrrolo[3,4-d]oxazole, a compound of formula (Ia) may be prepared using procedures exemplified in Scheme 13. Pyrrole is reduced by Zn/HCl and protected with Boc to yield tert-butyl 2,5-dihydro-1H-pyrrole-1-carboxylate, which is oxidized with m-CPBA to render the corresponding epoxide. The epoxide is treated with aqueous ammonia to yield tert-butyl 3-amino-4-hydroxypyrrolidine-1-carboxylate, which is coupled to an acid under standard amid coupling conditions, such as EDCI, HOBt, and TEA in DCM. The resulting hydroxy-amide is treated with TFA to remove the Boc protecting group. The resulting amine is coupled to a heteroaryl halide, such as 2-chloro-3-cyanopyridine under basic condition. The product is oxidized to the corresponding ketone and subsequently cyclized to form the desired 5,6-dihydro-4H-pyrrolo[3,4-d]oxazole.

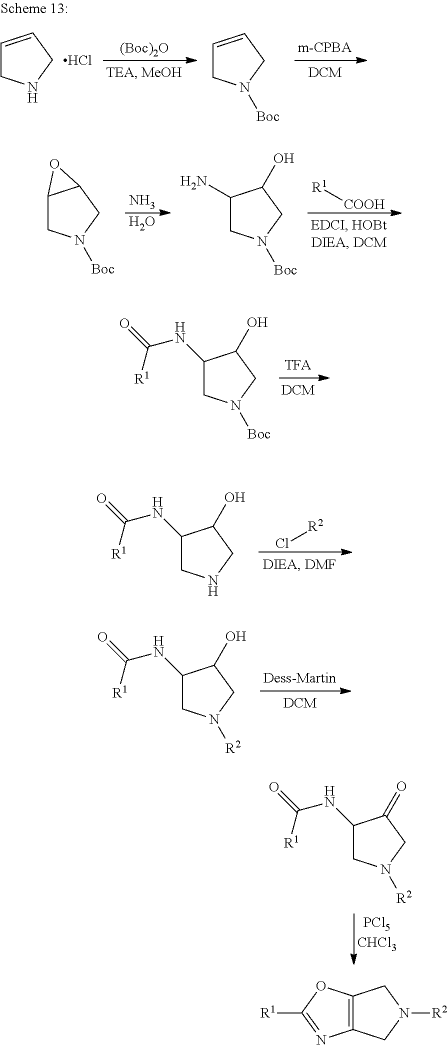

In another embodiment, the cyclic amine Ia-1 is a 2-position substituted 5,6,7,8-tetrahydro-4H-oxazolo[5,4-d]azepine, which may be prepared using procedures exemplified in Scheme 14. tert-Butyl 4-oxopiperidine-1-carboxylate is treated with ethyl diazoacetate in the presence of BF₃ etherate, to produce 1-tert-butyl 3-ethyl 4-oxoazepane-1,3-dicarboxylate. The ester is decarboxylated under acidic conditions to render azepan-4-one, which is protected with Cbz and reduced to the corresponding alcohol. The alcohol is converted to the corresponding mesylate and treated with DBU to yield a mixture of benzyl 2,3,6,7-tetrahydro-1H-azepine-1-carboxylate and benzyl 2,3,4,7-tetrahydro-1H-azepine-1-carboxylate. The mixture is treated with m-CPBA to provide benzyl 8-oxa-4-azabicyclo[5.1.0]octane-4-carboxylate. The epoxide is treated with sodium azide. The resulting product is reduced with triphenylphosphine to render a hydroxy-amine, which is coupled to an acid, such as picolinic acid, under standard amid coupling conditions, such as EDCI, HOBt, and TEA in DCM. The resulting hydroxy-amide is oxidized and subsequently cyclized to form a Cbz-protected 5,6,7,8-tetrahydro-4H-oxazolo[5,4-d]azepine. The Cbz protecting group may be removed by treatment with TMSI to render the desired cyclic amine Ia-1.

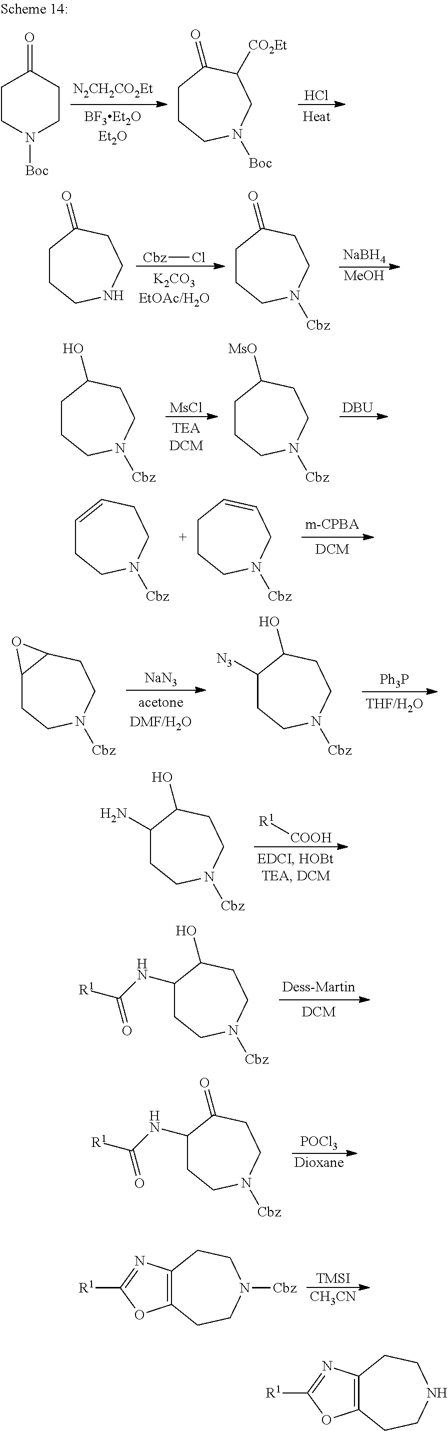

In another embodiment, the cyclic amine Ia-1 is a 2-position substituted 5,6,7,8-tetrahydro-4H-oxazolo[5,4-c]azepine, which may be prepared using procedures exemplified in Scheme 15. Prop-2-en-1-amine is protected with Cbz and alkylated with 5-bromopent-1-ene. The resulting diene is treated with Grubb's catalysis and undergoes ring closing metathesis to yield benzyl 2,3,4,7-tetrahydro-1H-azepine-1-carboxylate, which is treated with m-CPBA to provide the corresponding epoxide. The epoxide is treated with sodium azide. The resulting product is reduced with triphenylphosphine to render a hydroxy-amine, which is coupled to an acid, such as picolinic acid, under standard amid coupling conditions, such as EDCI, HOBt, and TEA in DCM. The resulting hydroxy-amide is oxidized and subsequently cyclized to form a Cbz-protected 5,6,7,8-tetrahydro-4H-oxazolo[5,4-d]azepine. The Cbz protecting group may be removed by treatment with TMSI to render the desired cyclic amine Ia-1.

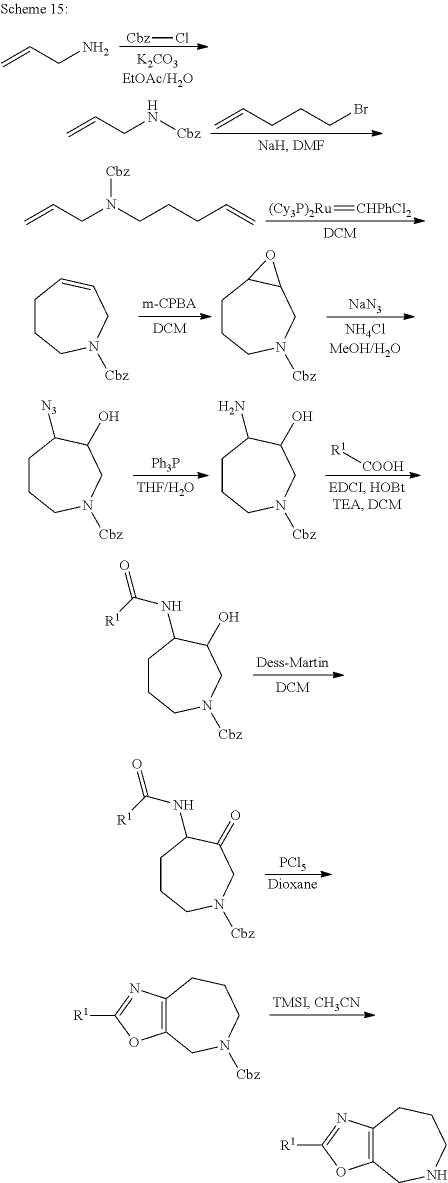

In another embodiment, the cyclic amine Ia-1 is a 2-position substituted 5,6,7,8-tetrahydro-4H-oxazolo[4,5-c]azepine, which may be prepared using procedures exemplified in Scheme 16. Benzyl 4-oxoazepane-1-carboxylate is brominated to yield a bromo-ketone, which is reduced to the corresponding bromo-alcohol. The bromo-alcohol is reacted with sodium azide. The resulting product is reduced with triphenylphosphine to render a hydroxy-amine, which is coupled to an acid, such as picolinic acid, under standard amid coupling conditions, such as EDCI, HOBt, and TEA in DCM. The resulting hydroxy-amide is oxidized and subsequently cyclized to form a Cbz-protected 5,6,7,8-tetrahydro-4H-oxazolo[4,5-c]azepine. The Cbz protecting group may be removed by treatment with TMSI to render the desired cyclic amine Ia-1.

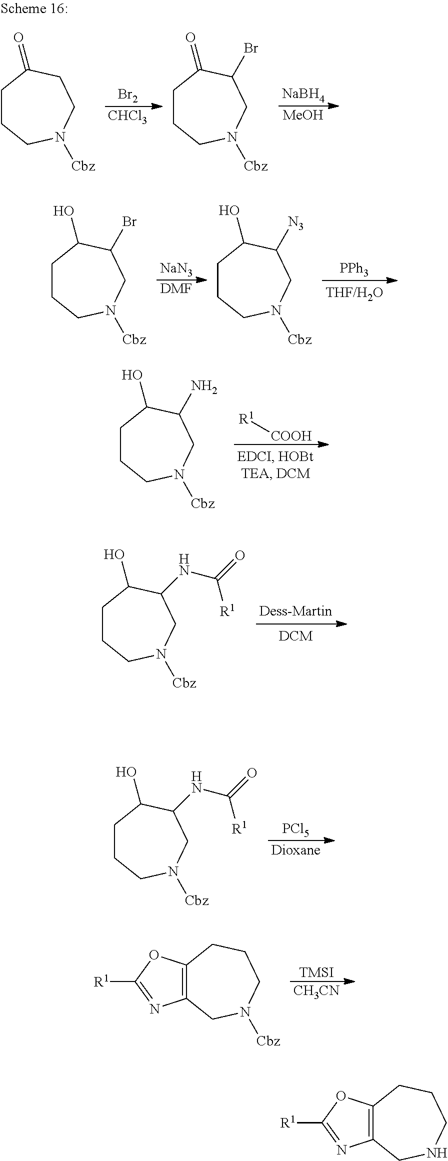

In another embodiment, the cyclic amine Ia-1 is a 2-position substituted 4,5,6,7-tetrahydrothiazolo[5,4-c]pyridine, which may be prepared using procedures exemplified in Scheme 17. tert-Butyl 4-oxopiperidine-1-carboxylate is brominated to yield a bromo-ketone, which is reacted with a suitable thioamide to render the 2-substituted 4,5,6,7-tetrahydrothiazolo[5,4-c]pyridine.

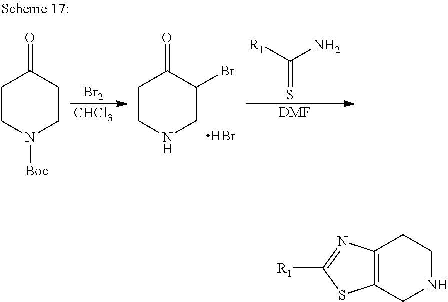

In another embodiment, the cyclic amine Ia-1 is a 2-position substituted 5,6,7,8-tetrahydroimidazo[1,2-a]pyrazine, which may be prepared using procedures exemplified in Scheme 18. Cbz-protected glycine is reacted with a bromomethyl ketone to yield an imidazole intermediate, which is alkylated with ethyl bromoacetate. The resulting product is hydrogenate under acidic conditions to remove the Cbz protecting group and cyclize. The resulting cyclic amide is reduced with BH₃ in THF to render the 2-substituted 5,6,7,8-tetrahydroimidazo[1,2-a]pyrazine.

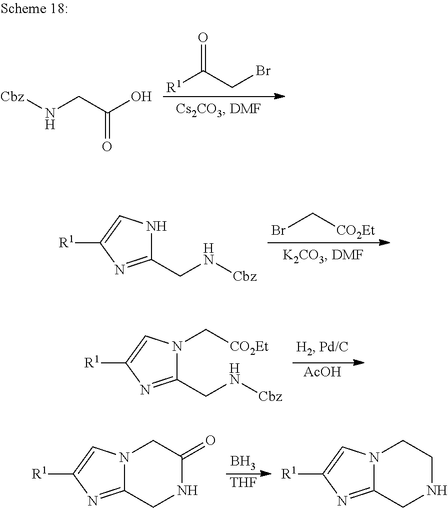

In another embodiment, the cyclic amine Ia-1 is a 2-position substituted 4,5,6,7-tetrahydro-3H-imidazo[4,5-c]pyridine, and the compound of formula (Ia) may be prepared using procedures exemplified in Scheme 19. Benzyl 4-azido-3-hydroxypiperidine-1-carboxylate, which is prepared according to the procedures in WO1994/20062, is converted to the corresponding mesylate, which is reacted with sodium azide to form a di-azide. The di-azide is reduced with triphenylphosphine, and the resulting di-amine is coupled with a suitable ethyl imidate to form the imidazoline intermediate, which is oxidized to the corresponding imidazole. The imidazole is protected with para-methoxy benzyl (PMB) group, and the Cbz protecting group is removed via hydrogenation. The amine is coupled to a suitable aryl or heteroaryl halide and the PMB protecting group is removed by TFA to render the desired compound of formula (Ia).

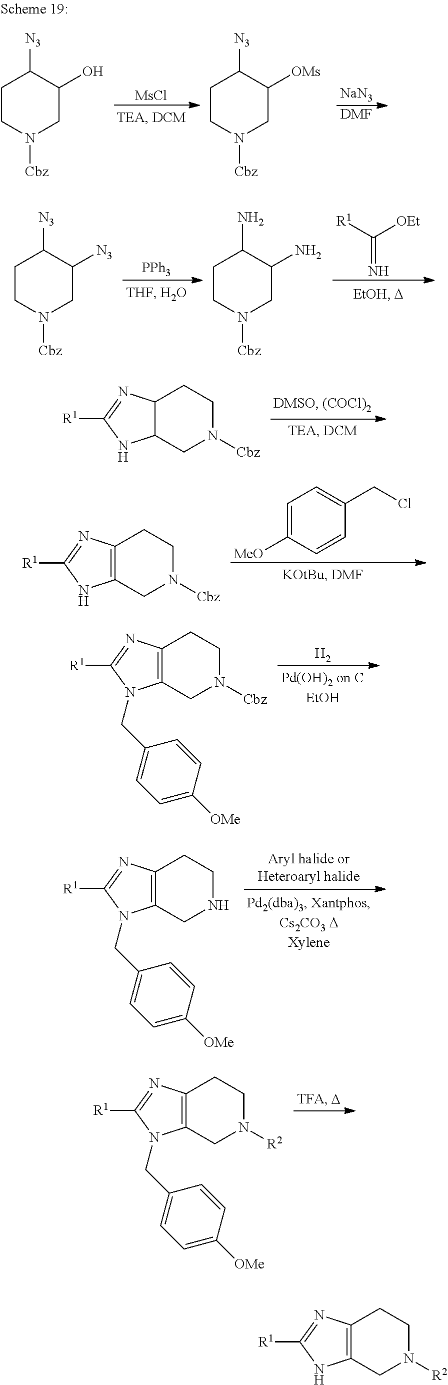

In another embodiment, the cyclic amine Ia-1 is a 2-position substituted 5,6,7,8-tetrahydro-[1,2,4]triazolo[1,5-a]pyrazine, which may be prepared using procedures exemplified in Scheme 20. Cbz-protected glycine is reacted with ethyl chloroformate in the presence of 4-methylmorpholine to yield an acyl hydrazine, which is coupled with a suitable ethyl imidate to form a triazole intermediate, which is alkylated with ethyl bromoacetate. The resulting product is hydrogenate to remove the Cbz protecting group and cyclize. The resulting cyclic amide is reduced with BH₃ in THF to render the 2-substituted 5,6,7,8-tetrahydro-[1,2,4]triazolo[1,5-a]pyrazine.

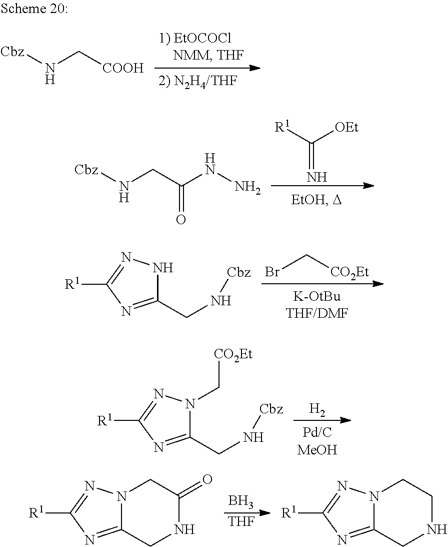

In another embodiment, a compound of formula (Ib) may be prepared following Scheme 21, using suitable starting material known in the art and/or available from a commercial source.

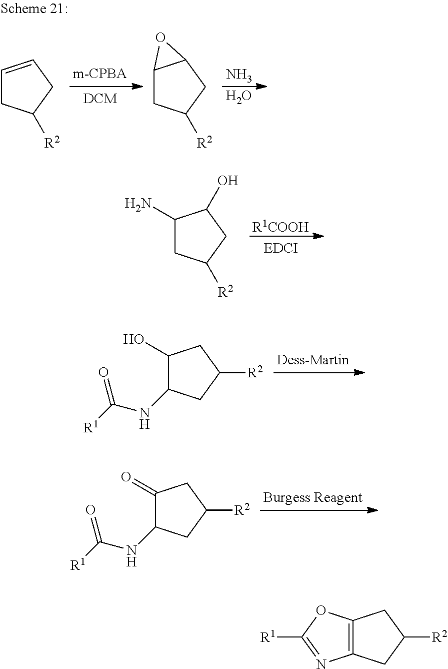

In another embodiment, a compound of formula (Ib) may be prepared following Scheme 22, using suitable starting material known in the art and/or available from a commercial source.

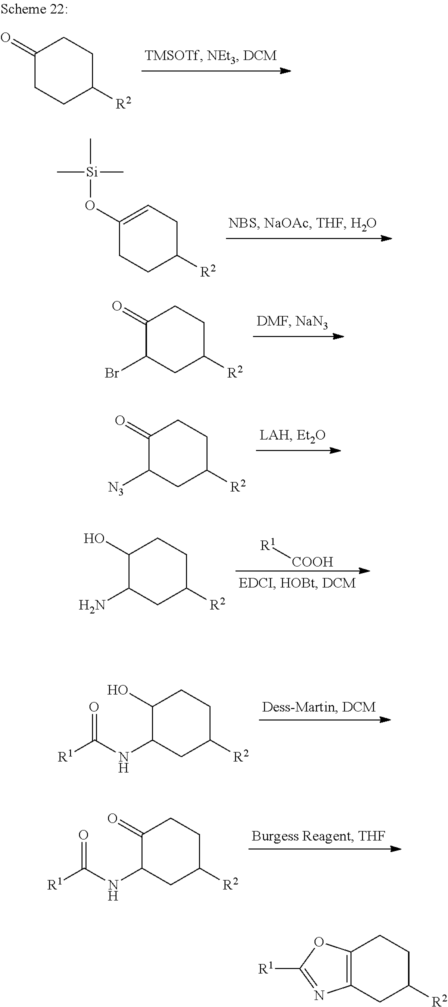

In another embodiment, a compound of formula (Ib) may be prepared following Scheme 23, using suitable starting material known in the art and/or available from a commercial source.

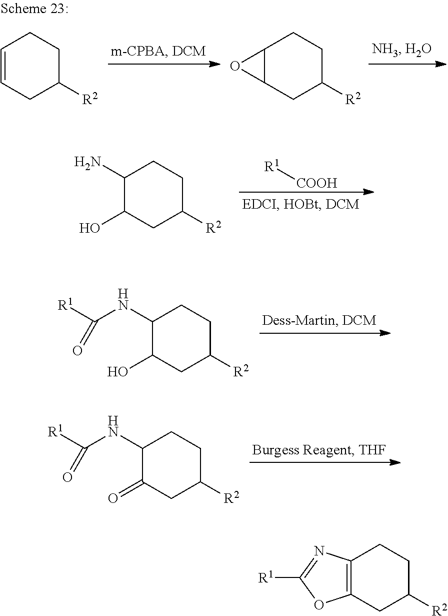

In another embodiment, a compound of formula (Ib) may be prepared following Scheme 24, using suitable starting material known in the art and/or available from a commercial source.

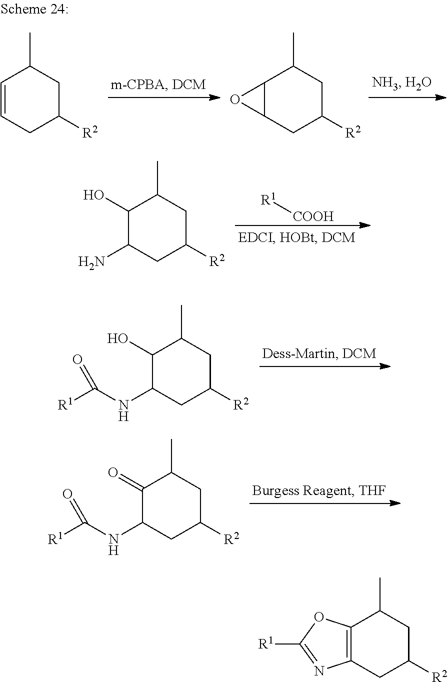

In another embodiment, a compound of formula (Ib) may be prepared following Scheme 25, using suitable starting material known in the art and/or available from a commercial source.

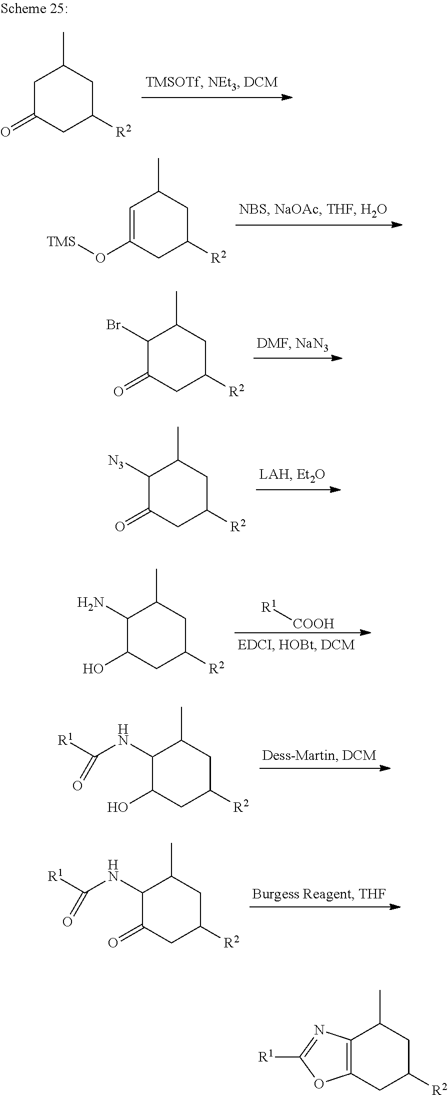

In another embodiment, a compound of formula (Ib) may be prepared following Scheme 26, using suitable starting material known in the art and/or available from a commercial source.

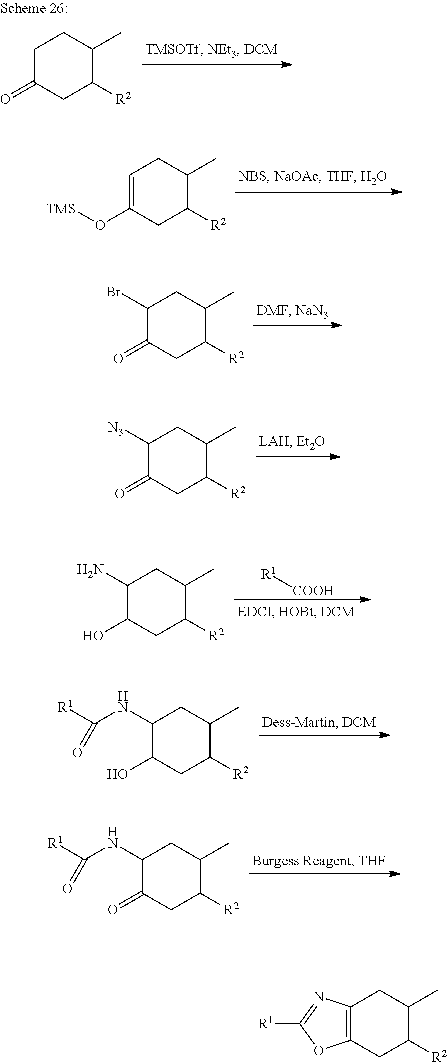

In one embodiment, a compound of formula (Ib) may be prepared following Scheme 27, using suitable starting material known in the art and/or available from a commercial source.

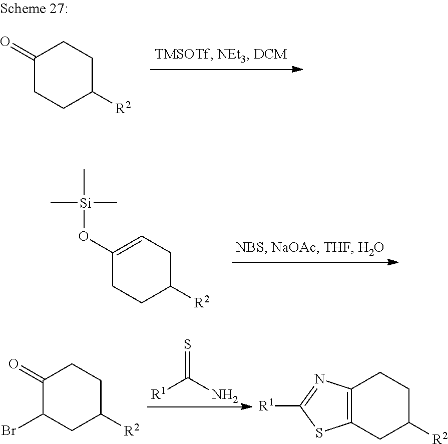

In another embodiment, a compound of formula (Ib) may be prepared following Scheme 28, using suitable starting material known in the art and/or available from a commercial source.

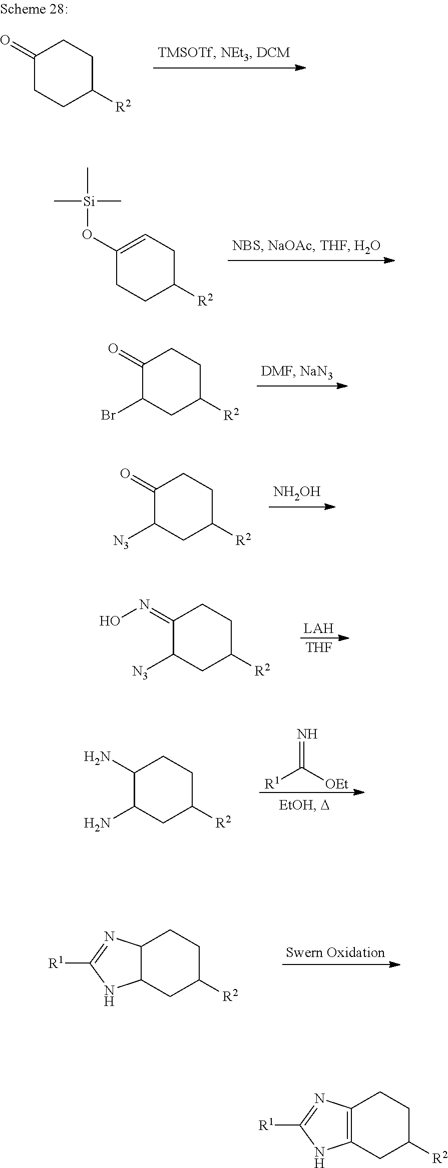

In another embodiment, a compound of formula (Ib) may be prepared following Scheme 29, using suitable starting material known in the art and/or available from a commercial source.

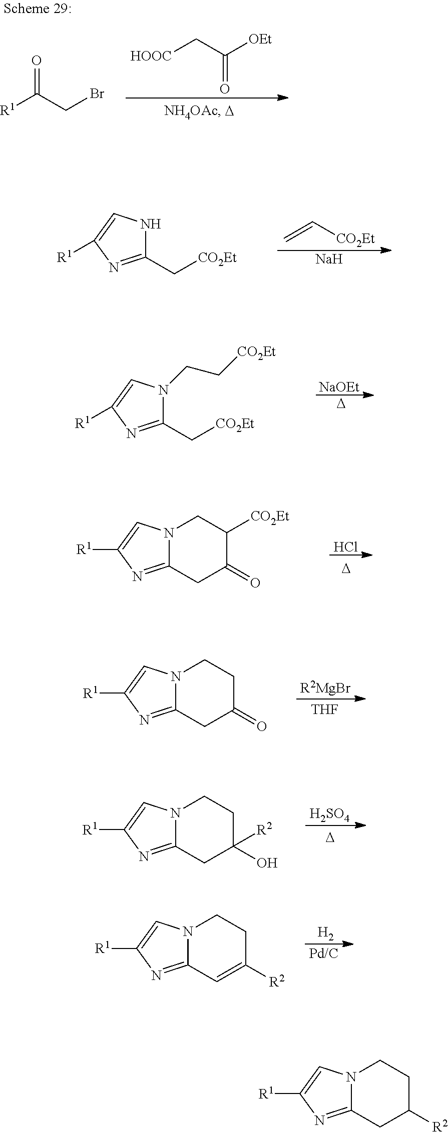

In one embodiment, a compound of formula (Ib) may be prepared following Scheme 30, using suitable starting material known in the art and/or available from a commercial source.

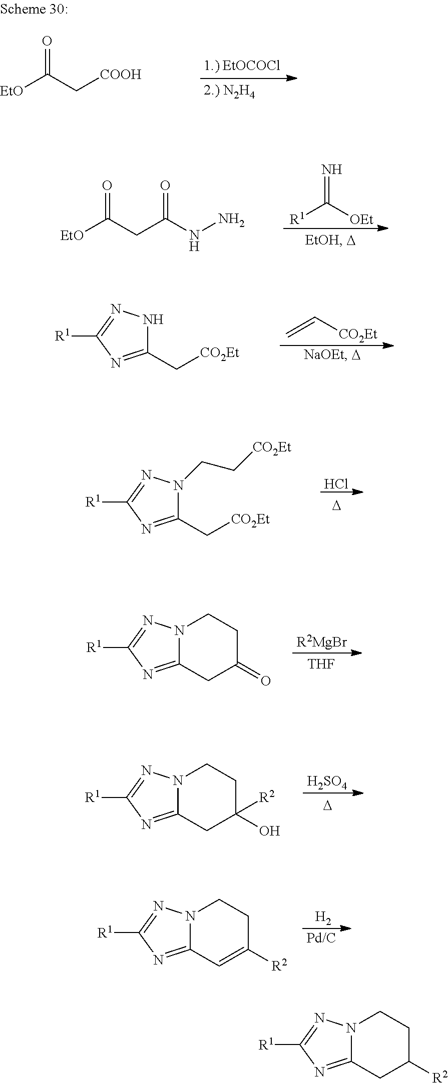

In another embodiment, a compound of formula (Ib) may be prepared following Scheme 31, using suitable starting material known in the art and/or available from a commercial source.

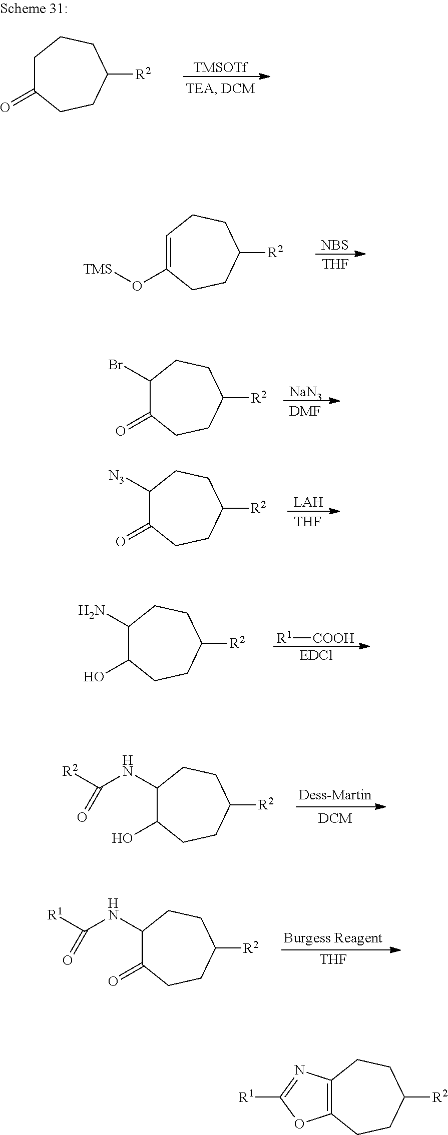

In another embodiment, a compound of formula (Ib) may be prepared following Scheme 32, using suitable starting material known in the art and/or available from a commercial source.

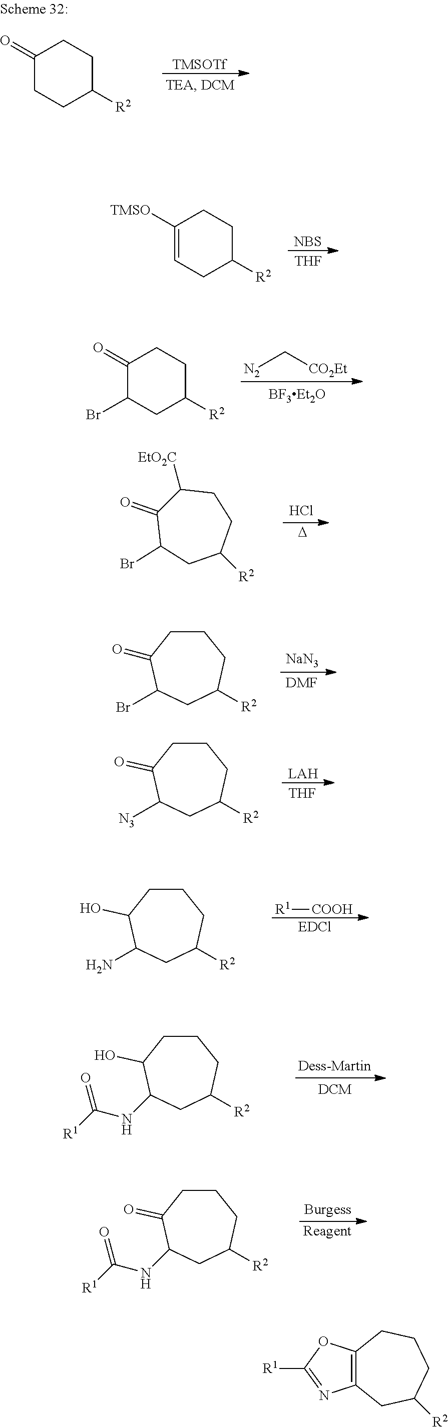

In another embodiment, a compound of formula (Ib) may be prepared following Scheme 33, using suitable starting material known in the art and/or available from a commercial source.

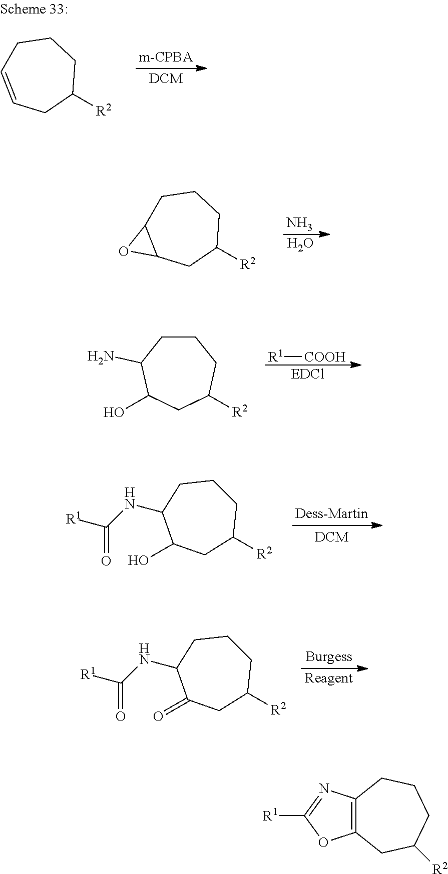

In certain embodiments, a compound of formula (I) is prepared as a mixture of two or more stereoisomers or diastereoisomers. In one embodiment, the stereoisomers or diastereoisomers are separated using techniques known to those skilled in the art, including but not limited to, chiral column chromatography and chiral resolution by forming a salt with a suitable chiral counterion. In certain embodiments, the compound of formula (I) is prepared following one or more stereoselective reaction(s). In some embodiment, the compound of formula (I) is prepared as a substantially pure stereoisomer.

### D. Methods of Treatment, Prevention, and/or Management

1. Binding to mGluR5Receptor

In various embodiments, provided herein is a method of binding a compound provided herein to a metabotropic glutamate receptor, such as mGluR5. The method comprises contacting mGluR with a compound provided herein.

In one embodiments, provided herein is a method of modulating the activity of mGluR5 via the binding of an mGluR5 ligand to mGluR5. The method comprises contacting mGluR5 with a compound provided herein. In one embodiment, the ligand is L-glutamate. In another embodiment, the ligand is a drug molecule or another small molecule known to have binding affinity to mGluR5. In another embodiment, the mGluR5 ligand is a radioactively labeled compound, known to bind to mGluR5. In other embodiments, binding to metabotropic glutamate receptor may be assessed using PET imaging known in the art, e.g. utilizing appropriate PET ligands. In some embodiments, the ligand is an allosteric modulator, antagonist, or inverse agonist of mGluR5.

2. Modulation of mGluR5 Receptor Activity

In various embodiments, provided herein is a method of modulating (e.g., inhibiting or augmenting) the activity of a metabotropic glutamate receptor, such as mGluR5. The method comprises contacting the receptor, such as mGluR5, with a compound provided herein, in vitro or in vivo. In one embodiment, mGluR5 is contacted with a compound provided herein by administering to a subject a therapeutically effective amount of the compound provided herein, or a pharmaceutically acceptable salt or solvate thereof. The subject may be a human.

In one embodiment, the compound provided herein inhibits or reduces the activity of metabotropic glutamate receptor, such as mGluR5. Inhibition of mGluR5 activity may be measured using assays known in the art. In some embodiments, the activity of mGluR5 is inhibited or reduced by about 1%, about 5%, about 10%, about 20%, about 30%, about 40%, about 50%, about 60%, about 70%, about 80%, about 90%, about 95%, about 99% or more, as compared with the activity obtained without contacting with the compounds provided herein. In one embodiment, the inhibition or reduction of receptor activity is dose-dependent. Exemplary assay methods include, but are not limited to, in vitro functional assays as described herein elsewhere. In one embodiment, the functional assay utilizes an appropriate cell-line expressing the desired metabotropic glutamate receptor, such as mGluR5. In other embodiments, the functional assay utilizes synaptosomes isolated from brain tissue of an appropriate organism. In other embodiments, inhibition of metabotropic glutamate receptor activity may be assessed using receptor binding experiments known in the art, e.g. utilizing appropriate membrane preparations. In one embodiment, the assay involves treatment of a test subject (e.g. a mice or a rat) with a compound provided herein as well as a reference compound, followed by isolation of brain tissue and ex vivo analysis of receptor occupancy.

In certain embodiments, provided herein are methods of inhibiting or reducing the activity of a metabotropic glutamate receptor, such as mGluR5, in a subject (e.g., human) comprising administering to the subject an effective amount of a compound provided herein. In some embodiments, the activity of mGluR5 is inhibited or reduced by about 1%, about 5%, about 10%, about 20%, about 30%, about 40%, about 50%, about 60%, about 70%, about 80%, about 90%, about 95%, about 99% or more, when measured using an assay known in the art.

In one embodiment, provided herein is a method of inhibiting or reducing the activity of a metabotropic glutamate receptor, such as mGluR5, by a metabotropic glutamate receptor ligand. In one embodiment, the method comprises contacting mGluR5 receptor with an antagonist, an inverse agonist, or an allosteric modulator of the mGluR5 receptor. In another embodiment, an antagonist, an inverse agonist, or an allosteric modulator of the mGluR5 receptor is a compound provided herein.

3. Treatment, Prevention, and/or Management of mGluR5 Receptor Related Disorders

In some embodiments, provided herein is a method of treating, preventing, and/or managing a disorder related to mGluR5, such as a neurological, psychiatric, or neuromuscular disorder. Without being limited by a particular theory, the treatment, prevention, and/or management is done by inhibiting or reducing the activity of mGluR5 using a composition or a compound provided herein. In one embodiment, provided herein is the use of a compound or a composition provided herein in the manufacture of a medicament for the treatment, prevention, and/or management of a disorder related to mGluR5, such as a neurological, psychiatric, or neuromuscular disorder provided herein. In one embodiment, provided herein is a compound or a composition for use in the treatment, prevention, and/or management of a disorder related to mGluR5, such as a neurological, psychiatric, or neuromuscular disorder provided herein.

In one embodiment, the method comprises administering to a subject (e.g., human) a therapeutically or prophylactically effective amount of a composition or a compound provided herein. In one embodiment, the subject is a human. In another embodiment, the compound provided herein inhibits the activity of mGluR5. In certain embodiments, the compounds provided herein are allosteric modulators of mGluR5. In other embodiments, the compounds provided herein are antagonists of mGluR5. In certain embodiments, the compounds provided herein are selective for mGluR5 over other CNS-related targets. In one embodiment, the compounds provided herein are highly brain penetrable in animals, such as rodents, and human. In some embodiments, inhibition of mGluR5 activity may be assessed by functional assays as described herein elsewhere. In certain embodiments, the efficacious concentration of the compounds provided herein is less than 10 nM, less than 100 nM, less than 1 μM, less than 10 μM, less than 100 μM, or less than 1 mM. In other embodiments, compound's activity may be assessed in various art-recognized animal models.

In some embodiments, provided herein is a method of treating, preventing, and/or managing migraine, comprising administering to a subject an effective amount of a compound provided herein. For example, without being limited by a particular theory, mGluR5 modulators may be effective in the treatment and prevention of migraine in human, and may have comparable efficacy to triptans in treating migraine. See, e.g., Jaeschke, G., et al., Expert Opin. Ther. Pat. 2008, 18, 123.

In some embodiments, provided herein is a method of treating, preventing, and/or managing a disorder related to anxiety (e.g., general anxiety disorder), comprising administering to a subject an effective amount of a compound provided herein. For example, without being limited by a particular theory, mGluR5 modulators may be effective in treating anxiety, and efficacious in a variety of animal models including stress-induced hyperthermia and fear-potentiated startle test. See e.g., Pecknold, J. C., et al., J. Clin. Neuropharmacol. 1982, 2, 129; Cosford, N. D. P., et al., J. Med. Chem. 2003, 46, 204.

In some embodiment, provided herein is a method of treating, preventing, and/or managing depression and other affective disorders, comprising administering to a subject an effective amount of a compound provided herein. For example, without being limited by a particular theory, mGluR5 modulators may be effective in treating depression, and efficacious in a variety of animal models for depression. See, e.g., Jaeschke, G., et al., Expert Opin. Ther. Pat. 2008, 18, 123.

In some embodiment, provided herein is a method of treating, preventing, and/or managing GERD, comprising administering to a subject an effective amount of a compound provided herein. For example, without being limited by a particular theory, mGluR5 modulators may be effective in treating GERD in human. See, e.g., Jaeschke, G., et al., Expert Opin. Ther. Pat. 2008, 18, 123; Bolea C., et al., WO 2004/78728 A1.

In some embodiment, provided herein is a method of treating, preventing, and/or managing a neurodegenerative disease, including but not limited to, Parkinson's disease, levodopa-induced dyskinesia, Huntington's disease, Alzheimer's disease, and amyotropic lateral sclerosis, comprising administering to a subject an effective amount of a compound provided herein. For example, without being limited by a particular theory, mGluR5 modulators may be effective in treating Parkinson's disease, and efficacious in a variety of animal models for Parkinson's disease. See, e.g., Jaeschke, G., et al., Expert Opin. Ther. Pat. 2008, 18, 123; Glatthar R., et al., WO2006/89700 A1.

In some embodiment, provided herein is a method of treating, preventing, and/or managing pain, including but not limited to, inflammatory pain, neuropathic pain, postoperative pain, acute thermal hyperalgesia, mechanical allodynia, visceral pain, and chronic pain, comprising administering to a subject an effective amount of a compound provided herein. See e.g., Jaeschke, G., et al., Expert Opin. Ther. Pat. 2008, 18, 123; Cosford, N. D. P., et al., WO 2003/51315 A2.

In some embodiment, provided herein is a method of treating, preventing, and/or managing post-traumatic stress disorder, comprising administering to a subject an effective amount of a compound provided herein. See e.g., Bach, P., et al., Expert Opin. Ther. Patents 2007, 17, 371.

In some embodiment, provided herein is a method of treating, preventing, and/or managing schizophrenia, comprising administering to a subject an effective amount of a compound provided herein. See e.g., Bach, P., et al., Expert Opin. Ther. Patents 2007, 17, 371; Jaeschke, G., et al., Expert Opin. Ther. Pat. 2008, 18, 123.

In some embodiment, provided herein is a method of treating, preventing, and/or managing fragile X syndrome, comprising administering to a subject an effective amount of a compound provided herein. See e.g., Jaeschke, G., et al., Expert Opin. Ther. Pat. 2008, 18, 123.

In some embodiment, provided herein is a method of treating, preventing, and/or managing substance abuse/addiction, including but not limited to the abuse/addiction of cocaine, morphine, opioid, nicotine, and alcohol, comprising administering to a subject an effective amount of a compound provided herein. See e.g., Jaeschke, G., et al., Expert Opin. Ther. Pat. 2008, 18, 123.

In some embodiment, provided herein is a method of treating, preventing, and/or managing epilepsy, comprising administering to a subject an effective amount of a compound provided herein. See e.g., Jaeschke, G., et al., Expert Opin. Ther. Pat. 2008, 18, 123.

In other embodiments, provided herein is a method of treating, preventing, and/or managing a neurological disorder as defined herein elsewhere, comprising administering to a subject an effective amount of a compound provided herein.

In other embodiments, provided herein is a method of treating, preventing, and/or managing a lower urinary tract disorder as defined herein elsewhere, comprising administering to a subject an effective amount of a compound provided herein.

In other embodiments, provided herein is a method of treating, preventing, and/or managing cancer, including but not limited to, oral cancer and glioneuronal cancer, comprising administering to a subject an effective amount of a compound provided herein.

In some embodiments, the compounds provided herein are active in at least one model, which can be used to measure the activity of the compounds and estimate their efficacy in treating a disorder related to mGluR5. For example, when the model is for depression (e.g., mean immobility), the compounds are active when they inhibit mean immobility of a test subject by about 5%, about 10%, about 20%, about 30%, about 40%, about 50%, about 60%, about 70%, about 80%, about 90%, about 95%, about 99%, or more, when compared to vehicle. In some embodiments, the compounds provided herein produce a similar disparity in measured endpoint between treated animals and animals administered vehicle.

In other embodiments, provided herein is a method of effecting a therapeutic effect as described herein elsewhere. The method comprises administering to a subject (e.g., a mammal) a therapeutically effective amount of a compound or a composition provided herein. The particular therapeutic effects may be measured using any model system known in the art or described herein, such as those involving an animal model of a disease.

In some embodiments, the disorder related to mGluR5 is migraine, anxiety (e.g., general anxiety disorder, social anxiety disorder, panic disorder, and dental phobia), depression (e.g., major depressive disorder, bipolar disorder, unipolar disorder, dysthymia and seasonal affective disorder), pain (e.g., inflammatory pain, neuropathic pain, postoperative pain, acute thermal hyperalgesia, mechanical allodynia, visceral pain, and chronic pain), neurodegenerative disease (e.g., Alzheimer's disease, Parkinson's disease, levodopa-induced dyskinesia, Huntington's disease, and amyotropic lateral sclerosis), epilepsy, seizure, psychosis, schizophrenia, substance abuse/addiction (e.g., cocaine, nicotine, morphine, opioid, or alcohol abuse/addiction), bulimia, anorexia, obsessive compulsive disorder, aggression, post-traumatic stress disorder, autism, fragile X syndrome, excessive tactile sensitivity, sensory hyper-excitability, attention deficit hyperactivity disorder, bipolar disorder, mood disorder, cognitive disorder, mental retardation, Down syndrome, memory deficit, dementia, GERD, acid reflux, irritable bowel syndrome, lower urinary tract disorder, overactive bladder, urinary incontinence, oral cancer, glioneuronal cancer, asthma, chronic pharyngitis, lung disease, dyspepsia, stroke, head trauma, anoxic and ischemic injuries. In another embodiment, the compounds provided herein are useful to treat, prevent, and/or manage two or more conditions/disorders, which are co-morbid, such as migraine and anxiety or migraine and depression.

Neurological disorders include cerebral function disorders, including without limitation, senile dementia, Alzheimer's type dementia, cognition, memory loss, amnesia/amnestic syndrome, epilepsy, disturbances of consciousness, coma, lowering of attention, speech disorders, Lennox syndrome, autism, and hyperkinetic syndrome.

Neuropathic pain includes without limitation post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

Other exemplary diseases and conditions that may be treated, prevented, and/or managed using the methods, compounds, and/or compositions provided herein include, but are not limited to: obesity; migraine or migraine headache; urinary incontinence, including without limitation involuntary voiding of urine, dribbling or leakage of urine, stress urinary incontinence (SUI), urge incontinence, urinary exertional incontinence, reflex incontinence, passive incontinence, and overflow incontinence; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In one embodiment, the neurological disorder is cognitive impairment. In another embodiment, the neurological disorder is mood disorders. In another embodiment, the neurological disorder is movement disorders. In another embodiment, the neurological disorder is schizophrenia. In another embodiment, the neurological disorder is attention disorders. In another embodiment, the neurological disorder is anxiety disorder. In another embodiment, the neurological disorder is seizure. In another embodiment, the neurological disorder is epilepsy. In another embodiment, the neurological disorder is vertigo. In another embodiment, the neurological disorder is pain. In another embodiment, the neurological disorder is neuropathic pain. In another embodiment, the neuropathic pain is diabetic neuropathy.

In one embodiment, the neurological disorder is a neurodegenerative disease. In one embodiment, the neurodegenerative disease is Parkinson's disease. In another embodiment, the neurodegenerative disorder is levodopa-induced dyskinesia. In another embodiment, the neurodegenerative disorder is Alzheimer's disease. In another embodiment, the neurodegenerative disorder is Huntington's disease.

In one embodiment, the compounds described herein treat, prevent, and/or manage a central nervous disorder, without causing addiction to said compounds.

Any suitable route of administration can be employed for providing the patient with a therapeutically or prophylactically effective dose of an active ingredient. For example, oral, mucosal (e.g., nasal, sublingual, buccal, rectal, vaginal), parenteral (e.g., intravenous, intramuscular), transdermal, and subcutaneous routes can be employed. Exemplary routes of administration include oral, transdermal, and mucosal. Suitable dosage forms for such routes include, but are not limited to, transdermal patches, ophthalmic solutions, sprays, and aerosols. Transdermal compositions can also take the form of creams, lotions, and/or emulsions, which can be included in an appropriate adhesive for application to the skin or can be included in a transdermal patch of the matrix or reservoir type as are conventional in the art for this purpose. An exemplary transdermal dosage form is a “reservoir type” or “matrix type” patch, which is applied to the skin and worn for a specific period of time to permit the penetration of a desired amount of active ingredient. The patch can be replaced with a fresh patch when necessary to provide constant administration of the active ingredient to the patient.

The amount to be administered to a patient to treat, prevent, and/or manage the disorders described herein will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount required. For example, the physician or veterinarian could start doses of the compounds employed at levels lower than that required in order to achieve the desired therapeutic effect and gradually increase the dosage until the desired effect is achieved.

In general, a suitable daily dose of a compound provided herein will be that amount of the compound which is the lowest dose effective to produce a therapeutic or prophylactic effect. Such an effective dose will generally depend upon the factors described above. Generally, oral, intravenous, intracerebroventricular, and subcutaneous doses of the compounds provided herein for a patient will range from about 0.005 mg per kilogram to about 5 mg per kilogram of body weight per day. In one embodiment, the oral dose of a compound provided herein will range from about 10 mg to about 300 mg per day. In another embodiment, the oral dose of a compound provided herein will range from about 20 mg to about 250 mg per day. In another embodiment, the oral dose of a compound provided herein will range from about 100 mg to about 300 mg per day. In another embodiment, the oral dose of a compound provided herein will range from about 10 mg to about 100 mg per day. In another embodiment, the oral dose of a compound provided herein will range from about 25 mg to about 50 mg per day. In another embodiment, the oral dose of a compound provided herein will range from about 50 mg to about 200 mg per day. Each of the above-recited dosage ranges may be formulated as a single or multiple unit dosage formulations.

In some embodiments, the compounds disclosed herein may be used in combination with one or more second active agents to treat, prevent, and/or manage disorders described herein.

4. Pharmaceutical Compositions and Dosage Forms

Pharmaceutical compositions can be used in the preparation of individual, single unit dosage forms. Pharmaceutical compositions and dosage forms provided herein comprise a compound provided herein, or a pharmaceutically acceptable salt, solvate, stereoisomer, clathrate, or prodrug thereof. Pharmaceutical compositions and dosage forms can further comprise one or more excipients.

Pharmaceutical compositions and dosage forms provided herein can also comprise one or more additional active ingredients. Examples of optional second, or additional, active ingredients are also disclosed herein.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th Ed., Mack Publishing, Easton Pa. (1990).

In one embodiment, pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Also provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are, in one embodiment, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In one embodiment, dosage forms comprise a compound provided herein in an amount of from about 0.10 to about 500 mg. In other embodiments, dosage forms comprise a compound provided herein in an amount of about 0.1, 1, 2, 5, 7.5, 10, 12.5, 15, 17.5, 20, 25, 50, 100, 150, 200, 250, 300, 350, 400, 450, or 500 mg.

In other embodiments, dosage forms comprise the second active ingredient in an amount of 1 to about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

4.1 Oral Dosage Forms

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's The Science and Practice of Pharmacy, 21st Ed., Lippincott Williams & Wilkins (2005).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In one embodiment, a solid oral dosage form comprises a compound provided herein, and optional excipients, such as anhydrous lactose, microcrystalline cellulose, polyvinylpyrrolidone, stearic acid, colloidal anhydrous silica, and gelatin.

4.2 Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In one embodiment, provided are single unit dosage forms suitable for oral administration such as, but not limited to, tablets, capsules, gelcaps, and caplets that are adapted for controlled-release.

In one embodiment, controlled-release pharmaceutical products improve drug therapy over that achieved by their non-controlled counterparts. In another embodiment, the use of a controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

In another embodiment, the controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In one embodiment, in order to maintain a constant level of drug in the body, the drug can be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.3 Parenteral Dosage Forms

Parenteral dosage forms can be administered to patients by various routes including, but not limited to, subcutaneous, intravenous (including bolus injection), intramuscular, and intraarterial. In some embodiments, administration of a parenteral dosage form bypasses patients' natural defenses against contaminants, and thus, in these embodiments, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated herein by reference.

4.4 Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In one embodiment, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other embodiments, stearates can serve as a lipid vehicle for the formulation, as an emulsifying agent or surfactant, or as a delivery-enhancing or penetration-enhancing agent. In other embodiments, salts, solvates, prodrugs, clathrates, or stereoisomers of the active ingredients can be used to further adjust the properties of the resulting composition.

5. Kits

In one embodiment, active ingredients provided herein are not administered to a patient at the same time or by the same route of administration. In another embodiment, provided are kits which can simplify the administration of appropriate amounts of active ingredients.

In one embodiment, a kit comprises a dosage form of a compound provided herein. Kits can further comprise one or more second active ingredients as described herein, or a pharmacologically active mutant or derivative thereof, or a combination thereof.

In other embodiments, kits can further comprise devices that are used to administer the active ingredients. Examples of such devices include, but are not limited to, syringes, drip bags, patches, and inhalers.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### V. EXAMPLES

Certain embodiments are illustrated by the following non-limiting examples.

A. Synthesis of Compounds

In the examples below, unless otherwise indicated, all temperatures are set forth in degrees Celsius and all parts and percentages are by weight. Reagents may be purchased from commercial suppliers, such as Sigma-Aldrich Chemical Company, and may be used without further purification unless otherwise indicated. Reagents may also be prepared following standard literature procedures known to those skilled in the art. Solvents may be purchased from Aldrich in Sure-Seal bottles and used as received. All solvents may be purified using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g., Still et al., J. Org. Chem., 43: 2923 (1978)) was performed using silica gel 60 (Merck Art 9385) or various MPLC systems.

The compound structures in the examples below were confirmed by one or more of the following methods: proton magnetic resonance spectroscopy, mass spectroscopy, and melting point. Proton magnetic resonance (¹H NMR) spectra were determined using an NMR spectrometer operating at 400 MHz field strength. Chemical shifts are reported in the form of delta (6) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz). Mass spectra (MS) data were obtained using a mass spectrometer with APCI or ESI ionization.

As used herein, and unless otherwise specified, “Me” means methyl, “Et” means ethyl, “Ac” means acetyl, “BINAP” means 2,2′-bis(diphenylphosphino)-1,1′-binaphthyl, “Dess-Martin reagent” means 1,1,1-tris(acetyloxy)-1,1-dihydro-1,2-benziodoxol-3-(1H)-one, “DCM” means dichloromethane, “DIEA” means diisopropylethylamine, “DMF” means dimethylformamide, “EDCI” means N-(3-dimethylaminopropyl)-N′-ethylcarbodiimide hydrochloride, “EtOAc” means ethyl acetate, “EtOH” means ethanol, “HOBt” means hydroxybenzotriazole, “m-CPBA” means 3-chloro-perbenzoic acid, “MeCN” means acetonitrile, “MeOH” means methanol, “PE” means petroleum ether, “RT” or “rt” means room temperature, “t-BuOH” means tert-butanol, “t-BuONa” means sodium tert-butoxide, “TBDMSCl” means tert-butyldimethylsilyl chloride, “TEA” means triethylamine, “THF” means tetrahydrofuran, “TMSI” means iodotrimethylsilane, “Xantphos” means 4,5-bis(diphenylphosphino)-9,9-dimethylxanthene, “h” or “hr” means hour(s), “min” means minute(s), “cat.” means catalytic, “aq” means aqueous, “TMSI” means trimethylsilyl iodide, and “TFA” means trifluoroacetic acid.

### 1. Compound 1: 3-(2-(Pyridin-2-yl)-6,7-dihydrooxazolo[5,4-c]pyridin-5(4H)-yl)benzonitrile

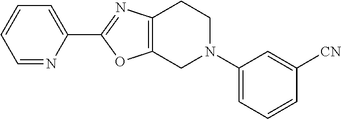

To a solution of 2-(pyridin-2-yl)-4,5,6,7-tetrahydrooxazolo[5,4-c]pyridine (I-1.4) (100 mg, 0.5 mmol) in t-BuOH (2 mL) was added 3-bromobenzonitrile (102 mg, 0.6 mmol), t-BuONa (100 mg, 1 mmol), Pd₂ dba₃ (5 mg, cat.), BINAP (5 mg, cat.). The mixture was heated to 110° C. via microwave and stirred for 1 h. The reaction mixture was cooled, dissolved in MeOH, and filtered. The filtrate was concentrated and the residue was purified by preparative HPLC to afford 3-(2-(pyridin-2-yl)-6,7-dihydro-oxazolo[5,4-c]pyridine-5(4H)-yl)benzonitrile (10 mg, 7%) as a yellow solid. ¹H NMR (400 MHz, CDCl₃): δ 8.71 (s, 1H), 8.10 (d, 1H), 7.85 (t, 1H), 7.40 (t, 1H), 7.30 (t, 1H), 7.07 (m, 3H), 4.40 (s, 2H), 3.68 (t, 2H), 2.82 (t, 2H); LC/MS: m/e=303 (M+H)⁺.

### 1.1 Benzyl 3-hydroxy-4-(picolinamido)piperidine-1-carboxylate (I-1.1)

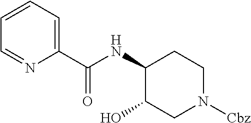

To a solution of picolinic acid (2.46 g, 20 mmol) and TEA (4.04 g, 40 mmol) in DCM (20 mL) was added EDCI (7.64 g, 40 mmol) and HOBt (5.4 g, 40 mmol). (±)-trans-Benzyl 4-amino-3-hydroxypiperidine-1-carboxylate (prepared according to the procedure in Hall, S. E., et al., WO1994/20062) (5 g, 20 mmol) in DCM (10 mL) was added and the mixture was stirred overnight at room temperature. The mixture was charged to a separatory funnel and washed with water. The organic phase was then washed with aq. NaHCO₃, 1N HCl, and brine. The combined organic phases were dried over anhydrous Na₂SO₄ and concentrated to afford benzyl 3-hydroxy-4-(picolinamido)-piperidine-1-carboxylate (5.8 g, 82%) as a white solid. ¹H NMR (400 MHz, DMSO-d₆): δ 8.60 (m, 2H) 8.00 (m, 2H), 7.58 (t, 1H), 7.25-7.40 (m, 5H), 5.07 (s, 2H), 4.06 (m, 1H), 3.91 (d, 1H), 3.80 (m, 1H), 3.59 (m, 1H), 2.90 (m, 1H), 2.70 (m, 1H), 1.85 (d, 1H), 1.51 (m, 1H).

### 1.2 Benzyl 3-oxo-4-(picolinamido)piperidine-1-carboxylate (I-1.2)

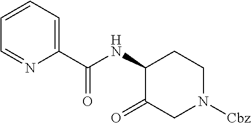

To a solution of benzyl 3-hydroxy-4-(picolinamido)piperidine-1-carboxylate (5 g, 14 mmol) in DCM was added Dess-Martin reagent (20 g, 43 mmol). The mixture was stirred overnight at room temperature. Then the mixture was washed with 0.5 N NaOH solution and extracted with DCM. The organic phase was dried over anhydrous Na₂SO₄ and concentrated to afford crude benzyl 3-oxo-4-(picolinamido)piperidine-1-carboxylate (4.9 g) as a yellow solid. The crude product was used next step without purification.

### 1.3 Benzyl 2-(pyridin-2-yl)-6,7-dihydrooxazolo[5,4-c]pyridine-5(4H)-carboxylate (I-1.3)

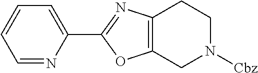

To a solution of POCl₃ (8.6 g, 56 mmol) in dioxane (80 mL), benzyl 3-oxo-4-(picolinamido)piperidine-1-carboxylate (4.9 g, 14 mmol) in dioxane (80 mL) was added. The reaction mixture was heated at reflux for 3 h with stirring. The mixture was poured into water and extracted with EtOAc. The organic phase was dried over anhydrous Na₂SO₄. The solvent was concentrated and the residue was purified by column on silica gel (PE:EtOAc=1:1) to afford benzyl 2-(pyridin-2-yl)-6,7-dihydrooxazolo[5,4-c]-pyridine-5(4H)-carboxylate as an off-red solid (1.35 g, 29%). ¹H NMR (400 MHz, CDCl₃): δ 8.07 (t, 1H) 8.00 (d, 1H), 7.75 (t, 1H), 7.20-7.38 (m, 6H), 5.11 (s, 2H), 4.62 (s, 2H), 3.76 (t, 2H), 2.68 (t, 2H).

### 1.4 2-(Pyridin-2-yl)-4,5,6,7-tetrahydrooxazolo[5,4-c]pyridine (I-1.4)

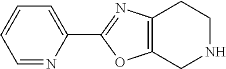

To a solution of benzyl 2-(pyridin-2-yl)-6,7-dihydrooxazolo[5,4-c]pyridine-5(4H)-carboxylate (1.35 g, 4 mmol) in MeOH was added Pd(OH)₂/C (500 mg). The mixture was stirred for 3 h at room temperature under H₂ atmosphere. The mixture was then filtered and concentrated to afford 2-(pyridin-2-yl)-4,5,6,7-tetrahydrooxazolo[5,4-c]pyridine (700 mg, 86%) as a yellow solid. ¹H NMR (400 MHz, DMSO-d₆): δ 8.65 (d, 1H), 8.02 (d, 1H), 7.93 (t, 1H), 7.49 (t, 1H), 3.87 (s, 1H), 3.75 (m, 1H), 3.57 (m, 1H), 2.96 (m, 1H), 2.71 (m, 1H), 2.60 (m, 1H), 2.51 (m, 1H).

### 2. Compound 2: 2,5-Di(pyridin-2-yl)-4,5,6,7-tetrahydro-oxazolo[5,4-c]pyridine

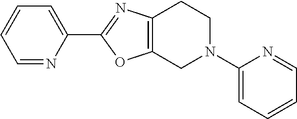

To a solution of 2-(pyridin-2-yl)-4,5,6,7-tetrahydrooxazolo[5,4-c]pyridine (I-1.4) (100 mg, 0.5 mmol) in t-BuOH was added 2-bromopyridine (120 mg, 0.6 mmol), t-BuONa (50 mg, 0.6 mmol), Pd₂ dba₃ (5 mg, cat.), BINAP (5 mg, cat.). The mixture was heated to 110° C. by microwave and stirred for 1 h. The mixture was cooled, dissolved in MeOH and filtered. The filtrate was concentrated and the residue was purified by preparative HPLC to afford 2,5-di(pyridin-2-yl)-4,5,6,7-tetrahydrooxazolo[5,4-c]-pyridine (76 mg, 55%) as a yellow liquid. ¹H NMR (400 MHz, CDCl₃): δ 8.71 (d, 1H); 8.20 (d, 1H), 8.10 (d, 1H), 7.90 (dd, 2H), 7.45 (t, 1H), 7.02 (d, 1H), 6.93 (t, 1H), 4.81 (s, 2H), 4.10 (t, 2H), 2.97 (t, 2H); LC/MS: m/e=279 (M+H)⁺.

### 3. Compound 3: 2-(2-(Pyridin-2-yl)-6,7-dihydrooxazolo[5,4-c]pyridin-5(4H)-yl)nicotinonitrile

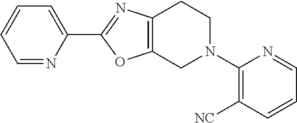

To a solution of 2-(pyridin-2-yl)-4,5,6,7-tetrahydrooxazolo[5,4-c]pyridine (I-1.4) (100 mg, 0.5 mmol) in DMF (5 mL) was added 2-chloronicotinonitrile (140 mg, 1 mmol) and DIEA (140 mg, 1 mmol). The mixture was heated to 100° C. and stirred for 6 h. The mixture was cooled to room temperature, poured into water and extracted with EtOAc. The organic phase was concentrated and the residue was purified by preparative TLC to afford 2-(2-(pyridin-2-yl)-6,7-dihydrooxazolo[5,4-c]pyridin-5(4H)-yl)nicotinonitrile (20 mg, 13%) as a yellow solid. ¹H NMR (400 MHz, CDCl₃): δ 8.65 (s, 1H); 8.30 (dd, 1H), 8.07 (d, 1H), 7.78 (m, 2H), 7.30 (t, 1H), 6.75 (q, 1H), 4.81 (s, 2H), 4.03 (t, 2H), 2.92 (t, 2H); LC/MS: m/e=304 (M+H)⁺.

### 4. Compound 4: 3-Fluoro-5-(2-(pyridin-2-yl)-6,7-dihydrooxazolo[5,4-c]pyridin-5(4H)-yl)benzonitrile

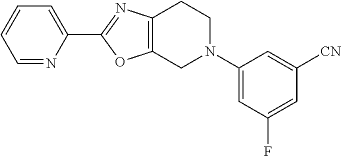

To a solution of 2-(pyridin-2-yl)-4,5,6,7-tetrahydrooxazolo[5,4-c]pyridine (20 mg, 0.1 mmol) in toluene was added 3-bromo-5-fluorobenzonitrile (30 mg, 0.15 mmol), Cs₂CO₃ (65 mg, 0.2 mmol), Pd(OAc)₂ (1 mg, cat.), and Xantphos (2 mg, cat.). The mixture was heated to 100° C. and stirred overnight. The mixture was cooled, dissolved in MeOH, and filtered. The filtrate was concentrated and the residue was purified by preparative TLC to afford 3-fluoro-5-(2-(pyridin-2-yl)-6,7-dihydrooxazolo-[5,4-c]pyridin-5(4H)-yl)benzonitrile (10 mg, 31%) as a yellow solid. ¹H NMR (400 MHz, CDCl₃): δ 8.68 (d, 1H); 8.05 (d, 1H), 7.77 (t, 1H), 7.32 (t, 1H), 6.90 (s, 1H), 6.75 (m, 2H), 4.40 (s, 2H), 3.68 (t, 2H), 2.80 (t, 2H); LC/MS: m/e=321 (M+H)⁺.

### 5. Compound 5: 2-(Pyridin-2-yl)-5-(pyridin-3-yl)-4,5,6,7-tetrahydrooxazolo[5,4-c]pyridine

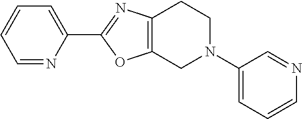

The title compound was prepared via the procedure used for Compound 1, using 3-bromopyridine instead of 3-bromobenzonitrile. The procedure afforded 2-(pyridin-2-yl)-5-(pyridin-3-yl)-4,5,6,7-tetrahydrooxazolo[5,4-c]pyridine (8 mg, 5%) as a yellow solid. ¹H NMR (400 MHz, CDCl₃): δ 8.70 (d, 1H), 8.61 (d, 1H), 8.12 (d, 1H), 8.07 (d, 1H), 7.85 (t, 1H), 7.65 (m, 2H), 7.38 (t, 1H), 4.55 (s, 2H), 3.82 (t, 2H), 2.90 (t, 2H); LC/MS: m/e=279 (M+H)⁺.

### 6. Compound 6: 6-(2-(Pyridin-2-yl)-6,7-dihydrooxazolo[5,4-c]pyridin-5(4H)-yl)picolinonitrile

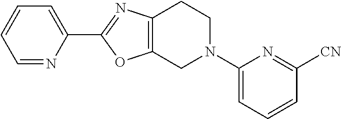

The title compound was prepared via the procedure used for Compound 4, using 6-bromopicolinonitrile instead of 3-bromo-5-fluorobenzonitrile. Preparative HPLC afforded 6-(2-(pyridin-2-yl)-6,7-dihydrooxazolo[5,4-c]pyridine-5(4H)-yl)-picolinonitrile (10 mg, 13%). ¹H NMR (400 MHz, CDCl₃): δ 8.71-8.74 (m, 1H), 8.08-8.12 (m, 1H), 7.73-7.92 (m, 1H), 7.34-7.60 (m, 2H), 7.04 (d, 1H), 6.88 (d, 1H), 4.78 (s, 2H), 4.05 (t, 2H), 2.85 (t, 2H); LC/MS: m/e=304 (M+H)⁺.

### 7. Compound 7: 2-(2-(Pyridin-2-yl)-6,7-dihydrooxazolo[5,4-c]pyridin-5(4H)-yl)isonicotinonitrile

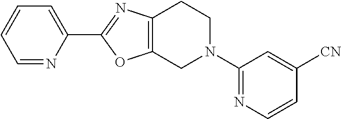

The title compound was prepared via the procedure used for Compound 4, using 2-bromo-isonicotinonitrile instead of 3-bromo-5-fluorobenzonitrile. Preparative HPLC afforded 2-(2-(pyridin-2-yl)-6,7-dihydrooxazolo[5,4-c]pyridin-5(4H)-yl)iso-nicotinonitrile (1.8 mg, 2.4%) as a yellow oil. ¹H NMR (400 MHz, CDCl₃): δ 8.87-8.70 (m, 1H), 8.27 (d, 1H), 8.09-8.07 (m, 1H), 7.80-7.76 (m, 1H), 7.34-7.41 (m, 2H), 4.48 (s, 2H), 3.72 (t, 2H), 2.85 (m, 2H); LC/MS: m/e=304 (M+H)⁺.

### 8. Compound 8: 5-(2-(Pyridin-2-yl)-6,7-dihydrooxazolo[5,4-c]pyridin-5(4H)-yl)nicotinonitrile

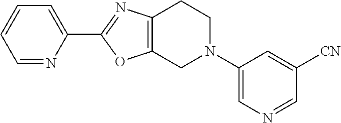

The title compound was prepared via the procedure used for Compound 4, using 5-bromo-nicotinonitrile instead of 3-bromo-5-fluorobenzonitrile. Preparative HPLC afforded 5-(2-(pyridin-2-yl)-6,7-dihydrooxazolo[5,4-c]pyridin-5(4H)-yl)nicotinonitrile (7 mg, 9.3%) as a yellow solid. ¹H NMR (400 MHz, CDCl₃): δ 8.67 (d, 1H), 8.48 (d, 1H), 8.27 (d, 1H), 7.85-7.80 (m, 1H), 7.39-7.24 (m, 1H), 6.90 (s, 1H), 6.80 (d, 1H), 4.79 (s, 2H), 3.90 (t, 2H), 2.85 (m, 2H); LC/MS: m/e=304 (M+H)⁺.

### 9. Compound 9: 4-(2-(Pyridin-2-yl)-6,7-dihydrooxazolo[5,4-c]pyridin-5(4H)-yl)picolinonitrile

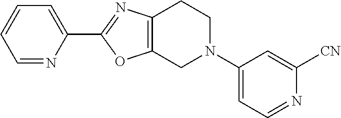

The title compound was prepared via the procedure used for Compound 4, using 4-bromopyridine-2-carbonitrile instead of 3-bromo-5-fluorobenzonitrile. Preparative TLC afforded 4-(2-(pyridin-2-yl)-6,7-dihydrooxazolo[5,4-c]pyridin-5(4H)-yl)picolinonitrile as a yellow solid (33 mg, 43.5%). ¹H NMR (400 MHz, CDCl₃): δ 8.67-8.65 (m, 1H), 8.30-8.27 (m, 1H), 8.05-8.01 (m, 1H), 7.77 (dt, 1H), 7.32 (ddd 1H), 7.07-7.05 (m, 1H), 6.80-6.77 (m, 1H), 4.50-4.49 (m, 2H), 3.83-3.76 (m, 2H), 2.86-2.82 (m, 2H); LC/MS: m/e=304 (M+H)⁺.

### 10. Compound 10: 3-Bromo-5-(2-(pyridin-2-yl)-6,7-dihydrooxazolo[5,4-c]pyridin-5(4H)-yl)benzonitrile

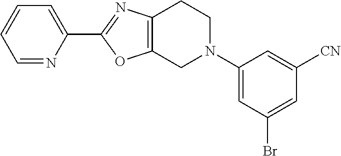

The title compound was prepared via the procedure used for Compound 4, using 3,5-dibromobenzonitrile instead of 3-bromo-5-fluorobenzonitrile. Preparative HPLC afforded 3-bromo-5-(2-(pyridin-2-yl)-6,7-dihydrooxazolo[5,4-c]pyridin-5(4H)-yl)benzonitrile (60 mg, 32%). ¹H NMR (400 MHz, CDCl₃): δ 8.72 (d, 1H), 8.10 (d, 1H), 7.81-7.88 (m, 1H), 7.38-7.42 (m, 1H), 7.20 (s, 1H), 7.19 (s, 1H), 6.97 (s, 1H), 4.38 (s, 2H), 3.65 (t, 2H), 2.82 (t, 2H); LC/MS: m/e=383 (M+H)⁺.

### 11. Compound 11: 3-Morpholino-5-(2-(pyridin-2-yl)-6,7-dihydrooxazolo[5,4-c]pyridin-5(4H)-yl)benzonitrile

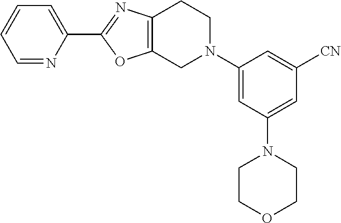

To a solution of 3-bromo-5-(2-(pyridin-2-yl)-6,7-dihydrooxazolo[5,4-c]pyridine-5(4H)-yl)benzonitrile (10) (30 mg, 78.7 mmol), morpholine (10.3 mg, 118 mmol), and t-BuONa (16 mg, 157.4 mmol) in toluene (2 mL) was added BINAP (4.89 mg, cat.) and Pd₂(dba)₃ (7 mg, cat.) under an argon atmosphere. The mixture was stirred at 100° C. overnight. The reaction was filtered and the filtrate was purified by preparative HPLC to afford 3-morpholino-5-(2-(pyridin-2-yl)-6,7-dihydrooxazolo[5,4-c]pyridin-5(4H)-yl)benzonitrile (5 mg, 16%). ¹H NMR (400 MHz, CDCl₃): δ 8.62-8.78 (m, 1H), 8.05-8.12 (m, 1H), 7.78-7.89 (m, 1H), 7.33-7.45 (m, 2H), 6.54-6.68 (m, 3H), 4.37-4.35 (m, 2H), 3.82-3.80 (m, 4H), 3.64-3.62 (m, 2H), 3.15-3.13 (m, 4H), 2.81-2.79 (m, 2H); LC/MS: m/e=388 (M+H)⁺.

### 12. Compound 12: 3-(2-(Pyridin-2-yl)-4,5,6,7-tetrahydrooxazolo[5,4-c]pyridine-5-carbonyl)benzonitrile

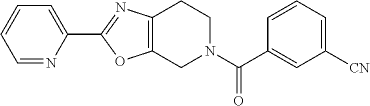

To a solution of 2-(pyridin-2-yl)-4,5,6,7-tetrahydrooxazolo[5,4-c]pyridine (I-1.4) (50 mg, 0.248 mmol) in DCM (5 mL) was added 3-cyanobenzoyl chloride (45 mg, 0.273 mmol) and TEA (38 mg, 0.372 mmol) in DCM. The mixture was stirred at room temperature for 1 h. The reaction was concentrated and purified by preparative TLC to afford 3-(2-(pyridin-2-yl)-4,5,6,7-tetrahydrooxazolo[5,4-c]pyridine-5-carbonyl)benzo-nitrile (13 mg, 14%). ¹H NMR (400 MHz, CDCl₃): δ 8.65 (s, 1H), 8.04 (d, 1H), 7.78-7.75 (m, 1H), 7.72-7.70 (m, 2H), 7.65 (d, 1H), 7.54 (t, 1H), 7.30 (dd, 1H), 4.85 (s, 2H), 3.64 (s, 2H), 2.74 (s, 2H); LC/MS: m/e=331 (M+H)⁺.

### 13. Compound 13: 3-(2-(Pyridin-2-yl)-6,7-dihydrooxazolo[5,4-c]pyridin-5(4H)-yl)methyl)benzonitrile

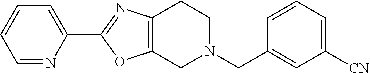

To a solution of 2-(pyridin-2-yl)-4,5,6,7-tetrahydrooxazolo[5,4-c]pyridine (I-1.4) (50 mg, 0.248 mmol) in dry DMF (5 mL) was added K₂CO₃ (69 mg, 0.496 mmol) and 3-(bromomethyl)benzonitrile (49 mg, 0.248 mmol). The mixture was stirred at room temperature overnight, and then concentrated. The residue was purified by preparative HPLC to afford 3-((2-(pyridin-2-yl)-6,7-dihydrooxazolo[5,4-c]pyridin-5(4H)-yl)methyl)benzonitrile (10 mg, 13%). ¹H NMR (400 MHz, CDCl₃): δ 8.65 (d, 1H), 8.40 (d, 1H), 7.83-7.79 (m, 1H), 7.74-7.67 (m, 3H), 7.54-7.51 (m, 1H), 7.39-7.37 (m, 1H), 4.25 (s, 2H), 4.20 (s, 2H), 3.42 (t, 2H), 2.98 (t, 2H); LC/MS: m/e=339 (M+H)⁺.

### 14. Compound 14: 5-(3-Fluoro-5-(pyridin-4-yl)phenyl)-2-(pyridin-2-yl)-4,5,6,7-tetrahydrooxazolo[5,4-c]pyridine

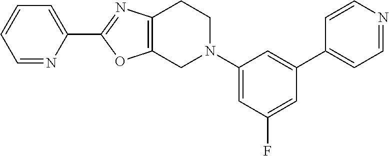

The title compound was prepared via the procedure used for Compound 4, using 4-(3-bromo-5-fluorophenyl)pyridine instead of 3-bromo-5-fluorobenzonitrile. Purification by preparative HPLC afforded 5-(3-fluoro-5-(pyridin-4-yl)phenyl)-2-(pyridin-2-yl)-4,5,6,7-tetrahydrooxazolo[5,4-c]pyridine (2 mg, 4%) as a yellow solid. ¹H NMR (400 MHz, CDCl₃): δ 8.74 (brs, 1H), 8.65 (d, 1H), 8.55-8.54 (m, 1H), 8.03 (d, 1H), 7.78-7.73 (m, 2H), 6.82 (s, 1H), 6.68 (d, 1H), 6.64-6.60 (m, 1H), 4.43 (s, 2H), 3.69 (t, 2H), 2.82 (t, 2H); LC/MS: m/e=373 (M+H)⁺.

### 14.1 4-(3-Bromo-5-fluorophenyl)pyridine (I-14.1)

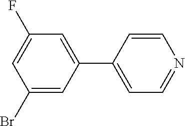

1,3-Dibromo-5-fluorobenzene (10.2 g, 40.7 mmol) was dissolved in 1,4-dioxane. Pyridine-4-ylboronic acid (5 g, 40.7 mmol), tetrakis(triphenylphosphine)-palladium(0) (462 mg, 0.41 mmol), and K₂CO₃ (11.22 g, 81 mmol) were added, and the mixture was heated at 90° C. for 4 h. The reaction was diluted with EtOAc, washed with aq. NaHCO₃ and dried and removed. Purification via column chromatography afforded 4-(3-bromo-5-fluorophenyl)pyridine (6 g, 59%). ¹H NMR (400 MHz, CDCl₃): δ 8.73 (d, 1H), 8.58 (d, 1H), 7.77 (dd, 1H), 7.45 (s, 1H), 7.34-7.31 (m, 1H), 7.22 (t, 1H), 7.18-7.15 (m, 1H).

### 15. Compound 15: 2-(5-(3-Cyanophenyl)-4,5,6,7-tetrahydrooxazolo[5,4-c]pyridin-2-yl)pyridine 1-oxide

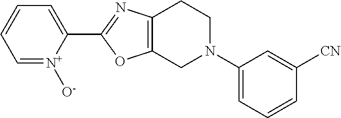

To a solution of 3-(2-(pyridin-2-yl)-6,7-dihydrooxazolo[5,4-c]pyridin-5(4H)-yl)benzonitrile (1) (54 mg, 0.178 mmol) in DCM (10 mL) was added m-CPBA (30 mg, 0.178 mmol), and the resulting solution was stirred at room temperature for 6 h. Solvent was removed under reduced pressure, and the residue was purified by preparative HPLC (3.7 mg, 6%). ¹H NMR (400 MHz, CDCl₃): δ 8.71 (s, 1H), 8.08 (d, 1H), 7.81-7.79 (m, 1H), 7.44-7.24 (m, 5H), 5.10 (s, 2H), 3.80 (s, 2H), 3.15 (s, 2H); LCMS: m/e=319 (M+H)⁺.

### 16. Compound 16: 5-(2-(Pyridin-2-yl)-6,7-dihydrooxazolo[5,4-c]pyridin-5(4H)-yl)isophthalonitrile

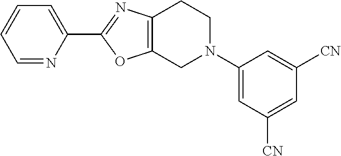

The title compound was prepared via the procedure used for Compound 4, using 5-bromoisophthalonitrile instead of 3-bromo-5-fluorobenzonitrile. Purification by HPLC afforded 5-(2-(pyridin-2-yl)-6,7-dihydrooxazolo[5,4-c]pyridin-5(4H)-yl)iso-phthalonitrile (10 mg, 9%) as a yellow solid. ¹H NMR (400 MHz, CDCl₃): δ 8.79-8.78 (m, 1H), 8.16 (d, 1H), 7.96-7.92 (m, 1H), 7.49 (t, 1H), 7.33 (brs, 5H), 4.50 (s, 2H), 3.82-3.77 (m, 2H), 2.99 (brs, 2H); LC/MS: m/e=328 (M+H)⁺.

### 17. Compound 17: 5-(3,5-Difluorophenyl)-2-(pyridin-2-yl)-4,5,6,7-tetrahydrooxazolo[5,4-c]pyridine

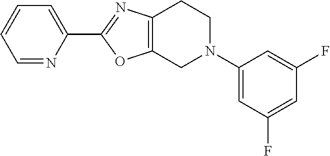

A mixture of 2-pyridin-2-yl-4,5,6,7-tetrahydrooxazolo[5,4-c]pyridine (I-1.4) (100 mg, 0.3 mmol), 1-bromo-3,5-difluorobenzene (115 mg, 0.6 mmol), t-BuONa (96 mg, 1 mmol), Xantphos (10 mg) and Pd(OAc)₂ (5 mg) in toluene (10 mL) was heated to reflux under nitrogen for 24 hours. The solid was removed by filtration and the reaction was purified by preparative TLC (61 mg, 64%). ¹H NMR (400 MHz, CDCl₃): δ 8.71 (d, 1H), 8.09 (d, 1H), 7.83 (d, 1H), 7.37 (d, 1H), 6.40 (d, 2H), 6.27 (t, 1H), 4.39 (s, 2H), 3.67 (t, 2H), 2.85 (t, 2H); LC/MS: m/e=314 (M+H)⁺.

### 18. Compound 18: 2-Phenyl-5-(pyrazin-2-yl)-4,5,6,7-tetrahydrooxazolo-[5,4-c]pyridine

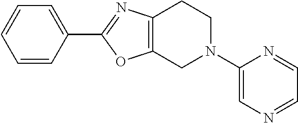

2-Phenyl-4,5,6,7-tetrahydro-oxazolo[5,4-c]pyridine hydrochloride (purchased from Anichem) (200 mg, 0.845 mmol), chloropyrazine (193 mg, 1.69 mmol) and DIEA (327 mg, 2.53 mmol) were combined in DMF (1.0 mL) and heated under microwaves at 160° C. for 1.5 h. The solvent was concentrated and removed under vacuum. The residue was then purified by silica gel column (EtOAc (10% MeOH)/hexanes), followed by recrystallization in ethanol to give a white solid (17 mg, 7%). ¹H NMR (400 MHz, CDCl₃): δ 8.23 (m, 1H), 8.10 (m, 1H), 8.01 (m, 2H), 7.89 (d, 1H), 7.45 (m, 3H), 4.61 (m, 2H), 4.13 (t, 2H), 2.96 (m, 2H); LC/MS: m/e=279 (M+H)⁺.

### 19. Compound 19: 2-(2-Phenyl-6,7-dihydrooxazolo[5,4-c]pyridin-5(4H)-yl)nicotinonitrile

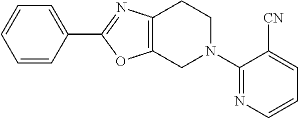

2-Phenyl-4,5,6,7-tetrahydro-oxazolo[5,4-c]pyridine hydrochloride (purchased from Anichem) (200 mg, 0.84 mmol), 2-chloro-3-pyridine-carbonitrile (234 mg, 1.69 mmol) and DIEA (327 mg, 2.53 mmol) were combined in DMF (1.0 mL) and heated under microwaves at 160° C. for 1.5 h. The solvent was concentrated and removed under vacuum. The residue was then purified by silica gel column (EtOAc (10% MeOH)/hexanes), followed by recrystallization in ethanol to give a white solid (54 mg, 21%). ¹H NMR (400 MHz, CDCl₃): δ 8.37 (dd, 1H), 8.02 (m, 2H), 7.80 (dd, 1H), 7.38-7.46 (m, 3H), 6.78 (dd, 1H), 4.73 (s, 2H), 4.10 (t, 2H), 3.09 (m, 2H); LC/MS: m/e=303 (M+H)⁺.

### 20. Compound 20: 2-Phenyl-5-(pyridin-2-yl)-4,5,6,7-tetrahydrooxazolo-[5,4-c]pyridine

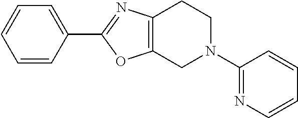

2-Phenyl-4,5,6,7-tetrahydro-oxazolo[5,4-c]pyridine hydrochloride (purchased from Anichem) (200 mg, 0.84 mmol), 2-bromopyridine (267 mg, 1.69 mmol) and DIEA (327 mg, 2.53 mmol) were combined in DMF (1.0 mL) and heated under microwaves at 160° C. for 1.5 h. The solvent was concentrated and removed under vacuum. The residue was then purified by silica gel column (EtOAc (10% MeOH)/hexanes) to give a pale yellow solid (49 mg, 21%). ¹H NMR (400 MHz, CDCl₃): δ 8.22 (m, 1H), 8.02 (m, 2H), 7.49-7.54 (m, 1H), 7.42-7.46 (m, 3H), 6.71 (d, 1H), 6.65 (m, 1H), 4.52 (s, 2H), 4.14 (t, 2H), 2.94 (m, 2H); LC/MS: m/e=278 (M+H)⁺.

### 21. Compound 21: 3-Fluoro-5-(2-phenyl-6,7-dihydrooxazolo[5,4-c]pyridin-5(4H)-yl)benzonitrile

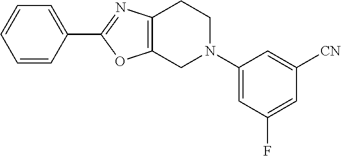

2-Phenyl-4,5,6,7-tetrahydrooxazolo[5,4-c]pyridine hydrochloride (purchased from Anichem) (150 mg, 0.63 mmol), tris(dibenzylideneacetone)dipalladium(0) (40 mg, 0.04 mmol), BINAP (97 mg, 0.16 mmol) and 3-bromo-5-fluorobenzonitrile (126 mg, 0.63 mmol) were combined in toluene (3 mL). The mixture was first flushed with nitrogen before and after the addition of sodium tert-butoxide (73 mg, 0.76 mmol). The solution was heated at 120° C. under nitrogen overnight. The solvent was concentrated and removed under vacuum. The residue was then purified by silica gel column (EtOAc (10% MeOH)/hexanes), followed by recrystallization in methanol to give a white solid (10 mg, 5%). ¹H NMR (400 MHz, CDCl₃): δ 8.01 (m, 2H), 7.46 (m, 3H), 6.97 (s, 1H), 6.81-6.85 (m, 1H), 6.78 (m, 1H), 4.32 (m, 2H), 3.77 (t, 2H), 2.95-2.98 (m, 2H); LC/MS: m/e=320 (M+H)⁺.

### 22. Compound 22: 2-(Pyridin-2-yl)-5-(thiazol-2-yl)-4,5,6,7-tetrahydrooxazolo[5,4-c]pyridine

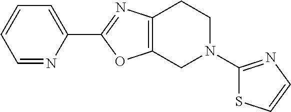

The title compound was prepared via the procedure used for Compound 4, using 2-bromothiazole instead of 3-bromo-5-fluorobenzonitrile. Preparative HPLC afforded 2-(pyridin-2-yl)-5-(thiazol-2-yl)-4,5,6,7-tetrahydrooxazolo[5,4-c]pyridine as a pale solid (20 mg, 14%). ¹H NMR (400 MHz, CDCl₃): δ 8.74 (m, 1H), 8.14 (d, 1H), 7.90 (t, 1H), 7.45 (m, 1H), 7.40 (d, 1H), 6.70 (d, 1H), 4.84 (s, 2H), 4.04 (t, 2H), 3.00 (m, 2H); LC/MS: m/e=285 (M+H)⁺.

### 23. Compound 23: 3-(2-(Pyridin-2-yl)-6,7-dihydrooxazolo[4,5-c]pyridin-5(4H)-yl)benzonitrile

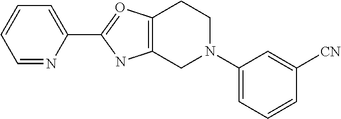

To a solution of 2-(pyridin-2-yl)-4,5,6,7-tetrahydrooxazolo[4,5-c]pyridine (1-23.4) (20 mg, 0.1 mmol) in toluene (2 mL) was added 3-bromobenzonitrile (27 mg, 0.15 mmol), Cs₂CO₃ (65 mg, 0.2 mmol), Pd(OAc)₂ (1 mg, cat.), Xantphos (2 mg, cat.). The reaction was heated to 100° C. and stirred overnight. The mixture was dissolved in MeOH and filtered. The filtrate was concentrated and the residue was purified by preparative HPLC to afford 3-(2-(pyridin-2-yl)-6,7-dihydrooxazolo[4,5-c]pyridin-5(4H)-yl)benzonitrile (5 mg, 17%) as a yellow solid. ¹H NMR (MeOH-d₄): δ 8.65 (d, 1H), 8.15 (d, 1H), 8.05 (t, 1H), 7.50-7.60 (m, 1H), 7.30-7.41 (m, 3H), 7.11 (d, 1H), 4.31 (s, 2H), 3.80 (t, 2H), 2.99 (t, 2H); LC/MS: m/e=303 (M+H)⁺.

### 23.1 4-Hydroxy-3-[(pyridine-2-carbonyl)-amino]-piperidine-1-carboxylic acid benzyl ester (I-23.1)

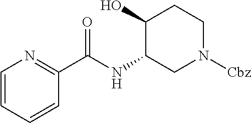

To a solution of pyridine-2-carboxylic acid (492 mg, 4 mmol) and TEA (1.21 g, 12 mmol) in DCM (25 mL) was added EDCI (1.53 g, 8 mmol) and HOBt (1.08 g, 8 mmol). (+/−)-trans-3-Amino-4-hydroxy-piperidine-1-carboxylic acid benzyl ester (prepared according to the procedure in Hall, S. E., et al., WO1994/20062) (1 g, 4 mmol) in DCM (5 mL) was added to the above mixture, and the reaction was stirred overnight at room temperature. The mixture was partitioned between DCM and water and the organic layer was washed with aq. NaHCO₃, 1N HCl solution and brine. The organic phase was dried over anhydrous Na₂SO₄ and concentrated to afford 4-hydroxy-3-[(pyridine-2-carbonyl)-amino]-piperidine-1-carboxylic acid benzyl ester (1.2 g, 84%) as a white solid. ¹H NMR (400 MHz, CDCl₃): δ 8.46 (m, 1H), 8.12 (m, 2H), 7.80 (m, 1H), 7.38 (m, 1H), 7.29 (m, 4H), 5.06 (m, 2H), 4.16 (m, 1H), 3.88 (m, 2H), 3.76 (m, 1H), 3.12 (m, 2H), 1.98 (m, 2H), 1.58 (m, 2H).

### 23.2 4-Oxo-3-[(pyridine-2-carbonyl)-amino]-piperidine-1-carboxylic acid benzyl ester (I-23.2)

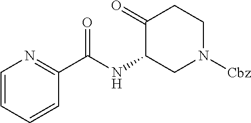

To a solution of 4-hydroxy-3-[(pyridine-2-carbonyl)-amino]-piperidine-1-carboxylic acid benzyl ester (500 mg, 1.4 mmol) in DCM (10 mL) was added Dess-Martin reagent (656 mg, 2.8 mmol). The mixture was stirred overnight at room temperature. The reaction was partitioned between 0.5N NaOH and DCM. The organic phase was dried over anhydrous Na₂SO₄ and concentrated to afford crude product. Purification by preparative TLC (PT/EtOAc=1:1) afforded 4-oxo-3-[(pyridine-2-carbonyl)-amino]-piperidine-1-carboxylic acid benzyl ester (400 mg, 81%). ¹H NMR (400 MHz, CDCl₃): δ 8.54 (m, 1H), 8.14 (m, 1H), 7.80 (m, 1H), 7.22-7.40 (m, 6H), 5.10-5.30 (m, 2H), 4.96 (m, 1H), 4.69 (m, 1H), 4.48 (m, 1H), 4.48 (m, 1H), 2.85 (m, 1H), 2.62 (m, 2H).

### 23.3 Benzyl 2-(pyridin-2-yl)-6,7-dihydrooxazolo[4,5-c]pyridine-5(4H)-carboxylate (I-23.3)

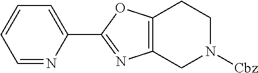

To a solution of POCl₃ (767 mg, 2.5 mmol) in dioxane (10 mL), 4-oxo-3-[(pyridine-2-carbonyl)-amino]-piperidine-1-carboxylic acid benzyl ester (0.9 g, 2.5 mmol) in dioxane (10 mL) was added. The reaction mixture was heated to reflux and stirred for 3 h. The mixture was quenched into water and extracted with EtOAc. The organic layer was dried over anhydrous Na₂SO₄. The solvent was concentrated and the residue was purified by silica gel chromato-graphy (PE:EtOAc=1:1) to afford benzyl 2-(pyridin-2-yl)-6,7-dihydrooxazolo[4,5-c]pyridine-5(4H)-carboxylate (430 mg, 51%). ¹H NMR (400 MHz, CDCl₃): δ 8.65 (m, 1H), 8.00 (m, 1H), 7.76 (m, 1H), 7.28 (m, 6H), 5.12 (s, 2H), 4.51 (s, 2H), 3.83 (m, 2H), 2.82 (m, 2H).

### 23.4 2-Pyridin-2-yl-4,5,6,7-tetrahydro-oxazolo[4,5-c]pyridine (I-23.4)

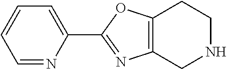

To a solution of benzyl 2-(pyridin-2-yl)-6,7-dihydrooxazolo[4,5-c]pyridine-5(4H)-carboxylate (430 mg, 1.28 mmol) in MeOH (3 mL) was added Pd(OH)₂ on carbon (20 mg). The mixture was stirred at room temperature under H₂ for 0.5 hour. The mixture was filtered and the filtrate was concentrated to afford 2-pyridin-2-yl-4,5,6,7-tetrahydro-oxazolo[4,5-c]-pyridine (230 mg, 89%). ¹H NMR: (400 MHz, CDCl₃): δ 8.69 (m, 1H), 8.08 (m, 1H), 7.78 (m, 1H), 7.32 (m, 1H), 3.84 (m, 1H), 3.68 (s, 1H), 3.49 (m, 1H), 3.14 (m, 1H), 3.06 (m, 1H), 2.86 (m, 2H).

### 24. Compound 24: 2-(2-(Pyridin-2-yl)-6,7-dihydrooxazolo[4,5-c]pyridin-5(4H)-yl)isonicotinonitrile

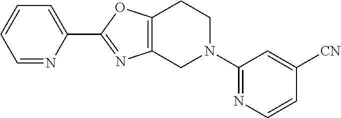

To a solution of 2-(pyridin-2-yl)-4,5,6,7-tetrahydrooxazolo[4,5-c]pyridine (1-23.4) (40 mg, 0.2 mmol) in DMF (2 mL) was added 2-bromoisonicotinonitrile (54 mg, 0.3 mmol), DIEA (52 mg, 0.4 mmol). The mixture was heated to 100° C. and stirred for 16 h. The mixture was cooled to room temperature and poured into water. The mixture was extracted with EtOAc. The combined organic phase was dried over anhydrous Na₂SO₄ and concentrated. The residue was purified by preparative TLC to afford 2-(2-(pyridin-2-yl)-6,7-dihydrooxazolo[4,5-c]pyridin-5(4H)-yl)isonicotinonitrile (10 mg, 17%) as a yellow solid. ¹H NMR (400 MHz, CDCl₃): δ 8.67 (d, 1H), 8.25 (d, 1H), 8.03 (d, 1H), 7.75 (t, 1H), 7.30 (t, 1H), 6.81 (s, 1H), 6.75 (d, 1H), 4.52 (s, 2H), 4.07 (t, 2H), 2.91 (t, 2H); LC/MS: m/e=304 (M+H)⁺.

### 25. Compound 25: 5-(2-(Pyridin-2-yl)-6,7-dihydrooxazolo[4,5-c]pyridin-5(4H)-yl)nicotinonitrile

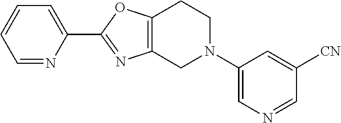

The title compound was prepared via the procedure used for Compound 23, using 5-bromo-nicotinonitrile instead of 3-bromobenzonitrile. Preparative HPLC afforded 5-(2-(pyridin-2-yl)-6,7-dihydrooxazolo[4,5-c]pyridin-5(4H)-yl)nicotinonitrile (10 mg, 13%) as a yellow solid. ¹H NMR (400 MHz, CDCl₃): δ 8.66 (d, 1H), 8.49 (d, 1H), 8.25 (s, 2H) 8.04-8.02 (m, 1H), 7.79-7.75 (m, 1H), 6.90 (s, 1H), 7.34-7.30 (m, 1H), 3.75 (s, 1H), 3.90 (t, 2H), 2.98 (t, 2H); LC/MS: m/e=304 (M+H)⁺.

### 26. Compound 26: 4-(2-(Pyridin-2-yl)-6,7-dihydrooxazolo[4,5-c]pyridin-5(4H)-yl)picolinonitrile

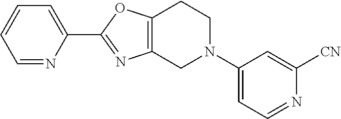

The title compound was prepared via the procedure used for Compound 23, using 4-bromo-pyridine-2-carbonitrile instead of 3-bromobenzonitrile. Preparative HPLC afforded 4-(2-(pyridin-2-yl)-6,7-dihydrooxazolo[4,5-c]pyridin-5(4H)-yl)picolinonitrile as a yellow solid (11 mg, 14.5%). ¹H NMR (400 MHz, CDCl₃): δ 8.69 (d, 1H), 8.32 (d, 1H), 8.06 (d, 1H), 7.83 (t, 1H), 7.38 (t, 1H), 7.10 (s, 1H), 6.86-6.84 (m, 1H), 4.43 (s, 2H), 3.85 (t, 2H), 3.00 (t, 2H); LCMS: m/e=304 (M+H)⁺.

### 27. Compound 27: 2,5-Di(pyridin-2-yl)-4,5,6,7-tetrahydrooxazolo[4,5-c]pyridine

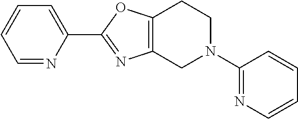

To a solution of 2-(pyridin-2-yl)-4,5,6,7-tetrahydrooxazolo[4,5-c]pyridine (1-23.4) (100 mg, 0.5 mmol) in t-BuOH was added 2-bromopyridine (120 mg, 0.6 mmol), t-BuONa (50 mg, 0.6 mmol), Pd₂ dba₃ (5 mg, cat.), and BINAP (5 mg, cat.). The mixture was heated to 110° C. by microwave and stirred for 1 h. Then the mixture was dissolved in MeOH and filtered. The filtrate was concentrated and the residue was purified by preparative HPLC to afford 2,5-di(pyridin-2-yl)-4,5,6,7-tetrahydrooxazolo[4,5-c]-pyridine (76 mg, 55%) as a yellow liquid. ¹H NMR (400 MHz, CDCl₃): δ 8.67 (m, 1H), 8.26 (m, 1H), 8.02 (d, 1H), 7.82 (m, 2H), 7.33 (m, 1H), 6.92 (m, 1H), 6.84 (m, 1H), 4.59 (s, 2H), 4.22 (m, 2H), 3.10 (m, 2H); LC/MS: m/e=279 (M+H)⁺.

### 28. Compound 28: 2-(2-(Pyridin-2-yl)-6,7-dihydrooxazolo[4,5-c]pyridin-5(4H)-yl)nicotinonitrile

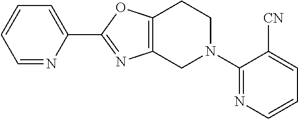

To a solution of 2-(pyridin-2-yl)-4,5,6,7-tetrahydrooxazolo[4,5-c]pyridine (1-23.4) (60 mg, 0.3 mmol) and DIEA (77 mg, 0.6 mmol) in DMF (2 mL) was added 2-chloro-nicotinonitrile (62 mg, 0.45 mmol). The mixture was heated to 100° C. overnight. The mixture was partitioned between EtOAc and H₂O, and the organic layer was dried over MgSO₄. The crude product was purified by preparative HPLC to afford 2-(2-(pyridin-2-yl)-6,7-dihydro-oxazolo[4,5-c]pyridin-5(4H)-yl)nicotinonitrile (1.5 mg, 2%). ¹H NMR (400 MHz, CDCl₃): δ 8.69 (t, 1H); 8.31 (dd, 1H), 8.08 (d, 1H), 7.84 (m, 1H), 7.76 (dd, 1H), 7.35 (t, 1H), 6.76 (dd, 1H), 4.69 (s, 2H), 4.06 (t, 2H), 3.08 (t, 2H); LC/MS: m/e=304 (M+H)⁺.

### 29. Compound 29: 6-(2-(Pyridin-2-yl)-6,7-dihydrooxazolo[4,5-c]pyridin-5(4H)-yl)picolinonitrile

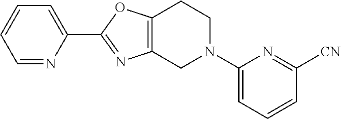

The title compound was prepared via the procedure used for Compound 23, using 6-bromopicolinonitrile instead of 3-bromobenzonitrile. Preparative HPLC afforded 6-(2-(pyridin-2-yl)-6,7-dihydrooxazolo[4,5-c]pyridin-5(4H)-yl)picolinonitrile (10 mg, 17%). ¹H NMR (400 MHz, CDCl₃): δ 8.65 (d, 1H), 8.03 (d, 1H) 7.70-7.80 (m, 1H), 7.46-7.55 (m, 1H), 7.24-7.32 (m, 1H), 6.95 (d, 1H), 6.73 (d, 1H), 4.55 (s, 2H), 4.10 (t, 2H), 2.94 (t, 2H); LC/MS: m/e=304 (M+H)⁺.

### 30. Compound 30: 3-Fluoro-5-(2-(pyridin-2-yl)-6,7-dihydrooxazolo[4,5-c]pyridin-5(4H)-yl)benzonitrile

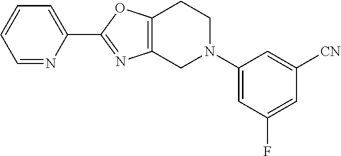

The title compound was prepared via the procedure used for Compound 23, using 3-bromo-5-fluorobenzonitrile instead of 3-bromobenzonitrile. Preparative HPLC afforded 3-fluoro-5-(2-(pyridin-2-yl)-6,7-dihydrooxazolo[4,5-c]pyridin-5(4H)-yl)benzo-nitrile (30 mg, 18%) as a yellow solid. ¹H NMR (400 MHz, CDCl₃): δ 8.74 (d, 1H), 8.12 (m, 1H), 7.91-7.96 (m, 1H), 7.45-7.48 (m, 1H), 6.91 (s, 1H), 6.72-6.79 (m, 2H), 4.29 (s, 2H), 3.71 (m, 2H), 2.94 (m, 2H); LC/MS: m/e=321 (M+H)⁺.

### 31. Compound 31: 5-(3,5-Difluorophenyl)-2-(pyridin-2-yl)-4,5,6,7-tetrahydrooxazolo[4,5-c]pyridine

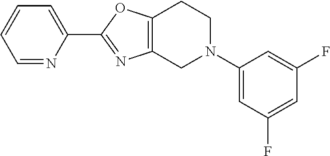

To a solution of 2-(pyridin-2-yl)-4,5,6,7-tetrahydrooxazolo[4,5-c]pyridine (1-23.4) (100 mg, 0.48 mmol) in toluene (10 mL) was added 1-bromo-3,5-difluorobenzene (191 mg, 0.99 mmol), Pd(OAc)₂ (5 mg), Xantphos (5 mg), and t-BuONa (158 mg, 1.44 mmol). The mixture was heated to 110° C. and stirred overnight. The reaction was cooled and filtered, and the filtrate was concentrated in vacuo. The crude residue was purified by HPLC to afford 5-(3,5-difluoro-phenyl)-2-(pyridin-2-yl)-4,5,6,7-tetrahydro-oxazolo[4,5-c]pyridine (10 mg, 6%) as a yellow solid. ¹H NMR (400 MHz, CDCl₃): δ 8.73 (d, 1H); 8.10 (d, 1H), 7.86 (t, 1H), 7.38-7.41 (m, 1H), 6.43-6.21 (m, 2H), 6.27 (t, 1H), 4.29 (s, 2H), 3.71 (t, 2H), 2.83 (t, 2H); LC/MS: m/e=314 (M+H)⁺.

### 32. Compound 32: 3-(2-(Pyridin-2-yl)-6,7-dihydrooxazolo[4,5-c]pyridin-5(4H)-yl)-5-(trifluoromethyl)benzonitrile

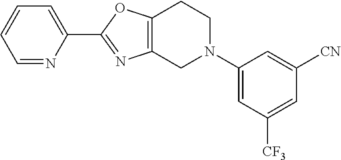

The title compound was prepared via the procedure used for Compound 23, using 3-bromo-5-(trifluoromethyl)benzonitrile instead of 3-bromobenzonitrile. Preparative HPLC afforded 3-(2-(pyridin-2-yl)-6,7-dihydrooxazolo[4,5-c]pyridin-5(4H)-yl)-5-(trifluoromethyl)-benzonitrile (10 mg, 17%). ¹H NMR (400 MHz, CDCl₃): δ 8.83 (d, 1H), 8.22 (d, 1H), 8.08-8.03 (m, 1H), 7.60-7.56 (m, 1H), 7.35-7.32 (m, 3H), 4.41 (s, 2H), 3.84 (t, 2H), 3.05 (t, 2H); LC/MS: m/e=371 (M+H)⁺.

### 33. Compound 33: 3-Fluoro-5-(2-(5-fluoropyridin-2-yl)-6,7-dihydrooxazolo[4,5-c]pyridin-5(4H)-yl)benzonitrile

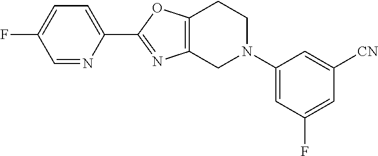

The title compound was prepared via the procedure used for Compound 23, using 5-fluoropicolinic acid instead of picolinic acid, using 3-bromo-5-fluoro-benzonitrile instead of 3-bromobenzonitrile, and using K₂CO₃ instead of Cs₂CO₃. Preparative HPLC afforded 3-fluoro-5-(2-(5-fluoropyridin-2-yl)-6,7-dihydrooxazolo[4,5-c]pyridin-5(4H)-yl)benzonitrile (5 mg, 6%). ¹H NMR (400 MHz, CDCl₃): δ 8.58 (brs, 1H), 8.12 (brs, 1H), 7.50-7.60 (m, 1H), 6.92-6.98 (m, 1H), 6.78-6.88 (m, 2H), 4.32 (s, 2H) 3.77 (t, 2H), 3.00 (t, 2H); LC/MS: m/e=339 (M+H)⁺.

### 34. Compound 34: 3-(2-(Pyridin-2-yl)-6,7-dihydrooxazolo[4,5-c]pyridin-5(4H)-yl)methyl)benzonitrile

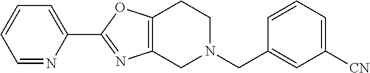

To a solution of 2-(pyridin-2-yl)-4,5,6,7-tetrahydrooxazolo[4,5-c]pyridine (1-23.4) (30 mg, 0.15 mmol, crude) in MeCN (5 mL) was added 3-(bromomethyl)benzo-nitrile (19.5 mg, 0.6 mmol) and Na₂CO₃ (47.7 mg, 0.45 mmol). The mixture was stirred overnight at RT. The reaction was filtered, and the filtrate was concentrated in vacuo. The residue was purified by preparative TLC to afford 3-((2-(pyridin-2-yl)-6,7-dihydro-oxazolo[4,5-c]pyridin-5(4H)-yl)-methyl)benzonitrile (6 mg, 13%) as a pale yellow oil. ¹H NMR (400 MHz, CDCl₃): δ 8.64 (d, 1H); 7.94 (d, 1H), 7.71-7.75 (m, 2H), 7.64 (s, 1H), 7.58-7.56 (m, 1H), 7.53-7.51 (d, 1H), 7.41-7.37 (m, 1H), 7.26-7.29 (m, 1H), 3.74 (s, 2H), 3.56 (t, 2H), 2.84 (t, 4H); LC/MS: m/e=317 (M+H)⁺.

### 35. Compound 35: 3-Fluoro-5-((2-(pyridin-2-yl)-6,7-dihydrooxazolo-[4,5-c]pyridin-5(4H)-yl)methyl)benzonitrile

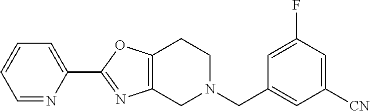

To a solution of 3-bromomethyl-5-fluoro-benzonitrile (42.6 mg, 0.2 mmol) in MeCN (10 mL) was added Na₂CO₃ (64 mg, 0.6 mmol) and 2-(pyridin-2-yl)-4,5,6,7-tetrahydrooxazolo[4,5-c]pyridine (I-23.4) (40 mg, 0.2 mmol). The mixture was stirred at RT for 2 h. The reaction was filtered and the filtrate was purified by preparative HPLC to afford 3-fluoro-5-((2-(pyridin-2-yl)-6,7-dihydrooxazolo[4,5-c]pyridin-5(4H)-yl)methyl)benzonitrile (10 mg, 10%). ¹H NMR (400 MHz, CDCl₃): δ 8.72 (d, 1H), 8.09 (d, 1H), 7.91 (m, 1H), 7.38-7.55 (m, 4H), 4.34 (s, 2H), 4.12 (s, 2H), 3.58 (t, 2H), 3.18 (t, 2H); LC/MS: m/e=335 (M+H)⁺.

### 36. Compound 36: 2-Fluoro-4-(2-(pyridin-2-yl)-6,7-dihydrooxazolo[4,5-c]pyridin-5(4H)-yl)benzonitrile

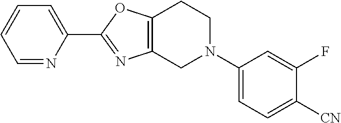

The title compound was prepared via the procedure used for Compound 23, using 4-bromo-2-fluoro-benzonitrile instead of 3-bromobenzonitrile. Preparative HPLC afforded 2-fluoro-4-(2-(pyridin-2-yl)-6,7-dihydrooxazolo[4,5-c]pyridin-5(4H)-yl)benzo-nitrile (15 mg, 19%). ¹H NMR (400 MHz, CDCl₃): δ 8.70 (d, 1H), 8.06 (d, 1H), 7.83 (t, 1H), 7.36-7.40 (m, 2H), 6.65 (dd, 1H), 6.58 (dd, 1H), 4.36 (s, 2H), 3.78 (t, 2H), 2.96 (t, 2H); LC/MS: m/e=321 (M+H)⁺.

### 37. Compound 37: 2-(2-Phenyl-6,7-dihydrooxazolo[4,5-c]pyridin-5(4H)-yl)nicotinonitrile

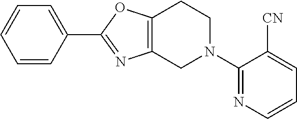

2-Phenyl-4,5,6,7-tetrahydro[1,3]oxazolo[4,5-c]pyridine hydrochloride (purchased from Anichem) (200 mg, 0.84 mmol), 2-chloro-3-pyridine-carbonitrile (176 mg, 1.27 mmol,) and DIEA (218 mg, 1.69 mmol) were combined in DMF (1.5 mL) and heated under microwave at 100° C. for 40 minutes. The solvent was concentrated and removed under vacuum. The residue was then purified by silica gel column (EtOAc (10% MeOH)/Hexanes), followed by HPLC purification to give a white solid (62 mg, 24%). ¹H NMR (400 MHz, CDCl₃): δ 8.36 (m, 1H), 8.01 (m, 2H), 7.83 (m, 1H), 7.45 (m, 3H), 6.78 (m, 1H), 4.73 (s, 2H), 4.10 (t, 2H), 3.09 (m, 2H); LC/MS: m/e=303 (M+H)⁺.

### 38. Compound 38: 2-Phenyl-5-(pyridin-2-yl)-4,5,6,7-tetrahydrooxazolo-[4,5-c]pyridine

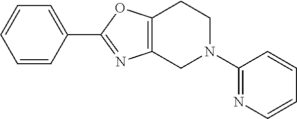

The title compound was prepared via the procedure used for Compound 37, using 2-bromopyridine instead of 2-chloro-3-pyridine-carbonitrile. Silica gel chromatography (EtOAc (10% MeOH)/hexanes) afforded 2-phenyl-5-(pyridin-2-yl)-4,5,6,7-tetrahydrooxazolo[4,5-c]pyridine as an orange oil (5 mg, 4%). ¹H NMR (400 MHz, CDCl₃): δ 8.21 (d, 1H), 8.01 (m, 2H), 7.52 (t, 1H), 7.46 (m, 3H), 6.71 (d, 1H), 6.65 (t, 1H), 4.52 (s, 2H), 4.13 (t, 2H), 2.94 (m, 2H); LC/MS: m/e=278 (M+H)⁺.

### 39. Compound 39: 2-(2-(3-Chlorophenyl)-6,7-dihydrooxazolo[4,5-c]pyridin-5(4H)-yl)nicotinonitrile

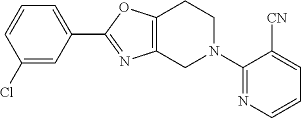

2-(3-Chlorophenyl)-4,5,6,7-tetrahydro[1,3]oxazolo[4,5-c]pyridine hydrochloride (purchased from Anichem) (200 mg, 0.66 mmol), 2-chloro-3-pyridine-carbonitrile (134 mg, 1.27 mmol) and DIEA (251 mg, 1.69 mmol) were combined in DMF (2 mL) and heated under microwave at 120° C. for 40 minutes. The solvent was concentrated and removed under vacuum. The residue was then purified by silica gel column (EtOAc (10% MeOH)/Hexanes). The fractions were concentrated and the resulting solid was purified by precipitation from methanol, then filtered and dried under vacuum giving a white solid (34 mg, 16%). ¹H NMR (400 MHz, CDCl₃): δ 8.36 (m, 1H), 8.01 (s, 1H), 7.89 (m, 1H), 7.81 (dd, 1H), 7.37 (m, 2H), 6.79 (m, 1H), 4.72 (s, 2H), 4.09 (t, 2H), 3.09 (m, 2H); LC/MS: m/e=337 (M+H)⁺.

### 40. Compound 40: 2-(3-Chlorophenyl)-5-(pyridin-2-yl)-4,5,6,7-tetrahydrooxazolo[4,5-c]pyridine

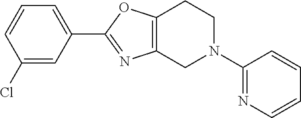

The title compound was prepared via the procedure used for Compound 39, using 2-bromopyridine instead of 2-chloro-3-pyridine-carbonitrile. Silica gel chromatography (EtOAc (10% MeOH)/hexanes) afforded 2-(3-chlorophenyl)-5-(pyridin-2-yl)-4,5,6,7-tetrahydrooxazolo-[4,5-c]pyridine as a white solid (8 mg, 4%). ¹H NMR (400 MHz, CDCl₃): δ 8.22 (d, 1H), 8.01 (s, 1H), 7.89 (d, 1H), 7.52 (t, 1H), 7.38 (m, 2H), 6.71 (d, 1H), 6.66 (t, 1H), 4.52 (s, 2H), 4.13 (t, 2H), 2.94 (s, 2H); LC/MS: m/e=312 (M+H)⁺.

### 41. Compound 41: 2-(3-Chlorophenyl)-5-(pyrazin-2-yl)-4,5,6,7-tetrahydrooxazolo[4,5-c]pyridine

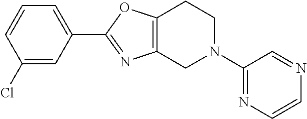

The title compound was prepared via the procedure used for Compound 39, using chloropyrazine instead of 2-chloro-3-pyridine-carbonitrile. Silica gel chromatography (EtOAc (10% MeOH)/hexanes) afforded 2-(3-chlorophenyl)-5-(pyrazin-2-yl)-4,5,6,7-tetrahydrooxazolo-[4,5-c]pyridine as a white solid (12 mg, 10%). ¹H NMR (400 MHz, CDCl₃): δ 8.23 (s, 1H), 8.10 (d, 1H), 8.01 (s, 1H), 7.90 (m, 2H), 7.38 (m, 2H), 4.614 (s, 2H), 4.12 (m, 2H), 2.96 (m, 2H); LC/MS: m/e=313 (M+H)⁺.

### 42. Compound 42: 2-(4-Methoxyphenyl)-5-(pyrazin-2-yl)-4,5,6,7-tetrahydrooxazolo[4,5-c]pyridine

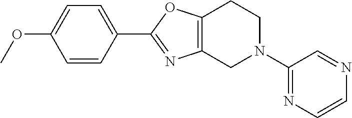

2-(4-Methoxyphenyl)-4,5,6,7-tetrahydro[1,3]oxazolo[4,5-c]pyridine hydrochloride (purchased from Anichem) (100 mg, 0.37 mmol), chloropyrazine (86 mg, 0.75 mmol), and DIEA (145 mg, 1.12 mmol) were combined in DMF (1.5 mL) and heated under microwave at 160° C. for 1.5 h. The solvent was concentrated and removed under vacuum. The residue was then purified by silica gel column (EtOAc (10% MeOH)/hexanes) to give an orange solid (11 mg, 10%). ¹H NMR (400 MHz, CDCl₃): δ 8.22 (s, 1H), 8.09 (m, 1H), 7.94 (m, 2H), 7.89 (d, 1H), 6.96 (m, 2H), 4.59 (m, 2H), 4.12 (t, 2H), 3.86 (s, 3H), 2.94 (m, 2H); LC/MS: m/e=308 (M+H)⁺.

### 43. Compound 43: 2-(2-(4-Methoxyphenyl)-6,7-dihydrooxazolo[4,5-c]pyridin-5(4H)-yl)nicotinonitrile

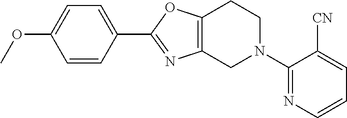

The title compound was prepared via the procedure used for Compound 42, using 2-chloro-3-pyridine-carbonitrile instead of chloropyrazine. Silica gel chromatography (EtOAc (10% MeOH)/hexanes) afforded 2-(2-(4-methoxyphenyl)-6,7-dihydrooxazolo[4,5-c]pyridin-5(4H)-yl)nicotinonitrile as a white solid. (94 mg, 38%). ¹H NMR (400 MHz, CDCl₃): δ 8.36 (dd, 1H), 7.96 (m, 2H), 7.80 (dd, 1H), 6.95 (m, 2H), 6.77 (m, 1H), 4.71 (m, 2H), 4.09 (t, 2H), 3.86 (s, 3H), 3.06 (m, 2H); LC/MS: m/e=333 (M+H)⁺.

### 44. Compound 44: 2-(2-(4-(Trifluoromethyl)phenyl)-6,7-dihydrooxazolo[4,5-c]pyridin-5(4H)-yl)nicotinonitrile

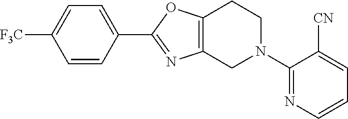

2-[4-(Trifluoromethyl)phenyl]-4,5,6,7-tetrahydro[1,3]oxazolo[4,5-c]pyridine hydrochloride (purchased from Anichem) (200 mg, 0.66 mmol), 2-chloro-3-pyridine-carbonitrile (182 mg, 1.31 mmol), and DIEA (254 mg, 1.97 mmol) were combined in DMF (1.5 mL) and heated under microwaves at 150° C. for 1 h. The solvent was concentrated and removed under vacuum. Silica gel chromatography (EtOAc (10% MeOH)/hexanes), followed by preparative HPLC and precipitation of the impure fractions in methanol:ethanol (1:2) afforded a white solid (42 mg, 17%). ¹H NMR (400 MHz, CDCl₃): δ 8.37 (m, 1H), 8.12 (d, 2H), 7.81 (d, 1H), 7.70 (d, 2H), 6.80 (m, 1H), 4.73 (s, 2H), 4.10 (m, 2H), 3.11 (s, 2H); LC/MS: m/e=371 (M+H)⁺.

### 45. Compound 45: 5-(Pyrazin-2-yl)-2-(4-(trifluoromethyl)phenyl)-4,5,6,7-tetrahydrooxazolo[4,5-c]pyridine

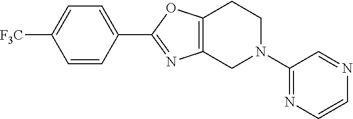

The title compound was prepared via the procedure used for Compound 44, using chloropyrazine instead of 2-chloro-3-pyridine-carbonitrile. Silica gel chromatography (EtOAc (10% MeOH)/hexanes) followed by precipitation in methanol:ethanol (1:2) afforded 5-(pyrazin-2-yl)-2-(4-(trifluoromethyl)phenyl)-4,5,6,7-tetrahydrooxazolo[4,5-c]pyridine as a white solid (23 mg, 10%). ¹H NMR (400 MHz, CDCl₃): δ 8.24 (s, 1H), 8.12 (m, 3H), 7.91 (d, 1H), 7.71 (d, 2H), 4.63 (m, 2H), 4.13 (t, 2H), 2.98 (m, 2H); LC/MS: m/e=347 (M+H)⁺.

### 46. Compound 46: 2-(2-(4-Fluorophenyl)-6,7-dihydrooxazolo[4,5-c]pyridin-5(4H)-yl)nicotinonitrile

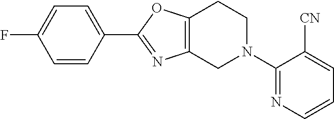

2-(4-Fluorophenyl)-4,5,6,7-tetrahydrooxazolo[4,5-c]pyridine hydrochloride (purchased from Anichem) (200 mg, 0.78 mmol), 2-chloro-3-pyridine-carbonitrile (218 mg, 1.57 mmol), and DIEA (304 mg, 2.35 mmol) were combined in DMF (1.0 mL) and heated under microwave at 150° C. for 1.5 h. The solvent was concentrated and removed under vacuum. The residue was then purified by silica gel column (EtOAc (10% MeOH)/hexanes) to give a white solid (39 mg, 15%). ¹H NMR (400 MHz, CDCl₃): δ 8.36 (m, 1H), 8.01 (m, 2H), 7.80 (dd, 1H), 7.13 (m, 2H), 6.80 (dd, 1H), 4.71 (m, 2H), 4.09 (m, 2H), 3.08 (m, 2H); LC/MS: m/e=321 (M+H)⁺.

### 47. Compound 47: 2-(4-Fluorophenyl)-5-(pyrazin-2-yl)-4,5,6,7-tetrahydrooxazolo[4,5-c]pyridine

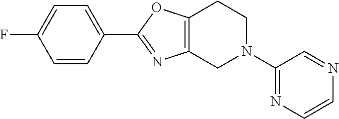

The title compound was prepared via the procedure used for Compound 46, using chloropyrazine instead of 2-chloro-3-pyridine-carbonitrile. Silica gel chromatography (EtOAc (10% MeOH)/hexanes), followed by precipitation from ethanol gave 2-(4-fluorophenyl)-5-(pyrazin-2-yl)-4,5,6,7-tetrahydrooxazolo[4,5-c]pyridine as a white solid (32 mg, 14%). ¹H NMR (400 MHz, CDCl₃): δ 8.23 (m, 1H), 8.10 (m, 1H), 8.00 (m, 2H), 7.90 (d, 1H), 7.14 (m, 2H), 4.60 (m, 2H), 4.12 (t, 2H), 2.95 (m, 2H); LC/MS: m/e=297 (M+H)⁺.

### 48. Compound 48: 2-(4-Fluorophenyl)-5-(pyridin-2-yl)-4,5,6,7-tetrahydrooxazolo[4,5-c]pyridine

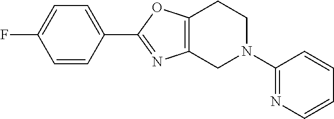

The title compound was prepared via the procedure used for Compound 46, using 2-bromopyridine instead of 2-chloro-3-pyridine-carbonitrile. Silica gel chromatography (EtOAc (10% MeOH)/hexanes), followed by precipitation from ethanol gave 2-(4-fluorophenyl)-5-(pyridin-2-yl)-4,5,6,7-tetrahydrooxazolo[4,5-c]pyridine as a white solid (8 mg, 4%). ¹H NMR (400 MHz, CDCl₃): δ 8.21 (m, 1H), 8.00 (m, 2H), 7.52 (m, 1H), 7.13 (m, 2H), 6.71 (d, 1H), 6.65 (m, 1H), 4.51 (m, 2H), 4.13 (m, 2H), 2.93 (m, 2H); LC/MS: m/e=296 (M+H)⁺.

### 49. Compound 49: 2-Phenyl-5-(pyrazin-2-yl)-4,5,6,7-tetrahydrooxazolo[4,5-c]pyridine

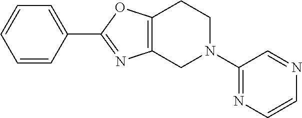

2-Phenyl-4,5,6,7-tetrahydro[1,3]oxazolo[4,5-c]pyridine hydrochloride (purchased from Anichem) (200 mg, 0.84 mmol), chloropyrazine (193 mg, 1.69 mmol), and DIEA (327 mg, 2.53 mmol) were combined in DMF (1.0 mL) and heated under microwave at 160° C. for 1.5 h. The solvent was concentrated and removed under vacuum. The residue was then purified by silica gel column (EtOAc (10% MeOH)/hexanes), followed by precipitation from ethanol to give a white solid (38 mg, 16%). ¹H NMR (400 MHz, CDCl₃): δ 8.23 (m, 1H), 8.10 (m, 1H), 8.01 (m, 2H), 7.89 (d, 1H), 7.45 (m, 3H), 4.61 (m, 2H), 4.13 (t, 2H), 2.96 (m, 2H); LC/MS: m/e=279 (M+H)⁺.

### 50. Compound 50: 2-(3-Chlorophenyl)-5-(pyridin-2-ylmethyl)-4,5,6,7-tetrahydrooxazolo[4,5-c]pyridine

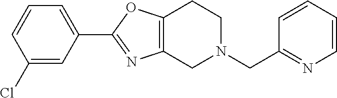

2-(3-Chlorophenyl)-4,5,6,7-tetrahydro[1,3]oxazolo[4,5-c]pyridine hydrochloride (purchased from Anichem) (100 mg, 0.37 mmol), 2-(bromomethyl)-pyridine hydrobromide (93 mg, 0.37 mmol), and DIEA (95 mg, 0.74 mmol) were combined in DMF (1.0 mL) and heated under microwaves at 150° C. for 1 h. The solvent was concentrated and removed under vacuum. The residue was then purified by silica gel column (EtOAc (10% MeOH)/hexanes) to give a yellow oil (34 mg, 28%). ¹H NMR (400 MHz, CDCl₃): δ 8.58 (m, 1H), 7.98 (s, 1H), 7.87-7.85 (m, 1H), 7.66-7.70 (m, 1H), 7.46 (d, 1H), 7.36 (m, 2H), 7.20 (m, 1H), 3.93 (s, 2H), 3.62 (m, 2H), 2.98 (t, 2H), 2.85 (m, 2H); LC/MS: m/e=326 (M+H)⁺.

### 51. Compound 51: 2-(3-Chlorophenyl)-5-(pyridin-3-yl)-4,5,6,7-tetrahydrooxazolo[4,5-c]pyridine

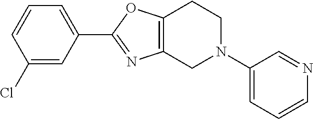

2-(3-Chlorophenyl)-4,5,6,7-tetrahydro[1,3]oxazolo[4,5-c]pyridine hydrochloride (purchased from Anichem) (200 mg, 0.74 mmol), tris(dibenzylidene-acetone)dipalladium(0) (48 mg, 0.05 mmol), tris-tert-butyl phosphonium tetrafluoro-borate (53 mg, 0.18 mmol), and 3-iodopyridine (151 mg, 0.74 mmol) were combined in toluene (5 mL). The mixture was flushed with nitrogen and sodium tert-butoxide (142 mg, 1.47 mmol) was added. The solution was heated at reflux under nitrogen for 4 h. An additional amount of 3-iodopyridine (151 mg, 0.74 mmol) was added and the solution was heated at reflux under nitrogen for 2 h. The solvent was concentrated and removed under vacuum. The residue was purified by silica gel column (EtOAc (10% MeOH)/hexanes), followed by HPLC purification to give an orange oil (56 mg, 25%). ¹H NMR (400 MHz, CDCl₃): δ 8.41 (d, 1H), 8.12 (m, 1H), 8.01 (m, 1H), 7.88-7.91 (m, 1H), 7.38-7.40 (m, 2H), 7.24 (m, 1H), 7.19 (m, 1H), 4.31 (m, 2H), 3.75 (t, 2H), 2.96 (m, 2H); LC/MS: m/e=312 (M+H)⁺.

### 52. Compound 52: 3-(2-Phenyl-6,7-dihydrooxazolo[4,5-c]pyridin-5(4H)-yl)pyrazine-2-carbonitrile

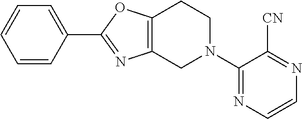

The title compound was prepared via the procedure used for Compound 49, using 3-chloropyrazine-2-carbonitrile instead of chloropyrazine. Silica gel chromatography (EtOAc (10% MeOH)/hexanes) afforded 3-(2-phenyl-6,7-dihydro-oxazolo[4,5-c]pyridin-5(4H)-yl)pyrazine-2-carbonitrile as an orange/yellow solid (75 mg, 59%). ¹H NMR (400 MHz, CDCl₃): δ 8.30 (d, 1H), 8.07 (d, 1H), 8.01 (m, 2H), 7.45 (m, 3H), 4.77 (m, 2H), 4.18 (t, 2H), 3.10 (m, 2H); LC/MS: m/e=304 (M+H)⁺.

### 53. Compound 53: 3-(2-(3-Chlorophenyl)-6,7-dihydrooxazolo[4,5-c]pyridin-5(4H)-yl)pyrazine-2-carbonitrile

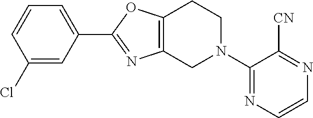

The title compound was prepared via the procedure used for Compound 39, using 3-chloropyrazine-2-carbonitrile instead of 2-chloro-3-pyridine-carbonitrile. Silica gel chromatography (EtOAc (10% MeOH)/hexanes) afforded 3-(2-(3-chlorophenyl)-6,7-dihydrooxazolo[4,5-c]pyridin-5(4H)-yl)pyrazine-2-carbonitrile as a yellow oil (16 mg, 18%). ¹H NMR (400 MHz, CDCl₃): δ 8.30 (d, 1H), 8.08 (d, 1H), 8.01 (m, 1H), 7.88-7.91 (m, 1H), 7.40 (m, 2H), 4.76 (m, 2H), 4.17 (t, 2H), 3.08-3.12 (m, 2H); LC/MS: m/e=338 (M+H)⁺.

### 54. Compound 54: (4-Fluorophenyl)(2-(4-fluorophenyl)-6,7-dihydrooxazolo[4,5-c]pyridin-5(4H)-yl)methanone

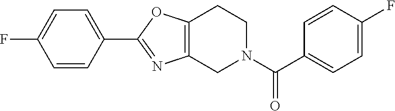

2-(4-Fluorophenyl)-4,5,6,7-tetrahydro[1,3]oxazolo[4,5-c]pyridine hydrochloride (purchased from Anichem) (75 mg, 0.29 mmol), 4-fluorobenzoic acid (41 mg, 0.29 mmol), and EDCI (87 mg, 0.29 mmol) were combined in DCM and stirred at room temperature for 4 h. The solvent was concentrated and removed under vacuum. The residue was dissolved in DCM, washed twice with water and once with brine. The combined organic layers were dried over MgSO₄. The mixture was then filtered, the solvent was concentrated and removed under vacuum to afford a yellow solid (32 mg, 32%). ¹H NMR (400 MHz, CDCl₃): δ 8.16-8.19 (m, 1H), 7.97 (s, 1H), 7.47-7.50 (m, 2H), 7.19-7.23 (m, 1H), 7.11-7.15 (m, 3H), 4.52-4.72 (m, 2H), 3.78-4.11 (m, 2H), 2.94 (s, 2H); LC/MS: m/e=341 (M+H)⁺.

### 55. Compound 55: 2-(Pyridin-2-yl)-5-(thiazol-2-yl)-4,5,6,7-tetrahydrooxazolo[4,5-c]pyridine

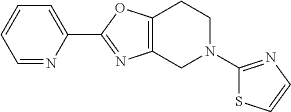

The title compound was prepared via the procedure used for Compound 23, using 2-bromothiazole instead of 3-bromobenzonitrile. Preparative HPLC afforded 2-(pyridin-2-yl)-5-(thiazol-2-yl)-4,5,6,7-tetrahydrooxazolo[4,5-c]pyridine as a pale solid (30 mg, 21%). ¹H NMR (400 MHz, CDCl₃): δ 8.59 (d, 1H), 7.98 (d, 1H), 7.75 (td, 1H), 7.29 (m, 1H), 7.16 (d, 1H), 6.57 (d, 1H), 4.78 (s, 2H), 3.97 (t, 2H), 2.98 (m, 2H); LC/MS: m/e=285 (M+H)⁺.

### 56. Compound 56: 5-(3-Fluoro-5-methoxyphenyl)-2-(pyridin-2-yl)-4,5,6,7-tetrahydrooxazolo[4,5-c]pyridine

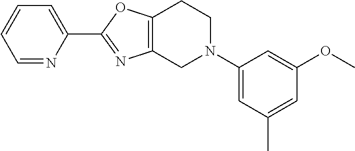

The title compound was prepared via the procedure used for Compound 23, using 1-bromo-3-fluoro-5-methoxybenzene instead of 3-bromobenzonitrile. Preparative HPLC afforded 5-(3-fluoro-5-methoxyphenyl)-2-(pyridin-2-yl)-4,5,6,7-tetrahydro-oxazolo[4,5-c]pyridine as a pale solid (32 mg, 19%). ¹H NMR (400 MHz, CDCl₃): δ 8.77 (d, 1H), 8.16 (d, 1H), 8.01 (td, 1H), 7.52 (m, 1H), 6.26 (m, 1H), 6.24 (m, 1H), 6.11 (m, 1H), 4.26 (s, 2H), 3.72 (s, 3H), 3.65 (t, 3H), 2.93 (t, 3H); LC/MS: m/e=326 (M+H)⁺.

### 57. Compound 57: 2-Fluoro-5-(2-(pyridin-2-yl)-6,7-dihydrooxazolo[4,5-c]pyridin-5(4H)-yl)benzonitrile

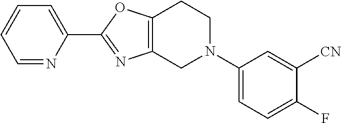

The title compound was prepared via the procedure used for Compound 23, using 5-bromo-2-fluorobenzonitrile instead of 3-bromobenzonitrile. Preparative HPLC afforded 2-fluoro-5-(2-(pyridin-2-yl)-6,7-dihydrooxazolo[4,5-c]pyridin-5(4H)-yl)benzo-nitrile as a pale solid (2 mg, 2%). ¹H NMR (400 MHz, CDCl₃): δ 8.77 (d, 1H), 8.15 (d, 1H), 7.91 (t, 1H), 7.45 (m, 1H), 7.19 (m, 1H), 7.14 (m, 2H), 4.29 (s, 2H), 3.71 (t, 2H), 3.01 (t, 3H); LC/MS: m/e=321 (M+H)⁺.

### 58. Compound 58: 5-(Pyridin-2-yl)-2-m-tolyl-4,5,6,7-tetrahydrooxazolo[5,4-c]pyridine

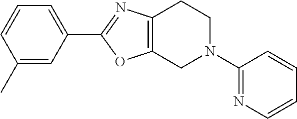

To a solution of 2-m-tolyl-4,5,6,7-tetrahydrooxazolo[5,4-c]pyridine (I-58.1) (50 mg, 0.23 mmol) in t-BuOH was added 2-bromopyridine (55 mg, 0.35 mmol), t-BuONa (46 mg, 0.5 mmol), Pd₂ dba₃ (5 mg, cat.), and BINAP (5 mg, cat.). The mixture was heated to 110° C. by microwave and stirred for 1 h. The mixture was dissolved in MeOH and filtered. The filtrate was concentrated and the residue was purified by preparative TLC to afford 5-(pyridin-2-yl)-2-m-tolyl-4,5,6,7-tetrahydrooxazolo[5,4-c]-pyridine (30 mg, 44%) as a yellow solid. ¹H NMR (400 MHz, CDCl₃): δ 8.15 (d, 1H), 7.79 (s, 1H), 7.72 (d, 1H), 7.45 (t, 1H), 7.26 (t, 1H), 7.15 (d, 1H), 6.68 (d, 1H), 6.59 (q, 1H), 4.68 (s, 2H), 3.80 (t, 2H), 2.75 (t, 2H), 2.35 (s, 3H); LC/MS: m/e=308 (M+H)⁺.

### 58.1 2-m-Tolyl-4,5,6,7-tetrahydrooxazolo[5,4-c]pyridine (I-58.1)

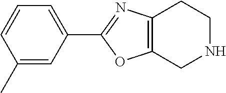

The title compound was prepared via the procedure used for I-1.4, using 3-methylbenzoic acid instead of picolinic acid. Preparative TLC afforded 2-m-tolyl-4,5,6,7-tetrahydrooxazolo[5,4-c]pyridine (500 mg, 81%) as a yellow solid. ¹H NMR (400 MHz, DMSO-d₆): δ 7.72 (s, 1H), 7.69 (d, 1H), 7.35 (t, 1H), 7.25 (d, 1H), 3.78 (s, 2H), 2.90 (t, 2H), 2.51 (t, 2H), 2.33 (s, 3H).

### 59. Compound 59: 2-(2-m-Tolyl-6,7-dihydrooxazolo[5,4-c]pyridin-5(4H)-yl)nicotinonitrile

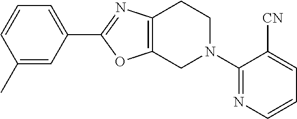

To a solution of 2-m-tolyl-4,5,6,7-tetrahydrooxazolo[5,4-c]pyridine (I-58.1) (100 mg, 0.46 mmol) in DMF was added 2-chloronicotinonitrile (96 mg, 0.67 mmol), and DIEA (119 mg, 0.92 mmol). The mixture was heated to 100° C. and stirred for 6 h. The mixture was cooled to room temperature and poured into water. The mixture was extracted with EtOAc. The organic phase was dried over anhydrous Na₂SO₄, concentrated, and the residue was purified by preparative HPLC to afford 2-(2-m-tolyl-6,7-dihydrooxazolo[5,4-c]pyridin-5(4H)-yl)nicotino-nitrile (40 mg, 28%) as a yellow solid. ¹H NMR (400 MHz, CDCl₃): δ 8.32 (d, 1H), 7.75-7.81 (m, 2H), 7.72 (d, 1H), 7.30 (t, 1H), 7.25 (d, 1H), 6.78 (dd, 1H), 4.77 (s, 2H), 4.00 (t, 2H), 2.95 (t, 2H), 2.35 (s, 1H); LC/MS: m/e=317 (M+H)⁺.

### 60. Compound 60: 5-(Pyridin-2-yl)-2-m-tolyl-4,5,6,7-tetrahydrooxazolo-[4,5-c]pyridine

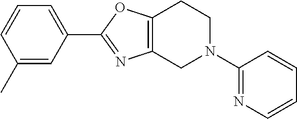

To a solution of 2-m-tolyl-4,5,6,7-tetrahydrooxazolo[4,5-c]pyridine (I-60.2) (50 mg, 0.23 mmol) in t-BuOH (5 mL) was added 2-bromopyridine (55 mg, 0.34 mmol), Pd₂(dba)₃ (5 mg), BINAP (5 mg), and t-BuONa (46 mg, 0.46 mmol). The mixture was stirred at 100° C. in microwave for 1 h. The reaction was filtered, concentrated, and the residue purified by preparative HPLC to afford 5-(pyridin-2-yl)-2-m-tolyl-4,5,6,7-tetrahydrooxazolo[4,5-c]pyridine (1.2 mg, 2%) as a white solid. ¹H NMR (400 MHz, CDCl₃): δ 8.15-8.14 (m, 1H), 7.83 (s, 1H), 7.73 (d, 1H), 7.47-7.43 (m, 1H), 7.43 (d, 1H), 7.27 (t, 1H), 7.19-7.14 (m, 1H), 6.64 (d, 1H), 6.59-6.56 (m, 1H), 4.45 (s, 2H), 4.07 (t, 1H), 2.89-2.85 (m, 2H), 2.34 (s, 3H); LC/MS: m/e=292 (M+H)⁺.

### 60.1 Benzyl 2-m-tolyl-6,7-dihydrooxazolo[4,5-c]pyridine-5(4H)-carboxylate (I-60.1)

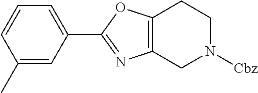

The title compound was prepared via the procedure used for I-23.3, using 3-methylbenzoic acid instead of picolinic acid. Silica gel chromatography (PE:EtOAc=1:1) afforded benzyl 2-m-tolyl-6,7-dihydrooxazolo[4,5-c]pyridine-5(4H)-carboxylate (250 mg, 52%). ¹H NMR (400 MHz, DMSO-d₆): δ=7.76 (s, 1H), 7.71 (d, 1H), 7.31-7.24 (m, 7H), 5.11 (s, 1H), 4.53-4.50 (m, 2H), 3.83 (brs, 2H), 2.78 (s, 2H), 2.34 (s, 3H).

### 60.2 2-m-Tolyl-4,5,6,7-tetrahydrooxazolo[4,5-c]pyridine (I-60.2)

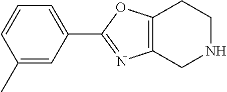

To a solution of benzyl 2-m-tolyl-6,7-dihydrooxazolo[4,5-c]pyridine-5(4H)-carboxylate (100 mg, 0.29 mmol) in MeCN was added TMSI (57.2 g, 2.87 mmol). The reaction was stirred for 3 h at room temperature. The mixture was filtered and the filtrate was washed with ether to afford 2-m-tolyl-4,5,6,7-tetrahydrooxazolo[4,5-c]-pyridine (30 mg, 49%). ¹H NMR (400 MHz, DMSO-d₆): δ 7.73-7.67 (m, 2H), 7.28-7.24 (m, 1H), 4.17 (s, 2H), 3.55 (t, 2H), 3.06 (t, 1H), 2.31 (s, 1H).

### 61. Compound 61: 2-(2-m-Tolyl-6,7-dihydrooxazolo[4,5-c]pyridin-5(4H)-yl)nicotinonitrile

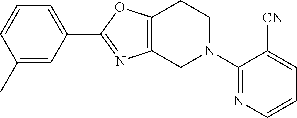

To a solution of 2-m-tolyl-4,5,6,7-tetrahydrooxazolo[4,5-c]pyridine (I-60.2) (50 mg, 0.24 mmol) and DIEA (62 mg, 0.48 mmol) in DMF (5 mL) was added 2-chloro-nicotinonitrile (62 mg, 0.45 mmol). The mixture was heated to 100° C. overnight. The reaction mixture was partitioned between EtOAc and H₂O, and the organic layer was separated and dried over MgSO₄. Purification, first by preparative TLC, and then by preparative HPLC afforded 2-(2-m-tolyl-6,7-dihydro-4H-oxazolo[4,5-c]pyridin-5-yl)-nicotinonitrile (7 mg, 9%). ¹H NMR: (400 MHz, CDCl₃): δ 8.37 (dd, 1H); 7.86 (s, 1H), 7.81 (dd, 2H), 7.32-7.36 (m, 1H), 6.76 (dd, 1H), 4.73 (m, 2H), 4.10 (m, 2H), 3.10 (m, 2H), 2.42 (s, 3H), 3.10 (m, 2H), 4.10 (m, 2H), 4.73 (m, 2H), 6.76 (dd, 1H), 7.32-7.36 (m, 1H), 7.81 (dd, 2H), 7.86 (s, 1H), 8.37 (dd, 1H); LC/MS: m/e=317 (M+H)⁺.

### 62. Compound 62: 3-(5-(Pyridin-2-yl)-4,5,6,7-tetrahydrooxazolo[4,5-c]pyridin-2-yl)benzonitrile

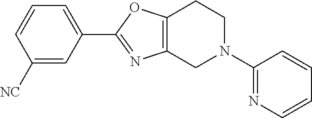

The title compound was prepared via the procedure used for Compound 60, using benzyl 2-(3-cyanophenyl)-6,7-dihydrooxazolo[4,5-c]pyridine-5(4H)-carboxylate instead of benzyl 2-m-tolyl-6,7-dihydrooxazolo[4,5-c]pyridine-5(4H)-carboxylate. Benzyl 2-(3-cyanophenyl)-6,7-dihydrooxazolo[4,5-c]pyridine-5(4H)-carboxylate was prepared via the procedure used for I-60.1, using 3-cyanobenzoic acid instead of 3-methylbenzoic acid. Preparative TLC, followed by preparative HPLC afforded 3-(5-pyridin-2-yl-4,5,6,7-tetrahydrooxazolo[4,5-c]pyridin-2-yl)benzonitrile (3 mg, 4%) as a yellow oil. ¹H NMR (400 MHz, CDCl₃): δ 8.23 (s, 1H); 8.18 (m, 2H), 7.64 (m, 1H), 7.50 (m, 2H), 6.68 (m, 2H), 4.49 (s, 2H), 4.16-4.24 (m, 2H), 2.93-3.06 (m, 2H); LC/MS: m/e=303 (M+H)⁺.

### 63. Compound 63: 2-(2-(3-Cyanophenyl)-6,7-dihydrooxazolo[4,5-c]pyridin-5(4H)-yl)nicotinonitrile

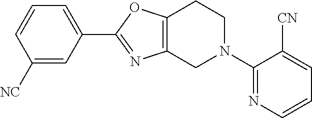

To a solution of 3-(4,5,6,7-tetrahydrooxazolo[4,5-c]pyridin-2-yl)-benzonitrile (50 mg crude, 0.22 mmol), prepared via the procedure used for I-60.2, using 3-cyanobenzoic acid instead of 3-methylbenzoic acid, and DIEA (57 mg, 0.44 mmol) in DMF (5 mL) was added 2-chloro-nicotinonitrile (46 mg, 0.33 mmol). The mixture was heated to 100° C. overnight. The mixture was partitioned between EtOAc and H₂O. The organic layer was dried over MgSO₄ and purified, first by preparative TLC, and then by preparative HPLC to afford 2-[2-(3-cyano-phenyl)-6,7-dihydro-4H-oxazolo[4,5-c]-pyridin-5-yl]nicotinonitrile (3 mg, 5% in two steps) as a yellow oil. ¹H NMR: (400 MHz, CDCl₃): δ 8.40-8.42 (m, 1H), 8.32 (s, 1H), 8.26 (dd, 1H), 7.88 (dd, 1H), 7.71 (m, 1H), 7.59 (t, 1H), 6.84 (dd, 1H), 4.76 (s, 2H), 4.14 (m, 2H), 3.14 (m, 2H); LC/MS: m/e=328 (M+H)⁺.

### 64. Compound 64: 3,3′-(6,7-Dihydrooxazolo[4,5-c]pyridine-2,5(4H)-diyl)dibenzonitrile

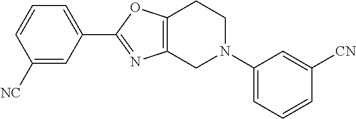

To a solution of 3-(4,5,6,7-tetrahydrooxazolo[4,5-c]pyridin-2-yl)benzonitrile (15 mg, 0.07 mmol) in toluene (2 mL) was added 3-bromobenzonitrile (18 mg, 0.1 mmol), Cs₂CO₃ (42 mg, 0.14 mmol), Pd(OAc)₂ (1 mg, cat.), and Xantphos (2 mg, cat.). The mixture was heated to 100° C. and stirred overnight. The reaction was cooled, diluted with MeOH and filtered. The filtrate was concentrated and the residue was purified by preparative TLC to afford 3,3′-(6,7-dihydrooxazolo[4,5-c]pyridine-2,5(4H)-diyl)dibenzonitrile (10 mg, 22%) as a yellow solid. ¹H NMR (400 MHz, MeOH-d₄): δ 8.21 (s, 1H), 8.18 (d, 1H), 7.72 (d, 1H), 7.60 (t, 1H), 7.22-7.35 (m, 3H), 7.03 (d, 1H), 4.21 (s, 2H), 3.72 (t, 2H), 2.89 (t, 2H); LC/MS: m/e=327 (M+H)⁺.

### 65. Compound 65: 3-(5-(Pyridin-2-yl)-4,5,6,7-tetrahydrooxazolo[5,4-c]pyridin-2-yl)benzonitrile

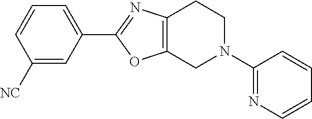

The title compound was prepared via the procedure used for Compound 60, using benzyl 2-(3-cyanophenyl)-6,7-dihydrooxazolo[5,4-c]pyridine-5(4H)-carboxylate instead of benzyl 2-m-tolyl-6,7-dihydrooxazolo[4,5-c]pyridine-5(4H)-carboxylate. Benzyl 2-(3-cyanophenyl)-6,7-dihydrooxazolo[5,4-c]pyridine-5(4H)-carboxylate was prepared via the procedure used for I-1.3, using 3-cyanobenzoic acid instead of picolinic acid. Preparative HPLC afforded 3-(5-pyridin-2-yl)-4,5,6,7-tetrahydrooxazolo[5,4-c]-pyridin-2-yl)benzonitrile (4.3 mg, 32%) as a yellow liquid. ¹H NMR (400 MHz, CDCl₃): δ=8.23 (s, 1H), 8.15 (s, 1H), 7.63-7.61 (m, 1H), 7.52-7.48 (m, 1H), 6.72-6.70 (d, 1H), 6.27 (s, 1H), 4.73 (s, 1H), 3.91 (brs, 2H), 2.74 (brs, 2H); LC/MS: m/e=303 (M+H)⁺.

### 66. Compound 66: 2-(2-(3-Cyanophenyl)-6,7-dihydrooxazolo[5,4-c]pyridin-5(4H)-yl)nicotinonitrile

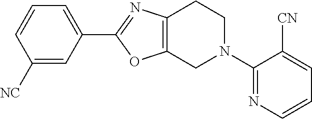

The title compound was prepared via the procedure used for Compound 63, using 3-(4,5,6,7-tetrahydrooxazolo[5,4-c]pyridin-2-yl)-benzonitrile instead of 3-(4,5,6,7-tetrahydro-oxazolo[4,5-c]pyridin-2-yl)-benzonitrile. 3-(4,5,6,7-Tetrahydrooxazolo[5,4-c]pyridin-2-yl)-benzonitrile was prepared via the procedure used for I-1.4, using 3-cyanobenzoic acid instead of picolinic acid. Preparative TLC afforded 2-(2-(3-cyanophenyl)-6,7-dihydrooxazolo[5,4-c]pyridin-5(4H)-yl)nicotinonitrile (2 mg, 3%) as a yellow solid. ¹H NMR (400 MHz, CDCl₃): δ 8.16 (dd, 1H), 8.24-8.23 (m, 1H), 8.17-8.15 (m, 1H), 7.78 (m, 1H), 7.77-7.60 (m, 1H), 7.65-7.53 (m, 1H), 7.532-7.493 (dd, 1H), 6.77 (m, 1H), 4.76 (t, 2H), 4.02-3.99 (m, 2H), 2.91-2.89 (m, 2H); LC/MS: m/e=328 (M+H)⁺.

### 67. Compound 67: 3,3′-(6,7-Dihydrooxazolo[5,4-c]pyridine-2,5(4H)-diyl)dibenzonitrile

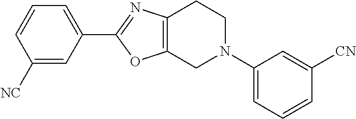

The title compound was prepared via the procedure used for Compound 64, using 3-(4,5,6,7-tetrahydrooxazolo[5,4-c]pyridin-2-yl)benzonitrile instead of 3-(4,5,6,7-tetrahydro-oxazolo[4,5-c]pyridin-2-yl)benzonitrile. Preparative TLC afforded 3,3′-(6,7-dihydrooxazolo-[5,4-c]pyridine-2,5(4H)-diyl)dibenzonitrile (1 mg, 7%) as a yellow solid. ¹H NMR (400 MHz, CDCl₃): δ 8.24 (s, 1H), 8.18-8.16 (m, 1H), 7.66-7.64 (m, 1H), 7.54-7.52 (m, 1H), 7.31 (d, 1H), 7.14-7.06 (m, 2H), 4.37 (s, 1H), 3.66 (t, 2H), 2.80 (t, 3H); LC/MS: m/e=327 (M+H)⁺.

### 68. Compound 68: 2-(2-Chlorophenyl)-5-(pyridin-2-yl)-4,5,6,7-tetrahydrooxazolo[5,4-c]pyridine

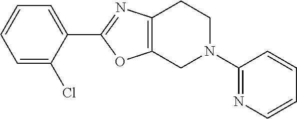

The title compound was prepared via the procedure used for Compound 60, using benzyl 2-(2-chlorophenyl)-6,7-dihydrooxazolo[5,4-c]pyridine-5(4H)-carboxylate instead of benzyl 2-m-tolyl-6,7-dihydrooxazolo[4,5-c]pyridine-5(4H)-carboxylate. Benzyl 2-(2-chlorophenyl)-6,7-dihydrooxazolo[5,4-c]pyridine-5(4H)-carboxylate was prepared via the procedure used for I-1.3, using 2-chlorobenzoic acid instead of picolinic acid. Preparative HPLC afforded 2-(2-chlorophenyl)-5-(pyridin-2-yl)-4,5,6,7-tetrahydrooxazolo[5,4-c]pyridine (18 mg, 28%). ¹H NMR (400 MHz, CDCl₃): δ 8.28 (d, 1H), 7.87-7.91 (m, 1H), 7.71-7.76 (m, 1H), 7.42-7.48 (m, 1H), 7.25-7.34 (m, 2H), 6.93 (d, 1H), 6.76-6.83 (m, 1H), 4.80 (s, 2H), 3.38-4.06 (m, 2H), 2.88-2.95 (m, 2H); LC/MS: m/e=312 (M+H)⁺.

### 69. Compound 69: 2-(2-(2-Chlorophenyl)-6,7-dihydrooxazolo[5,4-c]pyridin-5(4H)-yl)nicotinonitrile

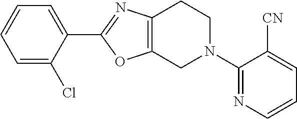

The title compound was prepared via the procedure used for Compound 63, using 2-(2-chlorophenyl)-4,5,6,7-tetrahydrooxazolo[5,4-c]pyridine instead of 3-(4,5,6,7-tetrahydro-oxazolo[4,5-c]pyridin-2-yl)-benzonitrile. 2-(2-Chlorophenyl)-4,5,6,7-tetrahydrooxazolo[5,4-c]pyridine was prepared via the procedure used for I-1.4, using 2-chlorobenzoic acid instead of picolinic acid. Preparative HPLC afforded 2-(2-(2-chlorophenyl)-6,7-dihydrooxazolo[5,4-c]pyridin-5(4H)-yl)nicotinonitrile (35 mg, 27%). ¹H NMR (400 MHz, CDCl₃): δ 8.29 (dd, 1H), 7.83-7.89 (m, 1H), 7.75 (dd, 1H), 7.41-7.49 (m, 1H), 7.25-7.35 (m, 2H), 6.75 (dd, 1H), 4.75 (s, 2H), 4.02 (t, 2H), 2.92 (t, 2H); LC/MS: m/e=337 (M+H)⁺.

### 70. Compound 70: 2-(4-Chlorophenyl)-5-(pyridin-2-yl)-4,5,6,7-tetrahydrooxazolo[5,4-c]pyridine

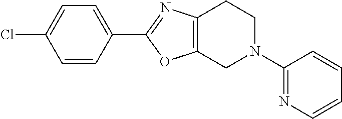

The title compound was prepared via the procedure used for Compound 60, using benzyl 2-(4-chlorophenyl)-6,7-dihydrooxazolo[5,4-c]pyridine-5(4H)-carboxylate instead of benzyl 2-m-tolyl-6,7-dihydrooxazolo[4,5-c]pyridine-5(4H)-carboxylate. Benzyl 2-(4-chlorophenyl)-6,7-dihydrooxazolo[5,4-c]pyridine-5(4H)-carboxylate was prepared via the procedure used for I-1.3, using 4-chlorobenzoic acid instead of picolinic acid. Preparative TLC afforded 2-(4-chlorophenyl)-5-(pyridin-2-yl)-4,5,6,7-tetrahydrooxazolo[5,4-c]pyridine (14 mg, 21%) as a yellow solid. ¹H NMR (400 MHz, CDCl₃): δ 8.14 (d, 1H); 7.86 (d, 2H), 7.45 (t, 1H), 7.35 (d, 2H), 6.68 (d, 1H), 6.59 (t, 1H), 4.68 (s, 2H), 3.88 (t, 2H), 2.75 (t, 2H); LC/MS: m/e=312 (M+H)⁺.

### 71. Compound 71: 2-(3-Methoxyphenyl)-5-(pyridin-2-yl)-4,5,6,7-tetrahydrooxazolo[5,4-c]pyridine

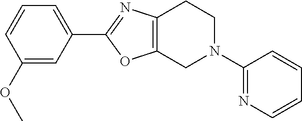

The title compound was prepared via the procedure used for Compound 1, using 3-methoxybenzoic acid instead of picolinic acid, and using 2-bromopyridine instead of 3-bromobenzonitrile. Preparative HPLC afford 2-(3-methoxyphenyl)-5-pyridin-2-yl-4,5,6,7-tetrahydrooxazolo[5,4-c]pyridine (1 mg, 1%) as a yellow liquid. ¹H NMR (400 MHz, CDCl₃): δ 8.20 (s, 1H), 7.88 (s, 1H), 7.52 (d, 1H), 7.45 (s, 1H), 7.30 (t, 1H), 7.04-7.02 (m, 1H), 6.96-6.89 (m, 2H), 4.82 (s, 1H), 4.03 (brs, 2H), 3.81 (s, 3H), 2.91 (brs, 2H); LC/MS: m/e=332 (M+H+Na)⁺.

### 72. Compound 72: 2-(2-(3-Methoxyphenyl)-6,7-dihydrooxazolo[5,4-c]pyridin-5(4H)-yl)nicotinonitrile

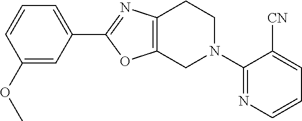

The title compound was prepared via the procedure used for Compound 63, using 2-(3-methoxyphenyl)-4,5,6,7-tetrahydrooxazolo[5,4-c]pyridine instead of 3-(4,5,6,7-tetrahydro-oxazolo[4,5-c]pyridin-2-yl)-benzonitrile. 2-(3-Methoxyphenyl)-4,5,6,7-tetrahydrooxazolo[5,4-c]pyridine was prepared via the procedure used for I-1.4, using 3-methoxybenzoic acid instead of picolinic acid. Preparative TLC afforded 2-(2-(3-methoxyphenyl)-6,7-dihydrooxazolo[5,4-c]pyridin-5(4H)-yl)nicotinonitrile (10.9 mg, 15%) as a yellow solid. ¹H NMR (400 MHz, CDCl₃): δ 8.32-8.31 (m, 1H), 7.79-7.75 (m, 1H), 7.53 (d, 2H), 7.48 (t, 1H), 6.99-6.96 (m, 1H), 6.79-6.76 (m, 1H), 4.77 (s, 1H), 4.01 (t, 2H), 2.96 (t, 2H); LC/MS: m/e=333 (M+H)⁺.

### 73. Compound 73: 2-(2-(3-Methoxyphenyl)-6,7-dihydrooxazolo[4,5-c]pyridin-5(4H)-yl)nicotinonitrile

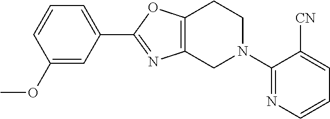

The title compound was prepared via the procedure used for Compound 63, using 2-(3-methoxyphenyl)-4,5,6,7-tetrahydrooxazolo[4,5-c]pyridine instead of 3-(4,5,6,7-tetrahydro-oxazolo[4,5-c]pyridin-2-yl)-benzonitrile. 2-(3-Methoxyphenyl)-4,5,6,7-tetrahydrooxazolo[4,5-c]pyridine was prepared via the procedure used for I-23.4, using 3-methoxybenzoic acid instead of picolinic acid. Preparative HPLC afforded 2-(2-(3-methoxyphenyl)-6,7-dihydrooxazolo[4,5-c]pyridin-5(4H)-yl)nicotinonitrile (50 mg, 35%). ¹H NMR (400 MHz, CDCl₃): δ 8.38 (dd, 1H), 7.83 (dd, 1H), 7.62 (d, 1H), 7.57-7.56 (m, 1H), 7.35 (t, 1H), 7.02-6.99 (m, 1H), 4.75 (t, 2H), 4.12 (t, 2H), 3.90 (s, 3H), 3.13-3.10 (m, 2H); LC/MS: m/e=333 (M+H)⁺.

### 74. Compound 74: 3-(2-(5-Methylpyridin-2-yl)-6,7-dihydrooxazolo[5,4-c]pyridin-5(4H)-yl)benzonitrile

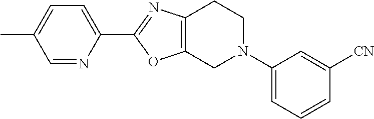

The title compound was prepared via the procedure used for Compound 64, using 2-(5-methylpyridin-2-yl)-4,5,6,7-tetrahydrooxazolo[5,4-c]pyridine instead of 3-(4,5,6,7-tetrahydrooxazolo[4,5-c]pyridin-2-yl)benzonitrile. 2-(5-Methylpyridin-2-yl)-4,5,6,7-tetrahydrooxazolo[5,4-c]pyridine was prepared via the procedure used for I-1.4, using 5-methylpicolinic acid instead of picolinic acid. Preparative HPLC afforded 3-(2-(5-methylpyridin-2-yl)-6,7-dihydrooxazolo[5,4-c]pyridin-5(4H)-yl)benzonitrile (1 mg, 2%) as a yellow solid. ¹H NMR (400 MHz, MeOH-d₄): δ 8.50 (s, 1H); 8.01 (d, 1H), 7.82 (d, 1H), 7.37 (m, 3H), 7.13 (d, 1H), 4.51 (s, 2H), 3.77 (t, 2H), 2.76-2.85 (m, 2H), 2.42 (s, 3H); LC/MS: m/e=317 (M+H)⁺.

### 75. Compound 75: 3-(2-(Pyridin-4-yl)-6,7-dihydrooxazolo[5,4-c]pyridin-5(4H)-yl)benzonitrile

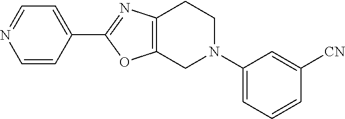

The title compound was prepared via the procedure used for Compound 64, using 2-(pyridin-4-yl)-4,5,6,7-tetrahydrooxazolo[5,4-c]pyridine instead of 3-(4,5,6,7-tetrahydro-oxazolo[4,5-c]pyridin-2-yl)benzonitrile. 2-(Pyridin-4-yl)-4,5,6,7-tetrahydro-oxazolo[5,4-c]pyridine was prepared via the procedure used for I-1.4, using isonicotinic acid instead of picolinic acid. Preparative TLC afforded 3-(2-(pyridin-4-yl)-6,7-dihydrooxazolo[5,4-c]pyridine-5(4H)-yl)benzonitrile (12 mg, 40%) as a yellow solid. ¹H NMR (400 MHz, CDCl₃): δ 8.68 (m, 2H), 7.84 (d, 2H), 7.30 (m, 1H), 7.10 (m, 3H), 4.48 (s, 2H), 3.66 (t, 2H), 2.80 (t, 2H); LC/MS: m/e=303 (M+H)⁺.

### 76. Compound 76: 3-(2-(4-Methylpyridin-2-yl)-6,7-dihydrooxazolo[5,4-c]pyridin-5(4H)-yl)benzonitrile

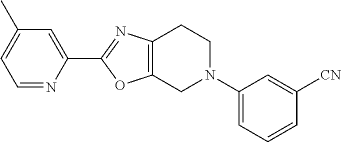

To a solution of 2-(4-methylpyridin-2-yl)-4,5,6,7-tetrahydrooxazolo[5,4-c]pyridine (I-76.2) (40 mg, 0.18 mmol) in toluene (2 mL) was added 3-bromobenzo-nitrile (51 mg, 0.28 mmol), Cs₂CO₃ (121 mg, 0.37 mmol), Pd(OAc)₂ (1 mg, cat.), and Xantphos (4 mg, cat.). The mixture was heated to 100° C. and stirred overnight. The mixture was dissolved in MeOH and filtered. The filtrate was concentrated and the residue was purified by preparative HPLC to afford 3-(2-(4-methylpyridin-2-yl)-6,7-dihydrooxazolo[5,4-c]pyridin-5(4H)-yl)benzonitrile (10 mg, 17%) as a yellow solid. ¹H NMR (400 MHz, MeOH-d₄): δ 8.53 (d, 1H), 8.10 (s, 1H), 7.50 (d, 1H), 7.40 (t, 1H), 7.31-7.40 (m, 2H), 7.13 (d, 1H), 4.51 (s, 2H), 3.77 (t, 2H), 2.80-2.90 (m, 2H), 2.53 (s, 3H); LC/MS: m/e=317 (M+H)⁺.

### 76.1 Benzyl 2-(4-methylpyridin-2-yl)-6,7-dihydrooxazolo[5,4-c]pyridine-5(4H)-carboxylate (I-76.1)

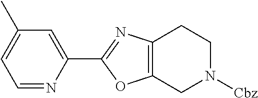

The title compound was prepared via the procedure used for I-1.3, using 4-methylpicolinic acid instead of picolinic acid. Preparative TLC afforded benzyl 2-(4-methylpyridin-2-yl)-6,7-dihydrooxazolo[5,4-c]pyridine-5(4H)-carboxylate (100 mg, 23%) as a yellow liquid. ¹H NMR (400 MHz, CDCl₃): δ 8.50 (d, 1H), 7.88 (s, 1H), 7.22-7.36 (m, 5H), 7.13 (d, 1H), 5.11 (s, 2H), 4.65 (s, 2H), 3.70-3.82 (m, 2H), 2.60-2.75 (m, 2H), 2.37 (s, 3H).

### 76.2 2-(4-Methylpyridin-2-yl)-4,5,6,7-tetrahydrooxazolo[5,4-c]pyridine (I-76.2)

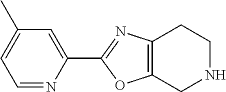

To a solution of benzyl 2-(4-methylpyridin-2-yl)-6,7-dihydrooxazolo[5,4-c]pyridine-5(4H)-carboxylate (100 mg, 0.29 mmol) in MeCN was added TMSI (570 mg, 2.9 mmol). The mixture was stirred for 1 h at room temperature, then concentrated to dryness. The residue was washed with ether to afford 2-(4-methylpyridin-2-yl)-4,5,6,7-tetrahydrooxazolo[5,4-c]pyridine (60 mg, 100%) as a yellow solid. ¹H NMR (400 MHz, MeOH-d₄): δ 8.69 (d, 1H), 8.40 (s, 1H), 7.90 (d, 1H), 4.61 (s, 2H), 3.68 (t, 2H), 3.06 (t, 2H), 2.71 (s, 3H).

### 77. Compound 77: 3-(2-(Pyridin-3-yl)-6,7-dihydrooxazolo[5,4-c]pyridin-5(4H)-yl)benzonitrile

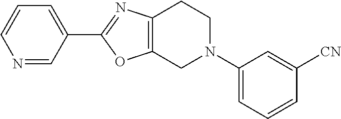

The title compound was prepared via the procedure used for Compound 76, using benzyl 2-(pyridin-3-yl)-6,7-dihydrooxazolo[5,4-c]pyridine-5(4H)-carboxylate instead of benzyl 2-(4-methylpyridin-2-yl)-6,7-dihydrooxazolo[5,4-c]pyridine-5(4H)-carboxylate. Benzyl 2-(pyridin-3-yl)-6,7-dihydrooxazolo[5,4-c]pyridine-5(4H)-carboxylate was prepared via the procedure used for I-1.3, using nicotinic acid instead of picolinic acid. ¹H NMR (400 MHz, CDCl₃): δ=9.33 (d, 1H), 8.75 (dd, 1H), 8.59 (dd, 1H), 7.69 (dd, 1H), 7.38-7.34 (m, 1H), 7.17-7.12 (m, 3H), 4.43 (s, 2H), 3.71 (t, 2H), 2.86 (t, 2H). LC/MS: m/e=303 (M+H)⁺.

### 78. Compound 78: 3-(2-(Pyridin-4-yl)-6,7-dihydrooxazolo[4,5-c]pyridin-5(4H)-yl)benzonitrile

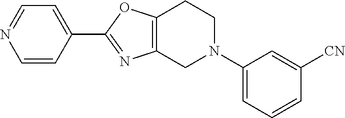

The title compound was prepared via the procedure used for Compound 76, using benzyl 2-(pyridin-4-yl)-6,7-dihydrooxazolo[4,5-c]pyridine-5(4H)-carboxylate instead of benzyl 2-(4-methylpyridin-2-yl)-6,7-dihydrooxazolo[5,4-c]pyridine-5(4H)-carboxylate. Benzyl 2-(pyridin-4-yl)-6,7-dihydrooxazolo[4,5-c]pyridine-5(4H)-carboxylate was prepared via the procedure used for I-23.3, using isonicotinic acid instead of picolinic acid. Preparative HPLC afforded 3-(2-(pyridin-4-yl)-6,7-dihydro-oxazolo[4,5-c]pyridin-5(4H)-yl)benzonitrile (10 mg, 13%). ¹H NMR (400 MHz, CDCl₃): δ 8.78 (d, 2H), 8.12 (d, 2H), 7.28-7.33 (m, 1H), 7.07-7.13 (m, 3H), 4.29 (s, 2H), 3.72 (t, 2H), 2.93-3.00 (t, 2H); LC/MS: m/e=303 (M+H)⁺.

### 79. Compound 79: 2-(3-Methylisoxazol-5-yl)-5-(pyridin-2-yl)-4,5,6,7-tetrahydrooxazolo[4,5-c]pyridine

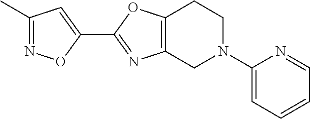

The title compound was prepared via the procedure used for Compound 60, using benzyl 2-(3-methylisoxazol-5-yl)-6,7-dihydrooxazolo[4,5-c]pyridine-5(4H)-carboxylate instead of benzyl 2-m-tolyl-6,7-dihydrooxazolo[4,5-c]pyridine-5(4H)-carboxylate. Benzyl 2-(3-methylisoxazol-5-yl)-6,7-dihydrooxazolo[4,5-c]pyridine-5(4H)-carboxylate was prepared via the procedure used for I-23.3 using 3-methylisoxazole-5-carboxylic acid instead of picolinic acid. Preparative HPLC afforded 2-(3-methylisoxazol-5-yl)-5-(pyridin-2-yl)-4,5,6,7-tetrahydro-oxazolo[4,5-c]pyridine (12 mg, 6.3%). ¹H NMR (400 MHz, MeOH-d₄): δ 8.07-8.03 (m, 2H), 7.47-7.45 (m, 1H), 7.04 (t, 1H), 6.89 (s, 1H), 4.88 (s, 2H), 4.08 (t, 2H), 2.91 (t, 2H), 2.35 (s, 3H). LC/MS: m/e=283 (M+H)⁺.

### 80. Compound 80: 3-Fluoro-5-(2-(oxazol-2-yl)-6,7-dihydrooxazolo[4,5-c]pyridin-5(4H)-yl)benzonitrile

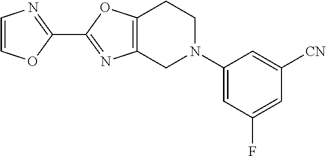

A mixture of 2-oxazol-2-yl-4,5,6,7-tetrahydro-oxazolo[4,5-c]pyridine (1-80.7) (103 mg, 0.32 mmol), 3-bromo-5-fluorobenzonitrile (200 mg, 1 mmol), Pd(OAc)₂ (5 mg, 0.02 mmol), Xantphos (40 mg, 0.03 mmol), and Cs₂CO₃ (200 mg, 0.61 mmol) in degassed toluene (10 mL) was heated at reflux for 12 h under nitrogen. The solids were removed by filtration and the filtrate was concentrated to dryness. The residue was purified by preparative TLC to give the product as yellow solid (44 mg, 44%). ¹H NMR (400 MHz, CDCl₃): δ 7.75 (d, 1H), 7.28 (d, 1H), 6.91-6.90 (m, 1H), 6.79-6.72 (m, 2H), 4.27-4.26 (m, 2H), 3.71 (t, 2H), 2.96-2.92 (m, 2H); LC/MS: m/e=311 (M+H)⁺.

### 80.1 3-(Ethoxyoxalyl-amino)-4-hydroxy-piperidine-1-carboxylic acid benzyl ester (I-80.1)

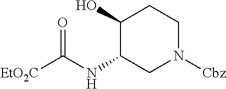

A mixture of 3-amino-4-hydroxy-piperidine-1-carboxylic acid benzyl ester (prepared according to the procedure in Hall, S. E., et al., WO1994/20062) (2.5 g, 10 mmol) and oxalic acid diethyl ester (10 mL) was stirred at room temperature overnight. Petroleum ether (50 mL) was added, and the white precipitate was collected by filtration (3.284 g, 94%). ¹H NMR (400 MHz, CDCl₃): δ 7.37-7.32 (m, 5H), 5.15 (s, 2H), 4.35 (q, 2H), 4.15-4.08 (m, 1H), 4.10-3.80 (m, 3H), 3.20 (brs, 2H), 1.97 (brs, 1H), 1.63-1.61 (m, 1H), 1.37 (t, 3H).

### 80.2 3-(Ethoxyoxalyl-amino)-4-oxo-piperidine-1-carboxylic acid benzyl ester (I-80.2)

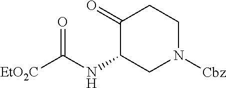

To a solution of 3-(ethoxyoxalyl-amino)-4-hydroxy-piperidine-1-carboxylic acid benzyl ester (3.28 g, 9.4 mmol) in DCM (60 mL) was added Dess-Martin reagent (6.5 g, 15.3 mmol). The resulting mixture was stirred at room temperature overnight. Aqueous sodium carbonate was added to basify the system, and, after 30-minute stirring, the DCM phase was separated, dried and concentrated. The crude product (2.4 g, 74%) was carried through to the next step without purification.

### 80.3 6,7-Dihydro-4H-oxazolo[4,5-c]pyridine-2,5-dicarboxylic acid 5-benzyl ester 2-ethyl ester (I-80.3)

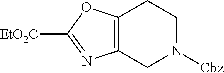

To a solution of 3-(ethoxyoxalyl-amino)-4-oxo-piperidine-1-carboxylic acid benzyl ester (2.4 g, 6.9 mmol) in anhydrous dioxane (80 mL) was added freshly distilled POCl₃ (2.4 mL, 15 mmol), and the resulting mixture was heated at 100° C. for 4 h. Then all volatiles were removed under reduced pressure, and the brown residue was treated with saturated aq. Na₂CO₃ until the pH reached 9. The product was extracted by DCM (30 mL) for 3 times, and the combined organic layers were dried over Na₂SO₄. After filtration and concentration, the product was purified by chromatography on silica gel (1.12 g, 49%). ¹H NMR (400 MHz, CDCl₃): δ 7.29 (brs, 5H), 5.10 (s, 2H), 4.44-4.37 (m, 2H), 3.80 (brs, 2H), 2.80 (brs, 2H), 1.39-1.35 (m, 3H).

### 80.4 2-(2,2-Dimethoxy-ethylcarbamoyl)-6,7-dihydro-4H-oxazolo[4,5-c]pyridine-5-carboxylic acid benzyl ester (I-80.4)

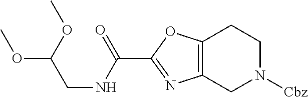

To a solution of 6,7-dihydro-4H-oxazolo[4,5-c]pyridine-2,5-dicarboxylic acid 5-benzyl ester 2-ethyl ester (1.12 g, 3.38 mmol) in methanol (10 mL) was added 2,2-dimethoxy-ethylamine (3.15 g, 30 mmol), and the solution was stirred at room temperature overnight. After removal of the volatiles, the residue was purified by chromatography on silica gel (0.99 g, 84%). ¹H NMR (400 MHz, CDCl₃): δ 7.38-7.34 (m, 5H), 7.11 (brs, 1H), 5.18 (s, 2H), 4.53-4.46 (m, 3H), 3.87 (s, 2H), 3.60-3.58 (m, 2H), 3.43 (s, 6H), 2.86 (s, 2H).

### 80.5 2-(2-oxo-ethylcarbamoyl)-6,7-dihydro-4H-oxazolo[4,5-c]pyridine-5-carboxylic acid benzyl ester (I-80.5)

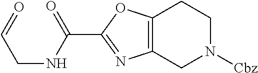

To a solution of 2-(2,2-dimethoxy-ethylcarbamoyl)-6,7-dihydro-4H-oxazolo[4,5-c]pyridine-5-carboxylic acid benzyl ester (0.45 g, 1.15 mmol) in DCM (10 mL) was added TFA (3 mL), and the resulting solution was stirred at room temperature. After the complete disappearance of the starting material, all volatiles were removed under reduced pressure. Aqueous NaHCO₃ was added to basify the system to pH 8, and the product was extracted with DCM. After drying, filtration and concentration, 0.44 g (100%) of the crude product was obtained and carried through to the next step without purification.

### 80.6 2-Oxazol-2-yl-6,7-dihydro-4H-oxazolo[4,5-c]pyridine-5-carboxylic acid benzyl ester (I-80.6)

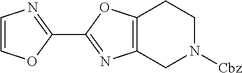

To the solution of 2-(2-oxo-ethylcarbamoyl)-6,7-dihydro-4H-oxazolo[4,5-c]pyridine-5-carboxylic acid benzyl ester (0.44 g, 1.15 mmol) in DCM (10 mL) was added Ph₃P (1.2 g, 4.6 mmol), TEA (1 mL), and I₂ (1.17 g, 4.6 mmol), and the resulting solution was stirred at room temperature for 30 minutes. The reaction was partitioned with aq. Na₂S₂O₃/Na₂CO₃, and the organic phase was separated. Purification yielded a crude reaction product that was contaminated with Ph₃PO, and this material was carried through to the next step.

### 80.7 2-Oxazol-2-yl-4,5,6,7-tetrahydro-oxazolo[4,5-c]pyridine (I-80.7)

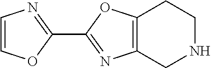

To the solution of 2-oxazol-2-yl-6,7-dihydro-4H-oxazolo[4,5-c]pyridine-5-carboxylic acid benzyl ester in acetonitrile (10 mL) was added TMSI (1.5 mL, 10.5 mmol) in one portion at room temperature, and the resulting mixture was stirred at room temperature for about 2 h. The solvent and excess TMSI were removed under reduced pressure. The brown solid residue was washed with ether (3×5 mL), filtered and dried (103 mg, 25%). ¹H NMR (400 MHz, DMSO-d₆): δ 9.18 (s, 2H), 8.38 (s, 1H), 7.52 (s, 1H), 4.25 (s, 2H), 3.53 (t, 2H), 3.07 (t, 2H).

### 81. Compound 81: (+/−)-3-Fluoro-5-(7-methyl-2-(pyridin-2-yl)-6,7-dihydrooxazolo[5,4-c]pyridin-5(4H)-yl)benzonitrile

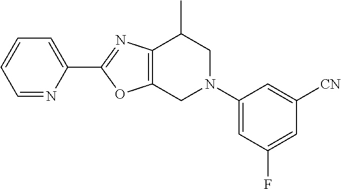

To a solution of 7-methyl-2-(pyridin-2-yl)-4,5,6,7-tetrahydrooxazolo[5,4-c]pyridine (I-81.11) (20 mg, 0.1 mmol) in toluene (2 mL) was added 3-bromo-5-fluorobenzonitrile (28 mg, 0.15 mmol), Cs₂CO₃ (65 mg, 0.2 mmol), Pd(OAc)₂ (1 mg, cat.), and Xantphos (2 mg, cat.). The mixture was heated to 100° C. and stirred overnight. The reaction was cooled, dissolved in MeOH and filtered. The filtrate was concentrated and the residue was purified by preparative TLC to afford 3-fluoro-5-(7-methyl-2-(pyridin-2-yl)-6,7-dihydrooxazolo[5,4-c]pyridin-5(4H)-yl)benzonitrile (8 mg, 26%) as a yellow solid. ¹H NMR (400 MHz, CDCl₃): δ 8.67 (d, 1H), 8.05 (d, 1H), 7.76 (t, 1H), 7.30 (t, 1H), 6.88 (s, 1H), 6.70-6.79 (m, 2H), 4.36 (q, 2H), 3.72 (dd, 1H), 3.20 (q, 1H), 3.00-3.10 (m, 1H), 1.31 (d, 3H); LC/MS: m/e=335 (M+H)⁺.

### 81.1 (+/−)-1-Benzyl-3-methylpiperidin-4-one (I-81.1)

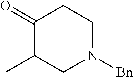

To a solution of NaH (15 g, 0.38 mol, 60% dispersion) in THF (600 mL) was added 1-benzylpiperidin-4-one (60 g, 0.3 mol) in THF (100 mL) at 0° C. The mixture was stirred for 30 min. Methyl iodide (67 g, 0.47 mol) was added and the reaction was stirred overnight at 60° C. The reaction was cooled to room temperature, filtered, and the filtrate washed with water and extracted with EtOAc (3×300 mL). The organic phase was dried over anhydrous Na₂SO₄ and concentrated. The residue was purified by column on silica gel (PE:EtOAc=5:1) to afford 1-benzyl-3-methylpiperidin-4-one (30 g, 47%) as a yellow oil. ¹H NMR (400 MHz, DMSO-d₆): δ 7.20-7.35 (m, 5H), 3.58 (s, 2H), 2.95-3.02 (m, 2H), 2.48-2.62 (m, 2H), 2.26-2.32 (m, 1H), 2.10-2.18 (m, 1H), 2.00 (q, 1H), 0.81 (d, 3H).

### 81.2 (+/−)-Benzyl 3-methyl-4-oxopiperidine-1-carboxylate (I-81.2)

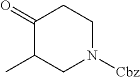

To a solution of 1-benzyl-3-methylpiperidin-4-one (20 g, 98 mmol) in toluene (150 mL) was added Cbz-Cl (25.1 g, 150 mmol). The mixture was heat to reflux and stirred overnight. The reaction was cooled and water was added. The mixture was extracted with EtOAc (3×150 mL). The organic phase was dried over anhydrous Na₂SO₄ and concentrated. The residue was purified by column on silica gel (PE:EtOAc=5:1) to afford benzyl 3-methyl-4-oxopiperidine-1-carboxylate (20 g, 83%) as a yellow oil. ¹H NMR (400 MHz, DMSO-d₆): δ 7.25-7.37 (m, 5H), 5.10 (s, 2H), 4.00-4.12 (m, 2H), 3.30-3.38 (m, 1H), 2.80-3.01 (m, 1H), 2.50-2.60 (m, 1H), 2.40-2.50 (m, 1H), 2.21-2.30 (m, 1H), 0.89 (d, 3H).

### 81.3 (+/−)-Benzyl 5-methyl-4-(trifluoromethylsulfonyloxy)-5,6-dihydropyridine-1(2H)-carboxylate (I-81.3)

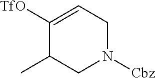

To a solution of LiHMDS (161 mL, 1M) in THF (100 ml) was added (+/−)-benzyl 3-methyl-4-oxopiperidine-1-carboxylate (20 g, 81 mmol) in THF (200 mL) at −78° C. The mixture was stirred for 2 h at −78° C. 1,1,1-Trifluoro-N-phenyl-N-(trifluoro-methyl-sulfonyl)methane-sulfonamide (43 g, 121 mmol) in THF (250 mL) was added dropwise at −78° C. The mixture was warmed to room temperature and stirred for 3 h. The reaction was quenched with aq. NH₄Cl solution and extracted with EtOAc (3×350 mL). The organic phase was dried over anhydrous Na₂SO₄ and concentrated. The residue was purified by column on silica gel (PE:EtOAc=10:1) to afford benzyl 5-methyl-4-(trifluoromethylsulfonyloxy)-5,6-dihydropyridine-1(2H)-carboxylate (20 g, 65%) as a yellow oil. ¹H NMR (400 MHz, DMSO-d₆): δ 7.25-7.40 (m, 5H), 5.90-6.00 (m, 1H), 5.10 (d, 2H), 3.90-4.15 (m, 2H), 3.62 (dd, 1H), 3.33-3.42 (m, 1H), 2.55-2.65 (m, 1H), 1.00 (d, 3H).

### 81.4 (+/−)-Benzyl 5-methyl-5,6-dihydropyridine-1(2H)-carboxylate (I-81.4)

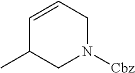

To a solution of benzyl 5-methyl-4-(trifluoromethylsulfonyloxy)-5,6-dihydro-pyridine-1(2H)-carboxylate (20 g, 53 mmol) in DMF (150 mL) was added Pd(OAc)₂ (237 mg, 1.05 mmol), PPh₃ (553 mg, 2.11 mmol), and TEA (17.4 g, 172 mmol). Formic acid (5 mL, 52.7 mmol) was added and the mixture was heated for 1 h at 60° C. The reaction was cooled, poured into water, and extracted with EtOAc (3×150 mL). The organic phase was dried over anhydrous Na₂SO₄ and concentrated. The residue was purified by silica gel chromatography (PE:EtOAc=10:1) to afford (+/−)-benzyl 5-methyl-5,6-dihydropyridine-1(2H)-carboxylate (10.4 g, 85%) as a yellow oil. ¹H NMR (400 MHz, DMSO-d₆): δ 7.28-7.40 (m, 5H), 5.53-5.70 (m, 2H), 5.15 (s, 2H), 4.05 (dd, 1H), 3.75-3.91 (m, 2H), 2.80-3.00 (m, 1H), 2.26-2.40 (m, 1H), 0.98 (d, 3H)

### 81.5 (+/−)-Benzyl 5-methyl-7-oxa-3-azabicyclo[4.1.0]heptane-3-carboxylate (I-81.5)

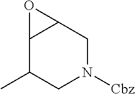

To a solution of (+/−)-benzyl 5-methyl-5,6-dihydropyridine-1(2H)-carboxylate (10.4 g, 45 mmol) in DCM (100 mL) was added m-CPBA (23 g, 135 mmol). The reaction was stirred overnight at RT. Then reaction was quenched with aq. Na₂S₂O₄ solution and extracted with EtOAc (3×100 mL). The organic phase was dried over anhydrous Na₂SO₄ and concentrated. The residue was purified by silica gel chromatography (PE:EtOAc=5:1) to afford (+/−)-benzyl 5-methyl-7-oxa-3-azabicyclo[4.1.0]heptane-3-carboxylate (4.3 g, 39%) as a yellow oil. ¹H NMR (400 MHz, CDCl₃): δ 7.27-7.40 (m, 5H), 5.07-5.18 (m, 2H), 3.00-4.20 (m, 5H), 2.57-2.70 (m, 1H), 2.05-2.30 (m, 1H), 1.00-1.02 (m, 3H).

### 81.6 (+/−)-Benzyl 4-azido-3-hydroxy-5-methylpiperidine-1-carboxylate (I-81.6a) and (+/−)-Benzyl 3-azido-4-hydroxy-5-methylpiperidine-1-carboxylate (I-81.6b)

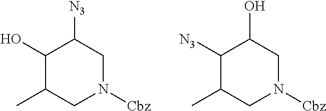

To a solution of (+/−)-benzyl 5-methyl-7-oxa-3-azabicyclo[4.1.0]heptane-3-carboxylate (3.5 g, 14 mmol) in MeOH/H₂O (10:1) (50 mL) was added NH₄Cl (1.5 g, 28 mmol) followed by sodium azide (1.82 g, 28 mmol) slowly. The reaction was then heated to reflux and stirred for 16 h. The reaction was cooled and aq.NaHCO₃ was added until the pH was 9. The aqueous phase was extracted with DCM (3×150 mL). The combined organic phases were dried over anhydrous Na₂SO₄ and concentrated. The residue was purified by silica gel chromatography (PE:EtOAc=3:1) to afford a mixture of benzyl 4-azido-3-hydroxy-5-methylpiperidine-1-carboxylate and benzyl 3-azido-4-hydroxy-5-methylpiperidine-1-carboxylate (1.7 g, 43%) as a yellow oil. The product was carried through to the next step as a mixture. ¹H NMR (400 MHz, DMSO-d₆): δ 7.23-7.40 (m, 5H), 5.31-5.50 (m, 1H), 5.00 (s, 2H), 3.20-3.70 (m, 5H), 2.90-3.20 (m, 1H), 1.75-2.10 (m, 1H), 0.80 (d, 3H).

### 81.7 (+/−)-Benzyl 4-amino-3-hydroxy-5-methylpiperidine-1-carboxylate (I-81.7a) and (+/−)-Benzyl 3-amino-4-hydroxy-5-methylpiperidine-1-carboxylate (I-81.7b)

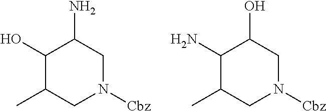

To a mixture of (+/−)-benzyl 4-azido-3-hydroxy-5-methylpiperidine-1-carboxylate and (+/−)-benzyl 3-azido-4-hydroxy-5-methylpiperidine-1-carboxylate (1.7 g, 6 mmol) in THF (20 mL) and H₂O (1 mL) was added Ph₃P (2.3 g, 9 mmol). The reaction was heated to reflux and stirred overnight. Concentration in vacuo afforded the crude product which was purified by silica gel chromatography (DCM:MeOH=5:1) to afford a mixture of (+/−)-benzyl 4-amino-3-hydroxy-5-methylpiperidine-1-carboxylate and (+/−)-benzyl 3-amino-4-hydroxy-5-methyl-piperidine-1-carboxylate (1.4 g, 90%) as a yellow liquid. The product was carried through to the next step as a mixture. ¹H NMR (400 MHz, DMSO-d₆): δ 7.23-7.40 (m, 5H), 5.02 (d, 2H), 4.65-4.91 (m, 1H), 3.50-3.70 (m, 1H), 2.80-3.25 (m, 4H), 2.55-2.67 (m, 1H), 1.85-1.98 (m, 1H), 0.75 (d, 3H).

### 81.8 (+/−)-Benzyl 4-hydroxy-3-methyl-5-(picolinamido)piperidine-1-carboxylate (I-81.8a) and (+/−)-Benzyl 3-hydroxy-5-methyl-4-(picolinamido)piperidine-1-carboxylate (I-81.8b)

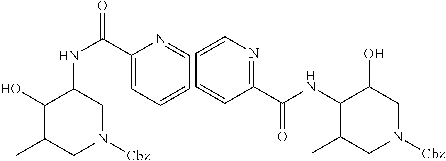

To a solution of picolinic acid (717 mg, 5.83 mmol) and TEA (1.07 g, 10.6 mmol) in DCM (15 mL) was added EDCI (1.95 g, 10.6 mmol) and HOBt (1.43 g, 10.6 mmol). A mixture of (+/−)-benzyl 4-amino-3-hydroxy-5-methylpiperidine-1-carboxylate and (+/−)-benzyl 3-amino-4-hydroxy-5-methylpiperidine-1-carboxylate (1.4 g, 5.3 mmol) in DCM (15 mL) was added, and the reaction was stirred overnight at room temperature. The reaction was diluted with DCM and washed with water. The organic phase was washed with aq. NaHCO₃, 1N HCl solution and brine. The combined organic phases were dried over anhydrous Na₂SO₄ and concentrated. The residue was purified by silica gel chromatography (PE:EtOAc=1:1) to afford (+/−)-benzyl 4-hydroxy-3-methyl-5-(picolinamido)piperidine-1-carboxylate (1 g, 50%) as a yellow oil, and (+/−)-benzyl 3-hydroxy-5-methyl-4-(picolinamido)piperidine-1-carboxylate (500 mg, 25%) as a yellow oil. For (+/−)-benzyl 4-hydroxy-3-methyl-5-(picolinamido)piperidine-1-carboxylate, ¹H NMR (400 MHz, DMSO-d₆): δ 8.60 (d, 1H), 8.32 (d, 1H), 7.90-8.06 (m, 2H), 7.60 (t, 1H), 7.15-7.40 (m, 5H), 5.13 (br, 1H), 5.04 (s, 2H), 3.85-3.92 (m, 1H), 3.60-3.75 (m, 2H), 3.40-3.60 (m, 2H), 3.00-3.20 (m, 1H), 1.80-1.92 (m, 1H), 0.84 (d, 3H). For (+/−)-benzyl 3-hydroxy-5-methyl-4-(picolinamido)piperidine-1-carboxylate, ¹H NMR (400 MHz, DMSO-d₆): δ 8.62 (d, 1H), 8.37 (d, 1H), 7.90-8.05 (m, 2H), 7.59 (t, 1H), 7.25-7.40 (m, 5H), 5.15 (br, 1H), 5.05 (s, 2H), 3.40-3.91 (m, 5H), 2.85-3.16 (m, 1H), 2.25-2.45 (m, 1H), 0.77 (d, 3H).

### 81.9 (+/−)-Benzyl 3-methyl-5-oxo-4-(picolinamido)piperidine-1-carboxylate (I-81.9)

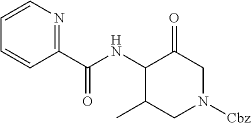

To a solution of (+/−)-benzyl 3-hydroxy-5-methyl-4-(picolinamido)-piperidine-1-carboxylate (500 mg, 1.35 mmol) in DCM (20 mL) was added Dess-Martin reagent (1.15 g, 2.7 mmol). The reaction was stirred overnight at room temperature. The reaction was diluted with DCM and washed with 0.5 N NaOH solution. The organic phase was dried over anhydrous Na₂SO₄ and concentrated to afford benzyl 3-methyl-5-oxo-4-(picolinamido)-piperidine-1-carboxylate (470 mg, 94%) as a yellow oil. ¹H NMR (400 MHz, DMSO-d₆): δ 8.60-8.72 (m, 2H), 7.95-8.10 (m, 2H), 7.61 (t, 1H), 7.25-7.40 (m, 5H), 5.10 (s, 2H), 5.00 (t, 1H), 4.30 (d, 1H), 3.95-4.15 (m, 1H), 3.92 (d, 1H), 3.60-3.77 (m, 1H), 2.75-2.85 (m, 1H), 0.70 (d, 3H).

### 81.10 (+/−)-Benzyl 7-methyl-2-(pyridin-2-yl)-6,7-dihydrooxazolo[5,4-c]pyridine-5(4H)-carboxylate (I-81.10)

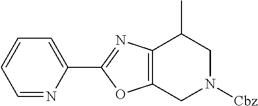

To a solution of POCl₃ (820 mg, 5.4 mmol) in dioxane (15 mL), benzyl 3-methyl-5-oxo-4-(picolinamido)piperidine-1-carboxylate (470 mg, 1.34 mmol) in dioxane (15 mL) was added. The reaction mixture was heated to reflux and stirred for 3 h. The reaction was cooled, poured into aq. NaHCO₃ and extracted with EtOAc (3×50 mL). The organic phase was dried over anhydrous Na₂SO₄ and concentrated. The residue was purified by silica gel chromatography (PE:EtOAc=1:1) to afford benzyl 7-methyl-2-(pyridin-2-yl)-6,7-dihydro-oxazolo[5,4-c]pyridine-5(4H)-carboxylate (150 mg, 34%). ¹H NMR (400 MHz, CDCl₃): δ 8.65 (d, 1H), 8.00 (d, 1H), 7.73 (t, 1H), 7.22-7.35 (m, 6H), 5.10 (s, 2H), 4.70 (d, 1H), 4.50-4.60 (m, 1H), 3.72-4.00 (m, 1H), 3.20-3.35 (m, 1H), 2.87-3.00 (m, 1H), 0.71 (d, 3H).

### 81.11 (+/−)-7-Methyl-2-(pyridin-2-yl)-4,5,6,7-tetrahydrooxazolo[5,4-c]pyridine (I-81.11)

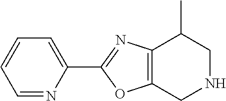

To a solution of benzyl 7-methyl-2-(pyridin-2-yl)-6,7-dihydrooxazolo[5,4-c]pyridine-5(4H)-carboxylate (150 mg, 0.43 mmol) in MeCN (5 mL) was added TMSI (860 mg, 4.3 mmol). The mixture was stirred for 1 h at room temperature. The reaction was concentrated, and the residue was washed with ether to afford 7-methyl-2-(pyridin-2-yl)-4,5,6,7-tetrahydro-oxazolo[5,4-c]pyridine (50 mg, 54%, crude) as a yellow solid. ¹H NMR (400 MHz, MeOH-d₄): δ 8.78 (d, 1H), 8.31-8.40 (m, 2H), 7.81 (t, 1H), 4.57 (s, 2H), 3.78 (dd, 1H), 3.33-3.40 (m, 1H), 3.16-3.22 (m, 1H), 1.41 (d, 3H).

### 82. Compound 82: (+/−)-3-Fluoro-5-(7-methyl-2-(pyridin-2-yl)-6,7-dihydrooxazolo[4,5-c]pyridin-5(4H)-yl)benzonitrile

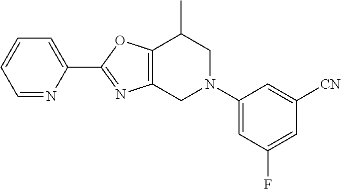

The title compound was prepared via the procedure used for Compound 81, using (+/−)-benzyl 3-hydroxy-5-methyl-4-(picolinamido)piperidine-1-carboxylate instead of (+/−)-benzyl 4-hydroxy-3-methyl-5-(picolinamido)piperidine-1-carboxylate. Preparative TLC afforded 3-fluoro-5-(7-methyl-2-(pyridin-2-yl)-6,7-dihydrooxazolo-[4,5-c]pyridin-5(4H)-yl)benzonitrile (15 mg, 19%) as a yellow solid. ¹H NMR (400 MHz, CDCl₃): δ 8.69 (d, 1H); 8.05 (d, 1H), 7.77 (t, 1H), 7.30 (t, 1H), 6.90 (s, 1H), 6.70-6.79 (m, 2H), 4.22 (q, 2H), 3.75 (dd, 1H), 3.18-3.30 (m, 2H), 1.35 (d, 3H); LC/MS: m/e=335 (M+H)⁺.

### 83. Compound 83: 3-Fluoro-5-(6-methyl-2-(pyridin-2-yl)-6,7-dihydrooxazolo[5,4-c]pyridin-5(4H)-yl)benzonitrile

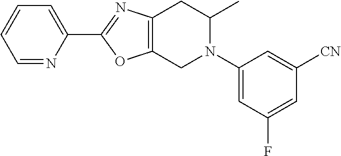

A mixture of 6-methyl-2-(pyridin-2-yl)-4,5,6,7-tetrahydrooxazolo[5,4-c]pyridine (I-83.9) (150 mg, 0.70 mmol), 3-bromo-5-fluorobenzonitrile (200 mg, 1 mmol), Pd(OAc)₂ (5 mg, 0.022 mmol), Xantphos (20 mg, 0.03 mmol), and Cs₂CO₃ (200 mg, 0.61 mmol) in degassed toluene (10 mL) was heated at reflux for 12 h under nitrogen. The solids were removed by filtration and the filtrate was concentrated. The residue was successively purified by preparative TLC and HPLC to give the product as yellow solid (10 mg, 7.2%). ¹H NMR (400 MHz, CDCl₃): δ 8.75 (d, 1H), 8.14 (d, 1H), 7.95-7.91 (m, 1H), 7.48-7.45 (m, 1H), 6.90 (s, 1H), 6.78-6.75 (m, 2H), 4.47-4.40 (m, 2H), 4.18 (d, 1H), 3.14-3.08 (m, 1H), 2.59 (d, 1H), 1.13 (d, 3H); LCMS: m/e=335 (M+H)⁺.

### 83.1 1-Benzyl-2-methylpyridinium bromide (I-83.1)

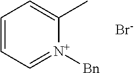

To a solution of 2-methylpyridine (18.6 g, 0.2 mol) in acetone (50 mL) was added benzyl bromide (34.2 g, 0.2 mol) in one portion, and the resulting mixture was heated at reflux overnight. The product precipitated as white solid, and was collected by filtration (52.8 g, 100%). ¹H NMR (400 MHz, DMSO-d₆): δ 9.14 (d, 1H), 8.58-8.54 (m, 1H), 8.11-8.04 (m, 2H), 7.45-7.38 (m, 3H), 7.26 (d, 2H), 5.92 (s, 2H), 2.75 (s, 3H).

### 83.2 1-Benzyl-2-methyl-1,2,3,6-tetrahydropyridine (I-83.2)

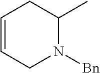

Sodium borohydride (5.6 g, 148 mmol) was added in portions to an ice-cooled solution of 1-benzyl-2-methylpyridinium bromide (20 g, 0.074 mol) in ethanol (500 mL) over 30 minutes. After the complete addition, the reaction was stirred at room temperature for another 1.5 h. Water (300 mL) was added, and ethanol was removed under reduced pressure. The aqueous phase was extracted with DCM (3×200 mL), and the combined organic phases were dried over anhydrous Na₂SO₄. The crude product was obtained as light yellow oil, and used in the next step without further purification (9.8 g, 69%). ¹H NMR (400 MHz, CDCl₃): δ 7.30-7.14 (m, 5H), 5.64-5.60 (m, 1H), 5.55-5.52 (m, 1H), 3.74 (d, 1H), 3.39 (d, 1H), 2.93-2.85 (m, 1H), 2.29-2.22 (m, 1H), 1.85-1.76 (m, 1H), 1.02 (d, 3H).

### 83.3 Benzyl 6-methyl-5,6-dihydropyridine-1(2H)-carboxylate (I-83.3)

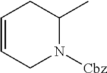

Crude 1-benzyl-2-methyl-1,2,3,6-tetrahydropyridine (9.8 g, 52.3 mmol) was dissolved in toluene (200 mL). Benzyl chloroformate (22 mL, 0.154 mol) was added, and the resulting mixture was heated at 90° C. for 4 hours, and then concentrated. The residue was purified by silica gel chromatography to give the product as colorless oil (4.3 g, 36%). ¹H NMR (400 MHz, CDCl₃): δ 7.44-7.27 (m, 5H), 5.81-5.62 (m, 2H), 5.20-5.13 (m, 2H), 4.66-4.52 (m, 1H), 4.27 (d, 1H), 3.62 (d, 1H), 2.56-2.42 (m, 1H), 1.95-1.84 (m, 1H), 1.16 (d, 3H).

### 83.4 Benzyl 4-methyl-7-oxa-3-azabicyclo[4.1.0]heptane-3-carboxylate (I-83.4)

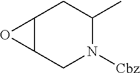

To an ice-cooled solution of benzyl 6-methyl-5,6-dihydropyridine-1(2H)-carboxylate (2.96 g, 12.7 mmol) in DCM (50 mL) was added m-CPBA (4.4 g, 19 mmol) in small portions. After completion of the addition, the mixture was stirred at room temperature overnight. Then the excess m-CPBA was consumed by the addition of aqueous Na₂S₂O₃. The organic phase was separated and dried over anhydrous Na₂SO₄. After concentration, the residue was purified by silica gel chromatography to give the product as colorless oil (2.02 g, 64%). ¹H NMR (400 MHz, CDCl₃): δ 7.31-7.22 (m, 5H), 5.08-5.02 (m, 2H), 4.39-4.26 (m, 2H), 3.27 (d, 1H), 3.17 (t, 1H), 3.09 (s, 1H), 2.11 (dd, 1H), 1.61 (dd, 1H), 1.10 (d, 3H).

### 83.5 Benzyl 4-amino-5-hydroxy-2-methylpiperidine-1-carboxylate (I-83.5)

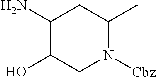

To a suspension of benzyl 4-methyl-7-oxa-3-azabicyclo[4.1.0]heptane-3-carboxylate (1.04 g, 4.21 mmol) in aqueous ammonia (>25%, 20 mL) and ethanol (10 mL) was added solid ammonium chloride (5.0 g, 93 mmol). The resulting mixture was heated at reflux overnight. The reaction was cooled, made basic by the addition of solid Na₂CO₃ and extracted three times with EtOAc. The combined organic phases were dried over Na₂SO₄. After concentration, the crude product was directly used in the next step without further purification (0.724 g, 65%). ¹H NMR (400 MHz, CDCl₃): δ 7.39-7.29 (m, 5H), 5.18-5.10 (m, 2H), 4.16-4.07 (m, 1H), 3.82-3.78 (m, 1H), 3.55-3.49 (m, 2H), 2.91-2.86 (m, 1H), 2.02-1.95 (m, 1H), 1.35-1.26 (m, 3H).

### 83.6 Benzyl 5-hydroxy-2-methyl-4-(picolinamido)piperidine-1-carboxylate (I-83.6)

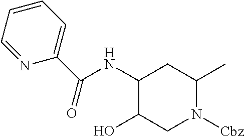

To an ice-cooled suspension of benzyl 4-amino-5-hydroxy-2-methyl-piperidine-1-carboxylate (0.724 g, 2.74 mmol) in DCM (20 mL) was added picolinic acid (0.337 g, 2.74 mmol) and HOBt (0.555 g, 4.11 mmol). EDCI (0.785 g, 4.11 mmol) and TEA (1 mL) were then added, and the resulting mixture was stirred at 0° C. for two hours, and at room temperature overnight. After concentration, the residue was purified by silica gel chromatography to give the product as a white solid (0.92 g, 86%). ¹H NMR (400 MHz, CDCl₃): δ 8.48-8.47 (m, 1H), 8.19-8.14 (m, 1H), 8.11 (d, 1H), 7.80 (dt, 1H), 7.39 (ddd, 1H), 7.33-7.22 (m, 5H), 5.14-5.07 (m, 2H), 4.20-4.14 (m, 1H), 3.93 (dd, 1H), 3.86-3.80 (m, 2H), 3.32 (dd, 1H), 2.14-2.06 (m, 1H), 1.57-1.48 (m, 1H), 1.23 (d, 3H).

### 83.7 Benzyl 2-methyl-5-oxo-4-(picolinamido)piperidine-1-carboxylate (I-83.7)

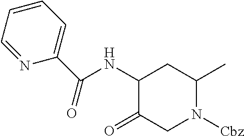

To a solution of benzyl 5-hydroxy-2-methyl-4-(picolinamido)piperidine-1-carboxylate (0.92 g, 2.5 mmol) in DCM (20 mL) was added Dess-Martin reagent (2.21 g, 5 mmol). The resulting mixture was stirred at room temperature overnight. Aqueous sodium carbonate was added until basic and the reaction was stirred for 30 minutes. The DCM phase was separated and concentrated to dryness. The residue was purified by silica gel chromatography to give the product as a white solid (0.594 g, 65%). ¹H NMR (400 MHz, CDCl₃): δ 8.68 (br, 1H), 8.61-8.60 (m, 1H), 8.18-8.16 (m, 1H), 7.88-7.84 (m, 1H), 7.47-7.44 (m, 1H), 7.42-7.28 (m, 5H), 5.24-5.16 (m, 2H), 4.89-4.81 (m, 2H), 4.69-4.62 (m, 1H), 3.87 (d, 1H), 2.92-2.85 (m, 1H), 1.56-1.43 (m, 1H), 1.26 (d, 3H).

### 83.8 Benzyl 6-methyl-2-(pyridin-2-yl)-6,7-dihydrooxazolo[5,4-c]pyridine-5(4H)-carboxylate (I-83.8)

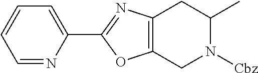

To a solution of 2-methyl-5-oxo-4-[(pyridine-2-carbonyl)-amino]-piperidine-1-carboxylic acid benzyl ester (1.1 g, 3 mmol) in anhydrous dioxane (40 mL) was added freshly distilled POCl₃ (1.4 mL, 15 mmol), and the resulting mixture was heated at 100° C. for 4 hours. All volatiles were removed under reduced pressure, and the brown residue was treated with saturated aq. Na₂CO₃ until the pH reached 9. The product was extracted with DCM (3×30 mL), and the combined organic layers were dried over Na₂SO₄. After filtration and concentration, the product was purified by silica gel chromatography (0.55 g, 53%). ¹H NMR (400 MHz, CDCl₃): δ 8.67-8.64 (m, 1H), 8.03-8.01 (m, 1H), 7.79-7.73 (m, 1H), 7.34-7.24 (m, 6H), 5.23-5.04 (m, 3H), 4.94-4.86 (m, 1H), 4.20-4.14 (m, 1H), 3.00-2.96 (m, 1H), 2.44 (d, 1H), 1.15 (d, 3H).

### 83.9 6-Methyl-2-(pyridin-2-yl)-4,5,6,7-tetrahydrooxazolo[5,4-c]pyridine (I-83.9)

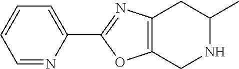

To the solution of benzyl 6-methyl-2-(pyridin-2-yl)-6,7-dihydrooxazolo[5,4-c]pyridine-5(4H)-carboxylate (141 mg, 0.4 mmol) in MeCN (10 mL) was added TMSI (0.4 mL, 2.8 mmol) in one portion at room temperature, and the resulting mixture was stirred at room temperature for about 2 h. The solvent and excess TMSI were removed under reduced pressure. The brown solid residue was washed with ether (3×5 mL), collected by filtration, dried under high vacuum, and used directly in the next step (150 mg, crude). ¹H NMR (400 MHz, DMSO-d₆): δ 9.47-9.36 (m, 2H), 8.77 (d, 1H), 8.16 (d, 1H), 8.09-8.05 (m, 1H), 7.64-7.61 (m, 1H), 4.65-4.59 (m, 2H), 3.86-3.78 (m, 1H), 3.07 (dd, 1H), 2.76-2.70 (m, 1H), 1.47 (d, 3H).

### 84. Compound 84: 3-(2-(Pyridin-2-yl)-4H-pyrrolo[3,4-d]oxazol-5(6H)-yl)benzonitrile

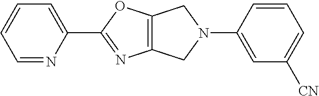

A mixture of 5,6-dihydro-2-(pyridin-2-yl)-4H-pyrrolo[3,4-d]oxazole (I-84.8) (20 mg, 0.11 mmol), 3-bromobenzonitrile (29 mg, 0.16 mmol), Pd(OAc)₂ (2 mg, 0.01 mmol), Xantphos (3 mg, 0.005 mol), and Cs₂CO₃ (104 mg, 0.32 mmol) in toluene (5 mL) was stirred at 100° C. overnight. The mixture was filtered and the filtrate was purified by preparative TLC (DCM:MeOH=50:1) to afford the product 3-(2-(pyridin-2-yl)-4H-pyrrolo[3,4-d]oxazol-5(6H)-yl)benzonitrile as a white solid (2.3 mg, yield: 10.6%). ¹H NMR (400 MHz, CDCl₃): δ 8.76 (brs, 1H), 8.15 (d, 1H), 7.87 (t, 1H), 7.42-7.34 (m, 2H), 7.05 (d, 1H), 6.84 (d, 2H), 4.59 (t, 2H), 4.47 (t, 2H). LC/MS: m/e=289 (M+H)⁺.

### 84.1 2,5-Dihydro-1H-pyrrole hydrochloride (I-84.1)

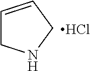

To a solution of 10N HCl (500 mL) was added Zn metal (200 g, 3.1 mol) at −10° C. After stirring for 45 min, 1H-pyrrole (50 g, 0.75 mol) was added slowly from a dropping funnel at −10° C. The resulting mixture was stirring at −10° C. for 30 min. Concentrated HCl (300 mL) was added and the stirring continued for 3 h at −10° C. The solid was filtered off and washed with H₂O (100 mL). The mixture was adjusted to pH 14 with NaOH (2 M) and then steam distilled until the distillate was no longer alkaline to litmus. The distillate was made acidic (pH 2) with concentrated HCl. The solvent was removed in vacuo to afford the pure product 2,5-dihydro-1H-pyrrole hydrochloride (53 g, yield: 77%), as a red solid. ¹H NMR (400 MHz, MeOH-d₄): δ 5.95 (s, 2H), 4.07 (s, 4H).

### 84.2 Benzyl 2H-pyrrole-1(5H)-carboxylate (I-84.2)

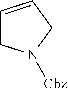

To a solution of 2,5-dihydro-1H-pyrrole hydrochloride (12 g, 0.11 mol) in H₂O (100 mL) was added EtOAc (100 mL) and K₂CO₃ (89 g, 0.64 mol). The mixture was stirred at RT for 30 min. Cbz-Cl (22 g, 0.13 mol) was added dropwise at 0° C. and the mixture was stirred for 1 h. The organic layer was separated and the aqueous layer was extracted with EtOAc (3×20 mL). The combined organic layers were dried over Na₂SO₄. The solvent was removed in vacuo to afford the crude product (25 g, 95%), as a light oil, which was carried directly to the next step.

### 84.3 Benzyl 6-oxa-3-aza-bicyclo[3.1.0]hexane-3-carboxylate (I-84.3)

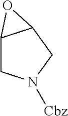

Benzyl 2H-pyrrole-1(5H)-carboxylate (25 g, 0.12 mol) was dissolved in DCM (500 mL) at 0° C. m-CPBA (42 g, 0.24 mol) was added in portions at 0° C., and the mixture was stirred at RT for 3 days. The reaction mixture was filtered, and the filtrate was washed Na₂S₂O₃ (3 M, 100 mL), NaHCO₃ (1 M, 100 mL) and brine (100 mL). The organic layer was dried over Na₂SO₄ and purified by silica gel chromatography (PE:EtOAc=10:1) to afford the product benzyl 6-oxa-3-aza-bicyclo[3.1.0]hexane-3-carboxylate (10 g, 37%) as a pale yellow oil. ¹H NMR (400 MHz, CDCl₃): δ 7.39-7.26 (m, 5H), 5.11 (d, 2H), 3.83 (dd, 2H), 3.68 (d, 2H), 3.39 (dd, 2H).

### 84.4 Benzyl 3-amino-4-hydroxypyrrolidine-1-carboxylate (I-84.4)

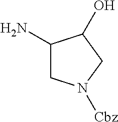

A mixture of benzyl 6-oxa-3-aza-bicyclo[3.1.0]hexane-3-carboxylate (5 g, 23 mmol) and aqueous ammonia (60 mL) was heated at reflux for 4 h. The solution was concentrated in vacuo to afford the crude product benzyl 3-amino-4-hydroxypyrrolidine-1-carboxylate as a light oil, which was used without further purification (5 g, 93%).

### 84.5 Benzyl 3-hydroxy-4-(picolinamido)pyrrolidine-1-carboxylate (I-84.5)

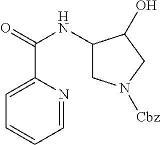

A solution of picolinic acid (1.5 g, 11 mmol), HOBT (3 g, 22 mmol), EDCI (4 g, 22 mmol), and TEA (3 g, 30 mmol) in DCM (200 mL) was stirred at RT for 15 min. A solution of benzyl 3-amino-4-hydroxypyrrolidine-1-carboxylate (2.6 g, 11 mmol) in DCM (20 mL) was added, and the reaction was stirred overnight at RT. The mixture was washed with HCl (0.5 M, 5×10 mL) and saturated Na₂CO₃ (5×10 mL), and the organic layer was dried over Na₂SO₄. The crude product was purified by silica gel chromatography (PE:EtOAc gradient=5:1-1:1) to afford the product benzyl 3-hydroxy-4-(picolinamido)pyrrolidine-1-carboxylate (2 g, 54%) as a white solid.

### 84.6 Benzyl 3-oxo-4-(picolinamido)pyrrolidine-1-carboxylate (I-84.6)

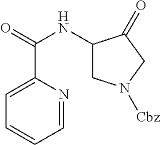

Benzyl 3-hydroxy-4-(picolinamido)pyrrolidine-1-carboxylate (1.8 g, 5.2 mmol) was dissolved in DCM (50 mL) and Dess-Martin reagent (6.7 g, 15.8 mmol) was added. The mixture was stirred at RT overnight. The reaction was diluted with DCM and washed with NaOH (0.5 M, 3×10 mL). The organic layer was dried over Na₂SO₄, filtered and concentrated in vacuo to afford the crude product, which was purified by preparative TLC (PE:EtOAc=1:1) to afford the product benzyl 3-oxo-4-(picolinamido)pyrrolidine-1-carboxylate (2.3 g, 77%), as a white solid.

### 84.7 Benzyl 2-(pyridin-2-yl)-4H-pyrrolo[3,4-d]oxazole-5(6H)-carboxylate (I-84.7)

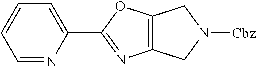

Benzyl 3-oxo-4-(picolinamido)pyrrolidine-1-carboxylate (350 mg, 1 mmol) was dissolved in CHCl₃ (5 mL). PCl₅(1.1 g, 5 mmol) was added portionwise at 50° C. and the stirring was continued for an additional 3 h. The reaction mixture was passed through a short column of alumina (DCM:MeOH=50:1) to afford the crude product. The crude product was washed with MeOH (3×3 mL) to afford the product benzyl 2-(pyridin-2-yl)-4H-pyrrolo[3,4-d]oxazole-5(6H)-carboxylate (35 mg, 10.5%) as a white solid.

### 84.8 3-(5,6-Dihydro-4H-pyrrolo[3,4-d]oxazol-2-yl)benzonitrile (I-84.8)

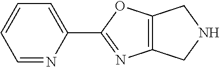

To a solution of benzyl 2-(pyridin-2-yl)-4H-pyrrolo[3,4-d]oxazole-5(6H)-carboxylate (32 mg, 0.1 mmol) in DCM (1 mL) was added MeCN (3 mL) and TMSI (200 mg, 1 mmol). The mixture was stirred for 3 h at RT. The solvent was removed in vacuo, and the residue was washed with ether (3×5 mL). The crude product (20 mg) was suitable for use without further purification.

### 85. Compound 85: 2-(2-(Pyridin-2-yl)-4H-pyrrolo[3,4-d]oxazol-5(6H)-yl)pyridine-3-carbonitrile

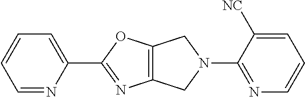

A mixture of 5,6-dihydro-2-(pyridin-2-yl)-4H-pyrrolo[3,4-d]oxazole (I-84.8) (11 mg, 0.06 mmol), 2-chloropyridine-3-carbonitrile (9.7 mg, 0.07 mmol), and TEA (18 mg, 0.18 mmol) in DMF (3 mL) was stirred at 90° C. for 20 h. The mixture was quenched with water (50 mL) and extracted with DCM (5×10 mL). The combined organic layers were washed with water and brine, and dried over Na₂SO₄. The resulting oil was purified by preparative TLC (DCM:MeOH=50:1) to afford the product 2-(2-(pyridin-2-yl)-4H-pyrrolo[3,4-d]oxazol-5(6H)-yl)pyridine-3-carbonitrile (3.1 mg, 12.3%) as a pale yellow solid. ¹H NMR (400 MHz, CDCl₃): δ 8.73 (d, 1H), 8.36 (dd, 1H), 8.14 (d, 1H), 7.87-7.79 (m, 2H), 7.41-7.38 (m, 1H), 6.73-6.70 (m, 1H), 5.07-5.00 (m, 4H). LC/MS: m/e=290 (M+H)⁺.

### 86. Compound 86: 3-(5-(Pyridin-2-yl)-5,6-dihydro-4H-pyrrolo[3,4-d]oxazol-2-yl)benzonitrile

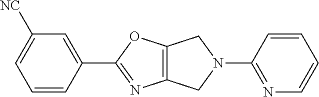

The title compound was prepared via the procedure used for Compound 84, using 3-cyanobenzoic acid instead of picolinic acid, and using 2-bromopyridine instead of 3-bromo-benzonitrile. Preparative TLC (DCM:MeOH=50:1) afforded 3-(5,6-dihydro-5-(pyridin-2-yl)-4H-pyrrolo[3,4-d]oxazol-2-yl)benzonitrile (1.3 mg, 3.2%) as a white solid. ¹H NMR (400 MHz, CDCl₃): δ 8.34 (s, 1H), 8.28-8.24 (m, 2H), 7.75-7.73 (m, 1H), 7.63-7.59 (m, 2H), 6.73 (brs, 1H), 6.54 (brs, 1H), 4.84 (s, 2H), 4.60 (s, 2H). LC/MS: m/e=289 (M+H)⁺.

### 87. Compound 87: 2-(2-(3-Cyanophenyl)-4H-pyrrolo[3,4-d]oxazol-5(6H)-yl)nicotinonitrile

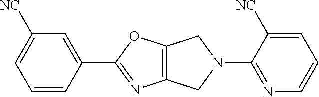

2-(3-oxo-4-((3-cyanobenzamido)pyrrolidin-1-yl)pyridine-3-carbonitrile (1-87.7) (0.24 g, 0.7 mmol) was dissolved in CHCl₃ (20 mL) and PCl₅ (0.9 g, 4.3 mmol) was added in portions at 50° C. The mixture was stirred at 50° C. for 3 h. The mixture was purified by short column chromatography on Al₂O₃ (PE:EtOAc=1:10) to afford the crude product, which was further purified by preparative TLC(CH₂Cl₂:MeOH=30:1) to afford the pure product 2-(2-(3-cyanophenyl)-4H-pyrrolo[3,4-d]oxazol-5(6H)-yl)pyridine-3-carbonitrile (4.7 mg, yield: 2.1%) as a white solid. ¹H NMR (400 MHz, CDCl₃): δ 8.37 (dd, 1H), 8.33 (s, 1H), 8.27 (d, 1H), 7.81 (d, 1H), 7.74 (d, 1H), 7.61 (t, 1H), 6.75-6.72 (m, 1H), 5.08 (t, 2H), 4.98 (t, 2H); LC/MS: m/e=314 (M+H)⁺.

### 87.1 tert-Butyl 2H-pyrrole-1(5H)-carboxylate (I-87.1)

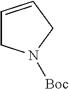

(Boc)₂O (1.53 g, 7 mmol) in MeOH (5 mL) was added dropwise to a solution of 2,5-dihydro-1H-pyrrole hydrochloride (0.66 g, 6 mmol) and TEA (5 g, 50 mmol) in MeOH (5 mL) at 0° C. The reaction mixture was stirred at RT overnight. The reaction was concentrated in vacuo and purified by silica gel chromatography (PE:EtOAc=10:1) to afford the product tert-butyl 2H-pyrrole-1(5H)-carboxylate (0.9 g, 85%) as a light oil. ¹H NMR (400 MHz, CDCl₃): δ 5.76 (s, 2H), 4.11 (s, 4H), 1.47-1.45 (m, 9H).

### 87.2 tert-Butyl 6-oxa-3-aza-bicyclo[3.1.0]hexane-3-carboxylate (I-87.2)

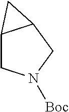

m-CPBA (48 g, 0.28 mol) was added to a solution of tert-butyl 2H-pyrrole-1(5H)-carboxylate (15 g, 0.07 mol) in DCM (500 mL) in portions at 0° C. The reaction was stirred at RT overnight. The mixture was filtered and the filtrate was washed with aqueous Na₂SO₃, followed by NaHCO₃ and saturated brine. The organic layer was dried over Na₂SO₄ and purified by silica gel chromatography (PE:EtOAc gradient=20:1 to 5:1) to afford the product tert-butyl 6-oxa-3-aza-bicyclo[3.1.0]hexane-3-carboxylate (10 g, 74%) as a light oil. ¹H NMR (400 MHz, CDCl₃): δ 3.80-3.64 (m, 4H), 3.29 (dd, 2H), 1.43-1.42 (m, 9H).

### 87.3 tert-Butyl 3-amino-4-hydroxypyrrolidine-1-carboxylate (I-87.3)

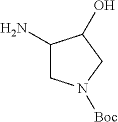

The mixture of tert-butyl 6-oxa-3-aza-bicyclo[3.1.0]hexane-3-carboxylate (20 g, 0.11 mol) and aqueous ammonia (200 mL) was heated overnight at reflux. The reaction was concentrated in vacuo to afford the product tert-butyl 3-amino-4-hydroxypyrrolidine-1-carboxylate (21 g, 96%) as a pale yellow oil. ¹H NMR (400 MHz, CDCl₃): δ 3.98-3.97 (m, 1H), 3.75-3.61 (m, 2H), 3.36-3.22 (m, 2H), 3.08-3.17 (m, 1H), 1.45 (s, 9H).

### 87.4 tert-Butyl 3-(3-cyanobenzamido)-4-hydroxypyrrolidine-1-carboxylate (I-87.4)

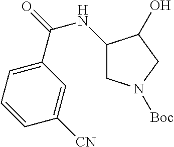

A solution of 3-cyanobenzoic acid (1.58 g, 0.01 mol), HOBT (3.03 g, 0.02 mol), EDCI (3.8 g, 0.02 mol), DIEA (2.5 g, 0.02 mol) in DCM (200 mL) was stirred at RT for 15 min. tert-Butyl 3-amino-4-hydroxypyrrolidine-1-carboxylate (2.3 g, 0.01 mol) was added, and the mixture was stirred at RT overnight. The reaction was diluted with DCM, washed with H₂O, and dried over Na₂SO₄. Purification by silica gel chromatography (PE:EtOAc gradient=8:1 to 2:1) afforded the product tert-butyl 3-(3-cyanobenzamido)-4-hydroxypyrrolidine-1-carboxylate (2 g, 62%) as a white solid. The product was carried directly to the next step.

### 87.5 3-Cyano-N-(4-hydroxypyrrolidin-3-yl)benzamide (I-87.5)

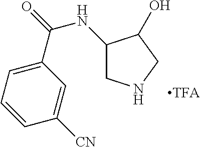

tert-Butyl 3-(3-cyanobenzamido)-4-hydroxypyrrolidine-1-carboxylate (1 g, 3 mmol) was dissolved in DCM (10 mL). TFA (2 mL) was added, and the reaction was stirred at RT for 1 h. The solvent was removed in vacuo and the resulting oil was used without further purification.

### 87.6 2-(3-Hydroxy-4-((3-cyanobenzamido)pyrrolidin-1-yl)pyridine-3-carbonitrile (I-87.6)

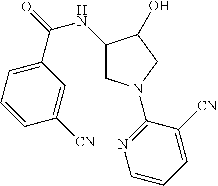

3-Cyano-N-(4-hydroxypyrrolidin-3-yl)benzamide (0.56 g, 2.4 mmol) was dissolved in DMF (5 mL). 2-Chloropyridine-3-carbonitrile (0.33 g, 2.4 mmol) and DIEA (1.25 g, 10 mmol) were added, and the reaction was stirred at 100° C. overnight. The reaction was quenched with water (50 mL). After stirring 0.5 h, the mixture was extracted with DCM (5×10 mL). The combined organic layers were dried over Na₂SO₄. Purification by silica gel chromatography (PE:EtOAc gradient=5:1 to 1:1) afforded the product 2-(3-hydroxy-4-((3-cyanobenzamido)-pyrrolidin-1-yl)pyridine-3-carbonitrile (0.32 g, 40%) as a white solid. ¹H NMR (400 MHz, CDCl₃): δ 8.34-8.32 (dd, 1H), 8.09 (s, 1H), 8.03-8.01 (m, 1H), 7.82 (d, 1H), 7.77 (dd, 1H), 7.60 (t, 1H), 6.72-6.69 (dd, 1H), 6.41 (s, 1H), 4.56-4.51 (m, 2H), 4.33-4.28 (m, 1H), 4.24-4.20 (m, 1H), 3.93-3.90 (m, 1H), 3.85-3.81 (m, 1H).

### 87.7 2-(3-oxo-4-((3-cyanobenzamido)pyrrolidin-1-yl)pyridine-3-carbonitrile (I-87.7)

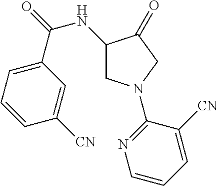

2-(3-Hydroxy-4-((3-cyanobenzamido)pyrrolidin-1-yl)pyridine-3-carbonitrile (0.3 g, 1 mmol) was dissolved in DCM (10 mL). Dess-Martin reagent (1.14 g, 2.7 mmol) was added, and the mixture was stirred at RT for 3 h. The crude reaction mixture was purified by short column chromatography on silica gel (PE:EtOAc=1:4) to afford the crude product 2-(3-oxo-4-((3-cyanobenzamido)pyrrolidin-1-yl)pyridine-3-carbonitrile (0.24 g, 80%) as a yellow oil, which was used for the next step.

### 88. Compound 88: 3-(2-(Pyridin-2-yl)-7,8-dihydro-4H-oxazolo[5,4-d]azepin-6(5H)-yl)benzonitrile

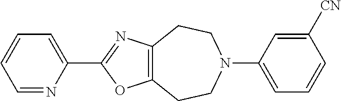

To a mixture of 7-pyridin-2-yl-1,2,3,4,5,6-hexahydro-cyclopenta[d]azepine (I-88.13) (70 mg, 0.32 mmol), 3-bromo-benzonitrile (87 mg, 0.48 mmol), Xantphos (8.9 mg, 0.016 mmol), Pd(OAc)₂ (3.9 mg, 0.016 mmol), and Cs₂CO₃ (312.9 mg, 0.96 mmol) in toluene (15 mL) was heated at 110° C. overnight. The mixture was concentrated, and the residue was purified by preparative TLC (PE:EtOAc=1:1) to give 3-(2-(pyridin-2-yl)-7,8-dihydro-4H-oxazolo[5,4-d]azepin-6(5H)-yl)benzonitrile (8 mg, 8%) as a yellow solid. ¹H NMR (400 MHz, CDCl₃): δ 8.62-8.83 (m, 1H), 7.95-7.97 (m, 1H), 7.69-7.73 (m, 1H), 7.14-7.27 (m, 2H), 6.86-6.92 (m, 3H), 3.82-3.86 (m, 4H), 3.10-3.15 (m, 2H), 2.94-2.97 (m, 2H); LC/MS: m/e=317 (M+H)⁺.

### 88.1 1-tert-Butyl 4-ethyl 5-oxoazepane-1,4-dicarboxylate (I-88.1)

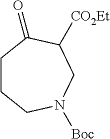

A solution of tert-butyl 4-oxopiperidine-1-carboxylate (40 g, 0.2 mol) in Et₂O (200 mL) was cooled to −30° C. in dry-ice/acetone bath. Ethyl diazoacetate (27 g, 240 mmol) was added, followed by the dropwise addition of BF₃.Et₂O (27 g, 240 mmol). The reaction was stirred for 30 min at −30° C. The reaction was warmed to room temperature and stirred for 1 h. The reaction was charged to a separatory funnel and washed with saturated NaHCO₃ (500 mL) and extracted with EtOAc (500 mL). The organic phase was dried over Na₂SO₄ and concentrated to give 1-tert-butyl 4-ethyl 5-oxoazepane-1,4-dicarboxylate (55 g, 96%) as an orange oil. ¹H NMR (400 MHz, CDCl₃): δ 4.11-4.28 (m, 2H), 3.58-3.87 (m, 3H), 3.21-3.42 (m, 2H), 2.60-2.90 (m, 2H) 1.91-2.08 (m, 2H), 1.43 (s, 9H), 1.21-1.26 (t, 3H).

### 88.2 Azepan-4-one (I-88.2)

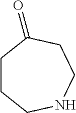

A mixture of 1-tert-butyl 4-ethyl 5-oxoazepane-1,4-dicarboxylate (55 g, 0.19 mol) in HCl (4.0 M, 300 mL) was heated to 140° C. for 8 h. The mixture was concentrated, and the residue was washed with ether (500 mL), and collected by vacuum filtration to give azepan-4-one (20 g, 95%) as a white solid. ¹H NMR (400 MHz, DMSO-d₆): δ 9.32-9.48 (m, 2H), 3.18-3.22 (m, 2H), 2.68-2.80 (m, 2H), 2.55-2.64 (m, 2H), 1.90-2.01 (m, 2H).

### 88.3 Benzyl 4-oxoazepane-1-carboxylate (I-88.3)

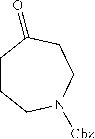

A mixture of azepan-4-one (30 g, 0.26 mol) and K₂CO₃ (71.8 g, 0.52 mol) was stirred in a mixture of EtOAc (200 mL) and water (200 mL). Cbz-Cl (37 mL, 0.26 mol) was added dropwise and the mixture was stirred at RT for 30 min. The reaction was diluted with water (300 mL) and extracted with EtOAc (300 mL). The organic phase was dried over Na₂SO₄ and concentrated, and the residue was purified by silica gel chromatography (PE:EtOAc gradient=20:1 to 5:1) to afford benzyl 4-oxoazepane-1-carboxylate (45 g, 70%) as a colorless oil. ¹H NMR (400 MHz, DMSO-d₆): δ 7.26-7.38 (m, 5H), 5.03 (s, 2H), 3.45-3.62 (m, 4H), 2.45-2.64 (m, 4H), 1.56-1.65 (m, 2H).

### 88.4 Benzyl 4-hydroxyazepane-1-carboxylate (I-88.4)

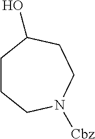

A solution of benzyl 4-oxoazepane-1-carboxylate (20 g, 0.08 mol) in MeOH (200 mL) was cooled to 0° C. NaBH₄ (6.1 g, 0.16 mol) was added in portions, and the mixture was warmed to room temperature and stirred for 30 min. The reaction was adjusted to pH=3 with 1N HCl and concentrated. The residue was diluted with water (200 mL) and extracted with EtOAc (200 mL). The organic phase was dried over Na₂SO₄, and concentrated to give benzyl 4-hydroxyazepane-1-carboxylate (19 g, 95%) as a colorless oil. ¹H NMR (400 MHz, DMSO-d₆): δ 7.21-7.48 (m, 5H), 5.02 (s, 2H), 4.47-4.52 (m, 1H), 3.55-3.65 (m, 1H), 3.30-3.42 (m, 1H), 3.17-3.30 (m, 2H), 1.94 (s, 1H), 1.70-1.81 (m, 2H), 1.56-1.66 (m, 1H), 1.40-1.56 (m, 3H).

### 88.5 Benzyl 4-(methylsulfonyloxy) azepane-1-carboxylate (I-88.5)

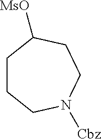

A solution of benzyl 4-hydroxyazepane-1-carboxylate (19 g, 76.2 mmol) in DCM (100 mL) was cooled to 0° C. TEA (15.4 g, 152 mmol) was added, followed by MsCl (8.73 g, 76.2 mmol). The reaction was stirred at RT for 30 min. The mixture was quenched into ice/water (200 mL) and extracted with DCM (200 mL). The organic phase was dried over Na₂SO₄ and concentrated to give benzyl 4-(methylsulfonyloxy)-azepane-1-carboxylate (20 g, 80%) as a yellow oil. ¹H NMR (400 MHz, DMSO-d₆): δ 7.22-7.38 (m, 5H), 5.02 (s, 2H), 4.75-4.85 (m, 1H), 3.458 (s, 3H), 3.30-3.49 (m, 4H), 1.91-2.10 (m, 2H), 1.72-1.91 (m, 4H), 1.52-1.72 (m, 1H).

### 88.6 Benzyl 2,3,6,7-tetrahydro-1H-azepine-1-carboxylate (I-88.6a) and Benzyl 2,3,4,7-tetrahydro-1H-azepine-1-carboxylate (I-88.6b)

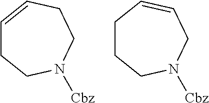

A mixture of benzyl 4-(methylsulfonyloxy)azepane-1-carboxylate (20 g, 61 mmol) in DBU (100 mL) was heated to 130° C. for 30 min. Then the mixture was poured into ice/water (100 mL), and extracted with DCM (200 mL). The organic phase was dried over Na₂SO₄ and concentrated to give a mixture of benzyl 2,3,6,7-tetrahydro-1H-azepine-1-carboxylate and benzyl 2,3,4,7-tetrahydro-1H-azepine-1-carboxylate as colorless oil (7 g, crude) which was used in the next step without further purification.

### 88.7 Benzyl 8-oxa-4-azabicyclo[5.1.0]octane-4-carboxylate (I-88.7)

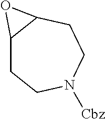

A mixture of benzyl 2,3,6,7-tetrahydro-1H-azepine-1-carboxylate and benzyl 2,3,4,7-tetrahydro-1H-azepine-1-carboxylate (7 g, 0.03 mol) in DCM (100 mL) was cooled to 0° C. m-CPBA (12.2 g (85%), 0.06 mol) was added in portions and the mixture was stirred at RT overnight. The mixture was diluted with saturated Na₂S₂O₃ until the aqueous phase became colorless. The mixture was extracted with DCM (300 mL), dried over Na₂SO₄ and concentrated. Purification by silica gel chromatography (EtOAc:PE gradient=1:10 to 1:5) to afford benzyl 8-oxa-4-azabicyclo[5.1.0]octane-4-carboxylate (2 g, 27%) as a colorless oil. ¹H NMR (400 MHz, DMSO-d₆): δ 7.25-7.39 (m, 5H), 5.03 (s, 2H), 3.60-3.71 (m, 2H), 3.06-3.11 (m, 2H), 2.57-2.76 (m, 2H), 2.08-2.19 (m, 2H), 1.78-2.02 (m, 2H).

### 88.8 Benzyl 4-azido-5-hydroxyazepane-1-carboxylate (I-88.8)

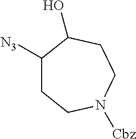

To a solution of benzyl 8-oxa-4-azabicyclo[5.1.0]octane-4-carboxylate (13 g, 52.5 mmol) in acetone (50 mL) was added water (50 mL), DMF (5 mL), and NaN₃ (5.13 g, 78.8 mmol). The mixture was heated to 90° C. and stirred overnight. The reaction was concentrated, diluted with water (200 mL) and extracted with EtOAc (200 mL). The organic phase was dried over Na₂SO₄ and concentrated to give benzyl 4-azido-5-hydroxyazepane-1-carboxylate (14 g, 91%) as a colorless oil. ¹H NMR (400 MHz, DMSO-d₆): δ 7.25-7.39 (m, 5H), 5.20-5.30 (m, 2H), 5.02 (s, 2H), 3.38-3.55 (m, 4H), 3.20-3.38 (m, 1H), 2.06-2.18 (m, 1H), 1.80-1.91 (m, 2H), 1.50-1.75 (m, 2H).

### 88.9 4-Amino-5-hydroxy-azepane-1-carboxylicacid benzyl ester (1-88.9)

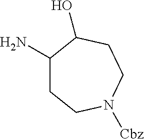

To a solution of benzyl 4-azido-5-hydroxyazepane-1-carboxylate (14 g, 48 mmol) in THF (70 mL) was added water (7 mL) and Ph₃P (25.8 g, 98 mmol). The mixture was heated to 80° C. for 2 h. The reaction was concentrated, and the residue was purified by silica gel chromatography (DCM:MeOH=100:1) to give 4-amino-5-hydroxy-azepane-1-carboxylic acid benzyl ester (2 g, 16%) as a colorless oil. ¹H NMR (400 MHz, DMSO-d₆): δ 7.25-7.40 (m, 5H), 5.03 (s, 1H), 3.51-3.11 (m, 2H), 2.74-2.92 (m, 2H), 2.09 (s, 2H), 1.75-1.98 (m, 4H).

### 88.10 4-Hydroxy-5-[(pyridine-2-carbonyl)-amino]-azepane-1-carboxylic acid benzyl ester (I-88.10)

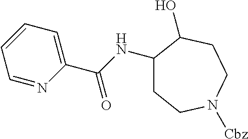

To a mixture of pyridine-2-carboxylic acid (280 mg, 2.27 mmol) in DCM (50 mL) was added EDCI (649 mg, 3.4 mmol), HOBt (460 mg, 3.4 mmol) and TEA (454 mg, 4.5 mmol). 4-Amino-5-hydroxy-azepane-1-carboxylic acid benzyl ester (600 mg, 2.27 mmol) in DCM (50 mL) was added, and the mixture was stirred at RT for 8 h. The solution was adjusted to pH=7 with saturated NaHCO₃, and extracted with DCM (200 mL). The organic phase was dried over Na₂SO₄ and concentrated. The residue was purified by preparative TLC (PE:EtOAc=1:1) to give 4-hydroxy-5-[(pyridine-2-carbonyl)-amino]-azepane-1-carboxylic acid benzyl ester (700 mg, 84%) as a colorless oil.

### 88.11 4-Oxo-5-[(pyridine-2-carbonyl)-amino]-azepane-1-carboxylic acid benzyl ester (I-88.11)

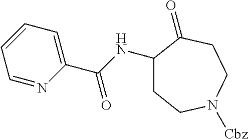

To a solution of 4-hydroxy-5-[(pyridine-2-carbonyl)-amino]-azepane-1-carboxylic acid benzyl ester (600 mg, 1.62 mmol) in DCM (100 mL) was added Dess-Martin Reagent (1.03 g, 2.44 mmol). The reaction was stirred at RT overnight. The solution was adjusted to pH=8 with saturated NaHCO₃ and extracted with DCM (200 mL). The organic phase was dried over Na₂SO₄ and concentrated to give 4-oxo-5-[(pyridine-2-carbonyl)-amino]-azepane-1-carboxylic acid benzyl ester (400 mg, 66%) as a yellow oil.

### 88.12 7-Pyridin-2-yl-1,4,5,6-tetrahydro-2H-cyclopenta[d]azepine-3-carboxylic acid benzyl ester (I-88.12)

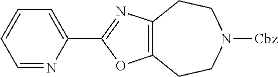

To a solution of 4-oxo-5-[(pyridine-2-carbonyl)-amino]-azepane-1-carboxylic acid benzyl ester (200 mg, 0.54 mmol) in dioxane (20 mL) was added POCl₃ (500 mg, 3.27 mmol). The reaction was heated to 80° C. for 2.5 h. The reaction was cooled to 0° C., adjusted to pH=8 with saturated NaHCO₃ and extracted with EtOAc (200 mL). The organic phase was dried over Na₂SO₄ and concentrated. The residue was purified by preparative TLC (PE:EtOAc=1:1) to give 7-pyridin-2-yl-1,4,5,6-tetrahydro-2H-cyclopenta[d]azepine-3-carboxylic acid benzyl ester (70 mg, 36%) as a yellow solid. ¹H NMR (400 MHz, CDCl₃): δ 8.63 (d, 1H), 7.97 (m, 1H), 7.69-7.73 (m, 1H), 7.20-7.32 (m, 6H), 5.11 (s, 1H), 3.60-3.71 (m, 4H), 2.82-3.06 (m, 4H).

### 88.13 7-Pyridin-2-yl-1,2,3,4,5,6-hexahydro-cyclopenta[d]azepine (I-88.13)

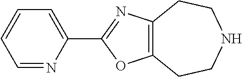

To a solution of 7-pyridin-2-yl-1,4,5,6-tetrahydro-2H-cyclopenta[d]azepine-3-carboxylic acid benzyl ester (70 mg, 0.2 mmol) in MeCN (10 mL) was added TMSI (0.4 mL, 0.28 mmol). The reaction was stirred at RT for 2 h. The reaction was concentrated, and the residue was triturated with Et₂O (50 mL) to give 7-pyridin-2-yl-1,2,3,4,5,6-hexahydro-cyclopenta[d]azepine (70 mg, crude) as a brown solid. ¹H NMR (400 MHz, DMSO-d₆): δ 8.80-8.92 (m, 2H), 8.62 (d, 1H), (m, 1H), 7.96-8.01 (m, 1H), 7.89-7.96 (m, 1H), 7.41-7.50 (m, 1H), 3.28-3.40 (m, 4H), 3.10-3.15 (m, 2H), 2.89-2.95 (m, 2H).

### 89. Compound 89: 3-Fluoro-5-(2-(pyridin-2-yl)-7,8-dihydro-4H-oxazolo[5,4-d]azepin-6(5H)-yl)benzonitrile

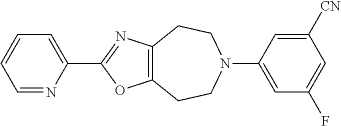

The title compound was prepared via the procedure used for Compound 88, using 3-bromo-5-fluoro-benzonitrile instead of 3-bromobenzonitrile. Purification by preparative HPLC gave 3-fluoro-5-(2-pyridin-2-yl-4,5,7,8-tetrahydro-oxazolo[4,5-d]azepin-6-yl)-benzonitrile (11 mg, 10%) as a yellow solid. ¹H NMR (400 MHz, CDCl₃): δ 8.66 (t, 1H), 8.01 (m, 1H), 7.80 (m, 1H), 7.33 (m, 1H), 6.70 (s, 1H), 6.53-6.64 (m, 2H), 3.79-3.88 (m, 4H), 3.08-3.12 (m, 2H), 2.72-2.80 (m, 2H); LC/MS: m/e=335 (M+H)⁺.

### 90. Compound 90: 3-Fluoro-5-(2-(pyridin-2-yl)-7,8-dihydro-4H-oxazolo[5,4-c]azepin-5(6H)-yl)benzonitrile

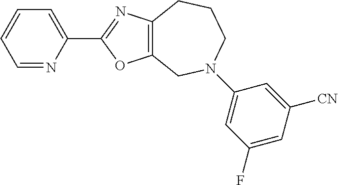

A mixture of 5,6,7,8-tetrahydro-2-(pyridin-2-yl)-4H-oxazolo[5,4-c]azepine (I-90.10) (20 mg, 0.09 mmol), 3-bromo-5-fluorobenzonitrile (18 mg, 0.09 mmol), Pd(OAc)₂ (1.0 mg, 0.005 mmol), Xantphos (3 mg, 0.005 mmol), and Cs₂CO₃ (91 mg, 0.28 mmol) in toluene (5 mL) was stirred at 100° C. overnight. The solvent was removed in vacuo and DCM (5 mL) was added. The mixture was filtered and the filtrate was purified by preparative TLC (PE:EtOAc=1:1) to afford the product 3-fluoro-5-(7,8-dihydro-2-(pyridin-2-yl)-4H-oxazolo[5,4-c]azepin-5(6H)-yl)benzonitrile (2.1 mg, 7%) as a pale yellow solid. ¹H NMR (400 MHz, CDCl₃): δ 8.73 (d, 1H), 8.07 (d, 1H), 7.83-7.79 (m, 1H), 7.38-7.31 (m, 1H), 6.82 (s, 1H), 6.72-6.63 (m, 2H), 4.64 (s, 1H), 3.80 (t, 2H), 2.87 (t, 2H), 2.03-1.97 (m, 2H); LC/MS: m/e=335 (M+H)⁺.

### 90.1 Benzyl allylcarbamate (I-90.1)

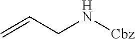

To a solution of prop-2-en-1-amine (50 g, 0.88 mol) in H₂O (400 mL) was added K₂CO₃ (301.8 g, 2.18 mol) and EtOAc (400 mL). The reaction was cooled to 0° C., and benzyl chloroformate (133.4 mL, 0.94 mol) was added dropwise. After 4-hour stiffing, the organic layer was separated, washed with aqueous HCl (1M, 5×20 mL) and brine (50 mL), and dried over Na₂SO₄. The mixture was filtered and the filtrate was concentrated in vacuo to afford the crude product benzyl allylcarbamate (160 g, 95%) as a colorless oil. ¹H NMR (400 MHz, DMSO-d₆): δ 7.42 (s, 1H), 7.38-7.28 (m, 5H), 5.82-5.75 (m, 1H), 5.13 (dd, 1H), 5.05-5.02 (m, 3H), 3.64-3.61 (m, 2H).

### 90.2 Benzyl allylpent-4-enyl carbamate (I-90.2)

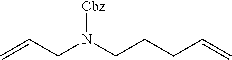

To a suspension of NaH (21 g, 65%, 0.56 mol) in DMF (500 mL) was added benzyl allylcarbamate (50 g, 0.26 mol) dropwise. The mixture was stirred for 30 min at RT. 5-Bromopent-1-ene (39 g, 0.26 mol) was added dropwise. The reaction mixture was heated to 40° C. and stirred for 6 h. DCM (200 mL) and water (200 mL) were added slowly. The organic layer was separated, washed with water (5×50 mL) and brine (3×20 mL), and dried over Na₂SO₄. The filtrate was concentrated in vacuo to afford the crude product, which was purified by silica gel column chromatography (PE:EtOAc gradient=100:1 to 30:1) to afford the product benzyl allylpent-4-enylcarbamate (20 g, 29%) as a yellow oil. ¹H NMR (400 MHz, DMSO-d₆): δ 7.36-7.21 (m, 5H), 5.83-5.74 (m, 2H), 5.08-4.89 (m, 4H), 5.11-4.92 (m, 2H), 4.43-3.79 (m, 2H), 3.43-3.12 (m, 2H), 2.10-1.96 (m, 2H), 1.65-1.48 (m, 2H).

### 90.3 (Z)-Benzyl 3,4-dihydro-2H-azepine-1(7H)-carboxylate (I-90.3)

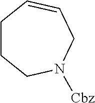

To a solution of benzyl allylpent-4-enylcarbamate (10 g, 0.037 mol) in DCM (300 mL) was added Grubbs catalyst (1 g). The reaction mixture was stirred at reflux overnight. The resulting mixture was concentrated in vacuo to afford the crude product, which was purified by silica gel chromatography (PE:EtOAc gradient=100:1 to 50:1) to afford the product (Z)-benzyl 3,4-dihydro-2H-azepine-1(7H)-carboxylate (4.1 g, 46%) as a colorless oil.

### 90.4 Benzyl 8-oxa-3-aza-bicyclo[5.1.0]octane-3-carboxylate (I-90.4)

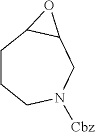

To a solution of (Z)-benzyl 3,4-dihydro-2H-azepine-1(7H)-carboxylate (4 g, 17 mmol) in DCM (100 mL) was added NaHCO₃ (13 g, 155 mmol) and then m-CPBA (9 g, 52 mmol) in portions. The reaction mixture was stirred at RT overnight. Then reaction was quenched with Na₂S₂O₄ (3M, 100 mL) and saturated brine (50 mL), and dried over Na₂SO₄. Concentration in vacuo afforded the crude product, which was purified by slica gel chromatography (PE:EtOAc=5:1) to afford the product benzyl 8-oxa-3-aza-bicyclo[5.1.0]-octane-3-carboxylate (1.3 g, 30%) as a colorless oil. ¹H NMR (400 MHz, DMSO-d₆): δ 7.40-7.29 (m, 5H), 5.06 (s, 2H), 4.12-3.99 (m, 1H), 3.72-3.61 (m, 1H), 3.48-3.37 (m, 1H), 3.06 (s, 2H), 2.73-2.62 (m, 1H), 2.12-2.07 (m, 1H), 1.97-1.87 (m, 1H), 1.51-1.44 (m, 2H).

### 90.5 Benzyl 4-azido-3-hydroxyazepane-1-carboxylate (I-90.5)

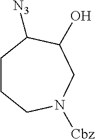

To a solution of benzyl 8-oxa-3-aza-bicyclo[5.1.0]octane-3-carboxylate (2.5 g, 10 mmol) in MeOH (70 mL) and H₂O (7 mL) was added NH₄Cl (1.6 g, 25 mmol) and NaN₃ (2 g, 37 mmol) slowly. The reaction was heated at reflux for 2 days. The majority of the solvent was removed in vacuo and the resulting solution was partitioned between DCM (50 mL) and H₂O (50 mL). The organic layer was separated, washed with NaHCO₃ solution (2M, 3×20 mL) and dried over Na₂SO₄. Concentration in vacuo afforded the crude product, which was purified by silica gel chromatography (PE:EtOAc gradient=5:1 to 1:1) to afford the product benzyl 4-azido-3-hydroxyazepane-1-carboxylate (2.1 g, 72%) as a light oil. ¹H NMR (400 MHz, DMSO-d₆): δ 7.37-7.30 (m, 5H), 5.57-5.54 (m, 1H), 5.10-5.06 (m, 2H), 3.75-3.67 (m, 1H), 3.60-3.50 (m, 1H), 3.48-3.37 (m, 1H), 3.16-2.95 (m, 2H), 1.80 (d, 2H), 1.66-1.57 (m, 1H), 1.27-1.16 (m, 1H).

### 90.6 Benzyl 4-amino-3-hydroxyazepane-1-carboxylate (I-90.6)

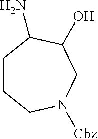

To a solution of benzyl 4-azido-3-hydroxyazepane-1-carboxylate (2 g, 7 mmol) in THF (50 mL) and H₂O (3 mL) was added PPh₃ (2.7 g, 10 mmol). The reaction mixture was heated at reflux for 2 days. The reaction was concentrated in vacuo to afford the crude product, which was purified by silica gel chromatography (DCM:MeOH gradient=30:1 to 5:1) to afford the product benzyl 4-amino-3-hydroxyazepane-1-carboxylate (1.4 g, 77%) as a light oil. ¹H NMR (400 MHz, DMSO-d₆): δ 7.39-7.30 (m, 5H), 5.09-5.05 (m, 2H), 3.72-3.68 (m, 1H), 3.58-3.50 (m, 1H), 3.18-3.03 (m, 2H), 2.90-2.80 (m, 1H), 2.42-2.40 (m, 1H), 1.80-1.77 (m, 1H), 1.66-1.55 (m, 2H), 1.23-1.12 (m, 1H).

### 90.7 Benzyl 3-hydroxy-4-(picolinamido)azepane-1-carboxylate (I-90.7)

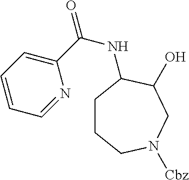

A solution of picolinic acid (0.66 g, 5 mmol), HOBt (1.33 g, 10 mmol), EDCI (2.83 g, 15 mmol), and TEA (1.5 g, 15 mmol) in DCM (80 mL) was stirred at RT for 0.5 h. A solution of benzyl 4-amino-3-hydroxyazepane-1-carboxylate (1.3 g, 4.9 mmol) in DCM (20 mL) was added dropwise at RT. The reaction mixture was stirred at RT overnight. The reaction was diluted with DCM, washed with water (5×20 mL) and saturated brine (20 mL), and dried over Na₂SO₄. Concentration in vacuo afforded the crude product, which was purified by column chromatography on silica gel (PE:EtOAc gradient=10:1 to 1:1) to afford the product benzyl 3-hydroxy-4-(picolinamido)azepane-1-carboxylate (1.2 g, 66%) as a light oil. ¹H NMR (400 MHz, CDCl₃): δ 8.53 (t, 1H), 8.23-8.16 (m, 1H), 7.87-7.82 (m, 1H), 7.45-7.37 (m, 1H), 7.36-7.28 (m, 5H), 5.22-5.10 (m, 2H), 4.05-3.83 (m, 2H), 3.78-3.72 (m, 2H), 3.63-3.50 (m, 1H), 3.39-3.29 (m, 1H), 2.07-1.71 (m, 3H), 1.65-1.61 (m, 1H).

### 90.8 Benzyl 3-oxo-4-(picolinamido)azepane-1-carboxylate (I-90.8)

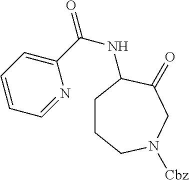

To a solution of benzyl 3-hydroxy-4-(picolinamido)azepane-1-carboxylate (1.2 g, 3 mmol) in DCM (100 mL) was added Dess-Martin reagent (3.8 g, 9 mmol) in portions. The reaction mixture was stirred at RT for 2 h. The reaction was diluted with DCM, washed with water (5×10 mL) and saturated brine (5×10 mL), and dried over Na₂SO₄. Concentration in vacuo afforded the crude product, which was purified by silica gel chromatography (DCM:MeOH gradient=100:1 to 30:1) to afford the product benzyl 3-oxo-4-(picolinamido)azepane-1-carboxylate (0.8 g, 67%) as a white oil. ¹H NMR (400 MHz, CDCl₃): δ 8.95 (dd, 1H), 8.59 (d, 1H), 8.14 (d, 1H), 7.85-7.81 (m, 1H), 7.44-7.28 (m, 6H), 5.27-5.15 (m, 2H), 5.08-4.75 (m, 2H), 4.31-4.22 (m, 1H), 3.70 (dd, 1H), 2.74-2.67 (m, 1H), 2.34-2.48 (m, 1H), 2.21-2.13 (m, 1H), 1.89-1.83 (m, 1H), 1.60-1.54 (m, 1H).

### 90.9 Benzyl 7,8-dihydro-2-(pyridin-2-yl)-4H-oxazolo[5,4-c]azepine-5(6H)-carboxylate (I-90.9)

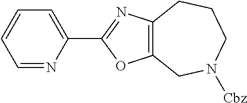

A solution of benzyl 3-oxo-4-(picolinamido)azepane-1-carboxylate (70 mg, 0.2 mmol) and PCl₅ (118 mg, 0.56 mmol) in 1,4-dioxane (5 mL) was stirred at 90° C. for 1 h. The mixture was cooled to 0° C. and quenched with water (10 mL). The solution was extracted with DCM (5×5 mL). The combined organic layers were washed with NaHCO₃ (1M, 5×10 mL), saturated brine (3×10 mL) and dried over Na₂SO₄. The mixture was filtered and concentrated in vacuo to afford the crude product, which was purified by preparative TLC (DCM:MeOH=50:1) to afford the pure product benzyl 7,8-dihydro-2-(pyridin-2-yl)-4H-oxazolo[5,4-c]azepine-5(6H)-carboxylate (7 mg, 10.5%) as a light oil. ¹H NMR (400 Mhz, CDCl₃): δ 8.70 (d, 1H), 8.04 (dd, 1H), 7.79 (t, 1H), 7.38-7.30 (m, 6H), 5.14 (d, 2H), 4.79 (d, 2H), 3.74 (d, 2H), 2.84 (t, 2H), 1.99 (d, 2H).

### 90.10 5,6,7,8-Tetrahydro-2-(pyridin-2-yl)-4H-oxazolo[5,4-c]azepine (I-90.10)

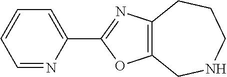

A mixture of benzyl 7,8-dihydro-2-(pyridin-2-yl)-4H-oxazolo[5,4-c]azepine-5(6H)-carboxylate (50 mg, 0.14 mmol) and TMSI (286 mg, 1.4 mmol) in MeCN (15 mL) was stirred for 1 h at RT. The solvent was removed in vacuo. The resulting oil was washed with ether and dried to afford the crude product as a brown solid, which was used without further purification (31 mg, 100%, crude).

### 91. Compound 91: 3-Methoxy-5-(2-(pyridin-2-yl)-7,8-dihydro-4H-oxazolo[5,4-c]azepin-5(6H)-yl)benzonitrile

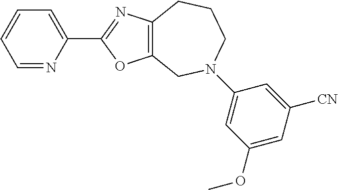

The title compound was prepared via the procedure used for Compound 90, using 3-bromo-5-methoxybenzonitrile instead of 3-bromo-5-fluorobenzonitrile. Preparative TLC (DCM:MeOH=30:1) afforded 3-methoxy-5-(2-(pyridin-2-yl)-7,8-dihydro-4H-oxazolo[5,4-c]azepin-5(6H)-yl)benzonitrile (19 mg, 12%) as a pale yellow solid. ¹H NMR (400 MHz, CDCl₃): δ 8.72 (d, 1H), 8.06 (d, 1H), 7.82-7.78 (m, 1H), 7.36-7.33 (m, 1H), 6.69 (s, 1H), 6.52 (s, 2H), 4.68 (s, 2H), 3.82-3.76 (m, 5H), 2.84 (t, 2H), 2.00-1.98 (m, 2H); LC/MS: m/e=347 (M+H)⁺.

### 92. Compound 92: 3-(2-(Pyrimidin-2-yl)-7,8-dihydro-4H-oxazolo[5,4-c]azepin-5(6H)-yl)benzonitrile

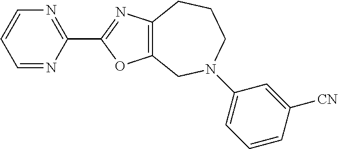

The title compound was prepared via the procedure used for Compound 90, using pyrimidine-2-carboxylic acid instead of picolinic acid, and using 3-bromobenzo-nitrile instead of 3-bromo-5-fluorobenzonitrile. Preparative HPLC afforded the product 3-(2-(pyrimidin-2-yl)-7,8-dihydro-4H-oxazolo[5,4-c]azepin-5(6H)-yl)benzonitrile (5 mg, 7%). ¹H NMR (400 MHz, CDCl₃): δ 8.85 (s, 2H), 7.34 (s, 1H), 7.22-7.18 (m, 1H), 6.96-6.92 (m, 3H), 4.67 (s, 2H), 3.79-3.77 (m, 2H), 2.90 (br s, 2H), 1.92 (s, 2H); LC/MS: m/e=318 (M+H)⁺.

### 93. Compound 93: 3-(2-(Pyridin-2-yl)-7,8-dihydro-4H-oxazolo[5,4-c]azepin-5(6H)-yl)benzonitrile

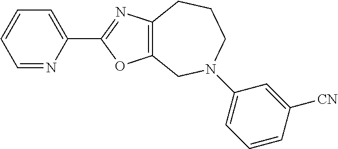

The title compound was prepared via the procedure used for Compound 90, using 3-bromobenzonitrile instead of 3-bromo-5-fluorobenzonitrile. Preparative TLC (DCM:MeOH=50:1) afforded the product 3-(7,8-dihydro-2-(pyridin-2-yl)-4H-oxazolo[5,4-c]azepin-5(6H)-yl)benzonitrile (7.5 mg, 16%) as a yellow oil. ¹H NMR (400 MHz, CDCl₃): δ 8.73 (d, 1H), 8.06 (d, 1H), 7.82-7.78 (m, 1H), 7.37-7.30 (m, 1H), 7.26 (t, 1H), 7.03-6.97 (m, 3H), 4.70 (s, 2H), 3.82 (t, 2H), 2.84 (t, 2H), 2.00-1.95 (m, 2H); LC/MS: m/e=317 (M+H)⁺.

### 94. Compound 94: 3-(2-(Pyridin-2-yl)-7,8-dihydro-4H-oxazolo[5,4-c]azepin-5(6H)-yl)-5-(trifluoromethyl)benzonitrile

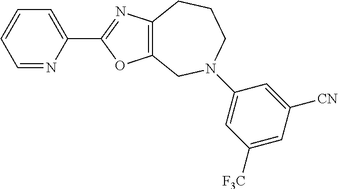

The title compound was prepared via the procedure used for Compound 90, using 3-bromo-5-(trifluoromethyl)benzonitrile instead of 3-bromo-5-fluorobenzonitrile. Preparative HPLC afforded 3-(trifluoromethyl)-5-(7,8-dihydro-2-(pyridin-2-yl)-4H-oxazolo[5,4-c]azepin-5(6H)-yl)benzonitrile (32 mg, 36%) as a brown oil. ¹H NMR (400 MHz, CDCl₃): δ 8.76 (d, 1H), 8.09 (d, 1H), 7.89-7.87 (m, 1H), 7.44-7.40 (m, 1H), 7.27-7.16 (m, 3H), 4.75 (s, 2H), 3.86-3.84 (t, 2H), 2.89-2.87 (t, 2H), 2.05-1.99 (m, 2H); LC/MS: m/e=385 (M+H)⁺.

### 95. Compound 95: 2-(Pyridin-2-yl)-5-(3-(trifluoromethyl)phenyl)-5,6,7,8-tetrahydro-4H-oxazolo[5,4-c]azepine

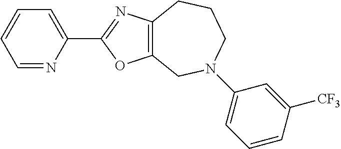

The title compound was prepared via the procedure used for Compound 90, using 1-bromo-3-(trifluoromethyl)benzene instead of 3-bromo-5-fluorobenzonitrile. Preparative HPLC afforded 2-(pyridin-2-yl)-5-(3-(trifluoromethyl)phenyl)-5,6,7,8-tetrahydro-4H-oxazolo[5,4-c]azepine (28 mg, 33%) as a brown oil. ¹H NMR (400 MHz, CDCl₃): δ 8.82 (d, 1H), 8.17 (d, 1H), 8.01-7.97 (m, 1H), 7.54-7.52 (m, 1H), 7.32-7.29 (m, 1H), 7.02-6.75 (m, 3H), 4.70 (s, 2H), 3.87-3.85 (m, 2H), 2.88-2.85 (m, 2H), 2.01-1.98 (m, 2H); LC/MS: m/e=360 (M+H)⁺.

### 96. Compound 96: 3-Fluoro-5-(2-(5-fluoropyridin-2-yl)-7,8-dihydro-4H-oxazolo[5,4-c]azepin-5(6H)-yl)benzonitrile

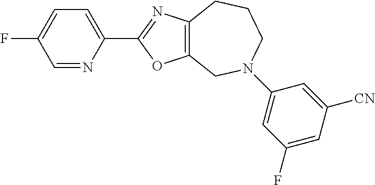

The title compound was prepared via the procedure used for Compound 90, using 5-fluoro-pyridine-2-carboxylic acid instead of picolinic acid. Preparative TLC (DCM:MeOH=100:1) afforded the product 3-fluoro-5-(2-(5-fluoropyridin-2-yl)-7,8-dihydro-4H-oxazolo[5,4-c]azepin-5(6H)-yl)benzonitrile (12 mg, 40%) as a white solid. ¹H NMR (400 MHz, CDCl₃): δ 8.57 (d, 1H), 8.12-8.08 (m, 1H), 7.55-7.50 (m, 1H), 6.81 (d, 1H), 6.71-6.67 (m, 2H), 4.67 (s, 2H), 3.79 (t, 2H), 2.85 (t, 2H), 2.02-1.98 (m, 2H); LC/MS: m/e=353 (M+H)⁺.

### 97. Compound 97: 3-(2-(5-Fluoropyridin-2-yl)-7,8-dihydro-4H-oxazolo[5,4-c]azepin-5(6H)-yl)benzonitrile

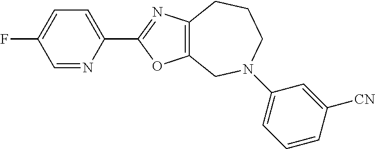

The title compound was prepared via the procedure used for Compound 90, using 5-fluoropicolinic acid instead of picolinic acid, and using 3-bromobenzonitrile instead of 3-bromo-5-fluorobenzonitrile. Preparative TLC (DCM:MeOH=100:1) afford 3-(2-(5-fluoropyridin-2-yl)-7,8-dihydro-4H-oxazolo[5,4-c]azepin-5(6H)-yl)benzonitrile (7 mg, 49%) as a pale yellow solid. ¹H NMR (400 MHz, CDCl₃): δ 8.57 (s, 1H), 8.11-8.08 (m, 1H), 7.55-7.50 (m, 1H), 7.28-7.18 (m, 1H), 7.00 (t, 3H), 4.69 (s, 2H), 3.82 (t, 2H), 2.83 (t, 2H), 2.04-1.95 (m, 2H); LC/MS: m/e=335 (M+H)⁺.

### 98. Compound 98: 3-Fluoro-5-(2-(pyridin-2-yl)-7,8-dihydro-4H-oxazolo[4,5-c]azepin-5(6H)-yl)benzonitrile

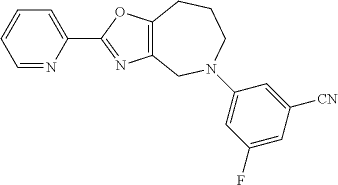

To a solution of 2-(pyridin-2-yl)-5,6,7,8-tetrahydro-4H-oxazolo[4,5-c]azepine (I-98.8) (50 mg, 0.23 mmol) in toluene (3 mL) was added 3-bromo-5-fluorobenzonitrile (69 mg, 0.35 mmol), Cs₂CO₃ (151 mg, 0.46 mmol), Pd(OAc)₂ (2 mg, cat.), and Xantphos (4 mg, cat.). The mixture was heated overnight at 100° C. The reaction was quenched into MeOH and filtered. The filtrate was concentrated and the residue purified by preparative TLC to afford 3-fluoro-5-(2-(pyridin-2-yl)-7,8-dihydro-4H-oxazolo[4,5-c]azepin-5(6H)-yl)benzonitrile (20 mg, 25%) as a yellow solid. ¹H NMR (400 MHz, CDCl₃): δ 8.65 (d, 1H), 8.00 (d, 1H), 7.75 (t, 1H), 7.27-7.31 (m, 1H), 6.77 (s, 1H), 6.62-6.69 (m, 1H), 6.59 (d, 1H), 4.47 (s, 2H), 3.65 (t, 2H), 2.95 (t, 2H), 1.90-2.00 (m, 2H); LC/MS: m/e=335 (M+H)⁺.

### 98.1 Benzyl 3-bromo-4-oxoazepane-1-carboxylate (I-98.1)

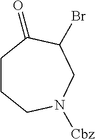

To a solution of benzyl 4-oxoazepane-1-carboxylate (I-88.3) (10 g, 40.4 mmol) in CHCl₃ (500 mL) was added Br₂ (6.5 g, 40.5 mmol) dropwise at 5-20° C. The mixture was stirred for 3 h. Aqueous NaHCO₃ solution was added and the mixture was charged to a separatory funnel. The mixture was extracted with DCM (3×150 mL). The organic phase was dried over anhydrous Na₂SO₄ and concentrated to afford crude benzyl 3-bromo-4-oxoazepane-1-carboxylate (13.2 g, 100%) as a red liquid. The crude product was used next step without purification.

### 98.2 Benzyl 3-bromo-4-hydroxyazepane-1-carboxylate (I-98.2)

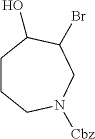

To a solution of benzyl 3-bromo-4-oxoazepane-1-carboxylate (13.2 g, 40.4 mmol) in MeOH (100 mL) was added NaBH₄ (2.05 g, 54 mmol) at 0° C. The reaction was stirred for 30 min at RT. The reaction was quenched with 1N HCl and extracted with DCM (3×150 mL). The organic phase was dried over anhydrous Na₂SO₄ and concentrated. The residue was purified by silica gel chromatography (PE:EtOAc=5:1) to afford benzyl 3-bromo-4-hydroxyazepane-1-carboxylate (3.8 g, 28%) as a yellow oil. ¹H NMR (400 MHz, DMSO-d₆): δ 7.20-7.40 (m, 5H), 5.96 (dd, 0.5H), 5.15 (t, 0.5H) 5.05 (d, 2H), 4.22-4.55 (m, 1H), 3.95-4.06 (m, 1H), 3.61-3.92 (m, 3H), 3.41-3.60 (m, 2H), 2.20-2.32 (m, 1H), 1.70-1.85 (m, 1H), 1.40-1.61 (m, 1H).

### 98.3 Benzyl 3-azido-4-hydroxyazepane-1-carboxylate (I-98.3)

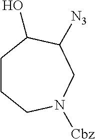

To a solution of benzyl 3-bromo-4-hydroxyazepane-1-carboxylate (4.8 g, 14.6 mmol) in DMF (50 mL) was added NaN₃ (1.43 g, 22 mmol). The mixture was heat to 70° C. and stirred overnight at this temperature. Then water was added to the mixture. The mixture was extracted with CH₂Cl₂ (50 mL×3). The organic phase was dried over anhydrous Na₂SO₄ and concentrated. The residue was purified by silica gel column (PE:EtOAc=5:1) to afford benzyl 3-azido-4-hydroxyazepane-1-carboxylate (1.1 g, 26%) as a yellow oil. ¹H NMR (400 MHz, DMSO-d₆): δ 7.25-7.40 (m, 5H), 5.05-5.25 (m, 3H), 3.41-3.73 (m, 6H), 3.00-3.21 (m, 1H), 1.70-1.80 (m, 1H), 1.30-1.65 (m, 2H).

### 98.4 Benzyl 3-amino-4-hydroxyazepane-1-carboxylate (I-98.4)

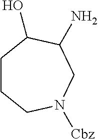

To a solution of benzyl 3-azido-4-hydroxyazepane-1-carboxylate (1.1 g, 3.79 mmol) in THF (20 mL) and H₂O (1 mL) was added Ph₃P (2.0 g, 7.58 mmol). The reaction was heated to reflux and stirred for 3 days. The reaction was concentrated to dryness, and the crude product was purified by silica gel chromatography (DCM:MeOH=5:1) to afford benzyl 3-amino-4-hydroxyazepane-1-carboxylate (450 mg, 45%) as a yellow liquid. ¹H NMR (400 MHz, DMSO-d₆): δ 7.25-7.40 (m, 5H), 5.09 (d, 2H), 4.90 (br, 1H), 3.60-3.65 (m, 1H), 3.41-3.55 (m, 2H), 3.10-3.22 (m, 2H), 2.90-3.00 (m, 1H), 2.55-2.67 (m, 2H), 1.70-1.83 (m, 2H), 1.40-1.55 (m, 1H), 1.26-1.40 (m, 1H).

### 98.5 Benzyl 4-hydroxy-3-(picolinamido)azepane-1-carboxylate (I-98.5)

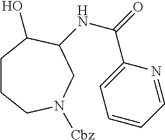

To a solution of picolinic acid (230 mg, 1.7 mmol) in DCM (7.5 mL), TEA (344 mg, 3.4 mmol), EDCI (600 mg, 3.4 mmol), and HOBt (460 mg, 3.4 mmol) was added a solution of benzyl 3-amino-4-hydroxyazepane-1-carboxylate (450 mg, 1.7 mmol) in DCM (7.5 mL). The mixture was stirred overnight at RT. The reaction was diluted with DCM and washed with water, aq.NaHCO₃, 1N HCl solution and brine. The combined organic phases were dried over anhydrous Na₂SO₄ and concentrated to dryness. The residue was purified by silica gel chromatography (PE:EtOAc=1:1) to afford benzyl 4-hydroxy-3-(picolinamido)azepane-1-carboxylate (410 mg, 65%) as a yellow oil. ¹H NMR (400 MHz, DMSO-d₆): δ 8.59 (d, 1H), 8.47 (q, 1H), 7.91-8.05 (m, 2H), 7.50-7.60 (m, 1H), 7.16-7.37 (m, 5H), 4.85-5.10 (m, 3H), 3.85-3.98 (m, 1H), 3.52-3.70 (m, 3H), 1.75-1.90 (m, 1H), 1.65-1.75 (m, 1H), 1.49-1.63 (m, 2H).

### 98.6 Benzyl 4-oxo-3-(picolinamido)azepane-1-carboxylate (I-98.6)

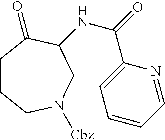

To a solution of benzyl 4-hydroxy-3-(picolinamido)azepane-1-carboxylate (410 mg, 1.1 mmol) in DCM (15 mL) was added Dess-Martin reagent (2.22 g, 940 mmol). The mixture was stirred overnight at RT. The reaction was diluted with DCM and washed with 0.5 N NaOH solution. The organic phase was dried over anhydrous Na₂SO₄ and concentrated to afford benzyl 4-oxo-3-(picolinamido)azepane-1-carboxylate (400 mg, 99%) as a yellow oil. ¹H NMR (400 MHz, DMSO-d₆): δ 8.92 (q, 1H), 8.55-8.67 (m, 1H), 7.91-8.05 (m, 2H), 7.55-7.62 (m, 1H), 7.10-7.40 (m, 5H), 4.82-5.10 (m, 3H), 3.80-4.00 (m, 2H), 3.65-3.75 (m, 1H), 3.40-3.50 (m, 1H), 2.80-2.90 (m, 1H), 2.50-2.58 (m, 1H), 1.75-1.90 (m, 1H), 1.54-1.68 (m, 1H).

### 98.7 Benzyl 2-(pyridin-2-yl)-7,8-dihydro-4H-oxazolo[4,5-c]azepine-5(6H)-carboxylate (I-98.7)

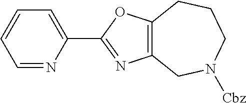

To a solution of benzyl 4-oxo-3-(picolinamido)azepane-1-carboxylate (380 mg, 1.04 mmol) in dioxane (20 mL) was added PCl₅ (1.1 g, 5.2 mmol). The reaction mixture was heated to 70° C. and stirred for 3 h. The reaction was quenched into aq. NaHCO₃ and extracted with EtOAc (3×20 mL). The organic phase was dried over anhydrous Na₂SO₄ and concentrated to dryness. The residue was purified by silica gel chromatography (PE:EtOAc=1:1) to afford benzyl 2-(pyridin-2-yl)-7,8-dihydro-4H-oxazolo[4,5-c]azepine-5(6H)-carboxylate (42 mg, 22%) as a yellow oil. ¹H NMR (400 MHz, CDCl₃): δ 8.63 (d, 1H), 7.96-8.02 (m, 1H), 7.72 (t, 1H), 7.40-7.50 (m, 1H), 7.20-7.35 (m, 5H), 5.10 (d, 2H), 4.61 (d, 2H), 3.58-3.65 (m, 2H), 3.05 (t, 2H), 1.90-2.05 (m, 2H).

### 98.8 2-(Pyridin-2-yl)-5,6,7,8-tetrahydro-4H-oxazolo[4,5-c]azepine (I-98.8)

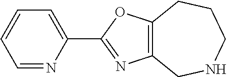

To a solution of benzyl 2-(pyridin-2-yl)-7,8-dihydro-4H-oxazolo[4,5-c]azepine-5(6H)-carboxylate (160 mg, 0.46 mmol) in MeCN (5 mL) was added TMSI (916 mg, 4.6 mmol). The mixture was stirred for 30 min at RT. The mixture was concentrated and washed with Et₂O to afford 2-(pyridin-2-yl)-5,6,7,8-tetrahydro-4H-oxazolo[4,5-c]azepine (120 mg, 100%) as a yellow solid. ¹H NMR (400 MHz, MeOH-d₄): δ 8.71 (d, 1H), 8.15-8.30 (m, 2H), 7.70 (t, 1H), 4.48 (s, 2H), 3.59 (t, 2H), 3.13 (t, 2H), 2.15-2.25 (m, 2H).

### 99. Compound 99: 3-(2-(Pyridin-2-yl)-7,8-dihydro-4H-oxazolo[4,5-c]azepin-5(6H)-yl)benzonitrile

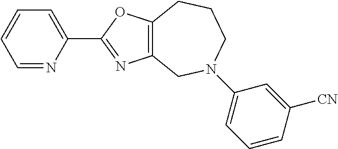

The title compound was prepared via the procedure used for Compound 98, using 3-bromobenzonitrile instead of 3-bromo-5-fluorobenzonitrile. Preparative TLC afforded 3-(2-(pyridin-2-yl)-7,8-dihydro-4H-oxazolo[4,5-c]azepin-5(6H)-yl)benzonitrile (5 mg, 17%) as a yellow solid. ¹H NMR (400 MHz, CDCl₃): δ 8.65 (d, 1H), 8.00 (d, 1H), 7.75 (t, 1H), 7.27 (t, 1H), 7.15-7.20 (m, 1H), 6.95-7.00 (m, 2H), 6.80 (d, 1H), 4.49 (s, 2H), 3.78 (t, 2H), 2.94 (t, 2H), 1.87-1.96 (m, 2H); LC/MS: m/e=317 (M+H)⁺.

### 100. Compound 100: 5-(3-Fluorophenyl)-2-(pyridin-2-yl)-5,6,7,8-tetrahydro-4H-oxazolo[4,5-c]azepine

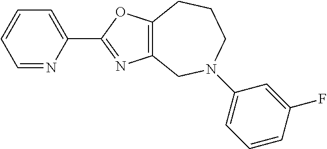

The title compound was prepared via the procedure used for Compound 98, using 1-bromo-3-fluorobenzene instead of 3-bromo-5-fluorobenzonitrile. Preparative TLC afforded 5-(3-fluorophenyl)-2-(pyridin-2-yl)-5,6,7,8-tetrahydro-4H-oxazolo[4,5-c]azepine (15 mg, 21%) as a yellow solid. ¹H NMR (400 MHz, CDCl₃): δ 8.65 (d, 1H), 7.98 (d, 1H), 7.72 (t, 1H), 7.26 (t, 1H), 7.05 (q, 1H), 6.51 (d, 1H), 6.45 (tt, 1H), 6.30 (t, 1H), 4.48 (s, 2H), 3.65 (t, 2H), 2.93 (t, 2H), 1.90-2.00 (m, 2H); LC/MS: m/e=309 (M+H)⁺.

### 101. Compound 101: 5-(3,5-Difluorophenyl)-2-(pyridin-2-yl)-5,6,7,8-tetrahydro-4H-oxazolo[4,5-c]azepine

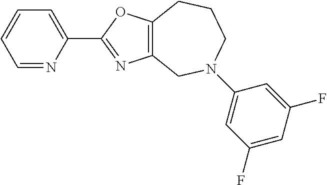

The title compound was prepared via the procedure used for Compound 98, using 1-bromo-3,5-difluorobenzene instead of 3-bromo-5-fluorobenzonitrile. Preparative TLC afforded 5-(3,5-difluorophenyl)-2-(pyridin-2-yl)-5,6,7,8-tetrahydro-4H-oxazolo[4,5-c]azepine as a brown solid (2.4 mg, 3%). ¹H NMR (400 MHz, CDCl₃): δ 8.68 (d, 1H), 8.05 (d, 1H), 7.83 (t, 1H), 7.37 (m, 1H), 6.25 (m, 2H), 6.08 (m, 1H), 4.44 (s, 2H), 3.71 (m, 2H), 2.93 (t, 2H), 1.95 (m, 2H); LC/MS: m/e=328 (M+H)⁺.

### 102. Compound 102: 5-(3-Chlorophenyl)-2-(pyridin-2-yl)-5,6,7,8-tetrahydro-4H-oxazolo[4,5-c]azepine

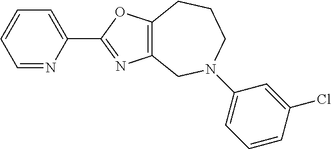

The title compound was prepared via the procedure used for Compound 98, using 1-bromo-3-chlorobenzene instead of 3-bromo-5-fluorobenzonitrile. Preparative HPLC afforded 5-(3-chlorophenyl)-2-(pyridin-2-yl)-5,6,7,8-tetrahydro-4H-oxazolo[4,5-c]azepine as a pale yellow solid (6.4 mg, 4%). ¹H NMR (400 MHz, CDCl₃): δ 8.71 (s, 1H), 8.04 (d, 1H), 7.80 (t, 1H), 7.33 (m, 1H), 7.10 (t, 1H), 6.80 (s, 1H), 6.71 (d, 1H), 6.67 (d, 1H), 4.52 (s, 2H), 3.79 (s, 2H), 3.00 (s, 2H), 1.98 (s, 2H); LC/MS: m/e=326, 328 (M+H)⁺.

### 103. Compound 103: 5-(6-Methoxypyridin-2-yl)-2-(pyridin-2-yl)-5,6,7,8-tetrahydro-4H-oxazolo[4,5-c]azepine

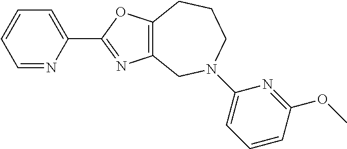

The title compound was prepared via the procedure used for Compound 98, using 2-bromo-6-methoxypyridine instead of 3-bromo-5-fluorobenzonitrile. Preparative HPLC afforded 5-(6-methoxypyridin-2-yl)-2-(pyridin-2-yl)-5,6,7,8-tetrahydro-4H-oxazolo[4,5-c]azepine as a pale solid (37 mg, 24%). ¹H NMR (400 MHz, CDCl₃): δ 8.69 (m, 1H), 8.04 (d, 1H), 7.78 (td, 1H), 7.35 (m, 1H), 7.31 (m, 1H), 6.16 (d, 1H), 5.98 (d, 1H), 4.83 (s, 2H), 3.89 (m, 5H), 3.00 (t, 2H), 2.04 (m, 2H); LC/MS: m/e=323 (M+H)⁺.

### 104. Compound 104: 5-(5-Fluoropyridin-3-yl)-2-(pyridin-2-yl)-5,6,7,8-tetrahydro-4H-oxazolo[4,5-c]azepine

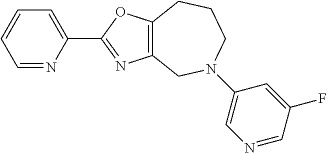

The title compound was prepared via the procedure used for Compound 98, using 3-bromo-5-fluoropyridine instead of 3-bromo-5-fluorobenzonitrile. Preparative HPLC afforded 5-(5-fluoropyridin-3-yl)-2-(pyridin-2-yl)-5,6,7,8-tetrahydro-4H-oxazolo[4,5-c]azepine as a pale solid (9.6 mg, 7%). ¹H NMR (400 MHz, CDCl₃): δ 8.70 (d, 1H), 8.05 (m, 2H), 7.81 (m, 2H), 7.34 (m, 1H), 6.82 (d, 1H), 4.54 (s, 2H), 3.03 (t, 2H), 2.00 (m, 2H); LC/MS: m/e=311 (M+H)⁺.

### 105. Compound 105: 3-Fluoro-5-(2-(pyridin-2-yl)-6,7-dihydro-thiazolo[5,4-c]pyridin-5(4H)-yl)benzonitrile

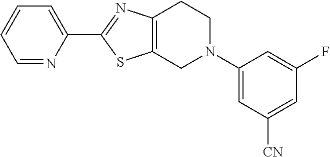

2-(Pyridin-2-yl)-4,5,6,7-tetrahydrothiazolo[5,4-c]pyridine (I-105.2) (107 mg, 0.49 mmol), tris(dibenzylideneacetone)dipalladium(0) (31 mg, 0.034 mmol), BINAP (77 mg, 0.12 mmol), and 3-bromo-5-fluorobenzonitrile (97 mg, 0.49 mmol) were combined in toluene (4 mL). The reaction was flushed with nitrogen. Sodium tert-butoxide (56 mg, 0.59 mmol) was added, the reaction flushed once again with nitrogen and heated at 80° C. under nitrogen overnight. The solvent was concentrated removed under vacuum. The residue was purified by silica gel chromatography (EtOAc (10% MeOH)/Hexanes), followed by recrystallization in MeOH to give the product as a yellow solid (5.3 mg, 3%). ¹H NMR (400 MHz, CDCl₃) δ 8.60 (d, 1H), 8.12 (d, 1H), 7.76-7.81 (m, 1H), 7.30-7.33 (m, 1H), 6.99 (s, 1H), 6.84-6.88 (m, 1H), 6.78 (d, 1H), 4.57 (s, 2H), 3.77 (t, 2H), 3.08 (t, 2H); LC/MS: m/e=337 (M+H)⁺.

### 105.1 3-Bromopiperidin-4-one hydrobromide (I-105.1)

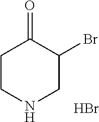

tert-Butyl 4-oxopiperidine-1-carboxylate (purchased from Fluka) (10 g, 50.2 mmol) was dissolved in chloroform (350 mL). Bromine (8.02 g, 50.2 mmol) was added dropwise and the reaction was stirred at RT for 1 hour. The mixture was cooled in an ice bath to 0° C. and stirred for an additional hour. The precipitate was filtered and washed with cold DCM, then dried under reduced pressure to yield a pink solid (5.32 g, 41%). LC/MS: m/e=177 (M+H)⁺.

### 105.2 2-(Pyridin-2-yl)-4,5,6,7-tetrahydrothiazolo[5,4-c]pyridine (I-105.2)

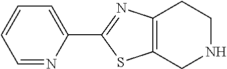

3-Bromopiperidin-4-one hydrobromide (1 g, 3.86 mmol) and pyridine-2-carbothioic acid amide (800 mg, 5.79 mmol) were combined in DMF (20 mL) and stirred at RT under nitrogen for 24 hours. The solvent was removed under reduced pressure, and the residue was purified by preparative HPLC to give a yellow solid (70 mg, 8%). ¹H NMR (400 MHz, CDCl₃): δ 8.59 (m, 1H), 8.12 (d, 1H), 7.74-7.78 (m, 1H), 7.27-7.30 (m, 1H), 4.12 (m, 2H), 3.23 (t, 2H), 2.88-2.92 (m, 2H); LC/MS: m/e=218 (M+H)⁺.

### 106. Compound 106: 2-(3-Chlorophenyl)-7-(pyridin-3-yl)-5,6,7,8-tetrahydroimidazo[1,2-a]pyrazine

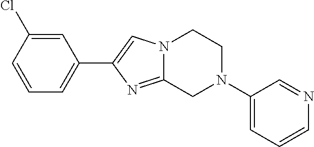

2-(3-Chlorophenyl)-5,6,7,8-tetrahydroimidazo[1,2-a]pyrazine (I-106.4) (200 mg, 0.86 mmol), tris(dibenzylideneacetone)dipalladium(0) (55 mg, 0.06 mmol), tris-tert-butyl phosphonium tetrafluoroborate (62 mg, 0.2 mmol), and 3-iodopyridine (263 mg, 1.28 mmol) were combined in toluene (5 mL). The mixture was flushed with nitrogen. Sodium tert-butoxide (164 mg, 1.71 mmol) was added and the solution was purged a second time with nitrogen. The reaction was heated at 120° C. under nitrogen for 4 hours. The solvent was concentrated and removed under vacuum. The residue was then purified by silica gel chromatography (EtOAc (10% MeOH)/Hexanes), followed by reverse phase purification by Gilson GX-281 (NH₄HCO₃/Acetonitrile) to give a yellow oil (53 mg, 20%). ¹H NMR (400 MHz, CDCl₃): δ 8.42 (m, 1H), 8.19 (m, 1H), 7.75 (m, 1H), 7.62 (m, 1H), 7.27-7.31 (m, 1H), 7.19-7.25 (m, 2H), 4.56 (s, 2H), 4.18 (m, 2H), 3.78 (m, 2H); LC/MS: m/e=311 (M+H)⁺.

### 106.1 Benzyl-4-(3-chlorophenyl)-1H-imidazol-2-ylcarbamate (I-106.1)

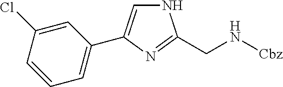

Cbz-glycine (10 g, 47.8 mmol) and Cs₂CO₃ (7.79 g, 23.9 mmol) were combined in 2:1 DMF:H₂O (66 mL), and the mixture was swirled until homogeneous. Solvents were removed under reduced pressure, the white residue was dissolved in DMF (75 mL) and 2-bromo-3′-chloroacetophenone (11.16 g, 47.8 mmol) in DMF (55 mL) was added. The mixture was stirred for 15 minutes at room temperature (the solution turned orange) and then concentrated under reduced pressure. The resulting orange solid was dissolved in xylenes (250 mL) and filtered. NH₄OAc (33.14 g, 0.43 mol) was added to the filtrate, and the reaction was fitted with a Dean-Stark trap and heated at reflux for 3 hours. The reaction mixture was concentrated under reduced pressure. Saturated NaHCO₃ solution (100 mL) was added and the product was extracted with DCM (3×50 mL). The combined organic layers were dried over Na₂SO₄, and filtered. A first crop precipitated and was filtered to give a white solid. The mother liquor was purified by silica gel chromatography (EtOAc (10% MeOH)/Hexanes) to give a yellow solid. The two lots were combined to yield a yellow/white solid (3.48 g, 21%). ¹H NMR (400 MHz, CDCl₃): δ 7.73 (s, 1H), 7.59 (m, 1H), 7.28-7.35 (m, 5H), 7.19-7.23 (m, 3H), 5.80 (s, 1H), 5.15 (s, 2H), 4.41 (d, 2H); LC/MS: m/e=342 (M+H)⁺.

### 106.2 Ethyl 2-(2-((benzyloxycarbonylamino)methyl)-4-(3-chlorophenyl)-1H-imidazol-1-yl)acetate (I-106.2)

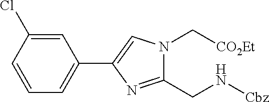

Benzyl-4-(3-chlorophenyl)-1H-imidazol-2-ylcarbamate (3.48 g, 10.2 mmol) was dissolved in DMF (20 mL). K₂CO₃ (2.81 g, 20.4 mmol) and ethyl bromoacetate (5.10 g, 30.54 mmol) were added, and the mixture was heated at 55° C. for 2 hours. The mixture was concentrated, dissolved in ether (40 mL) and washed with saturated NaHCO₃ (20 mL) and saturated NaCl solution (20 mL). The ether layer was dried over Na₂SO₄, filtered and concentrated to a yellow solid (4.10 g, 94%) which was used without further purification. ¹H NMR (400 MHz, CDCl₃): δ 7.73 (m, 1H), 7.58 (d, 1H), 7.31-7.35 (m, 5H), 7.28 (m, 1H), 7.19 (m, 1H), 7.16 (s, 1H), 5.53 (m, 1H), 5.11 (s, 2H), 4.84 (s, 2H), 4.46 (d, 2H), 4.19-4.25 (m, 2H), 1.27-1.34 (m, 3H); LC/MS: m/e=429 (M+H)⁺.

### 106.3 2-(3-Chlorophenyl)-7,8-dihydroimidazo[1,2,a]pyrazin-6(5H)-one (I-106.3)

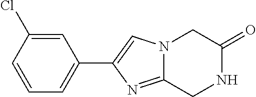

Ethyl 2-(2-((benzyloxycarbonylamino)methyl)-4-(3-chlorophenyl)-1H-imidazol-1-yl)acetate (4.09 g, 9.56 mmol) and 5% Pd/C catalyst (200 mg, 0.05 eq) were combined in acetic acid (40 mL) and shaken under hydrogen atmosphere (30 psi of H₂) for 3 hours at room temperature. The catalyst was removed by filtration through Celite, washed with acetic acid and the filtrate warmed at 70° C. for 4 hours under nitrogen. The mixture was concentrated to a solid under reduced pressure, dissolved in DCM and washed with saturated NaHCO₃ solution. A solid precipitated and was filtered which was washed with DCM and water (1.50 g, 63%). ¹H NMR (400 MHz, DMSO-d₆): δ 8.48 (s, 1H), 7.75 (m, 1H), 7.67 (m, 2H), 7.34-7.38 (t, 1H), 7.23 (m, 1H), 4.63 (s, 2H), 4.45 (s, 2H), 1.55 (s, 2H); LC/MS: m/e=248 (M+H)⁺.

### 106.4 2-(3-Chlorophenyl)-5,6,7,8-tetrahydroimidazo[1,2-a]pyrazine (I-106.4)

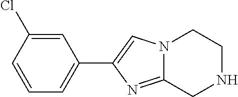

2-(3-Chlorophenyl)-7,8-dihydroimidazo[1,2,a]pyrazin-6(5H)-one (1.49 g, 6.01 mmol) was dissolved in THF (30 mL). BH₃/THF (1M, 24 mmol, 4 eq) was added at room temperature, and the reaction was heated at reflux for 4 hours under nitrogen. The mixture was cooled to room temperature and 4N HCl (15 mL) was added dropwise. The solution was stirred at room temperature for 1 hour and then heated at 70° C. for 30 minutes. After cooling to room temperature the mixture was made basic by careful portionwise addition of solid K₂CO₃. The solution was extracted with EtOAc (3×15 mL). The organic layers were combined, dried over NaSO₄, filtered and concentrated under reduced pressure to give a yellow solid (1.36 g, 97%). ¹H NMR (400 MHz, CDCl₃): δ 7.73 (s, 1H), 7.59 (d, 1H), 7.28 (m, 1H), 7.17 (m, 1H), 7.12 (s, 1H), 4.16 (s, 2H), 4.00 (t, 2H), 3.28 (t, 2H); LC/MS: m/e=234 (M+H)⁺.

### 107. Compound 107: 2-(3-Chlorophenyl)-7-(pyridin-2-yl)-5,6,7,8-tetrahydroimidazol[1,2-a]pyrazine

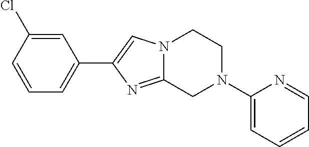

2-(3-Chlorophenyl)-5,6,7,8-tetrahydroimidazo[1,2-a]pyrazine (I-106.4) (200 mg, 0.86 mmol), 2-bromopyridine (203 mg, 1.28 mmol), and DIEA (221 mg, 1.71 mmol) were combined in DMF (2 mL) and heated under microwave at 150° C. for 70 minutes, and then at 180° C. for 2 hours. The solvent was removed under vacuum, and the residue was purified by silica gel chromatography (EtOAc (10% MeOH)/Hexanes) to give a yellow solid (39 mg, 15%). ¹H NMR (400 MHz, CDCl₃): δ 8.23 (m, 1H), 7.75 (m, 1H), 7.55-7.63 (m, 2H), 7.29 (m, 1H), 7.18-7.21 (m, 2H), 6.71-6.74 (m, 2H), 4.74 (s, 2H), 4.23 (m, 2H), 4.15 (m, 2H); LC/MS: m/e=311 (M+H)⁺.

### 108. Compound 108: 2-(2-(3-Chlorophenyl)-5,6-dihydroimidazo[1,2-a]pyrazin-7(8H)-yl)nicotinotrile

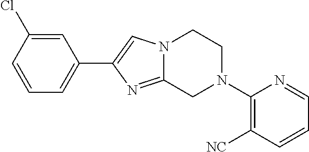

2-(3-Chlorophenyl)-5,6,7,8-tetrahydroimidazo[1,2-a]pyrazine (I-106.4) (200 mg, 0.86 mmol), 2-chloro-3-pyridine-carbonitrile (237 mg, 1.71 mmol), and DIEA (221 mg, 1.71 mmol) were combined in DMF (1 mL) and heated under microwaves at 180° C. for 1 hour. The solvent was removed under vacuum. The residue was purified by silica gel chromatography (EtOAc (10% MeOH)/Hexanes and flush with 9:1 DCM:MeOH) to give a yellow solid/oil (23 mg, 8%). ¹H NMR (400 MHz, CDCl₃): δ 8.41 (m, 1H), 7.85 (m, 1H), 7.74 (m, 1H), 7.61 (m, 1H), 7.26-7.30 (m, 1H), 7.20 (m, 2H), 6.88 (m, 1H), 4.98 (s, 2H), 4.27 (t, 2H), 4.17 (m, 2H); LC/MS: m/e=336 (M+H)⁺.

### 109. Compound 109: 3-Fluoro-5-(2(pyridin-2-yl)-5,6-dihydroimidazo-[1,2-a]pyrazin-7(8H)-yl)benzonitrile

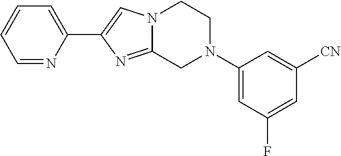

The title compound was prepared via the procedure used for Compound 106, using 2-bromo-1-(pyridin-2-yl)ethanone instead of 2-bromo-3′-chloroacetophenone, and using 3-bromo-5-fluorobenzonitrile instead of 3-iodopyridine. Purification by silica gel chromatography (EtOAc (10% MeOH)/Hexanes) gave a yellow solid (7.1 mg, 21%). ¹H NMR (400 MHz, CDCl₃): δ 8.55 (m, 1H), 7.90 (d, 1H), 7.71 (t, 1H), 7.58 (s, 1H), 7.15 (m, 1H), 6.99 (s, 1H), 6.85 (m, 2H), 4.59 (s, 2H), 4.20 (m, 2H), 3.81 (m, 2H); LC/MS: m/e=320 (M+H)⁺.

### 110. Compound 110: 3-(2(Pyridin-2-yl)-5,6-dihydroimidazo[1,2-a]pyrazin-7(8H)-yl)benzonitrile

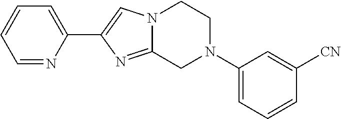

The title compound was prepared via the procedure used for Compound 106, using 2-bromo-1-(pyridin-2-yl)ethanone instead of 2-bromo-3′-chloroacetophenone, and using 3-bromobenzonitrile instead of 3-iodopyridine. Purification by silica gel chromatography (EtOAc (10% MeOH)/Hexanes) gave a yellow solid (16 mg, 22%). ¹H NMR (400 MHz, CDCl₃): δ 8.53 (m, 1H), 7.90 (d, 1H), 7.71 (t, 1H), 7.57 (s, 1H), 7.39 (t, 1H), 7.19 (m, 3H), 7.14 (m, 1H), 4.58 (s, 2H), 4.19 (m, 2H), 3.79 (m, 2H). LC/MS: m/e=302 (M+H)⁺.

### 111. Compound 111: 3-Fluoro-5-(2-(pyridin-2-yl)-6,7-dihydro-3H-imidazo[4,5-c]pyridin-5(4H)-yl)benzonitrile

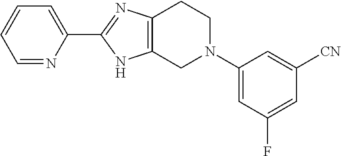

Trifluoroacetic acid (1 mL) was added to 3-fluoro-5-(3-(4-methoxybenzyl)-2-(pyridine-2-yl)-6,7-dihydro-3H-imidazo[4,5-c]pyridine-5(4H)-yl)benzonitrile (I-111.8) (20 mg, 0.045 mmol) and the reaction was heated to reflux. After 3 hours, the TFA was evaporated and the crude material was purified by reverse phase purification (Gilson GX-281 (NH₄HCO₃/Acetonitrile)) to give a white solid (2.1 mg, 14%). ¹H NMR (400 MHz, CDCl₃): δ 8.50 (d, 1H), 8.10 (d, 1H), 8.01 (s, 1H), 7.78 (t, 1H), 6.96 (s, 1H), 6.83 (d, 1H), 6.74 (s, 1H), 4.39 (s, 2H), 3.73 (t, 2H), 2.74 (s, 2H); LC/MS: m/e=320 (M+H)⁺.

### 111.1 Benzyl 4-azido-3-(methylsulfonyloxy)piperidine-1-carboxylate (I-111.1)

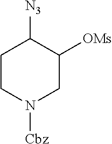

Benzyl 4-azido-3-hydroxypiperidine-1-carboxylate (10.6 g, 38 mmol) (prepared according to the procedure in Hall, S. E., et al., WO1994/20062) was dissolved in DCM (100 mL) and cooled in an ice bath. TEA (6.75 g, 67 mmol) was added, followed by the dropwise addition of methanesulfonyl chloride (5.56 g, 48 mmol). The mixture was warmed to room temperature and stirred for 90 min. The reaction was diluted with DCM and washed with 1N HCl, NaHCO₃, and brine. The organic layer was dried over Na₂SO₄, filtered and concentrated under vacuum to give a yellow oil (14.4 g, 100%). ¹H NMR (400 MHz, CDCl₃): δ 7.30-7.38 (m, 5H), 5.10 (s, 2H), 4.39 (m, 1H), 4.10 (m, 1H), 3.90 (m, 1H), 3.77 (s, 1H), 3.30 (m, 1H), 3.08 (s, 3H), 2.10 (m, 1H), 2.61 (m, 1H); LC/MS: m/e=355 (M+H)⁺.

### 111.2 Benzyl 3,4-diazidopiperidine-1-carboxylate (I-111.2)

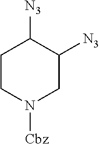

Benzyl 4-azido-3-(methylsulfonyloxy)piperidine-1-carboxylate (14.1 g, 40 mmol) was dissolved in DMF (200 mL). Sodium azide (6.91 g, 106 mmol) was added and the mixture was heated at 100° C. overnight. The reaction was diluted with EtOAc and the organic layer was washed with water (2×300 mL) followed by brine, and then dried over Na₂SO₄. Removal of the solvent under vacuum afforded a residue which was purified by silica gel chromatography (EtOAc/Hexanes) to give an orange oil (7.48 g, 62%). ¹H NMR (400 MHz, CDCl₃): δ 7.30-7.38 (m, 5H), 5.10 (s, 2H), 4.60-4.80 (d, 4H), 3.37 (m, 2H), 1.98 (s, 1H), 1.79 (s, 1H); LC/MS: m/e=302 (M+H)⁺.

### 111.3 Benzyl 3,4-diaminopiperidine-1-carboxylate (I-111.3)

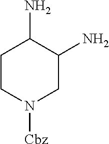

Benzyl 3,4-diazidopiperidine-1-carboxylate (7.48 g, 24.8 mmol) was dissolved in a mixture of THF (120 mL) and H₂O (7 mL). Triphenylphosphine (19.53 g, 74 mmol) was added and the mixture was heated at reflux overnight. The reaction mixture was concentrated to dryness and purified by silica gel chromatography (DCM/MeOH) to give a white solid (3.4 g, 55%). ¹H NMR (400 MHz, CDCl₃): δ 7.30-7.38 (m, 5H), 5.10 (m, 2H), 3.86 (s, 1H), 3.79 (m, 1H), 3.20 (d, 1H), 3.09 (s, 1H), 2.90 (m, 2H), 1.58 (m, 2H), 1.10-1.50 (m, 4H); LC/MS: m/e=250 (M+H)⁺.

### 111.4 Benzyl-2-(pyridine-2-yl)-3a,4,7,7a-tetrahydro-3H-imidazo[4,5-c]pyridine-5(6H) carboxylate (I-111.4)

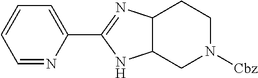

Benzyl 3,4-diaminopiperidine-1-carboxylate (3.06 g, 12.3 mmol) was dissolved in anhydrous ethanol (10 mL). Ethyl picolinimidate (3.69 g, 24.6 mmol) (prepared according to the procedure in Watanabe, H., et al., Chem. Pharm. Bull. 1973, 21, 465) was added, and the reaction was heated at 75° C. for 2 hours. The reaction mixture was cooled and partitioned between DCM (100 mL) and NaHCO₃ (100 mL). The aqueous layer was extracted a second time with DCM and the combined organic layers were dried over Na₂SO₄. Removal of the solvent afforded crude material that was purified by silica gel chromatography (DCM/MeOH) to give a yellow oil (2.98 g, 72%). ¹H NMR (400 MHz, CDCl₃): δ 8.56 (d, 1H), 8.11 (m, 1H), 7.76 (m, 1H), 7.32-7.40 (m, 5H), 6.02 (s, 1H), 5.10 (s, 2H), 4.49 (s, 1H), 4.15 (s, 1H), 3.70 (s, 1H), 3.5 (m, 2H), 2.02 (s, 2H), 2.85 (s, 1H); LC/MS: m/e=337 (M+H)⁺.

### 111.5 Benzyl-2-(pyridine-2-yl)-6,7-dihydro-3H-imidazo[4,5-c]pyridine-5(4H)-carboxylate (I-111.5)

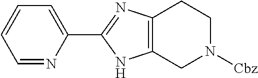

A solution of oxalyl chloride (2.53 g, 19.9 mmol) in DCM (9 mL) was cooled to −78° C. A solution of DMSO (2.90 g, 37.2 mmol) in DCM (1.6 mL) was added dropwise with stirring continued for 5 minutes. A solution of benzyl-2-(pyridine-2-yl)-3a,4,7,7a-tetrahydro-3H-imidazo[4,5-c]pyridine-5(6H)-carboxylate (2.98 g, 8.85 mmol) in DCM (6 mL) was added dropwise and the reaction stirred for 1 hour. TEA (9.67 g, 95.6 mmol) was added dropwise, and the reaction was allowed to warm to room temperature, and stirred for 2 hours. Water (60 mL) was added and the mixture was extracted with DCM (3×75 mL). The organic layers were combined, washed with brine and dried over Na₂SO₄. Removal of the solvent under vacuum afforded a crude residue that was purified by silica gel chromatography (DCM/MeOH) to give a red solid (2.49 g, 84%). ¹H NMR (400 MHz, CDCl₃): δ 8.48 (d, 1H), 8.06 (m, 1H), 7.74 (m, 1H), 7.32-7.40 (m, 5H), 7.21 (m, 1H), 5.18 (s, 2H), 4.65 (s, 2H), 3.86 (m, 2H), 2.78 (m, 2H); LC/MS: m/e=335 (M+H)⁺.

### 111.6 Benzyl-3-(4-methoxybenzyl)-2-(pyridine-2-yl)-6,7-dihydro-3H-imidazo[4,5-c]pyridine-5(4H)-carboxylate (I-111.6)

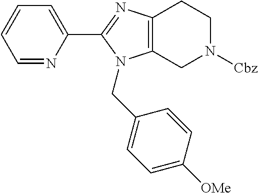

Benzyl-2-(pyridine-2-yl)-6,7-dihydro-3H-imidazo[4,5-c]pyridine-5(4H)-carboxylate (1.5 g, 4.48 mmol) was dissolved in DMF (15 mL) and cooled to 0° C. Potassium t-butoxide (500 mg, 3.2 mmol) was added. 4-Methoxybenzyl chloride (0.84 g, 5.38 mmol) was dissolved in DMF (4 mL) and added dropwise to the mixture at 0° C. After stirring for 3 hours, the mixture was partitioned between H₂O (15 mL) and EtOAc (30 mL). The organic layer was washed with brine and dried over Na₂SO₄Solvent removal afforded crude material that was purified by silica gel chromatography (EtOAc/Hexanes) to give a white powder (1.31 g, 64%). ¹H NMR (400 MHz, CDCl₃): δ 8.50 (s, 1H), 8.14 (m, 1H), 7.72 (m, 1H), 7.26-7.40 (m, 5H), 7.16 (m, 1H), 7.02 (d, 2H), 6.78 (d, 2H), 5.18 (s, 2H), 5.16 (s, 2H), 4.62 (m, 1H), 4.4 (s, 1H), 3.76 (s, 2H), 2.78 (s, 1H), 2.58 (s, 1H); LC/MS: m/e=455 (M+H)⁺.

### 111.7 3-(4-Methoxybenzyl)-2-(pyridine-2-yl)-4,5,6,7-tetrahydro-3H-imidazo[4,5-c]pyridine (I-111.7)

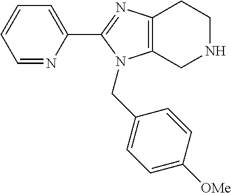

Benzyl-3-(4-methoxybenzyl)-2-(pyridine-2-yl)-6,7-dihydro-3H-imidazo[4,5-c]pyridine-5(4H)-carboxylate (1.31 g, 2.88 mmol) was dissolved in ethanol (60 mL). Palladium hydroxide on carbon catalyst (130 mg, 10% w/w) was added and the reaction was stirred under atmospheric pressure of H₂ at room temperature for 1 day. An additional 300 mg of palladium hydroxide on carbon catalyst were added, and the mixture was stirred for 1 day. The catalyst was removed by filtration through celite, washed with ethanol and the filtrate was concentrated to dryness. The residue was purified by reverse phase purification (Gilson GX-281 (NH₄HCO₃/Acetonitrile)) to give a yellow/brown oil (550 mg, 59%). ¹H NMR (400 MHz, CDCl₃): δ 8.50 (d, 1H), 8.14 (m, 1H), 7.72 (m, 1H), 7.16 (m, 1H), 7.05 (m, 2H), 6.78 (d, 2H), 5.85 (s, 1H), 5.8 (s, 1H), 5.72 (s, 1H), 3.95 (s, 1H), 3.1 (q, 2H), 2.75 (t, 1H), 2.50 (t, 1H); LC/MS: m/e=321 (M+H)⁺.

### 111.8 3-Fluoro-5-(3-(4-methoxybenzyl)-2-(pyridine-2-yl)-6,7-dihydro-3H-imidazo[4,5-c]pyridine-5(4H)-yl)benzonitrile (I-111.8)

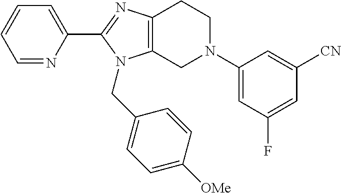

3-(4-Methoxybenzyl)-2-(pyridine-2-yl)-4,5,6,7-tetrahydro-3H-imidazo[4,5-c]pyridine (108 mg, 0.34 mmol), tris(dibenzylideneacetone)dipalladium(0) (6.2 mg, 0.007 mmol), 4,5-bis(diphenyl-phosphino)-9,9-dimethylxanthene (12 mg, 0.02 mmol), Cs₂CO₃ (220 mg, 0.67 mmol), and 3-bromo-5-fluorobenzonitrile were combined in xylene (4 mL). The mixture was heated under microwave at 150° C. for 30 minutes. The mixture was filtered and the filtrate was concentrated to dryness. The residue was purified by reverse phase purification (Gilson GX-281 (NH₄HCO₃/Acetonitrile)) to give a yellow/brown (20 mg, 13%). ¹H NMR (400 MHz, CDCl₃): δ 8.50 (d, 1H), 8.15 (m, 1H), 7.75 (t, 1H), 7.19 (m, 1H), 7.1 (m, 1H), 6.87 (s, 1H), 6.85 (s, 1H), 6.75-6.8 (d, 2H), 6.7 (d, 1H), 6.58 (m, 1H), 5.9 (s, 1H), 4.12 (s, 1H), 3.78 (s, 1H), 3.74 (s, 1H), 3.65 (t, 2H), 2.83 (t, 2H); LC/MS: m/e=440 (M+H)⁺.

### 112. Compound 112: 3-Fluoro-5-(2-(pyridin-2-yl)-5,6-dihydro-[1,2,4]triazolo[1,5-a]pyrazin-7(8H)-yl)benzonitrile

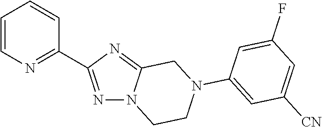

2-(Pyridin-2-yl)-5,6,7,8-tetrahydro-[1,2,4]-triazolo[1,5-a]pyrazine (I-112.5) (112 mg, 0.56 mmol), tris(dibenzylideneacetone)dipalladium(0) (10 mg, 0.011 mmol), Xantphos (19 mg, 0.033 mmol), Cs₂CO₃ (362 mg, 1.11 mmol), and 3-bromo-5-fluorobenzonitrile (245 mg, 1.22 mmol) were combined in xylene (4 mL). The mixture was stirred under microwaves at 150° C. for 30 minutes. The solvent was evaporated and the crude material was purified by silica gel chromatography (9/1 DCM/MeOH). The fractions containing the desired product were collected and combined and then purified by reverse phase purification (Gilson GX-281 (NH₄HCO₃/Acetonitrile)) to give the desired product as a colorless oil (1.1 mg, 0.6%). ¹H NMR (400 MHz, CDCl₃): δ 8.72 (s, 1H), 8.09 (d, 1H), 7.8 (t, 1H), 7.33 (t, 1H), 7.1 (s, 1H), 6.9 (t, 2H), 4.65 (s, 2H), 4.45 (s, 2H), 3.9 (s, 2H); LC/MS: m/e=321 (M+H)⁺.

### 112.1 Benzyl 2-hydrazinyl-2-oxoethylcarbamate (I-112.1)

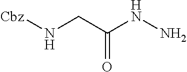

Cbz-glycine (30 g, 143 mmol), 4-methylmorpholine (18.8 g, 185 mmol) and ethyl chloroformate (17 g, 157 mmol) were dissolved in THF (500 mL) and stirred at RT for 2 hours. The mixture was filtered in order to remove the white precipitate. The filtrate was cooled at 0° C. and hydrazine (35.8 g, 715 mmol) in THF (50 mL) was added. The mixture was stirred at RT overnight. Saturated NaHCO₃ was added and the mixture was extracted with EtOAc. The organic layer was washed with brine, dried over MgSO₄, filtered, concentrated and dried under vacuum to give a white solid (18.3 g, 57%). ¹H NMR (400 MHz, CDCl₃): δ 7.28-7.38 (m, 5H), 5.10 (s, 2H), 3.82 (s, 2H); LC/MS: m/e=224 (M+H)⁺.

### 112.2 Benzyl (3-(pyridin-2-yl)-1H-1,2,4-triazol-5-yl)methylcarbamate (I-112.2)

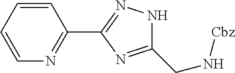

Benzyl 2-hydrazinyl-2-oxoethylcarbamate (16.4 g, 73.6 mmol) and ethyl picolinimidate (prepared according to the procedure in Watanabe, H., et al., Chem. Pharm. Bull. 1973, 21, 465) (13.3 g, 88.3 mmol) were combined in EtOH (150 mL). The mixture was heated at reflux overnight. Acetic acid (60 mL) was added and the mixture was stirred at reflux for 2 hours. The mixture was concentrated to dryness. Trituration using ether afforded the product as a brown solid (21 g, 92%). ¹H NMR (400 MHz, MeOH-d₄): δ 8.64 (s, 1H), 8.08 (d, 1H), 7.92 (t, 1H), 7.45 (s, 1H), 7.16-7.42 (m, 5H), 5.12 (s, 2H), 4.48 (s, 2H); LC/MS: m/e=310 (M+H)⁺.

### 112.3 Ethyl 2-(5-((benzyloxycarbonylamino)methyl)-3-(pyridin-2-yl)-1H-1,2,4-triazol-1-yl)acetate (I-112.3)

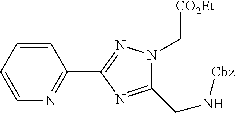

Benzyl (3-(pyridin-2-yl)-1H-1,2,4-triazol-5-yl)methylcarbamate (10.0 g, 32.3 mmol) was dissolved in DMF (100 mL) and cooled to 0° C. Potassium t-butoxide (3.63 g, 32.3 mmol) was added. Ethyl bromoacetate (5.4 g, 32.3 mmol) was dissolved in DMF and added dropwise, and the reaction was stirred at room temperature for 4 hours. DMF was removed in vacuo and the residue dissolved in EtOAc. The organic layer was washed with saturated NaHCO₃ solution and brine, then dried over Na₂SO₄. Purification by reverse phase chromatography (Gilson GX-281 (NH₄HCO₃/Acetonitrile)) gave a yellow oil (5.21 g, 41%). ¹H NMR (400 MHz, CDCl₃): δ 8.7 (d, 1H), 8.08 (d, 1H), 7.77 (t, 1H), 7.28-7.38 (m, 5H), 5.59 (s, 1H), 5.2 (s, 2H), 5.1 (s, 2H), 4.55 (d, 2H), 4.21 (q, 2H), 1.22-1.32 (t, 3H); LC/MS: m/e=396 (M+H)⁺.

### 112.4 2-(Pyridin-2-yl)-7,8-dihydro-[1,2,4]-triazolo[1,5-a]pyrazin-6(5H)-one (I-112.4)

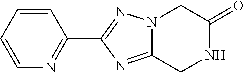

Ethyl 2-(5-((benzyloxycarbonylamino)methyl)-3-(pyridin-2-yl)-1H-1,2,4-triazol-1-yl)acetate (4.94 g, 12.4 mmol) was dissolved in MeOH (50 mL). Palladium on carbon catalyst (10%, 300 mg, 6% w/w) was added, and the reaction was stirred under hydrogen atmosphere (1 atm) for 3 hours. The catalyst was removed by filtration through Celite, washed several times with hot methanol and the combined filtrates concentrated to give a white solid (2.03 g, 76%). ¹H NMR (400 MHz, MeOH-d₄): δ 8.63 (d, 1H), 8.12 (d, 1H), 7.93 (m, 1H), 7.46 (m, 1H), 4.93 (s, 2H), 4.7 (s, 2H); LC/MS: m/e=216 (M+H)⁺.

### 112.5 2-(Pyridin-2-yl)-5,6,7,8-tetrahydro-[1,2,4]-triazolo[1,5-a]pyrazine (I-112.5)

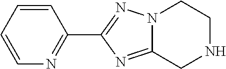

To a solution of 2-(pyridin-2-yl)-7,8-dihydro-[1,2,4]triazolo[1,5-a]pyrazin-6(5H)-one (1.63 g, 7.57 mmol) in THF (40 mL) was added dropwise a solution of BH₃/THF (30 mL, 1M, 30.0 mmol). The reaction was stirred at room temperature under N₂ for 15 minutes, then heated at reflux under N₂ for 4 hours. The reaction was cooled in an ice bath. 4N HCl in dioxane (8 mL) and MeOH (8 mL) were added dropwise, and the reaction was heated to reflux for 2 hours. The mixture was concentrated to dryness on the rotavap. MeOH was added to the crude and evaporated. This process of dilution and evaporation was repeated a total of 5 times. Brine (1 mL) was added and the solution was extracted with DCM (2×5 mL). The pH of the aqueous layer was adjusted to 14 using 6N NaOH and extracted with DCM (4×5 mL). The combined organic layers were dried over MgSO₄, filtered and concentrated to give a yellow solid (400 mg, 26%). ¹H NMR (400 MHz, CDCl₃): δ 8.7 (d, 1H), 8.08 (d, 1H), 7.77 (t, 1H), 7.30 (t, 1H), 4.25 (m, 4H), 3.38 (t, 2H); LC/MS: m/e=202 (M+H)⁺.

### 113. Compound 113: 7-(3-Chlorophenyl)-2-(pyridin-2-yl)-5,6,7,8-tetrahydro-[1,2,4]triazolo[1,5-a]pyrazine

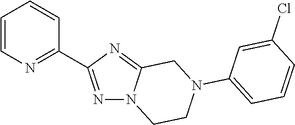

The title compound was prepared via the procedure used for Compound 112, using 1-bromo-3-chlorobenzene instead of 3-bromo-5-fluorobenzonitrile. Reverse phase purification (Gilson GX-281 (NH₄HCO₃/Acetonitrile)) afforded a white solid (4.8 mg, 3%). ¹H NMR (400 MHz, CDCl₃): δ 8.72 (d, 1H), 8.09 (d, 1H), 7.8 (t, 1H), 7.33 (t, 1H), 7.22 (s, 1H), 6.98 (s, 1H), 6.92 (d, 1H), 6.86 (d, 1H), 4.58 (s, 2H), 4.40 (m, 2H), 3.70 (m, 2H); LC/MS: m/e=312 (M+H)⁺.

### 114. Compound 114: 7-(3-Fluorophenyl)-2-(pyridin-2-yl)-5,6,7,8-tetrahydro-[1,2,4]triazolo[1,5-a]pyrazine

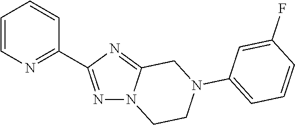

The title compound was prepared via the procedure used for Compound 112, using 1-bromo-3-fluorobenzene instead of 3-bromo-5-fluorobenzonitrile. Reverse phase purification (Gilson GX-281 (NH₄HCO₃/Acetonitrile)) gave a white solid (20 mg, 13%). ¹H NMR (400 MHz, CDCl₃): δ 8.72 (d, 1H), 8.09 (d, 1H), 7.78 (t, 1H), 7.32 (m, 1H), 6.76 (m, 1H), 6.62-6.72 (m, 2H), 4.58 (s, 2H), 4.40 (m, 2H), 3.70 (m, 2H); LC/MS: m/e=296 (M+H)⁺.

### 115. Compound 115: 5-(3-chlorophenyl)-2-(pyridin-2-yl)-4,5,6,7-tetrahydrooxazolo[4,5-c]pyridine

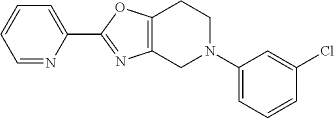

The title compound was prepared via the procedure used for the preparation of Compound 23, substituting 1-bromo-3-chlorobenzene for 3-bromobenzonitrile. Preparative HPLC afforded a brown solid (6 mg, 7%). ¹H NMR (400 MHz, CDCl₃): δ 8.73 (d, 1H), 8.10 (d, 1H), 7.83 (t, 1H), 7.37 (t, 1H), 7.21 (t, 1H), 6.96 (s, 1H), 6.84 (m, 2H), 4.31 (s, 2H), 3.72 (t, 2H), 3.00 (t, 2H); LC/MS: m/e=312 (M+H)⁺.

### 116. Compound 116: 5-(3-fluorophenyl)-2-(pyridin-2-yl)-4,5,6,7-tetrahydrooxazolo[4,5-c]pyridine

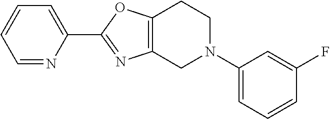

The title compound was prepared via the procedure used for the preparation of Compound 23, substituting 1-bromo-3-fluorobenzene for 3-bromobenzonitrile. Preparative HPLC afforded a brown solid (15 mg, 34%). ¹H NMR (400 MHz, CDCl₃): δ 8.71 (d, 1H), 8.10 (d, 1H), 7.83 (t, 1H), 7.37 (t, 1H), 7.28 (m, 1H), 6.76 (d, 1H), 6.68 (d, 1H), 6.56 (t, 1H), 4.31 (s, 2H), 3.73 (t, 2H), 3.00 (t, 2H); LC/MS: m/e=296 (M+H)⁺.

### 117. Compound 117: 3-fluoro-5-(2-(pyridin-2-ylmethyl)-6,7-dihydrooxazolo[4,5-c]pyridin-5(4H)-yl)benzonitrile

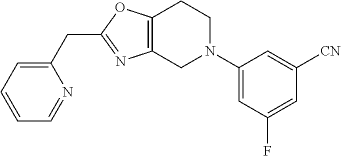

This compound is prepared using the procedures described herein elsewhere, such as example 23 and example 98, from suitable starting materials.

### 118. Compound 118: 3-fluoro-5-(2-(pyrimidin-4-yl)-6,7-dihydrooxazolo[4,5-c]pyridin-5(4H)-yl)benzonitrile

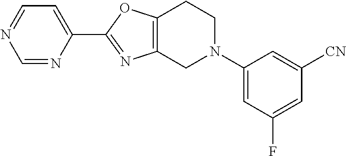

The title compound was prepared via the procedure used for the preparation of Compound 23, substituting pyrimidine-4-carboxylic acid for picolinic acid, and substituting 3-bromo-5-fluorobenzonitrile for 3-bromobenzonitrile. Preparative HPLC afforded a brown solid (15 mg, 44%). ¹H NMR (400 MHz, CDCl₃): δ 9.35 (s, 1H), 8.92 (s, 1H), 8.02 (d, 1H), 6.98 (s, 1H), 6.83 (m, 2H), 4.36 (s, 2H), 3.79 (t, 2H), 3.05 (t, 2H); LC/MS: m/e=322 (M+H)⁺.

### 119. Compound 119: 5-(3-fluorophenyl)-2-(pyrimidin-4-yl)-4,5,6,7-tetrahydrooxazolo[4,5-c]pyridine

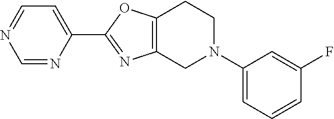

The title compound was prepared via the procedure used for the preparation of Compound 23, substituting pyrimidine-4-carboxylic acid for picolinic acid, and substituting 1-bromo-3-fluorobenzene for 3-bromobenzonitrile. Preparative HPLC afforded a brown solid (9 mg, 29%). ¹H NMR (400 MHz, CDCl₃): δ 9.33 (s, 1H), 8.90 (s, 1H), 8.01 (d, 1H), 7.23 (t, 1H), 6.75 (d, 1H), 6.68 (d, 1H), 6.57 (t, 1H), 4.33 (s, 2H), 3.75 (t, 2H), 3.02 (t, 2H); LC/MS: m/e=297 (M+H)⁺.

### 120. Compound 120: 5-(3-chlorophenyl)-2-(pyrimidin-4-yl)-4,5,6,7-tetrahydrooxazolo[4,5-c]pyridine

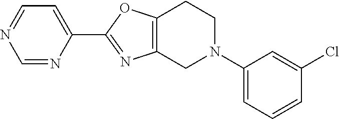

The title compound was prepared via the procedure used for the preparation of Compound 23, substituting pyrimidine-4-carboxylic acid for picolinic acid, and substituting 1-bromo-3-chlorobenzene for 3-bromobenzonitrile. Preparative HPLC afforded a brown solid (15 mg, 45%). ¹H NMR (400 MHz, CDCl₃): δ 9.33 (s, 1H), 8.90 (s, 1H), 8.02 (d, 1H), 7.20 (t, 1H), 6.96 (d, 1H), 6.86 (m, 2H), 4.32 (s, 2H), 3.74 (t, 2H), 3.00 (t, 2H); LC/MS: m/e=313 (M+H)⁺.

### 121. Compound 121: 3-fluoro-5-(2-(3-methylpyridin-2-yl)-6,7-dihydrooxazolo[4,5-c]pyridin-5(4H)-yl)benzonitrile

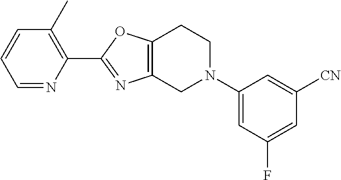

The title compound was prepared via the procedure used for the preparation of Compound 23, substituting 3-methylpicolinic acid for picolinic acid, and substituting 3-bromo-5-fluorobenzonitrile for 3-bromobenzonitrile. Preparative HPLC afforded a brown solid (4 mg, 5%). ¹H NMR (400 MHz, CDCl₃): δ 8.60 (s, 1H), 7.66 (d, 1H), 7.29 (m, 1H), 6.99 (s, 1H), 6.84 (dd, 2H), 4.36 (s, 2H), 3.78 (t, 2H), 3.03 (m, 2H), 2.75 (s, 3H); LC/MS: m/e=335 (M+H)⁺.

### 122. Compound 122: 5-(3-fluorophenyl)-2-(3-methylpyridin-2-yl)-4,5,6,7-tetrahydrooxazolo[4,5-c]pyridine

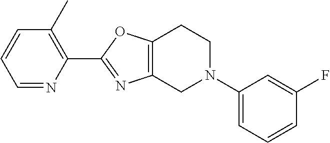

The title compound was prepared via the procedure used for the preparation of Compound 23, substituting 3-methylpicolinic acid for picolinic acid, and substituting 1-bromo-3-fluorobenzene for 3-bromobenzonitrile. Preparative HPLC afforded a brown solid (7 mg, 25%). ¹H NMR (400 MHz, CDCl₃): δ 8.59 (s, 1H), 7.66 (d, 1H), 7.28 (m, 1H), 7.20 (t, 1H), 6.76 (d, 1H), 6.69 (d, 1H), 6.54 (t, 1H), 4.34 (s, 2H), 3.73 (t, 2H), 3.02 (t, 2H), 2.75 (s, 3H); LC/MS: m/e=310 (M+H)⁺.

### 123. Compound 123: 5-(3-chlorophenyl)-2-(3-methylpyridin-2-yl)-4,5,6,7-tetrahydrooxazolo[4,5-c]pyridine

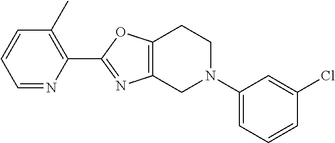

The title compound was prepared via the procedure used for the preparation of Compound 23, substituting 3-methylpicolinic acid for picolinic acid, and substituting 1-bromo-3-chlorobenzene for 3-bromobenzonitrile. Preparative HPLC afforded a brown solid (15 mg, 45%). ¹H NMR (400 MHz, CDCl₃): δ 8.59 (m, 1H), 7.63 (m, 1H), 7.21 (m, 1H), 7.19 (t, 1H), 6.97 (t, 1H), 6.87 (br, 2H), 4.33 (s, 2H), 3.72 (t, 2H), 3.01 (t, 2H), 2.75 (s, 3H); LC/MS: m/e=326 (M+H)⁺.

### 124. Compound 124: 5-(3-fluorophenyl)-2-(5-fluoropyridin-2-yl)-4,5,6,7-tetrahydrooxazolo[4,5-c]pyridine

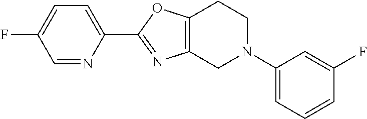

The title compound was prepared via the procedure used for the preparation of Compound 23, substituting 5-fluoropicolinic acid for picolinic acid, and substituting 1-bromo-3-fluorobenzene for 3-bromobenzonitrile. Preparative HPLC afforded a brown solid (9 mg, 32%). ¹H NMR (400 MHz, CDCl₃): δ 8.57 (s, 1H), 8.12 (m, 1H), 7.55 (m, 1H), 7.21 (m, 1H), 6.75 (d, 1H), 6.68 (d, 1H), 6.56 (t, 1H), 4.30 (s, 2H), 3.74 (t, 2H), 2.99 (t, 2H); LC/MS: m/e=314 (M+H)⁺.

### 125. Compound 125: 5-(3-chlorophenyl)-2-(5-fluoropyridin-2-yl)-4,5,6,7-tetrahydrooxazolo[4,5-c]pyridine

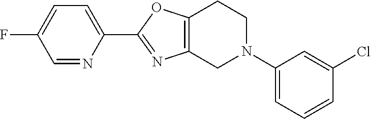

The title compound was prepared via the procedure used for the preparation of Compound 23, substituting 5-fluoropicolinic acid for picolinic acid, and substituting 1-bromo-3-chlorobenzene for 3-bromobenzonitrile. Preparative HPLC afforded a brown solid (13 mg, 43%). ¹H NMR (400 MHz, CDCl₃): δ 8.56 (s, 1H), 8.12 (m, 1H), 7.54 (m, 1H), 7.19 (t, 1H), 6.95 (s, 1H), 6.86 (m, 1H), 4.30 (s, 2H), 3.73 (t, 2H), 2.98 (t, 2H); LC/MS: m/e=330 (M+H)⁺.

### 126. Compound 126: 5-phenyl-2-(pyridin-2-yl)-4,5,6,7-tetrahydrooxazolo[4,5-c]pyridine

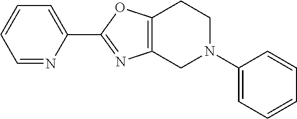

The title compound was prepared via the procedure used for the preparation of Compound 23, substituting bromobenzene for 3-bromobenzonitrile. Preparative HPLC afforded a brown solid (70 mg, 35%). ¹H NMR (400 MHz, CDCl₃): δ 8.71 (s, 1H), 7.98 (d, 1H), 7.81 (t, 1H), 7.40 (t, 1H), 7.26-7.32 (m, 2H), 7.00 (d, 2H), 6.88 (t, 1H), 4.30 (s, 2H), 3.70 (s, 2H), 2.97 (s, 2H); LC/MS: m/e=278 (M+H)⁺.

### 127. Compound 127: 5-(3-methoxyphenyl)-2-(pyridin-2-yl)-4,5,6,7-tetrahydrooxazolo[4,5-c]pyridine

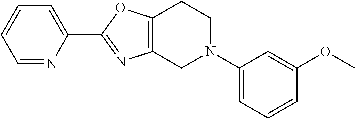

The title compound was prepared via the procedure used for the preparation of Compound 23, substituting 1-bromo-3-methoxybenzene for 3-bromobenzonitrile. Preparative HPLC afforded a red solid (83 mg, 37%). ¹H NMR (400 MHz, CDCl₃): δ 8.72 (s, 1H), 8.095 (d, 1H), 7.82 (t, 1H), 7.36 (t, 1H), 7.18 (t, 1H), 6.62 (d, 1H), 6.55 (s, 1H), 6.43 (d, 1H), 4.30 (s, 2H), 3.71 (t, 2H), 2.96 (s, 2H); LC/MS: m/e=308 (M+H)⁺.

### 128. Compound 128: 5-(3-fluorophenyl)-2-(pyrazin-2-yl)-4,5,6,7-tetrahydrooxazolo[4,5-c]pyridine

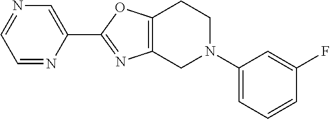

This compound is prepared using the procedures described herein elsewhere, such as example 23 and example 98, from suitable starting materials.

### 129. Compound 129: 5-(3-chlorophenyl)-2-(pyrazin-2-yl)-4,5,6,7-tetrahydrooxazolo[4,5-c]pyridine

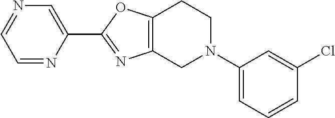

This compound is prepared using the procedures described herein elsewhere, such as example 23 and example 98, from suitable starting materials.

### 130. Compound 130: 3-fluoro-5-(2-(pyrazin-2-yl)-6,7-dihydrooxazolo[4,5-c]pyridin-5(4H)-yl)benzonitrile

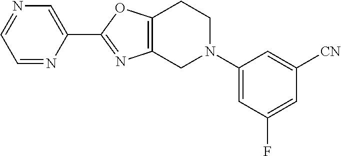

This compound is prepared using the procedures described herein elsewhere, such as example 23 and example 98, from suitable starting materials.

### 131. Compound 131: 2-(pyridin-2-yl)-5-(pyrimidin-5-yl)-4,5,6,7-tetrahydrooxazolo[4,5-c]pyridine

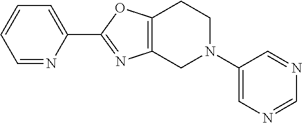

The title compound was prepared via the procedure used for the preparation of Compound 23, substituting 5-bromopyrimidine for 3-bromobenzonitrile. Preparative HPLC afforded a white/yellow solid (22 mg, 11%). ¹H NMR (400 MHz, CDCl₃): δ 8.72 (s, 2H), 8.46 (s, 2H), 8.095 (d, 1H), 7.82 (t, 1H), 7.365 (t, 1H), 6.54 (s, 1H), 4.28 (s, 2H), 3.78 (t, 2H), 3.78 (t, 2H), 3.05 (s, 2H); LC/MS: m/e=280 (M+H)⁺.

### 132. Compound 132: 2-(pyridin-2-yl)-5-(pyrimidin-2-yl)-4,5,6,7-tetrahydrooxazolo[4,5-c]pyridine

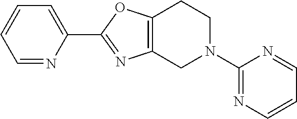

The title compound was prepared via the procedure used for the preparation of Compound 23, substituting 2-bromopyrimidine for 3-bromobenzonitrile. Preparative HPLC afforded a white solid (2 mg, 1%). ¹H NMR (400 MHz, CDCl₃): δ 8.70 (d, 1H), 8.5 (d, 2H), 8.1 (d, 1H), 7.80 (t, 1H), 7.35 (t, 1H), 6.54 (s, 1H), 4.88 (s, 2H), 4.30 (t, 2H), 3.78 (t, 2H), 2.95 (s, 2H); LC/MS: m/e=280 (M+H)⁺.

### 133. Compound 133: 5-(3-fluorophenyl)-2-(5-fluoropyridin-2-yl)-5,6,7,8-tetrahydro-4H-oxazolo[4,5-c]azepine

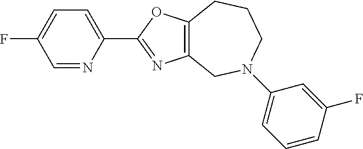

The title compound was prepared via the procedure used for the preparation of Compound 98, substituting 5-fluoropicolinic acid for picolinic acid, and substituting 1-bromo-3-fluorobenzene for 3-bromo-5-fluorobenzonitrile. Preparative HPLC afforded a solid (7 mg, 11%). ¹H NMR (400 MHz, CDCl₃): δ 8.54 (d, 1H), 8.07 (dd, 1H), 7.52 (td, 1H), 7.15-7.09 (m, 1H), 6.59 (dd, 1H), 6.52 (dt, 1H), 6.39 (td, 1H) 4.52 (s, 2H), 3.82-3.79 (m, 2H), 2.98 (t, 2H), 2.02-1.96 (m, 2H); LC/MS: m/e=328 (M+H)⁺.

### 134. Compound 134: 5-(3-chlorophenyl)-2-(5-fluoropyridin-2-yl)-5,6,7,8-tetrahydro-4H-oxazolo[4,5-c]azepine

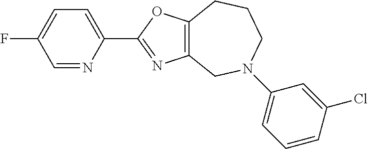

The title compound was prepared via the procedure used for the preparation of Compound 98, substituting 5-fluoropicolinic acid for picolinic acid, and substituting 1-bromo-3-chlorobenzene for 3-bromo-5-fluorobenzonitrile. Preparative HPLC afforded a solid (8 mg, 11%). ¹H NMR (400 MHz, CDCl₃): δ 8.55 (d, 1H), 8.07 (dd, 1H), 7.51 (td, 1H), 7.10 (t, 1H), 6.81 (t, 1H), 6.72˜6.66 (m, 2H), 4.52 (s, 2H), 3.81˜3.78 (m, 2H), 2.98 (t, 2H), 2.02˜1.96 (m, 2H); LC/MS: m/e=344 (M+H)⁺.

### 135. Compound 135: 3-fluoro-5-(2-(5-fluoropyridin-2-yl)-7,8-dihydro-4H-oxazolo[4,5-c]azepin-5(6H)-yl)benzonitrile

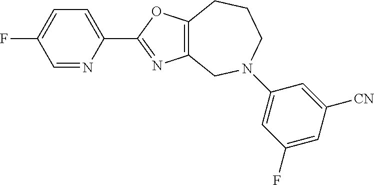

The title compound was prepared via the procedure used for the preparation of Compound 98, substituting 5-fluoropicolinic acid for picolinic acid. Preparative HPLC afforded a solid (10 mg, 9%). ¹H NMR (400 MHz, CDCl₃): δ 8.55 (d, 1H), 8.09 (dd, 1H), 7.53 (td, 1H), 6.83˜6.82 (m, 1H), 6.71 (dt, 1H), 6.67˜6.64 (m, 1H), 4.52 (s, 2H), 3.83˜3.80 (m, 2H), 3.01 (t, 2H), 2.03˜1.97 (m, 2H); LC/MS: m/e=353 (M+H)⁺.

### 136. Compound 136: 5-(3-fluorophenyl)-2-(pyrimidin-4-yl)-5,6,7,8-tetrahydro-4H-oxazolo[4,5-c]azepine

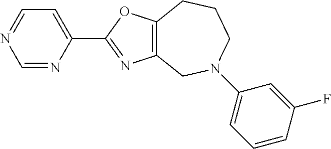

The title compound was prepared via the procedure used for the preparation of Compound 98, substituting pyrimidine-4-carboxylic acid for picolinic acid, and substituting 1-bromo-3-fluorobenzene for 3-bromo-5-fluorobenzonitrile. Preparative HPLC afforded a solid (6 mg, 10%). ¹H NMR (400 MHz, CDCl₃): δ 9.32 (s, 1H), 8.88 (s, 1H), 7.97 (d, 1H), 7.16˜7.10 (m, 1H), 6.59 (dd, 1H), 6.52 (dt, 1H), 6.41 (td, 1H), 4.55 (s, 2H), 3.84˜3.81 (m, 2H), 3.02 (t, 2H), 2.04˜1.98 (m, 2H); LC/MS: m/e=311 (M+H)⁺.

### 137. Compound 137: 5-(3-chlorophenyl)-2-(pyrimidin-4-yl)-5,6,7,8-tetrahydro-4H-oxazolo[4,5-c]azepine

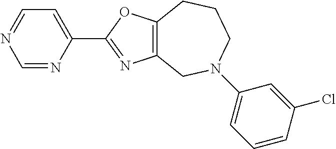

The title compound was prepared via the procedure used for the preparation of Compound 98, substituting pyrimidine-4-carboxylic acid for picolinic acid, and substituting 1-bromo-3-chlorobenzene for 3-bromo-5-fluorobenzonitrile. Preparative HPLC afforded a solid (7 mg, 15%). ¹H NMR (400 MHz, CDCl₃): δ 9.31 (d, 1H), 8.87 (d, 1H), 7.98 (dd, 1H), 7.10 (t, 1H), 6.80 (t, 1H), 6.71˜6.67 (m, 2H), 4.54 (s, 2H), 3.83˜3.81 (m, 2H), 3.01 (t, 2H), 2.04˜1.98 (m, 2H); LC/MS: m/e=327 (M+H)⁺.

### 138. Compound 138: 3-fluoro-5-(2-(pyrimidin-4-yl)-7,8-dihydro-4H-oxazolo[4,5-c]azepin-5(6H)-yl)benzonitrile

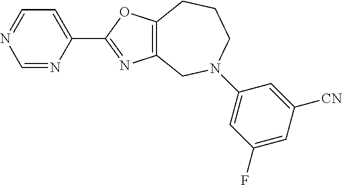

The title compound was prepared via the procedure used for the preparation of Compound 98, substituting pyrimidine-4-carboxylic acid for picolinic acid. Preparative HPLC afforded a solid (21 mg, 33%). ¹H NMR (400 MHz, CDCl₃): δ 9.31 (d, 1H), 8.89 (d, 1H), 7.98 (dd, 1H), 6.83 (d, 1H), 6.73˜6.65 (m, 2H), 4.55 (s, 2H), 3.85˜3.82 (m, 2H), 3.05 (t, 2H), 2.05˜1.99 (m, 2H); LC/MS: m/e=336 (M+H)⁺.

### 139. Compound 139: 5-(3-fluorophenyl)-2-(pyrazin-2-yl)-5,6,7,8-tetrahydro-4H-oxazolo[4,5-c]azepine

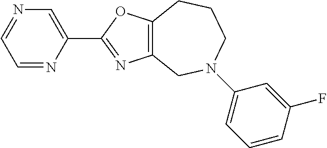

The title compound was prepared via the procedure used for the preparation of Compound 98, substituting pyrazine-2-carboxylic acid for picolinic acid, and substituting 1-bromo-3-fluorobenzene for 3-bromo-5-fluorobenzonitrile. Preparative HPLC afforded a solid (53 mg, 51%). ¹H NMR (400 MHz, CDCl₃): δ 9.29 (d, 1H), 8.6˜8.65 (m, 2H), 7.12 (q, 1H), 6.59 (dd, 1H), 6.53 (td, 1H), 6.39 (dt, 1H), 4.54 (s, 2H), 3.79˜3.82 (m, 2H), 3.00 (t, 2H), 1.98˜2.03 (m, 2H); LC/MS: m/e=311 (M+H)⁺.

### 140. Compound 140: 5-(3-chlorophenyl)-2-(pyrazin-2-yl)-5,6,7,8-tetrahydro-4H-oxazolo[4,5-c]azepine

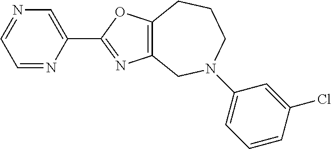

The title compound was prepared via the procedure used for the preparation of Compound 98, substituting pyrazine-2-carboxylic acid for picolinic acid, and substituting 1-bromo-3-chlorobenzene for 3-bromo-5-fluorobenzonitrile. Preparative HPLC afforded a solid (33 mg, 41%). ¹H NMR (400 MHz, CDCl₃): δ 9.29 (d, 1H), 8.60˜8.65 (m, 2H), 7.10 (t, 1H), 6.81 (t, 1H), 6.67˜6.72 (m, 2H), 4.54 (s, 2H), 3.80˜3.82 (m, 2H), 3.00 (t, 2H), 1.99˜2.02 (m, 2H); LC/MS: m/e=327 (M+H)⁺.

### 141. Compound 141: 3-fluoro-5-(2-(pyrazin-2-yl)-7,8-dihydro-4H-oxazolo[4,5-c]azepin-5(6H)-yl)benzonitrile

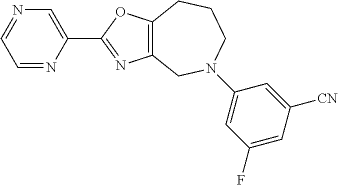

The title compound was prepared via the procedure used for the preparation of Compound 98, substituting pyrazine-2-carboxylic acid for picolinic acid. Preparative HPLC afforded a solid (11 mg, 14%). ¹H NMR (400 MHz, CDCl₃): δ 9.31 (d, 1H), 8.63˜8.66 (m, 2H), 6.84 (s, 1H), 6.72 (td, 1H), 6.67 (dd, 1H), 4.55 (s, 2H), 3.82˜3.85 (m, 2H), 3.04 (t, 2H), 2.01˜2.03 (m, 2H); LC/MS: m/e=336 (M+H)⁺.

### 142. Compound 142: 2-(pyridin-2-yl)-5-(pyrimidin-5-yl)-5,6,7,8-tetrahydro-4H-oxazolo[4,5-c]azepine

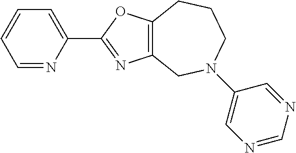

This compound is prepared using the procedures described herein elsewhere, such as example 23 and example 98, from suitable starting materials.

### 143. Compound 143: 2-(pyridin-2-yl)-5-(pyrimidin-2-yl)-5,6,7,8-tetrahydro-4H-oxazolo[4,5-c]azepine

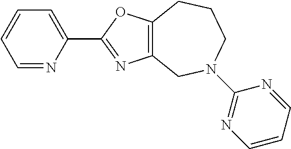

This compound is prepared using the procedures described herein elsewhere, such as example 23 and example 98, from suitable starting materials.

### 144. Compound 144: 5-(3-chloro-4-fluorophenyl)-2-(pyridin-2-yl)-5,6,7,8-tetrahydro-4H-oxazolo[4,5-c]azepine

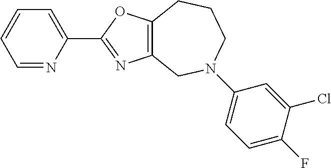

The title compound was prepared via the procedure used for the preparation of Compound 98, substituting 4-bromo-2-chloro-1-fluorobenzene for 3-bromo-5-fluorobenzonitrile. Preparative HPLC afforded a solid (53 mg, 51%). ¹H NMR (400 MHz, CDCl₃): δ 8.71 (d, 1H), 8.05 (d, 1H), 7.80 (dt, 1H), 7.32-7.35 (m, 1H), 6.95 (t, 1H), 6.82 (q, 1H), 6.65 (td, 1H), 4.48 (s, 2H), 3.76-3.79 (m, 2H), 2.99 (t, 2H), 1.93-1.99 (m, 2H); LC/MS: m/e=344 (M+H)⁺.

### 145. Compound 145: 3-fluoro-5-(2-(2-methylthiazol-4-yl)-7,8-dihydro-4H-oxazolo[4,5-c]azepin-5(6H)-yl)benzonitrile

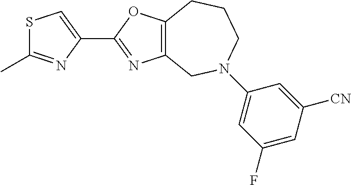

The title compound was prepared via the procedure used for the preparation of Compound 98, substituting 2-methylthiazole-4-carboxylic acid for picolinic acid. Preparative HPLC afforded a solid (80 mg, 60%). ¹H NMR (400 MHz, CDCl₃): δ 7.72 (s, 1H), 6.83 (s, 1H), 6.70 (td, 1H), 6.65 (td, 1H), 4.50 (s, 2H), 3.78˜3.81 (m, 2H), 2.97 (t, 2H), 2.80 (s, 3H), 1.97˜2.00 (m, 2H); LC/MS: m/e=355 (M+H)⁺.

### 146. Compound 146: 5-(3-chloro-4-fluorophenyl)-2-(pyridin-2-yl)-4,5,6,7-tetrahydrooxazolo[4,5-c]pyridine

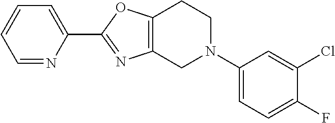

The title compound was prepared via the procedure used for the preparation of Compound 23, substituting 4-bromo-2-chloro-1-fluorobenzene for 3-bromobenzonitrile. Preparative HPLC afforded a yellow solid (50 mg, 21%). ¹H NMR (400 MHz, CDCl₃): δ 8.72 (d, 1H), 8.1 (d, 1H), 7.82 (m, 1H), 7.36 (m, 1H), 6.98-7.08 (m, 2H), 6.84 (m, 1H), 4.25 (s, 2H), 3.65 (t, 2H), 2.97 (t, 2H); LC/MS: m/e=330 (M+H)⁺.

### 147. Compound 147: 3-fluoro-5-(2-(2-methylthiazol-4-yl)-6,7-dihydrooxazolo[4,5-c]pyridin-5(4H)-yl)benzonitrile

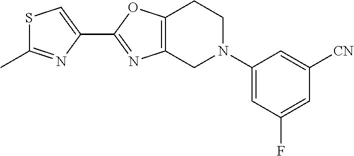

This compound is prepared using the procedures described herein elsewhere, such as example 23 and example 98, from suitable starting materials.

### 148. Compound 148: 3-(2-Phenyl-6,7-dihydrooxazolo[5,4-c]pyridin-5(4H)-yl)benzonitrile

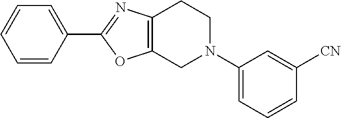

The title compound was prepared via the procedure used for the preparation of Compound 1, substituting benzoic acid for picolinic acid. Preparative HPLC afforded a brown solid (18 mg, 15%). ¹H NMR (400 MHz, CDCl₃): δ 7.99 (m, 2H), 7.50 (m, 3H), 7.39 (m, 3H), 7.13 (m, 1H), 4.47 (t, 2H, J=2.0 Hz), 3.76 (m, 2H), 2.80 (m, 2H); LC/MS: m/e=302 (M+H)⁺.

### 149. Compound 149: 3-(2-Cyclohexyl-6,7-dihydrooxazolo[5,4-c]pyridin-5(4H)-yl)benzonitrile

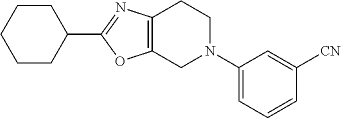

The title compound was prepared via the procedure used for the preparation of Compound 1, substituting cyclohexanecarboxylic acid for picolinic acid. Preparative HPLC afforded a tan solid (80 mg, 53%). ¹H NMR (400 MHz, CDCl₃): δ 7.34 (m, 1H), 7.10 (m, 3H), 4.29 (m, 2H), 3.64 (m, 2H), 2.73 (m, 3H), 2.07 (m, 2H), 1.83 (m, 2H), 1.55-1.75 (m, 5H), 1.33 (m, 3H); LC/MS: m/e=308 (M+H)⁺.

### 150. Compound 150:1-(4-Fluorophenyl)-2-(2-(pyridin-2-yl)-6,7-dihydrooxazolo[5,4-c]pyridin-5(4H)-yl)ethanone

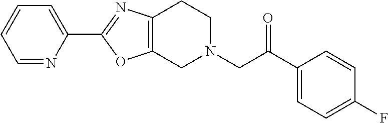

The title compound was prepared via the procedure used for the preparation of Compound 13, substituting 2-bromo-1-(4-fluorophenyl)ethanone for 3-(bromomethyl)benzonitrile. Preparative HPLC afforded a tan solid (14 mg, 30%). ¹H NMR (400 MHz, CDCl₃): δ: 8.70 (m, 1H), 8.04-8.10 (m, 3H), 7.80 (m, 1H), 7.34 (m, 1H), 7.14 (m, 2H), 4.09 (s, 2H), 3.93 (s, 2H), 3.07 (m, 2H), 2.81 (m, 2H); LC/MS: m/e=338 (M+H)⁺.

### 151. Compound 151: Methyl 2-(pyridin-2-yl)-6,7-dihydrooxazolo[4,5-c]pyridine-5(4H)-carboxylate

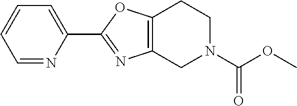

To a solution of 2-(pyridin-2-yl)-4,5,6,7-tetrahydrooxazolo[4,5-c]pyridine (100 mg, 0.50 mmole) in THF (10 ml) was added methyl carbonochloridate (50 mg, 0.50 mmol), triethylamine (100 mg, 1.00 mmole). The mixture was stirred at room temperature for 2 h. The solvent was evaporated in vacuo and the residue was purified using reverse phase chromatography to give the desired product (75 mg, 60%) as a white solid. ¹H NMR (400 MHz, CDCl₃): δ 8.69 (m, 1H), 8.09 (d, 1H), 7.84 (m, 1H), 7.27 (m, 1H), 4.55 (m, 2H), 3.90 (m, 2H), 3.76 (s, 3H), 2.86 (m, 2H); LC/MS: m/e=260 (M+H)⁺.

### 152. Compound 152: 5-(3,4-Difluorophenyl)-2-(pyridin-2-yl)-4,5,6,7-tetrahydrooxazolo[4,5-c]pyridine

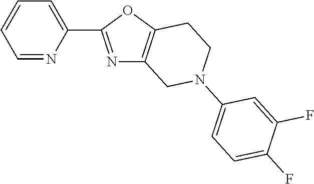

The title compound was prepared via the procedure used for the preparation of Compound 23, substituting 4-bromo-1,2-difluorobenzene for 3-bromobenzonitrile. Preparative HPLC afforded a yellow solid (41 mg, 18%). ¹H NMR (400 MHz, CDCl₃): δ 8.72 (d, 1H), 8.095 (d, 1H), 7.82 (m, 1H), 7.36 (m, 1H), 7.02-7.1 (q, 1H), 6.78 (m, 1H), 6.66 (m, 1H), 4.25 (s, 2H), 3.65 (t, 2H), 2.97 (t, 2H); LC/MS: m/e=314 (M+H)⁺.

### 153. Compound 153: 2-(Pyridin-2-yl)-5-(3,4,5-trifluorophenyl)-4,5,6,7-tetrahydrooxazolo[4,5-c]pyridine

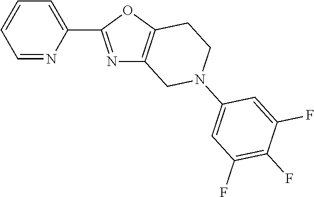

The title compound was prepared via the procedure used for the preparation of Compound 23, substituting 1-bromo-3,4,5-trifluorobenzene for 3-bromobenzonitrile. Preparative HPLC afforded a yellow powder (40 mg, 17%). ¹H NMR (400 MHz, CDCl₃): δ 8.72 (d, 1H), 8.095 (d, 1H), 7.82 (m, 1H), 7.38 (m, 1H), 6.50-6.58 (q, 2H), 4.25 (s, 2H), 3.65 (t, 2H), 2.97 (t, 2H); LC/MS: m/e=332 (M+H)⁺.

### 154. Compound 154: 2-(Pyridin-2-yl)-5-(pyridin-3-yl)-4,5,6,7-tetrahydrooxazolo[4,5-c]pyridine

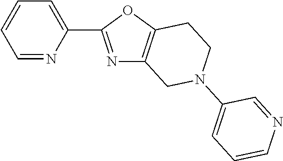

The title compound was prepared via the procedure used for the preparation of Compound 23, substituting 3-bromopyridine for 3-bromobenzonitrile. Preparative HPLC afforded a brown oil (2.4 mg, 1.2%). ¹H NMR (400 MHz, CDCl₃): δ 8.72 (s, 1H), 8.42 (d, 1H), 8.08-8.14 (m, 2H), 7.82 (t, 1H), 7.35 (t, 1H), 7.24 (s, 1H), 7.18 (m, 1H), 4.35 (s, 2H), 3.78 (t, 2H), 3.00 (s, 2H); LC/MS: m/e=279 (M+H)⁺.

### 155. Compound 155: 3-(2-Benzyl-6,7-dihydrooxazolo[4,5-c]pyridin-5(4H)-yl)-5-fluorobenzonitrile

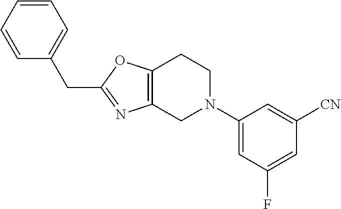

The title compound was prepared via the procedure used for the preparation of Compound 23, substituting phenylacetic acid for picolinic acid. Preparative HPLC afforded a solid (100 mg, 60%). ¹H NMR (400 MHz, CDCl₃): δ 7.31 (m, 5H), 6.92 (m, 1H), 6.76 (m, 2H), 4.20 (m, 2H), 4.09 (s, 2H), 3.68 (m, 2H), 2.82 (m, 2H); LC/MS: m/e=334 (M+H)⁺.

### 156. Compound 156: 5-(2-Chlorophenyl)-2-(pyridin-2-yl)-4,5,6,7-tetrahydrooxazolo[4,5-c]pyridine

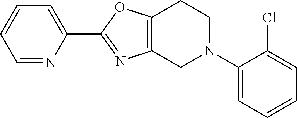

The title compound was prepared via the procedure used for the preparation of Compound 23, substituting 2-chloro-1-bromobenzene for 3-bromobenzonitrile. Preparative HPLC afforded a solid (15 mg, 32%). ¹H NMR (400 MHz, CDCl₃): δ: 8.72 (d, 1H), 8.10 (d, 1H), 7.81 (m, 1H), 7.40 (m, 1H), 7.35 (m, 1H), 7.23 (m, 1H), 7.14 (d, 1H), 7.00 (m, 1H), 4.22 (s, 2H), 3.53 (t, 2H), 3.00 (t, 2H); LC/MS: m/e=312 (M+H)⁺.

### 157. Compound 157: 5-(4-Chlorophenyl)-2-(pyridin-2-yl)-4,5,6,7-tetrahydrooxazolo[4,5-c]pyridine

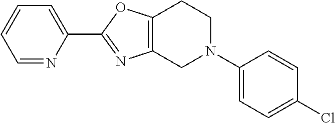

The title compound was prepared via the procedure used for the preparation of Compound 23, substituting 1-bromo-3-chlorobenzene for 3-bromobenzonitrile. Preparative HPLC afforded a solid (10 mg, 29%). ¹H NMR (400 MHz, CDCl₃): δ 8.72 (d, 1H), 8.09 (d, 1H), 7.81 (m, 1H), 7.35 (m, 1H), 7.22 (m, 2H), 6.91 (m, 2H), 4.27 (s, 2H), 3.68 (t, 2H), 2.97 (t, 2H); LC/MS: m/e=312 (M+H)⁺.

### 158. Compound 158: 5-(3-Chloro-5-fluorophenyl)-2-(pyridin-2-yl)-4,5,6,7-tetrahydrooxazolo[4,5-c]pyridine

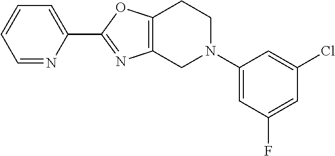

The title compound was prepared via the procedure used for the preparation of Compound 23, substituting 1-bromo-3-chloro-5-fluorobenzene for 3-bromobenzonitrile. Preparative HPLC afforded a solid (10 mg, 32%). ¹H NMR (400 MHz, CDCl₃): δ 8.72 (d, 1H), 8.09 (d, 1H), 7.83 (m, 1H), 7.36 (m, 1H), 6.71 (s, 1H), 6.54 (m, 2H), 4.30 (s, 2H), 3.73 (t, 2H), 2.98 (t, 2H); LC/MS: m/e=330 (M+H)⁺.

### 159. Compound 159: 5-(3-Chloro-5-fluorophenyl)-2-(5-fluoropyridin-2-yl)-4,5,6,7-tetrahydrooxazolo[4,5-c]pyridine

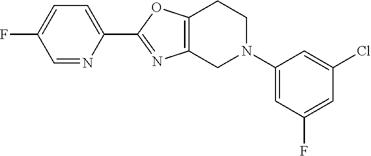

The title compound was prepared via the procedure used for the preparation of Compound 23, substituting 5-fluoropicolinic acid for picolinic acid, and substituting 1-bromo-3-chloro-5-fluorobenzene for 3-bromobenzonitrile. Preparative HPLC afforded a solid (43 mg, 53%). ¹H NMR (400 MHz, CDCl₃): δ 8.55 (d, 1H), 8.10˜8.09 (m, 1H), 7.53˜7.49 (m, 1H), 6.70 (s, 1H), 6.54˜6.50 (m, 2H), 4.27 (s, 2H), 3.72 (t, J=12, 2H), 2.97˜2.94 (m, 2H), 2.99 (2H, m); LC/MS: m/e=348 (M+H)⁺.

### 160. Compound 160: 3-Fluoro-5-(2-(pyrimidin-5-yl)-6,7-dihydrooxazolo[4,5-c]pyridin-5(4H)-yl)benzonitrile

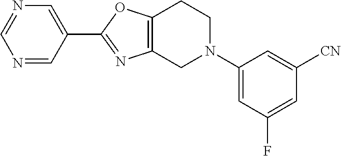

The title compound was prepared via the procedure used for the preparation of Compound 23, substituting pyrimidine-5-carboxylic acid for picolinic acid. Preparative HPLC afforded a solid (15 mg, 50%). ¹H NMR (400 MHz, CDCl₃): δ 9.30 (d, 3H), 6.99 (d, 1H), 6.84-6.87 (m, 2H), 4.34 (m, 2H), 3.79 (m, 2H), 3.01 (m, 2H), 4.13 (m, 2H), 2.95 (m, 2H); LC/MS: m/e=322 (M+H)⁺.

### 161. Compound 161: 2-(2-Methylthiazol-4-yl)-5-(pyridin-2-yl)-4,5,6,7-tetrahydrooxazolo[4,5-c]pyridine

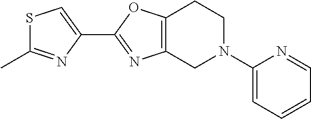

The title compound was prepared via the procedure used for the preparation of Compound 23, substituting 2-methylthiazole-4-carboxylic acid for picolinic acid, and substituting 2-bromopyridine for 3-bromobenzonitrile. Preparative HPLC afforded a solid (45 mg, 74%); ¹H NMR (400 MHz, CDCl₃): δ 8.19 (s, 1H), 7.74 (s, 1H), 7.51 (t, 1H), 6.70 (d, 1H), 6.63 (t, 1H), 4.49 (s, 2H), 4.10 (t, 2H), 2.92 (m, 2H), 2.77 (s, 3H); LC/MS: m/e=299 (M+H)⁺.

### 162. Compound 162: 5-(3,5-Dichlorophenyl)-2-(pyridin-2-yl)-4,5,6,7-tetrahydrooxazolo[4,5-c]pyridine

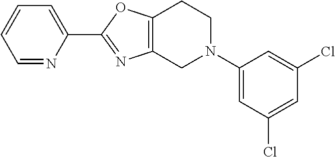

The title compound was prepared via the procedure used for the preparation of Compound 23, substituting 1-bromo-3,5-dichlorobenzene for 3-bromobenzonitrile. Preparative HPLC afforded a solid (50 mg, 67%). ¹H NMR (400 MHz, CDCl₃): δ 8.73 (d, 1H), 8.10 (d, 1H), 7.85˜7.80 (m, 1H), 7.38˜7.35 (m, 1H), 6.81 (s, 3H), 4.30 (s, 2H), 3.74 (t, 2H), 3.00˜2.97 (m, 2H); LC/MS: m/e=346 (M+H)⁺.

### 163. Compound 163: 5-(2-Methylthiazol-4-yl)-2-(pyridin-2-yl)-4,5,6,7-tetrahydrooxazolo[4,5-c]pyridine

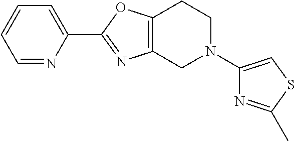

The title compound was prepared via the procedure used for the preparation of Compound 23, substituting 4-bromo-2-methylthiazole for 3-bromobenzonitrile. Preparative HPLC afforded a solid (15 mg, 30%). ¹H NMR (400 MHz, CDCl₃): δ 8.71 (m, 1H), 8.12 (m, 1H), 7.76 (m, 1H), 7.35 (m, 1H), 5.79 (s, 1H), 3.01 (m, 2H), 4.30 (m, 2H), 3.90 (m, 2H), 2.99 (m, 2H), 2.65 (s, 3H); LC/MS: m/e=299 (M+H)⁺.

### 164. Compound 164: 3-Fluoro-5-(5-(pyridin-2-yl)-4,5,6,7-tetrahydrooxazolo[4,5-c]pyridin-2-yl)benzonitrile

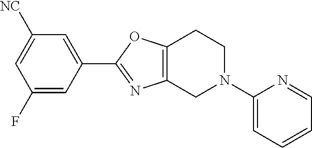

The title compound was prepared via the procedure used for the preparation of Compound 23, substituting 3-cyano-5-fluorobenzoic acid for picolinic acid, and substituting 2-bromopyridine for 3-bromobenzonitrile. Preparative HPLC afforded a solid (10 mg, 15%). ¹H NMR (400 MHz, CDCl₃): δ 8.23 (m, 1H), 8.11 (m, 1H), 7.95 (m, 1H), 7.50 (m, 1H), 7.40 (m, 1H), 6.65-6.75 (m, 2H), 4.13 (m, 2H), 2.95 (m, 2H); LC/MS: m/e=321 (M+H)⁺.

### 165. Compound 165: 5-Fluoro-2-(2-(pyridin-2-yl)-6,7-dihydrooxazolo[4,5-c]pyridin-5(4H)-yl)benzonitrile

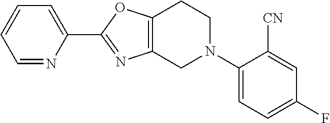

The title compound was prepared via the procedure used for the preparation of Compound 23, substituting 2-bromo-5-fluorobenzonitrile for 3-bromobenzonitrile. Preparative HPLC afforded a solid (20 mg, 32%). ¹H NMR (400 MHz, MeOH-d₄): δ 8.66 (d, 1H), 8.13 (d, 1H), 7.98 (m, 1H), 7.49 (m, 2H), 7.36 (m, 2H), 4.25 (s, 2H), 3.70 (m, 2H), 3.07 (m, 2H); LC/MS: m/e=321 (M+H)⁺.

### 166. Compound 166: 5-(2-Fluorophenyl)-2-(pyridin-2-yl)-4,5,6,7-tetrahydrooxazolo[4,5-c]pyridine

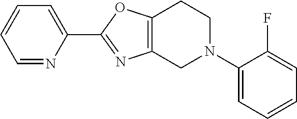

The title compound was prepared via the procedure used for the preparation of Compound 23, substituting 1-bromo-2-fluorobenzene for 3-bromobenzonitrile. Preparative HPLC afforded a solid (20 mg, 41%). ¹H NMR (400 MHz, CDCl₃): δ 8.66 (m, 1H), 8.02 (m, 1H), 7.76 (m, 1H), 7.30 (m, 1H), 6.99 (m, 4H), 4.18 (s, 2H), 3.50 (m, 2H), 3.00 (m, 2H); LC/MS: m/e=296 (M+H)⁺.

### 167. Compound 167: 5-(2-Chlorophenyl)-2-(5-fluoropyridin-2-yl)-4,5,6,7-tetrahydrooxazolo[4,5-c]pyridine

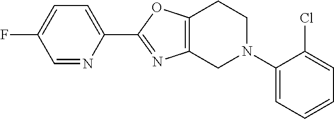

The title compound was prepared via the procedure used for the preparation of Compound 23, substituting 5-fluoropicolinic acid for picolinic acid, and substituting 1-bromo-2-chlorobenzene for 3-bromobenzonitrile. Preparative HPLC afforded a solid (25 mg, 37%). ¹H NMR (400 MHz, MeOH-d₄): δ 8.56 (s, 1H), 8.15 (m, 1H), 7.77 (m, 1H), 7.40 (m, 1H), 7.26 (m, 2H), 7.03 (m, 1H), 4.13 (s, 2H), 3.52 (m, 2H), 3.01 (m, 2H); LC/MS: m/e=330 (M+H)⁺.

### 168. Compound 168: 5-(2-Chlorophenyl)-2-(pyrimidin-4-yl)-4,5,6,7-tetrahydrooxazolo[4,5-c]pyridine

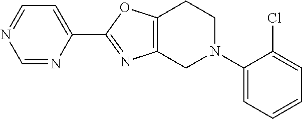

The title compound was prepared via the procedure used for the preparation of Compound 23, substituting pyrimidine-4-carboxylic acid for picolinic acid, and substituting 1-bromo-2-chlorobenzene for 3-bromobenzonitrile. Preparative HPLC afforded a solid (15 mg, 23%). ¹H NMR (400 MHz, MeOH-d₄): δ 9.24 (s, 1H), 8.94 (m, 1H), 8.10 (m, 1H), 7.41 (m, 1H), 7.29 (m, 2H), 7.06 (m, 1H), 4.17 (s, 2H), 3.52 (m, 2H), 3.03 (m, 2H); LC/MS: m/e=312 (M+H)⁺.

### 169. Compound 169: 5-(2,5-Difluorophenyl)-2-(pyridin-2-yl)-4,5,6,7-tetrahydrooxazolo[4,5-c]pyridine

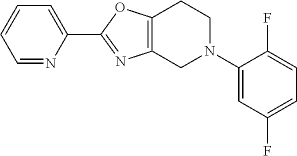

The title compound was prepared via the procedure used for the preparation of Compound 23, substituting 2-bromo-1,4-difluorobenzene for 3-bromobenzonitrile. Preparative HPLC afforded a solid (21 mg, 25%). ¹H NMR (400 MHz, MeOH-d₄): δ 8.66 (m, 1H), 8.13 (m, 1H), 7.99 (m, 1H), 7.52 (m, 1H), 7.11 (m, 1H), 6.92 (m, 1H), 6.70 (m, 1H), 4.20 (s, 2H), 3.61 (m, 2H), 3.01 (m, 2H); LC/MS: m/e=313 (M+H)⁺.

### 170. Compound 170: 5-(2,3-Dichlorophenyl)-2-(pyridin-2-yl)-4,5,6,7-tetrahydrooxazolo[4,5-c]pyridine

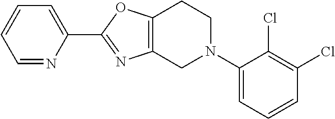

The title compound was prepared via the procedure used for the preparation of Compound 23, substituting 1-bromo-2,3-dichlorobenzene for 3-bromobenzonitrile. Preparative HPLC afforded a solid (12 mg, 17%). ¹H NMR (400 MHz, MeOH-d₄): δ 8.66 (m, 1H), 8.13 (m, 1H), 7.99 (m, 1H), 7.52 (m, 1H), 7.22 (m, 3H), 4.16 (s, 2H), 3.51 (m, 2H), 3.01 (m, 2H); LC/MS: m/e=346 (M+H)⁺.

### 171. Compound 171: 5-(2-Chloro-4-fluorophenyl)-2-(pyridin-2-yl)-4,5,6,7-tetrahydrooxazolo[4,5-c]pyridine

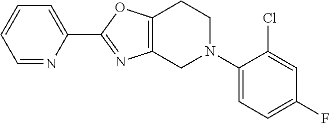

The title compound was prepared via the procedure used for the preparation of Compound 23, substituting 1-bromo-2-chloro-4-fluorobenzene for 3-bromobenzonitrile. Preparative HPLC afforded a solid (22 mg, 33%). ¹H NMR (400 MHz, MeOH-d₄): δ 8.66 (m, 1H), 8.13 (m, 1H), 7.99 (m, 1H), 7.52 (m, 1H), 7.30 (m, 2H), 7.08 (m, 1H), 4.12 (s, 2H), 3.49 (m, 2H), 3.01 (m, 2H); LC/MS: m/e=330 (M+H)⁺.

### 172. Compound 172: 5-(2-Chlorophenyl)-2-(2-methylthiazol-4-yl)-4,5,6,7-tetrahydrooxazolo[4,5-c]pyridine

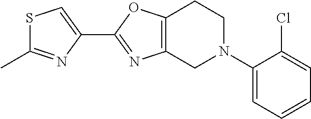

The title compound was prepared via the procedure used for the preparation of Compound 23, substituting 2-methylthiazole-4-carboxylic acid for picolinic acid, and substituting 1-bromo-2-chlorobenzene for 3-bromobenzonitrile. Preparative HPLC afforded a solid (18 mg, 27%). ¹H NMR (400 MHz, MeOH-d₄): δ 8.00 (m, 1H), 7.42 (m, 1H), 7.28 (m, 2H), 7.06 (m, 1H), 4.12 (s, 2H), 3.50 (m, 2H), 2.96 (m, 2H), 2.80 (s, 3H); LC/MS: m/e=332 (M+H)⁺.

### 173. Compound 173: 5-(2,5-Dichlorophenyl)-2-(pyridin-2-yl)-4,5,6,7-tetrahydrooxazolo[4,5-c]pyridine

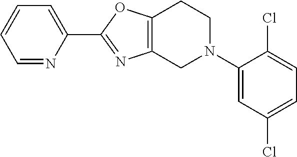

The title compound was prepared via the procedure used for the preparation of Compound 23, substituting 2-bromo-1,4-dichlorobenzene for 3-bromobenzonitrile. Preparative HPLC afforded a solid (15 mg, 20%). ¹H NMR (400 MHz, MeOH-d₄): δ 8.72 (m, 1H), 8.10 (m, 1H), 7.82 (m, 1H), 7.35 (m, 1H), 7.27 (m, 1H), 7.09 (m, 1H), 7.00 (m, 1H), 4.12 (s, 2H), 3.49 (m, 2H), 3.01 (m, 2H); LC/MS: m/e=346 (M+H)⁺.

### 174. Compound 174: 5-(2,6-Dichlorophenyl)-2-(pyridin-2-yl)-4,5,6,7-tetrahydrooxazolo[4,5-c]pyridine

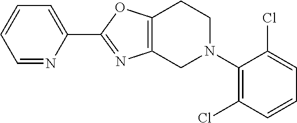

The title compound was prepared via the procedure used for the preparation of Compound 23, substituting 2-bromo-1,3-dichlorobenzene for 3-bromobenzonitrile. Preparative HPLC afforded a solid (11 mg, 16%). ¹H NMR (400 MHz, MeOH-d₄): δ 8.66 (m, 1H), 8.12 (d, 1H), 7.98 (m, 1H), 7.51 (m, 1H), 7.40 (d, 2H), 7.17 (m, 1H), 4.20 (s, 2H), 3.59 (m, 2H), 2.98 (m, 2H); LC/MS: m/e=346 (M+H)⁺.

### 175. Compound 175: 5-(2-Chloro-5-fluorophenyl)-2-(pyridin-2-yl)-4,5,6,7-tetrahydrooxazolo[4,5-c]pyridine

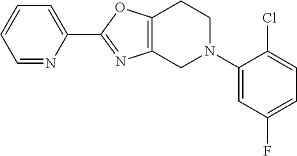

The title compound was prepared via the procedure used for the preparation of Compound 23, substituting 2-bromo-1-chloro-4-fluorobenzene for 3-bromobenzonitrile. Preparative HPLC afforded a solid (20 mg, 30%). ¹H NMR (400 MHz, MeOH-d₄): δ 8.51 (d, 1H), 7.96 (d, 1H), 7.84 (m, 1H), 7.37 (m, 1H), 7.27 (m, 1H), 6.88 (m, 1H), 6.67 (m, 1H), 3.99 (s, 2H), 3.38 (m, 2H), 2.87 (m, 2H); LC/MS: m/e=330 (M+H)⁺.

### 176. Compound 176: 4-Fluoro-2-(2-(pyridin-2-yl)-6,7-dihydrooxazolo[4,5-c]pyridin-5(4H)-yl)benzonitrile

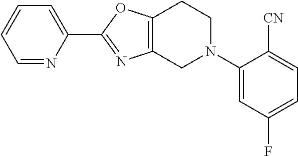

The title compound was prepared via the procedure used for the preparation of Compound 23, substituting 2-bromo-4-fluorobenzonitrile for 3-bromobenzonitrile. Preparative HPLC afforded a solid (20 mg, 32%). ¹H NMR (400 MHz, MeOH-d₄): δ 8.68 (m, 1H), 8.16 (m, 1H), 8.00 (m, 1H), 7.73 (m, 1H), 7.54 (m, 1H), 7.06 (m, 1H), 6.86 (m, 1H), 4.34 (m, 2H), 3.85 (m, 2H), 3.13 (m, 2H); LC/MS: m/e=321 (M+H)⁺.

### 177. Compound 177: 5-(2-Methoxyphenyl)-2-(pyridin-2-yl)-4,5,6,7-tetrahydrooxazolo[4,5-c]pyridine

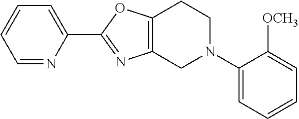

The title compound was prepared via the procedure used for the preparation of Compound 23, substituting 1-bromo-2-methoxybenzene for 3-bromobenzonitrile. Preparative HPLC afforded a solid (110 mg, 70%). ¹H NMR (400 MHz, MeOH-d₄): δ 8.63 (m, 1H), 8.10 (m, 1H), 7.98 (m, 1H), 7.50 (m, 1H), 7.03 (m, 2H), 6.91 (m, 2H), 4.12 (s, 2H), 3.83 (s, 3H), 3.50 (m, 2H), 2.94 (m, 2H); LC/MS: m/e=308 (M+H)⁺.

### 178. Compound 178: 2-(2-(Pyridin-2-yl)-6,7-dihydrooxazolo[4,5-c]pyridin-5(4H)-yl)phenol

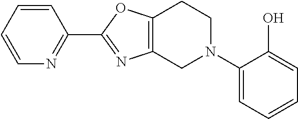

5-(2-Methoxyphenyl)-2-(pyridin-2-yl)-4,5,6,7-tetrahydrooxazolo[4,5-c]pyridine (110 mg, 0.35 mmol) was dissolved in DCM (6 mL) and cooled to 0° C. Boron tribromide (0.2 ml) was added and the mixture was stirred for 2 h. Methanol (0.5 mL) and water were added to the reaction. The organic layer was washed with brine, dried over Na₂SO₄, filtered and concentrated to give the title compound (95 mg, 91%) as a solid. ¹H NMR (400 MHz, MeOH-d₄): δ 8.68 (d, 1H), 8.18 (s, 1H), 8.03 (m, 1H), 7.57 (m, 1H), 7.43 (m, 1H), 7.28 (m, 1H), 7.05 (m, 1H), 6.97 (m, 1H), 4.62 (s, 2H) 3.98 (t, 2H) 3.24 (t, 2H); LC/MS: m/e=294 (M+H)⁺.

### 179. Compound 179: 5-(5-Fluoro-2-methoxyphenyl)-2-(pyridin-2-yl)-4,5,6,7-tetrahydrooxazolo[4,5-c]pyridine

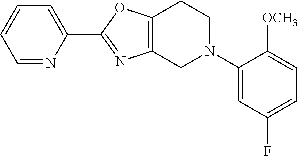

The title compound was prepared via the procedure used for the preparation of Compound 23, substituting 2-bromo-4-fluoro-1-methoxybenzene for 3-bromobenzonitrile. Preparative HPLC afforded a solid (22 mg, 34%). ¹H NMR (400 MHz, MeOH-d₄): δ 8.63 (m, 1H), 8.12 (m, 1H), 7.98 (m, 1H), 7.50 (m, 1H), 6.95 (m, 1H), 6.82 (m, 1H), 6.72 (m, 1H), 4.12 (s, 2H), 3.85 (s, 3H), 3.52 (m, 2H), 2.96 (m, 2H); LC/MS: m/e=326 (M+H)⁺.

### 180. Compound 180: 2-(2-(Pyridin-2-yl)-6,7-dihydrooxazolo[4,5-c]pyridin-5(4H)-yl)benzonitrile

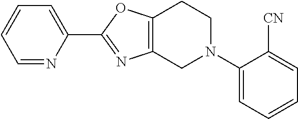

The title compound was prepared via the procedure used for the preparation of Compound 23, substituting 2-bromobenzonitrile for 3-bromobenzonitrile. Preparative HPLC afforded a solid (22 mg, 36%). ¹H NMR (400 MHz, MeOH-d₄): δ 8.63 (m, 1H), 8.12 (m, 1H), 7.98 (m, 1H), 7.66 (m, 1H), 7.58 (m, 1H), 7.50 (m, 1H), 7.26 (m, 1H), 7.07 (m, 1H), 4.28 (m, 2H), 3.75 (m, 2H), 3.06 (m, 2H); LC/MS: m/e=303 (M+H)⁺.

### 181. Compound 181: 5-(Naphthalen-1-yl)-2-(pyridin-2-yl)-4,5,6,7-tetrahydrooxazolo[4,5-c]pyridine

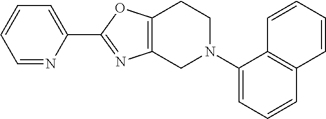

The title compound was prepared via the procedure used for the preparation of Compound 23, substituting 1-bromonaphthalene for 3-bromobenzonitrile. Preparative HPLC afforded a solid (25 mg, 38%). ¹H NMR (400 MHz, MeOH-d₄): δ 8.66 (m, 1H), 8.22 (m, 1H), 8.12 (m, 1H), 7.98 (m, 1H), 7.84 (m, 1H), 7.60 (m, 1H), 7.58 (m, 1H), 7.50 (m, 4H), 7.25 (m, 1H), 4.14 (m, 2H), 3.50 (m, 2H), 3.00 (m, 2H); LC/MS: m/e=328 (M+H)⁺.

### 182. Compound 182: 2-(Pyridin-2-yl)-5-(2-(trifluoromethyl)phenyl)-4,5,6,7-tetrahydrooxazolo[4,5-c]pyridine

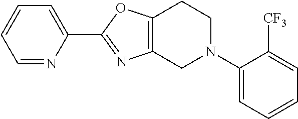

The title compound was prepared via the procedure used for the preparation of Compound 23, substituting 1-bromo-2-(trifluoromethyl)benzene for 3-bromobenzonitrile. Preparative HPLC afforded a solid (14 mg, 16%). ¹H NMR (400 MHz, CDCl₃): δ 8.72 (d, 1H), 8.09 (d, 1H), 7.81 (t, 1H), 7.68 (d, 1H), 7.56 (t, 1H), 7.45 (d, 1H), 7.29˜7.36 (m, 2H), 4.08 (s, 2H), 3.33 (t, 2H), 2.97 (t, 2H); LC/MS: m/e=346 (M+H)⁺.

### 183. Compound 183: 5-(2-Methylthiazol-4-yl)-2-(pyridin-2-yl)-4,5,6,7-tetrahydrooxazolo[4,5-c]pyridine

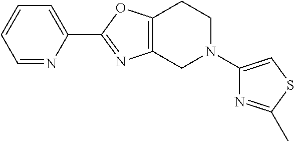

The title compound was prepared via the procedure used for the preparation of Compound 23, substituting 4-bromo-2-methylthiazole for 3-bromobenzonitrile. Preparative HPLC afforded a solid (8 mg, 10%). ¹H NMR (400 MHz, CDCl₃): δ 8.71 (d, 1H), 8.09 (d, 1H), 7.82 (t, 1H), 7.36 (m, 1H), 6.85 (s, 1H), 4.50 (s, 2H), 3.96 (t, 2H), 3.02 (t, 2H), 2.32 (s, 3H); LC/MS: m/e=299 (M+H)⁺.

### 184. Compound 184: 5-(3-Chloropyridin-4-yl)-2-(pyridin-2-yl)-4,5,6,7-tetrahydrooxazolo[4,5-c]pyridine

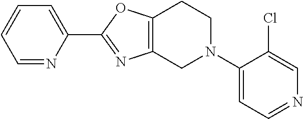

The title compound was prepared via the procedure used for the preparation of Compound 23, substituting 4-bromo-3-chloropyridine for 3-bromobenzonitrile. Preparative HPLC afforded a solid (32 mg, 41%). ¹H NMR (400 MHz, CDCl₃): δ 8.70 (d, 1H), 8.42 (s, 1H), 8.29 (d, 1H), 8.07 (d, 1H), 7.80 (t, 1H), 7.34 (t, 1H), 6.89 (d, 1H), 4.30 (s, 2H), 3.71 (t, 2H), 3.02 (t, 2H); LC/MS: m/e=313 (M+H)⁺.

### 185. Compound 185: 4-Fluoro-3-(2-(pyridin-2-yl)-6,7-dihydrooxazolo[4,5-c]pyridin-5(4H)-yl)benzonitrile

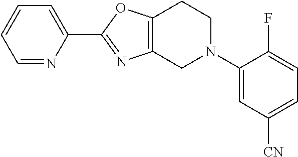

The title compound was prepared via the procedure used for the preparation of Compound 23, substituting 3-bromo-4-fluorobenzonitrile for 3-bromobenzonitrile. Preparative HPLC afforded a solid (10 mg, 16%). ¹H NMR (400 MHz, CDCl₃): δ 8.75 (brs, 1H), 8.12 (d, 1H), 7.85 (t, 1H), 7.40 (t, 1H), 7.24˜7.29 (m, 2H), 7.12˜7.17 (m, 1H), 4.27 (s, 2H), 3.62 (t, 2H), 3.00 (t, 2H); LC/MS: m/e=321 (M+H)⁺.

### 186. Compound 186: 3-Chloro-5-(2-(pyridin-2-yl)-6,7-dihydrooxazolo[4,5-c]pyridin-5(4H)-yl)benzonitrile

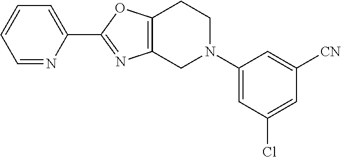

The title compound was prepared via the procedure used for the preparation of Compound 23, substituting 3-bromo-5-chlorobenzonitrile for 3-bromobenzonitrile. Preparative HPLC afforded a solid (6 mg, 12%). ¹H NMR (400 MHz, CDCl₃): δ 8.74 (s, 1H), 8.10 (d, 1H), 7.83 (t, 1H), 7.40˜7.36 (m, 1H), 7.12˜7.11 (m, 1H), 7.07˜7.06 (m, 2H), 4.34 (s, 2H), 3.78 (t, 2H), 3.03˜3.00 (m, 2H); LC/MS: m/e=337 (M+H)⁺.

### 187. Compound 187: 5-(3-Chloropyridin-2-yl)-2-(pyridin-2-yl)-4,5,6,7-tetrahydrooxazolo[4,5-c]pyridine

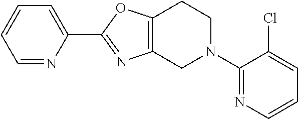

The title compound was prepared via the procedure used for the preparation of Compound 23, substituting 2-bromo-3-chloropyridine for 3-bromobenzonitrile. Preparative HPLC afforded a solid (7 mg, 4%). ¹H NMR (400 MHz, CDCl₃): δ 8.72˜8.71 (m, 1H), 8.19˜8.18 (m, 1H), 8.11˜8.10 (m, 1H), 7.81˜7.80 (m, 1H), 7.63˜7.61 (m, 1H), 7.34˜7.33 (m, 1H), 6.86˜6.85 (m, 1H), 4.50 (s, 2H), 3.79˜3.77 (m, 2H), 3.10˜3.09 (m, 2H); LC/MS: m/e=313 (M+H)⁺.

### 188. Compound 188: 2-(Pyridin-2-yl)-5-(2,3,5-trifluorophenyl)-4,5,6,7-tetrahydrooxazolo[4,5-c]pyridine

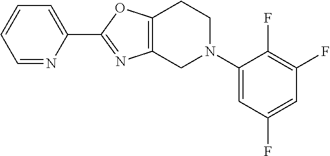

The title compound was prepared via the procedure used for the preparation of Compound 23, substituting 1-bromo-2,3,5-trifluorobenzene for 3-bromobenzonitrile. Preparative HPLC afforded a solid (9 mg, 5%). ¹H NMR (400 MHz, CDCl₃): δ 8.73˜8.72 (m, 1H), 8.10 (d, 1H), 7.84˜7.81 (m, 1H), 7.38˜7.36 (m, 1H), 6.54˜6.48 (m, 2H), 4.25 (s, 2H), 3.64˜3.62 (m, 2H), 3.00˜2.99 (m, 2H); LC/MS: m/e=332 (M+H)⁺.

### 189. Compound 189: 2-Fluoro-6-(2-(pyridin-2-yl)-6,7-dihydrooxazolo[4,5-c]pyridin-5(4H)-yl)benzonitrile

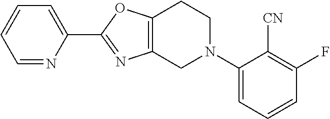

The title compound was prepared via the procedure used for the preparation of Compound 23, substituting 2-bromo-6-fluorobenzonitrile for 3-bromobenzonitrile. Preparative HPLC afforded a solid (33 mg, 68%). ¹H NMR (400 MHz, CDCl₃): δ 8.73 (d, 1H), 8.09 (d, 1H), 7.85˜7.81 (m, 1H), 7.46˜7.43 (m, 1H), 7.38˜7.35 (m, 1H), 6.84˜6.74 (m, 2H), 4.34 (s, 2H), 3.86˜3.83 (m, 2H), 3.13˜3.10 (m, 2H); LC/MS: m/e=321 (M+H)⁺.

### 190. Compound 190: 2-(2-(3-Chlorophenyl)-6,7-dihydrooxazolo[4,5-c]pyridin-5(4H)-yl)-1-(4-fluorophenyl)ethanone

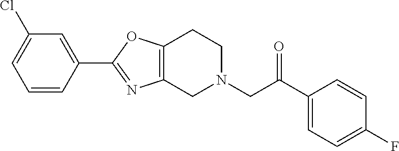

The title compound was prepared via the procedure used for the preparation of Compound 34, substituting 2-(3-chlorophenyl)-4,5,6,7-tetrahydrooxazolo[4,5-c]pyridine (purchased from Anichem) for 2-(pyridin-2-yl)-4,5,6,7-tetrahydrooxazolo[4,5-c]pyridine, and substituting 2-bromo-1-(4-fluorophenyl)ethanone for 3-(bromomethyl)benzonitrile. Preparative HPLC afforded a solid (5 mg, 18%). ¹H NMR (400 MHz, CDCl₃): δ: 8.08-8.06 (m, 2H), 7.99 (m, 1H), 7.87 (m, 1H), 7.38-7.37 (m, 2H), 7.16-7.12 (m, 2H), 4.08 (s, 2H), 3.78 (s, 2H), 3.08-3.06 (m, 2H), 2.89 (m, 2H); LC/MS: m/e=371 (M+H)⁺.

### 191. Compound 191: 1-(4-Fluorophenyl)-2-(2-(3-fluorophenyl)-6,7-dihydrooxazolo[4,5-c]pyridin-5(4H)-yl)ethanone

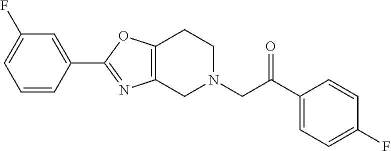

The title compound was prepared via the procedure used for the preparation of Compound 34, substituting 2-(3-fluorophenyl)-4,5,6,7-tetrahydrooxazolo[4,5-c]pyridine (purchased from Anichem) for 2-(pyridin-2-yl)-4,5,6,7-tetrahydrooxazolo[4,5-c]pyridine, and substituting 2-bromo-1-(4-fluorophenyl)ethanone for 3-(bromomethyl)benzonitrile. Preparative HPLC afforded a solid (2 mg, 12%). ¹H NMR (400 MHz, CDCl₃): δ: 8.27 (m, 1H), 8.16 (m, 1H), 8.09-8.05 (m, 2H), 7.66 (m, 1H), 7.59-7.57 (m, 1H), 7.17-7.13 (m, 2H), 4.12 (s, 2H), 3.82 (s, 2H), 3.12 (m, 2H), 2.93 (m, 2H); LC/MS: m/e=355 (M+H)⁺.

### 192. Compound 192: 1-(4-Fluorophenyl)-2-(2-(pyridin-2-yl)-6,7-dihydrooxazolo[4,5-c]pyridin-5(4H)-yl)ethanone

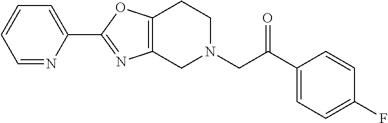

The title compound was prepared via the procedure used for the preparation of Compound 34, substituting 2-bromo-1-(4-fluorophenyl)ethanone for 3-(bromomethyl)benzonitrile. Preparative HPLC afforded a solid (9 mg, 18%). ¹H NMR (400 MHz, CDCl₃): δ: 8.72-8.70 (m, 1H), 8.09-8.06 (m, 3H), 7.83-7.79 (m, 1H), 7.36-7.34 (m, 1H), 7.16-7.12 (m, 2H), 4.09 (s, 2H), 3.81-3.80 (m, 2H), 3.09-3.07 (m, 2H), 2.95-2.93 (m, 2H); LC/MS: m/e=337 (M+H)⁺.

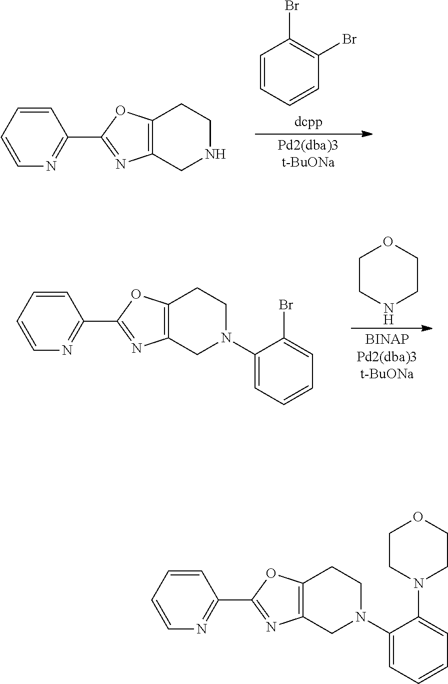

### 193. Compound 193: 5-(2-Morpholinophenyl)-2-(pyridin-2-yl)-4,5,6,7-tetrahydrooxazolo[4,5-c]pyridine

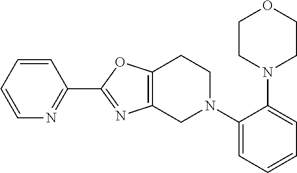

5-(2-bromophenyl)-2-(pyridin-2-yl)-4,5,6,7-tetrahydrooxazolo[4,5-c]pyridine (60 mg, 0.17 mmo), morpholine (22 mg, 0.26 mmol), Pd₂(dba)₃ (12 mg), BINAP (12 mg) and t-BuONa (34 mg, 0.34 mmol) were combined in toluene (5 ml) under an argon atmosphere. The reaction mixture was heated at 120° C. via microwave for 2 h. The reaction was cooled, filtered and concentrated to dryness. The residue was purified using Prep-TLC (PE:ethyl ether=1:1) to give the title compound as a white solid (30 mg, 48%). ¹H NMR (400 MHz, CDCl₃) δ: 8.73 (m, 1H), 8.10 (m, 1H), 7.80 (m, 1H), 7.33 (m, 1H), 6.96 (m, 4H), 4.28 (s, 2H), 3.80 (m, 4H), 3.64 (m, 2H), 3.14 (m, 4H), 2.95 (s, 2H); LC/MS: m/e=363 (M+H)⁺.

### (a) 5-(2-Bromophenyl)-2-(pyridin-2-yl)-4,5,6,7-tetrahydrooxazolo[4,5-c]pyridine (I-193.1)

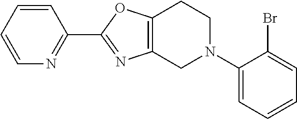

The title compound was prepared via the procedure used for the preparation of Compound 23, substituting 1,2-dibromobenzene for 3-bromobenzonitrile. Preparative TLC afforded a white solid (60 mg, 33%). LC/MS: m/e=356 (M+H)⁺.

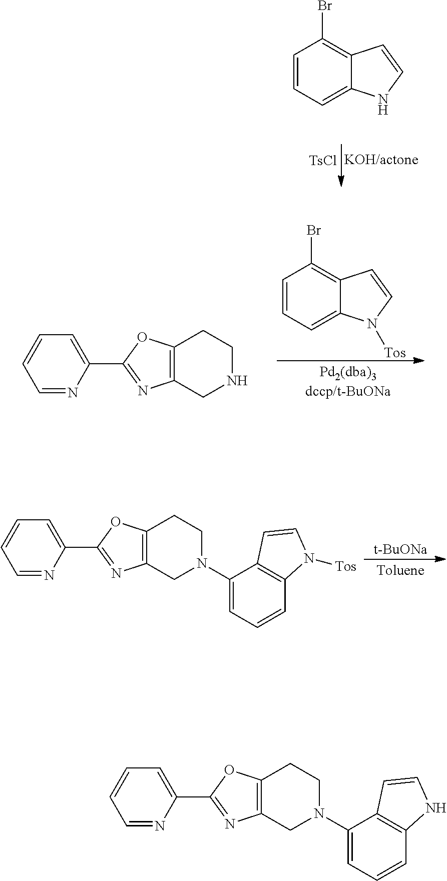

### 194. Compound 194: 5-(1H-Indol-4-yl)-2-(pyridin-2-yl)-4,5,6,7-tetrahydrooxazolo[4,5-c]pyridine

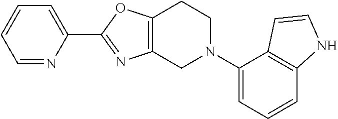

2-(Pyridin-2-yl)-5-(1-tosyl-1H-indol-4-yl)-4,5,6,7-tetrahydrooxazolo[4,5-c]pyridine (60 mg, 0.13 mmol) was dissolved in toluene (2.0 ml) and t-BuONa (20 mg, 0.20 mmol) was added under argon. The mixture was heated at 110° C. via microwave for 2 h. The reaction was filtered, concentrated, and the residue purified using Prep-TLC (PE:ethyl acetate=1:1) to give the title compound (20 mg, 50%) as a white solid. ¹H NMR (400 MHz, DMSO-d₆): δ: 11.11 (s, 1H), 8.68 (d, 1H), 8.08 (d, 1H), 7.96 (m, 1H), 7.49 (m, 1H), 7.28 (m, 1H), 7.05 (d, 1H), 6.98 (m, 1H), 6.59 (d, 1H) 6.49 (s, 1H) 4.23 (s, 2H) 3.68 (m, 2H) 3.03 (m, 2H); LC/MS: m/e=315 (M+H)⁺.

### (a) 2-(Pyridin-2-yl)-5-(1-tosyl-1H-indol-4-yl)-4,5,6,7-tetrahydrooxazolo[4,5-c]pyridine (I-194.1)

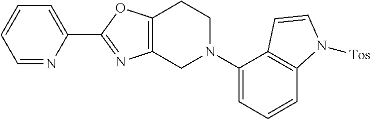

2-(Pyridin-2-yl)-4,5,6,7-tetrahydrooxazolo[4,5-c]pyridine (80 mg, 0.4 mmol) was mixed with 4-bromo-1-tosyl-1H-indole (220 mg, 0.6 mmol), Pd₂(dba)₃ (15 mg), dcpp (15 mg) and t-BuONa (80 mg, 0.8 mmol) in toluene (2.0 ml) under argon. The mixture was heated at 120° C. in a microwave tube for 2 h. The reaction was filtered and concentrated, and the residue was purified using Prep-TLC (PE:ethyl acetate=1:1) to give the title compound as a solid (60 mg, 32%). LC/MS: m/e=469 (M+H)⁺.

### (b) 4-Bromo-1-tosyl-1H-indole (I-194.2)

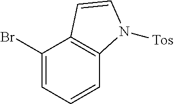

4-Bromo-1H-indole (1.95 g, 10.0 mmol) and KOH (0.84 g, 15.0 mmol) were dissolved in acetone (15 mL). Tosyl chloride (2.86 g, 15 mmol) was added in one portion. The mixture was stirred at room temperature for 2 h and concentrated to dryness. The residue was extracted with DCM and water, and washed with brine. The organic layer was dried over Na₂SO₄, filtered and concentrated. The residue was purified using silica gel chromatography with (PE:ethyl acetate=5:1 as mobile phase) to give the title compound as a solid (2.1 g, 60%). LC/MS: m/e=352 (M+H)⁺.

### 195. Compound 195: 3-Fluoro-5-(2-(2-(pyridin-2-yl)propan-2-yl)-6,7-dihydrooxazolo[4,5-c]pyridin-5(4H)-yl)benzonitrile

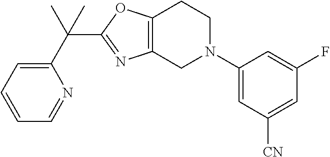

The title compound was prepared via the procedure used for the preparation of Compound 23, substituting 2-methyl-2-(pyridin-2-yl)propanoic acid for picolinic acid. Preparative HPLC afforded a solid (21 mg, 50%). ¹H NMR (400 MHz, CDCl₃): δ 8.56 (m, 1H), 7.63 (m, 1H), 7.15 (m, 2H), 6.93 (s, 1H), 6.77 (m, 2H), 4.26 (s, 2H), 3.69 (t, 2H), 2.83 (m, 2H), 1.82 (s, 6H); LC/MS: m/e=363 (M+H)⁺.

### (a) Methyl 2-methyl-2-(pyridin-2-yl)propanoate (I-195.1)

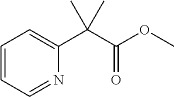

To a solution of methyl 2-(pyridin-2-yl)acetate (1.00 g, 6.62 mmole) in dry THF (25 mL) was added lithium diisopropylamide (16.6 ml, 33.1 mmole, 2M in THF) dropwise at −78° C. The reaction was stirred for 30 min at −78° C. Iodomethane (2.1 ml, 33.1 mmole) was added, the mixture was stirred at −78° C. for 1 h, warmed to room temperature and stirred for 8 h. The reaction was quenched with saturated aqueous NaHCO₃ (50 mL), and extracted with DCM (2×50 mL). The organic layer was dried over Na₂SO₄ and concentrated to dryness. The residue was purified with silica gel chromatography (DCM: methanol=10:1) to give the desired compound (590 mg, yield 50%) as a white solid. LC/MS: m/e=180 (M+H)⁺.

### (b) 2-Methyl-2-(pyridin-2-yl)propanoic acid (I-195.2)

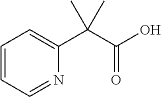

To solution of methyl 2-methyl-2-(pyridin-2-yl)propanoate (200 mg, 1.12 mmole) in methanol (5 mL) was added sodium hydroxide (54 mg, 1.34 mmole). The reaction was stirred at room temperature for 3 h; the resulting mixture was concentrated in vacuo. The residue was dissolved in DCM (50 mL), washed with water (40 mL), and dried over Na₂SO₄. Concentration in vacuo afford the desired product (165 mg, 90%) as a white solid. LC/MS: m/e=166 (M+H)⁺.

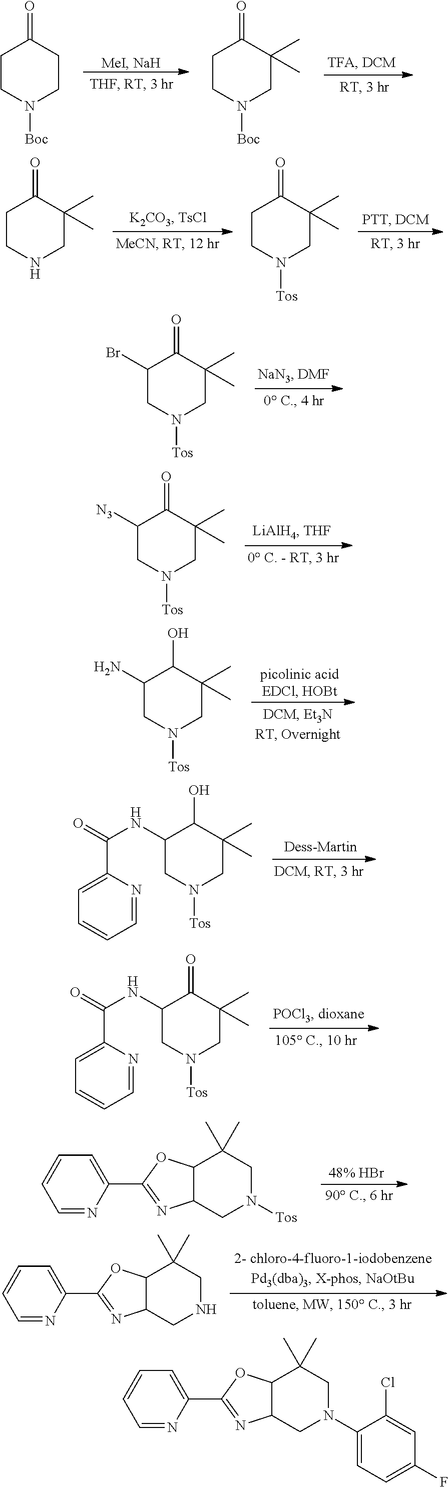

### 196. Compound 196: 5-(2-Chloro-4-fluorophenyl)-7,7-dimethyl-2-(pyridin-2-yl)-4,5,6,7-tetrahydrooxazolo[4,5-c]pyridine

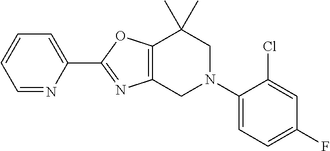

7,7-Dimethyl-2-(pyridin-2-yl)-4,5,6,7-tetrahydrooxazolo[4,5-c]pyridine (460 mg, 2.0 m mol), Pd₂(dba)₃ (92 mg, 0.1 mmol), X-phos (95 mg, 0.2 mmol), 2-chloro-4-fluoro-1-iodobenzene (768 mg, 3.0 mmol), and Na-OtBu (576 mg, 6.0 mmol) were combined in toluene (15 mL) and heated at 150° C. by microwave for 3 h. The reaction was evaporated to dryness, diluted with dichloromethane and extracted with water. The combined organic layer was dried using sodium sulfate, filtered, evaporated, and then purified by prep-TLC to obtain the title compound as a yellow solid. (6 mg, 0.87%). ¹H NMR (400 MHz, CDCl₃): δ 8.74-8.76 (m, 1H), 8.08-8.11 (m, 1H), 7.78-7.72 (m, 1H), 7.32-7.36 (m, 1H), 7.15-7.20 (m, 2H), 6.96-7.01 (m, 1H), 4.06 (s, 2H), 3.12 (s, 2H), 1.46 (s, 6H); LC/MS: m/e=358 (M+H)⁺.

### (a) tert-Butyl 3,3-dimethyl-4-oxopiperidine-1-carboxylate (I-196.1)

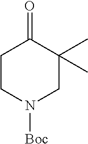

A solution of tert-butyl-4-oxopiperidine-1-carboxylate (50 g, 250 mmol) in THF (400 mL) was cooled to 0° C. Sodium hydride (22 g, 550 mmol) was added in portions. Iodomethane (90 g, 600 m mol) was added dropwise, and the reaction was allowed to gradually warm to room temperature and stirred overnight. The reaction was concentrated to dryness, dissolved in diethyl ether, washed with brine and dried over magnesium sulfate. The product was crystallized from hot pentane to give the title compound as a white solid. (23 g, 40%); LC/MS: m/e=128 (M+H-Boc)⁺.

### (b) 3,3-Dimethylpiperidin-4-one (I-196.2)

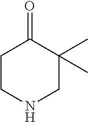

tert-Butyl 3,3-dimethyl-4-oxopiperidine-1-carboxylate (23 g, 100 mmol) was dissolved in DCM (300 mL). TFA (100 mL) was added and the reaction was stirred at RT for 3 h. The reaction was evaporated to dryness to obtain the title compound as a yellow oil. (13 g, 100%), LC/MS: m/e=128 (M+H)⁺.

### (c) 3,3-Dimethyl-1-tosylpiperidin-4-one (I-196.3)

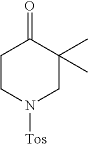

A mixture of 3,3-dimethylpiperidin-4-one (13 g, 0.1 mol), 4-methylbenzene-1-sulfonyl chloride (20.9 g, 0.11 mol) and potassium carbonate (27.6 g, 0.2 mol) in acetonitrile (300 mL) was stirred at RT for 12 h. The reaction mixture was filtered and the filtrate was concentrated to dryness under vacuum. The residue was dissolved in DCM and washed with water and brine. The organic layer was dried over sodium sulfate, filtered, and evaporated to obtain the crude product. The residue was recrystallized from petroleum ether and ethyl acetate (30/1 in volume) to obtain the title compound as a yellow solid. (22.5 g, 80%), LC/MS: m/e=282 (M+H)⁺.

### (d) 5-Bromo-3,3-dimethyl-1-tosylpiperidin-4-one (I-196.4)

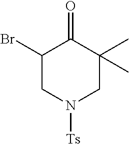

A solution of 3,3-dimethyl-1-tosylpiperidin-4-one (22.5 g, 80 mmol) in THF (400 mL), PTT (30 g, 80 mmol) was added in portions. The reaction mixture was stirred at RT for 3 h. The reaction mixture was filtered, and the filtrate was concentrated under vacuum to give the title compound as a yellow solid. (28.7 g, 100%); LC/MS: m/e=362 (M+H)⁺.

### (e) 5-Azido-3,3-dimethyl-1-tosylpiperidin-4-one (I-196.5)

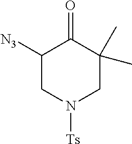

To a solution of 5-bromo-3,3-dimethyl-1-tosylpiperidin-4-one (28.7 g, 80 mmol) in dry DMF (300 mL) at 0° C., sodium azide (10.4 g, 160 mmol) was added. The reaction was stirred at 0° C. for 4 h. The reaction mixture was poured into water and extracted with ethyl acetate. The combined organic layers was washed with brine, dried, filtered and evaporated to obtain the title compound as a yellow solid. (25.8 g, 100%). LC/MS: m/e=323 (M+H)⁺.

### (f) 5-Amino-3,3-dimethyl-1-tosylpiperidin-4-ol (I-196.6)

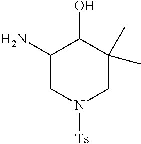

A suspension of LiAlH₄ (6.1 g, 160 mmol) in THF (300 mL) was cooled to 0° C. 5-Azido-3,3-dimethyl-1-tosylpiperidin-4-one (25.8 g, 80 mmol) in THF (100 mL) was added dropwise during which time the temperature was maintained at 0° C. After addition, the mixture was stirred at 0° C. for 30 minutes. The reaction was quenched with Na₂SO₄.10H₂O, followed by the addition of saturated sodium bicarbonate. The aqueous layer was extracted with ethyl acetate. The organic layer was dried over sodium sulfate, filtered and evaporated to obtain the crude product. The residue was purified by chromatography to give the title compound as a yellow oil (10.7 g, 45%). LC/MS: m/e=299 (M+H)⁺.

### (g) N-(4-Hydroxy-5,5-dimethyl-1-tosylpiperidin-3-yl)picolinamide (I-196.7)

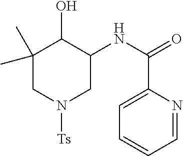

A solution of picolinic acid (3.7 g, 30 mmol) in DCM (100 mL) was added EDCI (5.8 g, 30 mmol), HOBt (4.1 g, 30 mmol), and then Et₃N (3.0 g, 30 mmol). The solution was stirred at room temperature for 30 minutes. 5-Amino-3,3-dimethyl-1-tosylpiperidin-4-ol (6.0 g, 20 mmol) in DCM (50 mL) was added, and the reaction was stirred at room temperature overnight. The reaction was diluted with DCM and washed with saturated sodium bicarbonate solution. The organic layer was dried over sodium sulfate, filtered and evaporated. Purification by chromatography gave the title compound as a yellow solid (6.1 g, 75%). LC/MS: m/e=404 (M+H)⁺.

### (h) N-(5,5-Dimethyl-4-oxo-1-tosylpiperidin-3-yl)picolinamide (I-196.8)

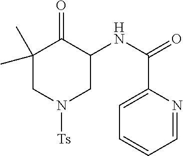

A solution of N-(4-hydroxy-5,5-dimethyl-1-tosylpiperidin-3-yl)picolinamide (6.1 g, 15 mmol) in DCM (100 mL) was added Dess-Martin periodinane (19.1 g, 45 mmol) in one portion. The reaction was stirred at room temperature for 3 h. The reaction was diluted with DCM and cooled to 0° C. Cold 0.5 N sodium hydroxide solution was added, and the biphasic mixture was stirred for 1 h. The organic layer was separated, dried over sodium sulfate and concentrated in vacuo to obtain the title compound as a yellow solid (6.1 g, 100%). LC/MS: m/e=402 (M+H)⁺.

### (i) 7,7-Dimethyl-2-(pyridin-2-yl)-5-tosyl-4,5,6,7-tetrahydrooxazolo[4,5-c]pyridine (I-196.9)

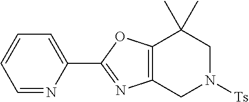

N-(5,5-Dimethyl-4-oxo-1-tosylpiperidin-3-yl)picolinamide (6.1 g, 15 m mol) was dissolved in POCl₃ (20 mL). Dioxane (150 mL) was added and the reaction was stirred at 105° C. for 10 h. The reaction was cooled to 0° C., diluted with water and extracted with DCM. The organic layer was discarded. Solid sodium bicarbonate was added to the aqueous layer until basic, and the mixture was diluted with DCM. The organic layer was separated and dried over sodium sulfate. Purification by chromatography gave the title compound as a yellow solid. (4.7 g, 82%), LC/MS: m/e=384 (M+H)⁺.

### (j) 7,7-Dimethyl-2-(pyridin-2-yl)-4,5,6,7-tetrahydrooxazolo[4,5-c]pyridine (I-196.10)

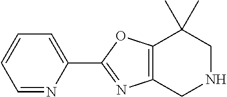

7,7-Dimethyl-2-(pyridin-2-yl)-5-tosyl-4,5,6,7-tetrahydrooxazolo[4,5-c]pyridine (3.8 g, 10 mmol) was dissolved in 48% aqueous HBr (100 mL) and heated to 90° C. for 6 h. The reaction was cooled to room temperature and extracted with tert-butyl methyl ether. The aqueous layer was adjusted to pH=13 using 6N sodium hydroxide, then extracted with DCM. The combined organic layers were dried over sodium sulfate, filtered and evaporated to obtain the title compound as a yellow solid (920 mg, 40%). LC/MS: m/e=230 (M+H)⁺.

### 197. Compound 197: 5-(3,4-Difluorophenyl)-2-(pyridin-2-yl)-5,6,7,8-tetrahydro-4H-oxazolo[4,5-c]azepine

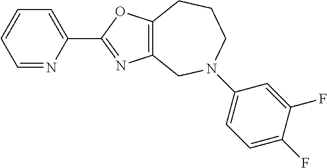

The title compound was prepared via the procedure used for the preparation of Compound 98, substituting 4-bromo-1,2-difluorobenzene for 3-bromo-5-fluorobenzonitrile. Preparative HPLC afforded a solid (72 mg, 73%). ¹H NMR (400 MHz, CDCl₃): δ 8.71 (d, 1H), 8.05 (d, 1H), 7.80 (td, 1H), 7.32˜7.35 (m, 1H), 6.96 (q, 1H), 6.62 (qd, 1H), 6.47˜6.50 (m, 1H), 4.48 (s, 2H), 3.76˜3.79 (m, 2H), 2.99 (t, 2H), 1.93˜1.99 (m, 2H); LC/MS: m/e=328 (M+H)⁺.

### 198. Compound 198: 5-(5-Chloropyridin-3-yl)-2-(pyridin-2-yl)-5,6,7,8-tetrahydro-4H-oxazolo[4,5-c]azepine

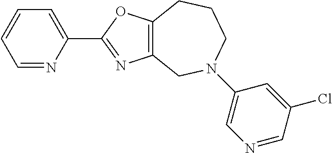

The title compound was prepared via the procedure used for the preparation of Compound 98, substituting 3-bromo-5-chloropyridine for 3-bromo-5-fluorobenzonitrile. Preparative HPLC afforded a solid (52 mg, 53%). ¹H NMR (400 MHz, CDCl₃): δ 8.70 (d, 1H), 8.12 (d, 1H), 8.05 (d, 1H), 7.91 (d, 1H), 7.81 (td, 1H), 7.33˜7.36 (m, 1H), 7.09 (t, 1H), 4.54 (s, 2H), 3.82˜3.85 (m, 2H), 3.02 (t, 2H), 1.98˜2.04 (m, 2H); LC/MS: m/e=327 (M+H)⁺.

### 199. Compound 199: 5-(3-Chloro-5-fluorophenyl)-2-(pyridin-2-yl)-5,6,7,8-tetrahydro-4H-oxazolo[4,5-c]azepine

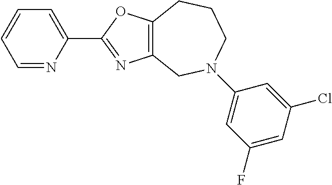

The title compound was prepared via the procedure used for the preparation of Compound 98, substituting 1-bromo-3-chloro-5-fluorobenzene for 3-bromo-5-fluorobenzonitrile. Preparative HPLC afforded a solid (82 mg, 80%). ¹H NMR (400 MHz, CDCl₃): δ 8.71 (d, 1H), 8.05 (d, 1H), 7.80 (td, 1H), 7.32˜7.36 (m, 1H), 6.58 (s, 1H), 6.43 (d, 1H), 6.40 (m, 1H), 4.51 (s, 2H), 3.76˜3.78 (m, 2H), 3.00 (t, 2H), 1.99˜2.02 (m, 2H); LC/MS: m/e=344 (M+H)⁺.

### 200. Compound 200: 5-(3,4-Difluorophenyl)-2-(pyrimidin-2-yl)-5,6,7,8-tetrahydro-4H-oxazolo[4,5-c]azepine

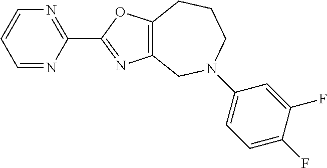

The title compound was prepared via the procedure used for the preparation of Compound 98, substituting pyrimidine-2-carboxylic acid for picolinic acid, and substituting 4-bromo-1,2-difluorobenzene for 3-bromo-5-fluorobenzonitrile. Preparative HPLC afforded a solid (42 mg, 69%). ¹H NMR (400 MHz, CDCl₃): δ 8.88 (d, 2H), 7.35 (t, 1H), 6.95 (q, 1H), 6.60˜6.65 (dq, 1H), 6.50 (m, 1H), 4.53 (s, 2H), 3.81˜3.78 (m, 2H), 3.03 (t, 2H), 1.98˜1.96 (m, 2H); LC/MS: m/e=329 (M+H)⁺.

### 201. Compound 201: 5-(5-Chloropyridin-3-yl)-2-(pyrimidin-2-yl)-5,6,7,8-tetrahydro-4H-oxazolo[4,5-c]azepine

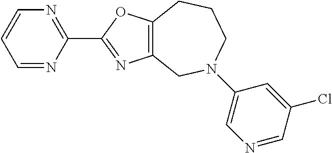

The title compound was prepared via the procedure used for the preparation of Compound 98, substituting pyrimidine-2-carboxylic acid for picolinic acid, and substituting 3-bromo-5-chloropyridine for 3-bromo-5-fluorobenzonitrile. Preparative HPLC afforded a solid (30 mg, 50%). ¹H NMR (400 MHz, CDCl₃): δ 8.88 (d, 2H), 8.12 (d, 1H), 7.90 (d, 1H), 7.34 (t, 1H), 7.09 (t, 1H), 4.58 (s, 2H), 3.86˜3.83 (m, 2H), 3.05 (t, 2H), 2.03˜2.00 (m, 2H); LC/MS: m/e=328 (M+H)⁺.

### 202. Compound 202: 5-(3-Chloro-5-fluorophenyl)-2-(pyrimidin-2-yl)-5,6,7,8-tetrahydro-4H-oxazolo[4,5-c]azepine

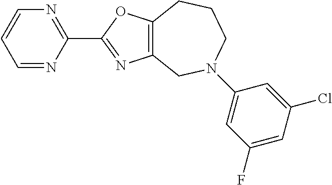

The title compound was prepared via the procedure used for the preparation of Compound 98, substituting pyrimidine-2-carboxylic acid for picolinic acid, and substituting 1-bromo-3-chloro-5-fluorobenzene for 3-bromo-5-fluorobenzonitrile. Preparative HPLC afforded a solid (9 mg, 14%). ¹H NMR (400 MHz, CDCl₃): δ 8.88 (d, 2H), 7.34 (t, 1H), 6.58 (s, 1H), 6.43-6.39 (m, 2H), 4.54 (s, 2H), 3.79-3.77 (m, 2H), 3.03 (t, 2H), 2.02-2.00 (m, 2H); LC/MS: m/e=345 (M+H)⁺.

### 203. Compound 203: 3-Fluoro-5-(2-(pyrimidin-2-yl)-7,8-dihydro-4H-oxazolo[4,5-c]azepin-5(6H)-yl)benzonitrile

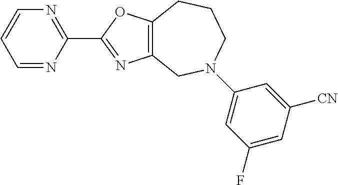

The title compound was prepared via the procedure used for the preparation of Compound 98, substituting pyrimidine-2-carboxylic acid for picolinic acid. Preparative HPLC afforded a solid (25 mg, 40%). ¹H NMR (400 MHz, CDCl₃): δ 8.88 (d, 2H), 7.35 (t, 1H), 6.83˜6.82 (s, 1H), 6.72 (td, 1H), 6.65˜6.22 (m, 1H), 4.56 (s, 2H), 3.83˜3.81 (m, 2H), 3.05 (t, 2H), 2.01˜1.99 (m, 2H); LC/MS: m/e=336 (M+H)⁺.

### 204. Compound 204: 5-(3-Chloro-4-fluorophenyl)-2-(pyrimidin-2-yl)-5,6,7,8-tetrahydro-4H-oxazolo[4,5-c]azepine

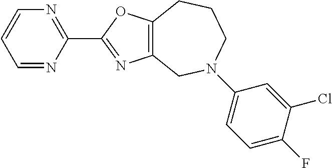

The title compound was prepared via the procedure used for the preparation of Compound 98, substituting pyrimidine-2-carboxylic acid for picolinic acid, and substituting 4-bromo-2-chloro-1-fluorobenzene for 3-bromo-5-fluorobenzonitrile. Preparative HPLC afforded a solid (40 mg, 63%). ¹H NMR (400 MHz, CDCl₃): δ 8.88 (d, 2H), 7.34 (t, 1H), 6.94 (t, 1H), 6.83˜6.81 (q, 1H), 6.65 (td, 1H), 4.52 (s, 2H), 3.80˜3.78 (m, 2H), 3.02 (t, 2H), 1.98˜1.95 (m, 2H); LC/MS: m/e=345 (M+H)⁺.

### 205. Compound 205: 5-(3-Chloro-5-fluorophenyl)-2-(5-fluoropyridin-2-yl)-5,6,7,8-tetrahydro-4H-oxazolo[4,5-c]azepine

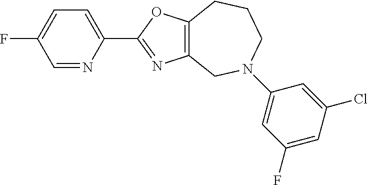

The title compound was prepared via the procedure used for the preparation of Compound 98, substituting 5-fluoropicolinic acid for picolinic acid, and substituting 1-bromo-3-chloro-5-fluorobenzene for 3-bromo-5-fluorobenzonitrile. Preparative HPLC afforded a solid (57 mg, 44%). ¹H NMR (400 MHz, CDCl₃): δ 8.56˜8.55 (m, 1H), 8.10˜8.06 (m, 1H), 7.55˜7.50 (m, 1H), 6.58˜6.57 (m, 1H), 6.43˜6.39 (m, 2H), 4.49 (s, 2H), 3.78˜3.76 (m, 2H), 3.01˜2.98 (m, 2H), 2.02˜1.99 (m, 2H); LC/MS: m/e=362 (M+H)⁺.

### 206. Compound 206: 3-Fluoro-5-(2-(pyrimidin-5-yl)-7,8-dihydro-4H-oxazolo[4,5-c]azepin-5(6H)-yl)benzonitrile

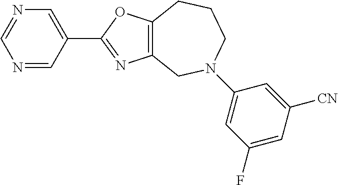

The title compound was prepared via the procedure used for the preparation of Compound 98, substituting pyrimidine-5-carboxylic acid for picolinic acid. Preparative HPLC afforded a solid (20 mg, 32%). ¹H NMR (400 MHz, CDCl₃): δ 9.28 (s, 2H), 9.26 (s, 1H), 6.83˜6.82 (m, 1H), 6.73˜6.66 (m, 2H), 4.52 (s, 2H), 3.84˜3.81 (m, 2H), 3.02˜2.99 (m, 2H), 2.05˜2.00 (m, 2H); LC/MS: m/e=336 (M+H)⁺.

### 207. Compound 207: 2-(2-Methylthiazol-4-yl)-5-(pyridin-2-yl)-5,6,7,8-tetrahydro-4H-oxazolo[4,5-c]azepine

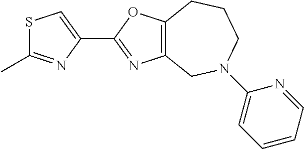

The title compound was prepared via the procedure used for the preparation of Compound 98, substituting 2-methylthiazole-4-carboxylic acid for picolinic acid, and substituting 2-bromopyridine for 3-bromo-5-fluorobenzonitrile. Preparative HPLC afforded a solid (80 mg, 60%). ¹H NMR (400 MHz, CDCl₃): δ 8.14˜8.16 (m, 1H), 7.69 (s, 1H), 7.40 (dt, 1H), 6.62 (d, 1H), 6.51˜6.54 (m, 1H), 4.72 (s, 2H), 3.92˜3.95 (m, 2H), 2.91 (t, 2H), 2.76 (s, 3H), 1.98˜2.04 (m, 2H); LC/MS: m/e=313 (M+H)⁺.

### 208. Compound 208: 5-(2-Methylthiazol-4-yl)-2-(pyridin-2-yl)-5,6,7,8-tetrahydro-4H-oxazolo[4,5-c]azepine

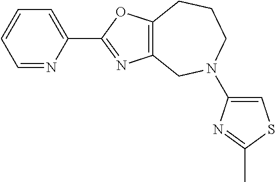

The title compound was prepared via the procedure used for the preparation of Compound 98, substituting 2-methylthiazole-4-carboxylic acid for picolinic acid, and substituting 4-bromo-2-methylthiazole for 3-bromo-5-fluorobenzonitrile. Preparative HPLC afforded a solid (28 mg, 15%). ¹H NMR (400 MHz, CDCl₃): δ 8.61˜8.63 (m, 1H), 7.98 (td, 1H), 7.71 (td, 1H), 7.23˜7.26 (m, 1H), 5.53 (s, 1H), 4.49 (s, 2H), 3.80˜3.82 (m, 2H), 2.90 (t, 2H), 2.52 (s, 3H), 1.87˜1.90 (m, 2H); LC/MS: m/e=313 (M+H)⁺.

### 209. Compound 209: 5-(3,5-Dichlorophenyl)-2-(pyridin-2-yl)-5,6,7,8-tetrahydro-4H-oxazolo[4,5-c]azepine

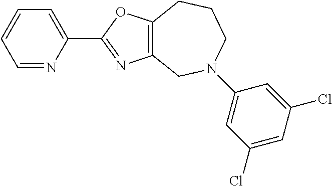

The title compound was prepared via the procedure used for the preparation of Compound 98, substituting 1-bromo-3,5-dichlorobenzene for 3-bromo-5-fluorobenzonitrile. Preparative HPLC afforded a solid (130 mg, 60%). ¹H NMR (400 MHz, CDCl₃): δ 8.71 (d, 1H), 8.06 (d, 1H), 7.80 (dt, 1H), 7.32˜7.36 (m, 1H), 6.68 (s, 3H), 4.51 (s, 2H), 3.76˜3.78 (m, 2H), 3.00 (t, 2H), 2.00˜2.04 (m, 2H); LC/MS: m/e=360 (M+H)⁺.

### 210. Compound 210: 2, 4,6-d₃-3-Fluoro-5-(2-(pyridin-2-yl)-7,8-dihydro-4H-oxazolo[4,5-c]azepin-5(6H)-yl)benzonitrile

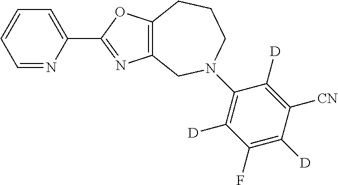

The title compound was prepared via the procedure used for the preparation of Compound 98, substituting 2,4,6-d₃-3-bromo-5-fluorobenzonitrile (purchased from C/D/N Isotopes, Inc.) for 3-bromo-5-fluorobenzonitrile. Preparative HPLC afforded a solid (160 mg, 47%). ¹H NMR (400 MHz, CDCl₃): δ 8.70˜8.72 (m, 1H), 8.06 (dd, 1H), 7.81 (dt, 1H), 7.33˜7.36 (m, 1H), 4.52 (s, 2H), 3.80˜3.82 (m, 2H), 3.02 (t, 2H), 1.97˜2.03 (m, 2H); LC/MS: m/e=338 (M+H)⁺.

### 211. Compound 211: 5-(2-Chlorophenyl)-2-(pyridin-2-yl)-5,6,7,8-tetrahydro-4H-oxazolo[4,5-c]azepine

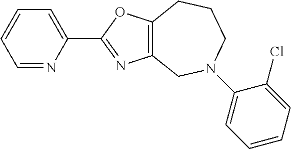

The title compound was prepared via the procedure used for the preparation of Compound 98, substituting 1-bromo-2-chlorobenzene for 3-bromo-5-fluorobenzonitrile. Preparative HPLC afforded a solid (9 mg, 27%). ¹H NMR (400 MHz, CDCl₃): δ 8.71˜8.73 (m, 1H), 8.07 (d, 1H), 7.80 (dt, 1H), 7.32˜7.37 (m, 2H), 7.09˜7.18 (m, 2H), 6.92˜6.96 (m, 1H), 4.40 (s, 2H), 3.56˜3.59 (m, 2H), 3.03 (t, 2H), 2.00˜2.05 (m, 2H); LC/MS: m/e=326 (M+H)⁺.

### 212. Compound 212: 5-(4-Chlorophenyl)-2-(pyridin-2-yl)-5,6,7,8-tetrahydro-4H-oxazolo[4,5-c]azepine

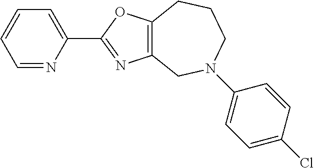

The title compound was prepared via the procedure used for the preparation of Compound 98, substituting 1-bromo-4-chlorobenzene for 3-bromo-5-fluorobenzonitrile. Preparative HPLC afforded a solid (80 mg, 62%). ¹H NMR (400 MHz, CDCl₃): δ 8.70 (td, 1H), 8.03 (d, 1H), 7.80 (dt, 1H), 7.32˜7.35 (m, 1H), 7.12˜7.16 (m, 2H), 6.75˜6.79 (m, 2H), 4.52 (s, 2H), 3.80˜3.83 (m, 2H), 2.96 (t, 2H), 1.94˜1.99 (m, 2H); LC/MS: m/e=326 (M+H)⁺.

### 213. Compound 213: 3-Methoxy-5-(2-(pyridin-2-yl)-7,8-dihydro-4H-oxazolo[4,5-c]azepin-5(6H)-yl)benzonitrile

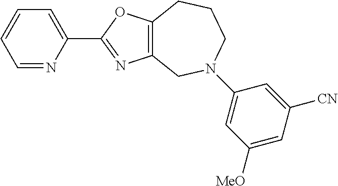

The title compound was prepared via the procedure used for the preparation of Compound 98, substituting 3-bromo-5-methoxybenzonitrile for 3-bromo-5-fluorobenzonitrile. Preparative HPLC afforded a solid (86 mg, 67%). ¹H NMR (400 MHz, CDCl₃): δ 8.70˜8.67 (m, 1H), 8.06˜8.04 (m, 1H), 7.82˜7.78 (m, 1H), 7.35˜7.26 (m, 1H), 6.70˜6.69 (m, 1H), 6.58˜6.48 (m, 2H), 4.52 (s, 2H), 3.81˜3.78 (m, 2H), 3.01˜2.98 (m, 2H), 1.99˜1.97 (m, 2H); LC/MS: m/e=347 (M+H)⁺.

### 214. Compound 214: 3-Hydroxy-5-(2-(pyridin-2-yl)-7,8-dihydro-4H-oxazolo[4,5-c]azepin-5(6H)-yl)benzonitrile

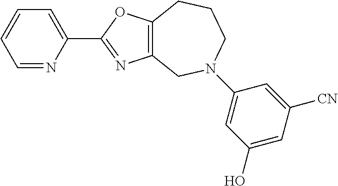

Boron tribromide (47 mg, 0.40 mmol) was added to a solution of 3-methoxy-5-(2-(pyridin-2-yl)-7,8-dihydro-4H-oxazolo[4,5-c]azepin-5(6H)-yl)benzonitrile (70 mg, 0.20 mmol) in DCM (6 mL), and stirred at room temperature for 48 h. Methanol (2 mL) was added and the mixture was concentrated to give the crude product. Purification by prep-TLC (PE:EA=1:1) gave the product as a yellow solid (12 mg, 18%). ¹H NMR (400 MHz, CDCl₃): δ 8.57˜8.56 (m, 1H), 7.91˜7.89 (m, 1H), 7.80˜7.78 (m, 1H), 7.32˜7.27 (m, 1H), 6.54 (s, 1H), 6.45 (s, 1H), 6.24 (s, 1H), 4.34 (s, 2H), 3.67˜3.66 (m, 2H), 2.90˜2.87 (m, 2H), 1.83 (m, 2H). LC/MS: m/e=333 (M+H)⁺.

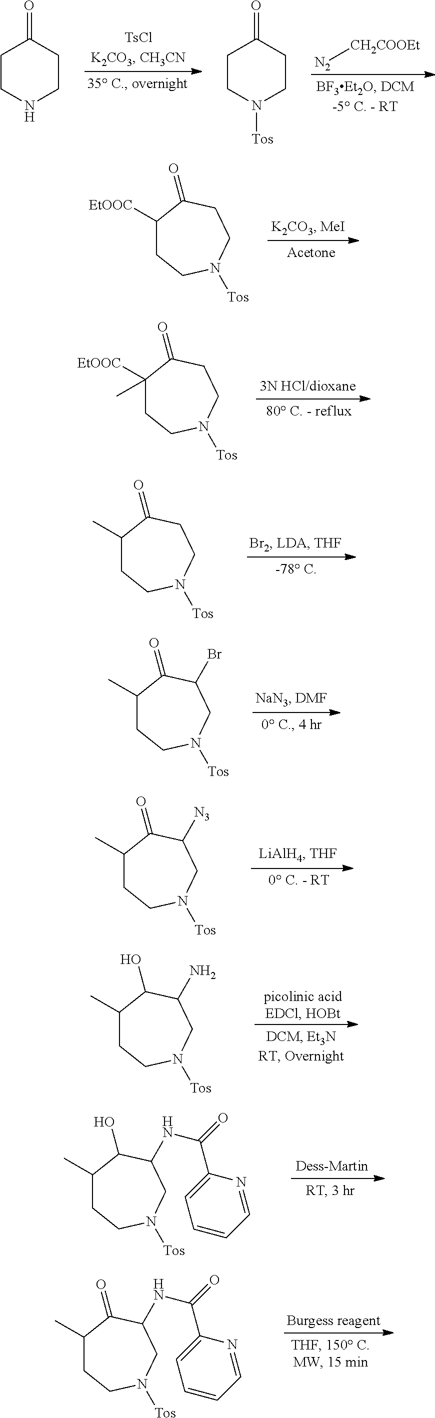

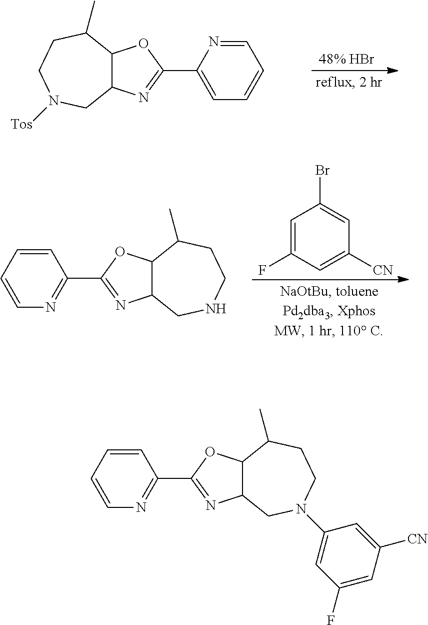

### 215. Compound 215: 3-Fluoro-5-(8-methyl-2-(pyridin-2-yl)-7,8-dihydro-4H-oxazolo[4,5-c]azepin-5(6H)-yl)benzonitrile

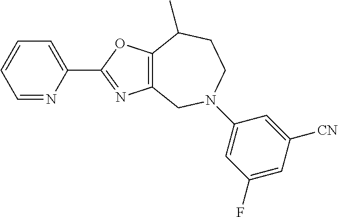

A mixture of 8-methyl-2-(pyridin-2-yl)-5,6,7,8-tetrahydro-4H-oxazolo[4,5-c]azepine (18 mg, 0.08 mmol), Pd₂(dba)₃ (18 mg, 0.02 mmol), Xphos (19 mg, 0.04 mmol), 3-bromo-5-fluorobenzonitrile (20 mg, 0.1 mmol), and Na-OtBu (30 mg, 0.3 mmol) in toluene (1 mL) was heated to 110° C. by microwave and stirred for 1 h. The mixture was evaporated to dryness. The residue was diluted with water and extracted with dichloromethane. The combined organic layers were dried with sodium sulfate, filtered, evaporated and purified by prep-TLC to obtain the title compound as a yellow solid (8 mg, 28%). ¹H NMR (400 MHz, MeOH-d₄): δ 8.63 (s, 1H), 8.04-8.13 (m, 2H), 7.56 (s, 1H), 6.97 (s, 1H), 6.85-6.89 (s, 1H), 6.71-6.72 (d, 1H), 4.51-4.63 (m, 2H), 3.99-4.05 (m, 1H), 3.75-3.81 (m, 1H), 3.27-3.30 (m, 1H), 2.03-2.12 (m, 1H), 1.76-1.84 (m, 1H), 1.39-1.42 (m, 1H); LC/MS: m/e=349 (M+H)⁺.

### (a) 1-Tosylpiperidin-4-one (I-215.1)

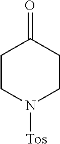

A mixture of piperidin-4-one hydrochloride (40.5 g, 0.3 mol), 4-methylbenzene-1-sulfonyl chloride (62.7 g, 0.33 mol) and potassium carbonate (82.8 g, 0.6 mol) in acetonitrile (800 mL) was stirred at 35° C. for 24 hours. The reaction mixture was filtered and the filtrate was concentrated under vacuum. The residue was dissolved in dichloromethane and washed with water and brine. The organic layer was dried over sodium sulfate, filtered, and evaporated to obtain the crude product. Recrystallization with petrol ether and ethyl acetate (15/1 in volume) gave the title compound (64 g, 85%). LC/MS: m/e=254 (M+H)⁺.

### (b) Ethyl 5-oxo-1-tosylazepane-4-carboxylate (I-215.2)

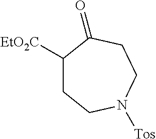

A solution of BF₃.Et₂O (18.5 g, 130 mmol) in dichloromethane (50 mL) was added dropwise to a solution of 1-tosylpiperidin-4-one (25.3 g, 100 mmol) in dry DCM (300 mL) at −5° C. under argon. The temperature of the reaction was maintained between −5 and 3° C. The reaction was stirred at −5° C. for 45 minutes after which a solution of ethyl diazoacetate (15.0 g, 130 mmol) in DCM (100 mL) was added dropwise over a 90 minute period. During this time, nitrogen evolution was observed. The solution was stirred at −5° C. for 1 h, after which it was diluted with water slowly and then stirred at room temperature for 30 minutes. The organic layer was separated, dried over sodium sulfate, filtered and concentrated in vacuo to give a pale yellow solid. Recrystallization from ethyl acetate gave the title compound as a yellow solid. (23.7 g, 70%). LC/MS: m/e=340 (M+H)⁺.

### (c) Ethyl 4-methyl-5-oxo-1-tosylazepane-4-carboxylate (I-215.3)

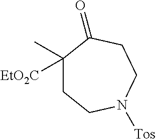

Ethyl 5-oxo-1-tosylazepane-4-carboxylate (23.7 g, 70 mmol) and potassium carbonate (48.3 g, 350 mmol) were combined and stirred in acetone (300 mL). Methyl iodide (10.9 g, 77 mmol) was added dropwise and the mixture was stirred at RT for 16 h. The reaction was filtered and the filtrate dried over sodium sulfate. Purification of the residue by column chromatography gave the title compound as a yellow solid (17.3 g, 70%). LC/MS: m/e=354 (M+H)⁺.

### (d) 5-Methyl-1-tosylazepan-4-one (I-215.4)

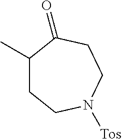

A suspension of ethyl 4-methyl-5-oxo-1-tosylazepane-4-carboxylate (17.3 g, 49 mmol) in dioxane (400 mL) was heated to 80° C., whereupon the solution became clear. 3N HCl (290 mL) was added and the resulting solution was heated to 100° C. for 6 h. The dioxane was removed by evaporation, and the aqueous layer was extracted with DCM. The combined organic layers were dried, filtered and evaporated to obtain the crude product. Purification by column chromatography gave the title compound as a yellow solid (5.6 g, 40%); LC/MS: m/e=282 (M+H)⁺.

### (e) 3-Bromo-5-methyl-1-tosylazepan-4-one (I-215.5)

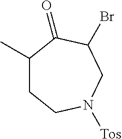

LDA (10 mL, 20 mmol) in dry THF (20 mL) was added dropwise to a solution of 5-methyl-1-tosylazepan-4-one (5.6 g, 20 mmol) in dry THF (100 mL) at −78° C. Bromine (3.2 g, 20 mmol) was added dropwise over a period of 1 h. The reaction was stirred at −78° C. for 3 h. Saturated NaHCO3 was added and the resulting biphasic solution was stirred for 30 minutes. The layers were then separated and the aqueous layer was extracted with DCM. The combined organic layers were dried over sodium sulfate, filtered and evaporated to obtain the crude product. Purification by column chromatography gave the title compound as a yellow solid (2.1 g, 30%); LC/MS: m/e=363 (M+H)⁺.

### (f) 3-Azido-5-methyl-1-tosylazepan-4-one (I-215.6)

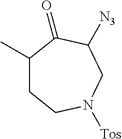

Sodium azide (754 mg, 11.6 mmol) was added to a solution of 3-bromo-5-methyl-1-tosylazepan-4-one (2.1 g, 5.8 mmol) in dry DMF (40 mL) at 0° C. The reaction was stirred at 0° C. for 4 h. The reaction was poured into ice and extracted with ethyl acetate. The combined organic layers were washed with brine, dried, filtered and evaporated to give the title compound as a yellow oil. (934 mg, 50%); LC/MS: m/e=323 (M+H)⁺.

### (g) 3-Amino-5-methyl-1-tosylazepan-4-ol (I-215.7)

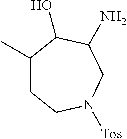

A suspension of LiAlH₄ (152 mg, 4.0 mmol) in THF (15 mL) was cooled to 0° C. 3-Azido-5-methyl-1-tosylazepan-4-one (636 mg, 2.0 mmol) in THF (15 mL) was added dropwise during which time the temperature was maintained at 0° C. The reaction was stirred at 0° C. for 30 minutes, then quenched with Na₂SO₄.10H₂O and saturated sodium bicarbonate. The aqueous layer was extracted with ethyl acetate, dried over sodium sulfate, filtered and evaporated to obtain the crude product. The residue was purified by chromatography to give the title compound as a yellow oil (180 mg, 30%); LC/MS: m/e=299 (M+H)⁺.

### (h) N-(4-Hydroxy-5-methyl-1-tosylazepan-3-yl)picolinamide (I-215.8)

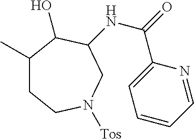

Picolinic acid (111 mg, 0.9 mmol) was dissolved in DCM (5 mL). EDCI (174 mg, 0.9 mmol), HOBt (122 mg, 0.9 mmol), and TEA (91 mg, 0.9 mmol) were added, and the solution was stirred at room temperature for 30 minutes. 3-Amino-5-methyl-1-tosylazepan-4-ol (180 mg, 0.6 mmol) in DCM (5 mL) was added and the reaction was stirred at room temperature overnight. The mixture was diluted with DCM and washed with saturated sodium bicarbonate solution. The organic layer was dried over sodium sulfate, filtered and evaporated. Purification by prep-TLC gave the title compound as a yellow oil (164 mg, 70%); LC/MS: m/e=404 (M+H)⁺.

### (i) N-(5-Methyl-4-oxo-1-tosylazepan-3-yl)picolinamide (1-215.9)

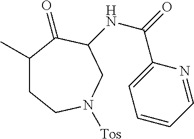

Dess-Martin periodinane (520 mg, 1.2 mmol) was added to a solution of N-(4-hydroxy-5-methyl-1-tosylazepan-3-yl)picolinamide (164 mg, 0.4 mmol) in DCM (10 mL). The reaction was stirred at room temperature for 3 h. The reaction was diluted with DCM and cooled to 0° C. Cold 0.5 N sodium hydroxide solution was added and the biphasic mixture was stirred for 1 h. The organic layer was separated, dried over sodium sulfate and concentrated in vacuo to obtain the title compound as a yellow oil (164 mg, 100%); LC/MS: m/e=402 (M+H)⁺.

### (j) 8-Methyl-2-(pyridin-2-yl)-5-tosyl-5,6,7,8-tetrahydro-4H-oxazolo[4,5-c]azepine (I-215.10)

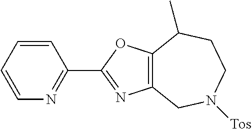

A mixture of N-(5-methyl-4-oxo-1-tosylazepan-3-yl)picolinamide (164 mg, 0.4 mmol) and Burgess Reagent (286 mg, 1.2 mmol) in dry THF (2 mL) was heated to 150° C. by microwave and stirred for 30 minutes. The reaction mixture was diluted with water, and extracted with DCM. The organic layer was dried over sodium sulfate and evaporated to dryness. The crude product was purified by prep-TLC to obtain the title compound as a yellow oil. (76 mg, 50%); LC/MS: m/e=384 (M+H)⁺.

### (k) 8-Methyl-2-(pyridin-2-yl)-5,6,7,8-tetrahydro-4H-oxazolo[4,5-c]azepine (I-215.11)

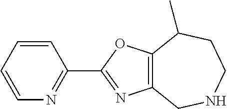

A solution of 8-methyl-2-(pyridin-2-yl)-5-tosyl-5,6,7,8-tetrahydro-4H-oxazolo[4,5-c]azepine (76 mg, 0.2 mmol) in 48% aqueous HBr (1 mL) was heated to 100° C. and stirred for 1 h. The reaction was cooled to room temperature and extracted with tert-butyl methyl ether. The aqueous layer was adjusted to pH 13 with sodium hydroxide and extracted with DCM. The combined organic layers were dried over sodium sulfate, filtered and evaporated to give the title compound as a yellow oil. (18 mg, 40%); LC/MS: m/e=230 (M+H)⁺.

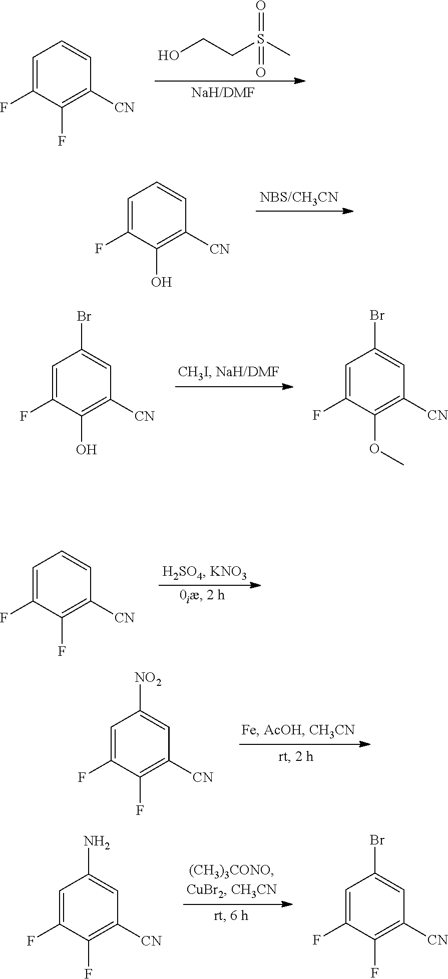

### 216. Compound 216: 2,3-Difluoro-5-(2-(pyridin-2-yl)-7,8-dihydro-4H-oxazolo[4,5-c]azepin-5(6H)-yl)benzonitrile

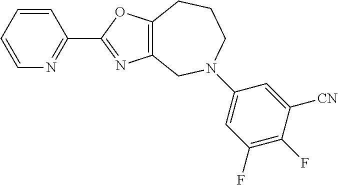

A microwave tube was flushed with argon. Sodium t-butoxide (9 mg, 0.092 mmol), Pd₂(dba)₃ (2 mg, 0.0023 mmol), Xantphos (2 mg, 0.0046 mmol), 2-(pyridin-2-yl)-5,6,7,8-tetrahydro-4H-oxazolo[4,5-c]azepine (10 mg, 0.046 mmol) and 5-bromo-2,3-difluorobenzonitrile (13 mg, 0.060 mmol) were combined in toluene (2 mL). The reaction mixture was heated to 100° C. using microwave for 1 h. The reaction mixture was diluted with ethyl acetate and washed with brine. The combined organic layers were dried over sodium sulfate, filtered and evaporated to dryness. Purification by prep-TLC to gave the title compound (8 mg, 20%). ¹H NMR (400 MHz, CDCl₃): δ 8.74 (m, 1H), 8.07˜8.05 (m, 1H), 7.85˜8.81 (m, 1H), 7.39˜7.36 (m, 1H), 7.27˜7.15 (m, 1H), 6.69˜6.65 (m, 1H), 4.48 (s, 2H), 3.97˜3.94 (m, 2H), 3.04˜3.01 (t, J=6.2 Hz, 2H), 2.06˜2.02 (m, 2H); LC/MS: m/e=353 (M+H)⁺.

### (a) 3-Fluoro-2-hydroxybenzonitrile (I-216.1)

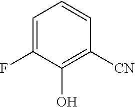

To a solution of 2,3-difluorobenzonitrile (1.39 g, 10 mmol) and 2-(methylsulfonyl)ethanol (1.24 g, 10 mmol) in DMF (30 mL) was added sodium hydride (960 mg, 50% in mineral oil, 20 mmol) in small portions at room temperature. After stirring at room temperature for 30 minutes, the reaction was poured onto ice water (100 mL). The aqueous solution was extracted with ethyl ether (60 mL) twice. The aqueous layer was acidified with conc. HCl to pH 2, then extracted with ether (2×60 mL). The organic layer was dried over sodium sulfate, filtered and evaporated to dryness to give the title compound as a pale yellow oil (2.0 g, 100%). LC/MS: m/e=138 (M+H)⁺.

### (b) 5-Bromo-3-fluoro-2-hydroxybenzonitrile (I-216.2)

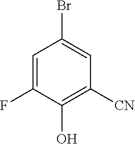

NBS (1.78 g, 10 mmol) was added to a solution of 3-fluoro-2-hydroxybenzonitrile (1.37 g, 10 mmol) in acetonitrile (40 mL) in one portion at room temperature. The reaction was stirred at room temperature for 1 hour. The solvent was removed in vacuo and the residue was diluted with saturated sodium carbonate solution (80 mL). The aqueous layer was washed with ether (2×40 mL). The aqueous layer was retained, acidified to pH 2, and extracted with ether (2×80 mL). The organic layer was dried over sodium sulfate, filtered and evaporated to dryness to give the title compound as a pale yellow solid (2.1 g, 98%). LC/MS: m/e=216 (M+H)⁺.

### (c) 5-Bromo-3-fluoro-2-methoxybenzonitrile (I-216.3)

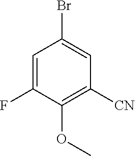

Sodium hydride (240 mg, 50% in mineral oil, 5 mmol) was added portionwise to a solution of 5-bromo-3-fluoro-2-hydroxybenzonitrile (540 mg, 2.5 mmol) in DMF (20 mL). The suspension was stirred at room temperature for 20 minutes. Iodomethane (710 mg, 5 mmol) was added and the mixture was stirred at room temperature for 12 hours. The mixture was poured onto ice water (80 mL). A precipitate appeared that was filtered and dried in vacuo to give the title compound as a yellow solid (470 mg, 82%). LC/MS: m/e=230 (M+H)⁺.

### (d) 2,3-Difluoro-5-nitrobenzonitrile (I-216.4)

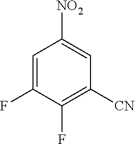

Potassium nitrate (404 mg, 4.0 mmol) to a solution of 2,3-difluorobenzonitrile (278 mg, 2.0 mmol) in sulfuric acid (2 mL) at 0° C. After stiffing at 0° C. for 2 h the reaction was quenched with ice water (5 mL). The mixture was extracted with ethyl acetate (3×10 mL). The organic layer was dried and concentrated to give the crude product which was purified by silica gel (PE:EA=40:1) to give the title compound as a yellow solid. (40 mg, 11%). ¹H NMR (400 MHz, CDCl₃): δ 8.25˜8.22 (m, 1H), 7.69˜7.63 (m, 1H). LC/MS: m/e=185 (M+H)⁺.

### (e) 5-Amino-2,3-difluorobenzonitrile (I-216.5)

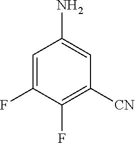

A solution of 2,3-difluoro-5-nitrobenzonitrile (35 mg, 0.19 mmol) in acetonitrile (2 mL) was cooled to 0° C. Acetic acid (228 mg, 3.80 mmol) and iron filings (75 mg, 1.33 mmol) were added, and the mixture was stirred at room temperature for 2 h. The reaction mixture was filtered and the filtrate was concentrated to give the crude product which was purified by prep-TLC (PE:EA=2:1) to give the title compound as a yellow solid. (14 mg, 48%); LC/MS: m/e=155 (M+H)⁺.

### (f) 5-Bromo-2,3-difluorobenzonitrile (I-216.6)

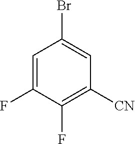

A solution of copper (I) bromide (25 mg, 0.11 mmol) in acetonitrile (1 mL) was purged with nitrogen and cooled to 0° C. Tert-butyl nitrite (15 mg, 0.14 mmol) and a solution of 5-amino-2,3-difluorobenzonitrile (14 mg, 0.090 mmol) in acetonitrile (1 mL) were added, and the reaction was stirred at room temperature for 6 h. The solvent was removed in vacuo and the residue was partitioned between ethyl acetate (5 mL) and saturated sodium bicarbonate aqueous solution (5 mL). The ethyl acetate layer was dried and concentrated to give the title compound as a yellow solid (18 mg, 93%); ¹H NMR (400 MHz, CDCl₃): δ 7.48˜7.45 (m, 1H), 7.38˜7.27 (m, 1H); LC/MS: m/e=218 (M+H)⁺.

### 217. Compound 217: 3-Fluoro-2-methoxy-5-(2-(pyridin-2-yl)-7,8-dihydro-4H-oxazolo[4,5-c]azepin-5(6H)-yl)benzonitrile

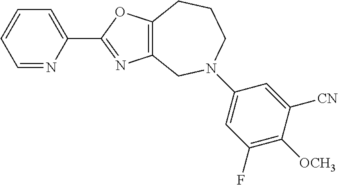

The title compound was synthesized using the method described for the synthesis of Compound A-95, substituting 5-bromo-3-fluoro-2-methoxybenzonitrile for 5-bromo-2,3-difluorobenzonitrile. Column chromatography afforded a yellow solid (100 mg, 61%) ¹H NMR (400 MHz, CDCl₃): δ 8.70˜8.72 (m, 1H), 8.06 (d, 1H), 7.82 (dt, 1H), 7.33˜7.37 (m, 1H), 6.78 (dd, 1H), 7.00 (m, 1H), 4.48 (s, 2H), 3.95 (s, 3H), 3.77˜3.79 (m, 2H), 3.01 (t, 2H), 1.96˜1.98 (m, 2H). LC/MS: m/e=365 (M+H)⁺.

### 218. Compound 218: 3-Fluoro-2-hydroxy-5-(2-(pyridin-2-yl)-7,8-dihydro-4H-oxazolo[4,5-c]azepin-5(6H)-yl)benzonitrile

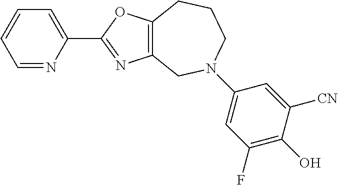

3-Fluoro-2-methoxy-5-(2-(pyridin-2-yl)-7,8-dihydro-4H-oxazolo[4,5-c]azepin-5(6H)-yl)benzonitrile (66 mg, 0.18 mmol) was dissolved in dichloromethane (20 mL) and cooled to 0° C. Boron tribromide (670 mg, 2.7 mmol) was added dropwise. The suspension was stirred at room temperature for 41 hours. The reaction mixture was cooled to −50° C. and quenched with water. The mixture was partitioned between DCM (30 mL) and water (15 mL). The biphasic mixture was adjusted to pH 9 with saturated sodium bicarbonate and the organic layer was separated. The aqueous layer was extracted with DCM (2×50 mL). The combined organic layers were dried over sodium sulfate, filtered and evaporated to dryness. Purification by prep-TLC (dichloromethane/methanol=25/1) gave the title compound as a pale yellow solid (35 mg, 65%). ¹H NMR (400 MHz, MeOH-d₄): δ 8.65 (d, 1H), 8.09 (d, 1H), 7.98 (t, 1H), 7.49˜7.52 (m, 1H), 6.89 (dd, 1H), 6.58 (s, 1H), 4.41 (s, 2H), 4.41 (s, 2H), 3.75 (m, 2H), 3.00 (td, 2H), 1.93 (m, 2H). MS m/z: 351 (M+H)⁺.

### 219. Compound 219: 5-(2-Chlorophenyl)-2-(pyridin-2-yl)-4,5,6,7-tetrahydrobenzo[d]oxazole

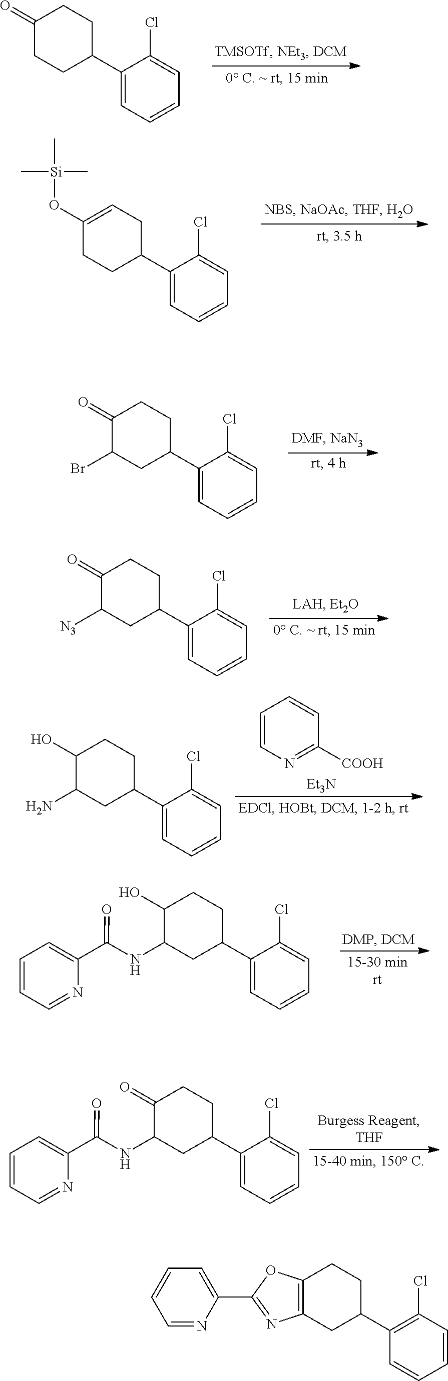

### (a) (4-(2-Chlorophenyl)cyclohex-1-enyloxy)trimethylsilane (I-219.1)

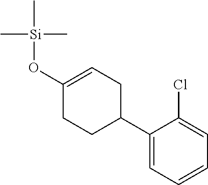

To a solution of 4-(2-chlorophenyl)cyclohexanone (3.4 g, 16.3 mmol, 1.0 eq) and triethylamine (5 g, 48.9 mol, 3.0 eq) in dichloromethane (50 mL) was added trimethylsilyltrifluoromethanesulfonate (7.2 g, 32.6 mmol, 2.0 eq) via dropwise addition at 0° C. The reaction was stirred at 0° C. for 15 min, at which point water (50 mL) was added. The organic layer was separated, washed with water (50 mL) and brine (25 mL) and dried over Na₂SO₄. Concentration in vacuo gave (4-(2-chlorophenyl)cyclohex-1-enyloxy)trimethylsilane (4.2 g, 93%), which was used directly in the next step without further purification. MS (ESI): 281 (M+H)⁺.

### (b) 2-Bromo-4-(2-chlorophenyl)cyclohexanone (I-219.2)

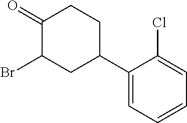

To a solution of N-bromosuccinimide (2.9 g, 16.4 mmol, 1.1 eq) and sodium acetate (120 mg, 1.5 mmol, 0.1 eq) in tetrahydrofuran/water (50 mL, v/v=1:1) was added (4-(2-chlorophenyl)cyclohex-1-enyloxy)trimethylsilane (4.2 g, 15 mmol, 1.0 eq). The reaction mixture was stirred at ambient temperature for 3.5 h. The reaction was quenched with an aqueous solution of Na₂SO₃ (10%) until the reaction became colorless. The reaction mixture was extracted with diethyl ether (2×60 mL). The combined organic phases were washed with aqueous NaHCO₃ solution (50 mL), water (50 mL), and brine (50 mL) successively, then dried over MgSO₄. After concentration in vacuo, the crude material was purified by column chromatography (petroleum ether:ethyl acetate=3:1) on silica gel to give 2-bromo-4-(2-chlorophenyl)cyclohexanone as a white solid, which was a mixture of isomers (2.8 g, 67%). ¹H NMR (400 MHz, CDCl₃) δ: 7.41-7.39 (m, 1H), 7.27-7.17 (m, 3H), 4.88-4.83 (m, 0.4H), 4.44-4.42 (m, 0.6H), 4.10-4.02 (m, 0.6H), 3.73-3.66 (m, 0.4H), 3.42-3.33 (m, 0.6H), 2.85-2.79 (m, 0.8H), 2.72-2.63 (m, 0.4H), 2.52-2.41 (m, 1.2H), 2.36-1.22 (m, 2H), 1.98-1.88 (m, 1H).

### (c) 2-Azido-4-(2-chlorophenyl)cyclohexanone (I-219.3)

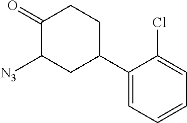

To a solution of 2-bromo-4-(2-chlorophenyl)cyclohexanone (2.8 g, 9.7 mmol, 1.0 eq) in dimethylformamide (50 mL) was added sodium azide (1.3 g, 19.4 mmol, 2.0 eq). The mixture was stirred at room temperature for 4 h. Water (100 mL) was added and the reaction was extracted with diethyl ether (3×40 mL). The combined organic phases were washed with water (2×40 mL) and brine (2×40 mL) successively, then dried over MgSO₄. The diethyl ether solution containing 2-azido-4-(2-chlorophenyl)cyclohexanone was used directly in the next step without further purification. MS (ESI): 221 (M−28)⁺.

### (d) 2-Amino-4-(2-chlorophenyl)cyclohexanol (I-219.4)

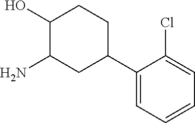

To a solution of 2-azido-4-(2-chlorophenyl)cyclohexanone (about 9.7 mmol, obtained from the above step) in diethyl ether was added lithium aluminum hydride (400 mg, 20 mmol) at 0° C. After 15 min, Na₂SO₄.10H₂O (1 g) was added to the reaction mixture slowly, followed by water (30 mL). The mixture was filtered and the pH adjusted to ˜5 with 1N aqueous HCl solution. The aqueous layer was separated and the pH adjusted to ˜8 with saturated aqueous NaHCO₃. The mixture was extracted with ethyl acetate (2×30 mL). The combined organic phases were dried over MgSO₄ and concentrated to give 2-amino-4-(2-chlorophenyl)cyclohexanol (550 mg, 16% over 2 steps) as a mixture of isomers. MS (ESI): 226 (M+H)⁺.

### (e) N-(5-(2-Chlorophenyl)-2-hydroxycyclohexyl)picolinamide (I-219.5)

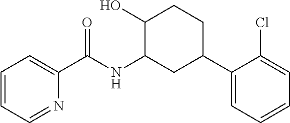

2-Amino-4-(2-chlorophenyl)cyclohexanol (550 mg, 2.4 mmol, 1.0 eq), picolinic acid (330 mg, 2.6 mmol, 1.1 eq), 1-ethyl-3-(3-dimethylaminopropyl)-carbodiimide hydrochloride (932 mg, 4.8 mmol, 2.0 eq) and hydroxybenzotriazole (746 mg, 4.8 mmol, 2.0 eq) were combined and stirred in dichloromethane. Triethylamine (370 mg, 3.6 mmol, 1.5 eq) was added and the reaction was stirred for 1-2 h at room temperature. Water (20 mL) was added and the reaction mixture was charged to a separatory funnel and separated. The organic layer was washed with water (20 mL) and brine (20 mL) successively. The organic layer was dried with Na₂SO₄, filtered, and concentrated to a crude solid, which was purified by silica gel chromatography (petroleum ether:ethyl acetate=5:1) to give N-(5-(2-chlorophenyl)-2-hydroxycyclohexyl)picolinamide (350 mg, 43%) as a white solid, which was a mixture of isomers. MS (ESI): 331 (M+H)⁺.

### (f) N-(5-(2-Chlorophenyl)-2-oxocyclohexyl)picolinamide (I-219.6)

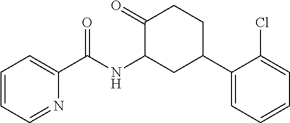

N-(5-(2-Chlorophenyl)-2-hydroxycyclohexyl)picolinamide (350 mg, 3.4 mmol 1.0 eq) was dissolved in dichloromethane (30 mL). Dess-Martin Periodinane (1.0 g, 10.3 mmol 3.0 eq) was added, and the reaction was stirred at RT for 15-30 min. The reaction mixture was washed with 0.5N aqueous NaOH and brine. The organic layer was dried over Na₂SO₄, filtered, and concentrated to give the crude product, which was purified by silica gel chromatography (petroleum ether:ethyl acetate=5:1) to give N-(5-(2-chlorophenyl)-2-oxocyclohexyl)picolinamide as a white solid (290 mg, 83%). MS (ESI): 329 (M+H)⁺.

### (g) Compound 219: 5-(2-Chlorophenyl)-2-(pyridin-2-yl)-4,5,6,7-tetrahydrobenzo[d]oxazole

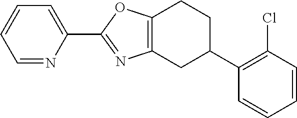

N-(5-(2-Chlorophenyl)-2-oxocyclohexyl)picolinamide (120 mg, 0.4 mmol, 1.0 eq) and methyl N-(triethylammoniumsulfonyl)carbamate (Burgess Reagent) (261 mg, 12 mmol, 3.0 eq) were combined in dry tetrahydrofuran (6 mL) in a 10 mL microwave vial. A stirred bar was added, and the container was flushed with nitrogen. The reaction was heated to 150° C. for 15-40 min. in a microwave synthesizer. After cooling, the solvent was removed in vacuo. Water (10 mL) was added, and the reaction was extracted with dichloromethane (3×10 mL). The combined organic layers were washed with brine, dried over Na₂SO₄, filtered and concentrated to give the crude product. Purification by silica gel chromatography (petroleum ether:ethyl acetate=5:1) gave 5-(2-chlorophenyl)-2-(pyridin-2-yl)-4,5,6,7-tetrahydrobenzo[d]oxazole (24 mg, 21%) as a white solid. MS (ESI): 311 (M+H)⁺; ¹H NMR (400 MHz, CDCl₃) δ: 8.72 (s, 1H), 8.11-8.09 (m, 1H), 7.82-7.77 (m, 1H), 7.40-7.38 (m, 1H), 7.32-7.23 (m, 3H), 7.20-7.16 (m, 1H), 3.65-3.58 (m, 1H), 3.04-2.91 (m, 1H), 2.99-2.85 (m, 2H), 2.76-2.70 (m, 1H), 2.21-2.05 (m, 2H).

### 220. Compound 220: (R)-5-(2-chlorophenyl)-2-(pyridin-2-yl)-4,5,6,7-tetrahydrobenzo[d]oxazole

### 221. Compound 221: (S)-5-(2-chlorophenyl)-2-(pyridin-2-yl)-4,5,6,7-tetrahydrobenzo[d]oxazole

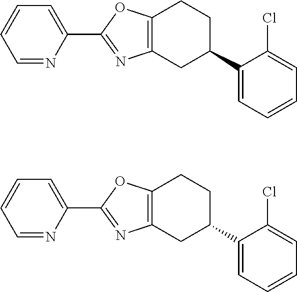

Racemic 5-(2-chlorophenyl)-2-(pyridin-2-yl)-4,5,6,7-tetrahydrobenzo[d]oxazole (219 mg) was dissolved in methanol and loaded to a ChiralPak AD-H column (3 cm×25 cm) (Chiral Technologies); the mobile phase consisted of supercritical CO₂ (125 bar) with hexane:isopropanol as cosolvent. (R)-5-(2-Chlorophenyl)-2-(pyridin-2-yl)-4,5,6,7-tetrahydrobenzo[d]oxazole (92 mg) eluted as Peak 1 (t_{R}=1.58 min), and (S)-5-(2-chlorophenyl)-2-(pyridin-2-yl)-4,5,6,7-tetrahydrobenzo[d]oxazole (91 mg) eluted as Peak 2 (t_{R}=2.32 min). ¹H NMR (400 MHz, CDCl₃): δ 8.72 (s, 1H), 8.11-8.09 (m, 1H), 7.82-7.77 (m, 1H), 7.40-7.38 (m, 1H), 7.32-7.23 (m, 3H), 7.20-7.16 (m, 1H), 3.65-3.58 (m, 1H), 3.04-2.91 (m, 1H), 2.99-2.85 (m, 2H), 2.76-2.70 (m, 1H), 2.21-2.05 (m, 2H); MS (ESI): 311 (M+H)⁺.

### 222. Compound 222: 3-Fluoro-5-(2-(pyridin-2-yl)-4,5,6,7-tetrahydrobenzo[d]oxazol-5-yl)benzonitrile

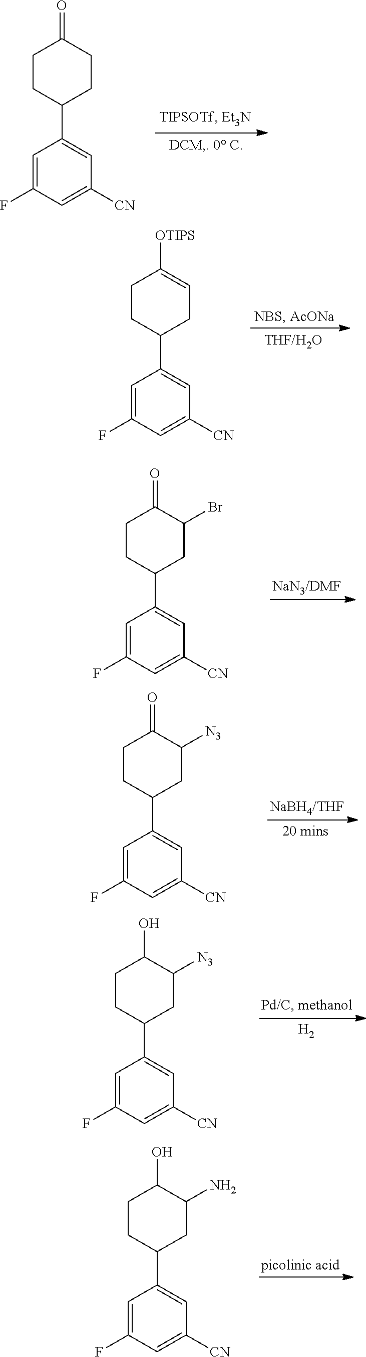

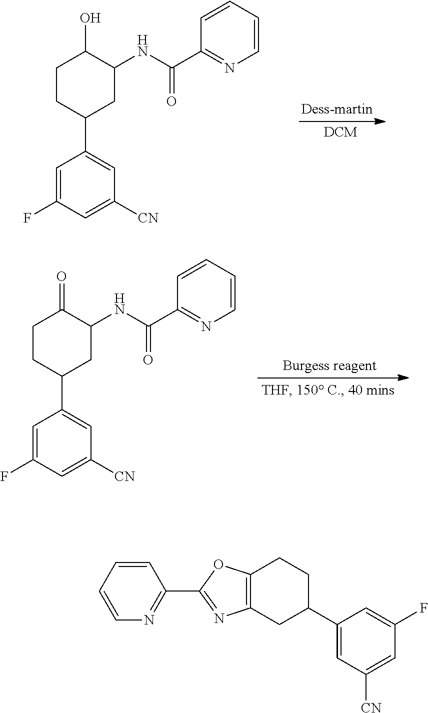

### (a) 3-Fluoro-5-(4-(triisopropylsilyloxy)cyclohex-3-enyl)benzonitrile (I-222.1)

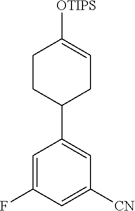

A solution of 3-fluoro-5-(4-oxocyclohexyl)benzonitrile (purchased from GLSyntech) (7.2 g, 33.1 mmol) in dichloromethane (60 mL) was cooled to 0° C. Triethylamine (6.69 g, 66.2 mmol) was added followed by the dropwise addition of triisopropylsilyl trifluoromethanesulfonate (15.2 g, 49.7 mmol). The mixture was stirred at room temperature for 1 hour. After completion, the reaction mixture was quenched with water (50 mL). The organic layer was separated and the aqueous layer was extracted with dichloromethane (50 mL). The organic layer was dried over sodium sulfate, filtered, and concentrated in vacuo. The residue was purified by column chromatography on silica gel to give 3-fluoro-5-(4-(triisopropylsilyloxy)cyclohex-3-enyl)benzonitrile as a yellow oil which solidified upon refridgeration. (12.5 g, 100%); MS (ESI): m/z=374 [M+H]⁺.

### (b) 3-(3-Bromo-4-oxocyclohexyl)-5-fluorobenzonitrile (I-222.2)

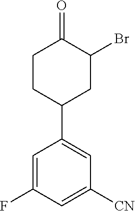

To a solution of 3-fluoro-5-(4-(triisopropylsilyloxy)cyclohex-3-enyl)benzonitrile (3.2 g, 8.58 mmol) in THF (50 mL) was added water (50 mL) and sodium acetate (100 mg). To this suspension was added 1-bromopyrrolidine-2,5-dione (1.53 g, 8.58 mmol) over a period of 5 minutes. The yellow color disappeared immediately. The reaction was stirred at room temperature for 30 minutes. The tetrahydrofuran was removed in vacuo, and the aqueous layer was extracted with ethyl acetate (2×40 mL). The combined organic layers was dried over sodium sulfate, filtered and evaporated to dryness. The residue was purified by column chromatography on silica gel (petrol ether/ethyl acetate=50/1) to give 3-(3-bromo-4-oxocyclohexyl)-5-fluorobenzonitrile as a white solid (700 mg, 23%). ¹H NMR (400 MHz, CDCl₃): δ 7.38 (s, 1H), 7.22-7.28 (m, 2H), 4.44 (s, 1H), 3.61 (m, 1H), 3.34 (dt, 1H), 2.37-2.49 (m, 3H), 2.23-2.27 (m, 1H), 1.91-1.95 (m, 1H); MS (ESI): m/z=296/298 [M+H]⁺.

### (c) 3-(3-Azido-4-oxocyclohexyl)-5-fluorobenzonitrile (I-222.3)

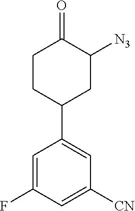

A solution of 3-(3-bromo-4-oxocyclohexyl)-5-fluorobenzonitrile (502 mg, 1.7 mmol) in dry DMF (10 mL) was cooled to 0° C. Sodium azide (332 mg, 5.1 mmol) was added, and the reaction was stirred at 0° C. for 30 minutes. The reaction mixture was poured into ice-water (80 mL), and the suspension was extracted with ethyl acetate (2×40 mL). The organic layer was washed with brine (2×30 mL). The organic layer was dried over sodium sulfate, filtered and evaporated to give 3-(3-azido-4-oxocyclohexyl)-5-fluorobenzonitrile as a colorless oil (450 mg, 100%); MS (ESI): m/z 281 [M+23]⁺.

### (d) 3-(3-Azido-4-hydroxycyclohexyl)-5-fluorobenzonitrile (I-222.4)

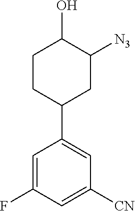

To a solution of 3-(3-azido-4-oxocyclohexyl)-5-fluorobenzonitrile (450 mg, 1.74 mmol) in THF (8 mL) was added sodium borohydride (132 mg, 3.48 mmol) at room temperature. The reaction was stirred at room temperature for 20 min. Methanol (3 mL) was added and the solvent was removed under reduced pressure. The residue was dissolved in dichloromethane (8 mL) and washed with water (8 mL). The organic layer was dried over sodium sulfate and concentrated to give 3-(3-azido-4-hydroxycyclohexyl)-5-fluorobenzonitrile as a yellow oil (450 mg, 99%); MS m/z: 233 (M+H-28).

### (e) 3-(3-Amino-4-hydroxycyclohexyl)-5-fluorobenzonitrile (I-222.5)

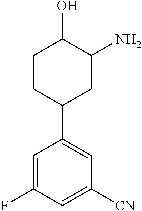

To a solution of 3-(3-azido-4-hydroxycyclohexyl)-5-fluorobenzonitrile (450 mg, 1.73 mmol) in methanol (8 mL) was added Pd/C (10%, 50 mg) under a nitrogen atmosphere. The reaction was stirred under H₂ atmosphere at room temperature for 20 min. The reaction was filtered, and the filtrate concentrated to give the product as yellow oil (360 mg, 89%); MS m/z: 235 (M+H)⁺.

(f) N-(5-(3-Cyano-5-fluorophenyl)-2-hydroxycyclohexyl)picolinamide (I-222.6)

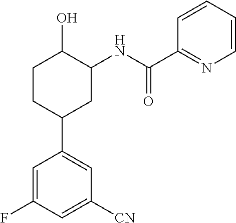

A suspension of picolinic acid (118 mg, 096 mmol) in dichloromethane (15 mL) was cooled to 0° C. HOBt (294 mg, 1.92 mmol), EDCI (367 mg, 1.92 mmol) and triethylamine (145 mg, 1.44 mmol) were added in sequence, followed by a solution of 3-(3-amino-4-hydroxycyclohexyl)-5-fluorobenzonitrile (225 mg, 0.96 mmol) in dichloromethane (2 mL). The dark reaction mixture was stirred at room temperature for 1 h. The reaction was washed with saturated sodium bicarbonate solution (15 mL). The organic layer was dried over sodium sulfate, filtered, evaporated to dryness. The residue was purified by chromatography on silica gel (dichloromethane/methanol=100/1) to give N-(5-(3-cyano-5-fluorophenyl)-2-hydroxycyclohexyl)picolinamide (I-7) as a yellow solid (260 mg, 80%); MS (ESI): m/z 340 [M+1]⁺.

### (g) N-(5-(3-Cyano-5-fluorophenyl)-2-oxocyclohexyl)picolinamide (I-222.7)

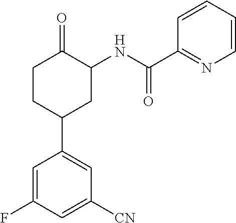

A mixture of N-(5-(3-cyano-5-fluorophenyl)-2-hydroxycyclohexyl) picolinamide (260 mg, 0.77 mmol) and Dess-martin periodinane (980 mg, 2.3 mmol) in dichloromethane (15 mL) was stirred at room temperature for 3 h. 0.5 N sodium hydroxide (10 mL) was added and the mixture was stirred for 20 minutes. The organic layer was separated, dried over sodium sulfate, filtered and evaporated to give N-(5-(3-cyano-5-fluorophenyl)-2-oxocyclohexyl)picolinamide as a yellow solid (230 mg, 89%); MS (ESI): m/z 338 [M+1]⁺.

### (h) Compound 222: 3-Fluoro-5-(2-(pyridin-2-yl)-4,5,6,7-tetrahydrobenzo[d]oxazol-5-yl)benzonitrile

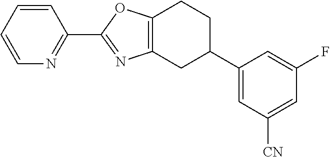

A mixture of N-(5-(3-cyano-5-fluorophenyl)-2-oxocyclohexyl)picolinamide (260 mg, 0.77 mmol) and Burgess reagent (643 mg, 2.7 mmol) was heated to 150° C. by microwave and stirred for 40 minutes. The reaction mixture was cooled and diluted with ethyl acetate (5 mL). The organic layer was washed with brine (3 mL), dried over sodium sulfate, filtered and evaporated to dryness. The residue was purified by prep-TLC (dichloromethane/methanol=10/1) to give 3-fluoro-5-(2-(pyridin-2-yl)-4,5,6,7-tetrahydrobenzo[d]oxazol-5-yl)benzonitrile as a yellow solid (120 mg, 49%). ¹H NMR (400 MHz, CDCl₃): δ 8.72 (d, 1H), 8.10 (d, 1H), 7.82 (t, 1H), 7.40 (s, 1H), 7.32˜7.37 (m, 1H), 7.25˜7.27 (m, 2H), 3.14˜3.16 (m, 1H), 2.97 (dd, 1H), 2.89 (brs, 2H), 2.73˜2.79 (m, 1H), 2.21˜2.24 (m, 1H), 2.06˜2.14 (m, 1H); MS (ESI): m/z 320 [M+1]⁺.

### 223. Compound 223: 5-(2-chloro-4-fluorophenyl)-2-(pyridin-2-yl)-4,5,6,7-tetrahydrobenzo[d]oxazole

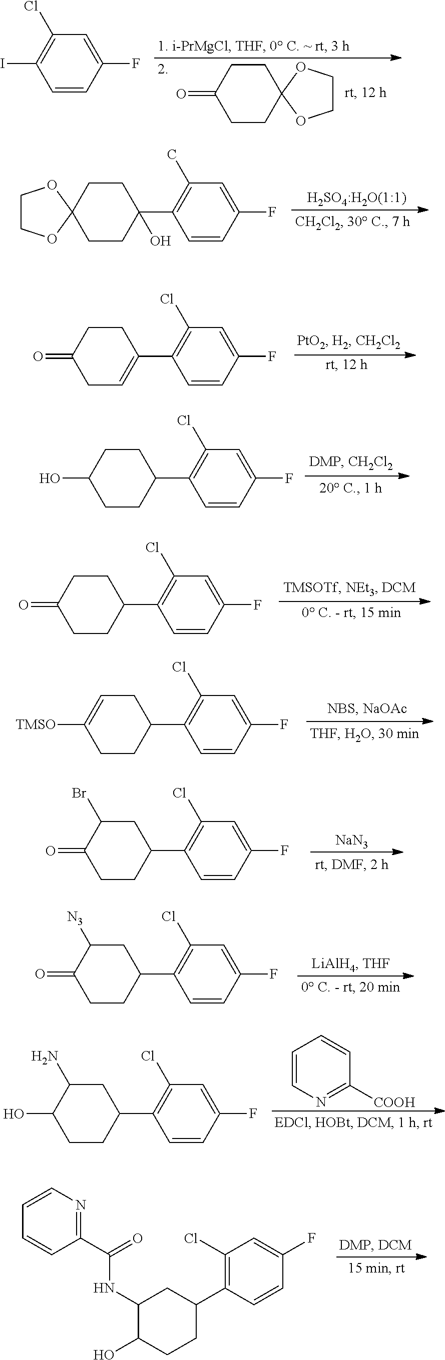

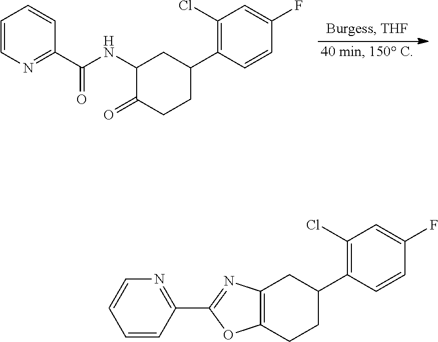

### (a) 8-(2-Chloro-4-fluorophenyl)-1,4-dioxaspiro[4.5]decan-8-ol (I-223.1)

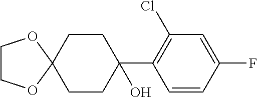

To a solution of 2-chloro-4-fluoro-1-iodobenzene (10.0 g, 39.0 mmol) in dry THF (150 mL) was added i-PrMgCl (29.3 mL, 2.0 N, 58.5 mmol) dropwise at 0° C. After stirring for 1 hr at 0° C., the reaction was warmed to room temperature and stirred for 2 h. 1,4-cyclohexanedione monoethylene acetal (4.87 g, 31.2 mmol) was added and the reaction was stirred at room temperature for 12 h. Water (100 mL) was added and the mixture was extracted with ethyl acetate (3×100 mL). The combined organic phases were washed with brine (100 mL), then dried over Na₂SO₄. After concentration in vacuo, the crude material was purified by column chromatography (petroleum ether:ethyl acetate=5:1) on silica gel to give the desired product as a white solid (5.38 g, 48%). ¹H NMR (400 MHz, CDCl₃): δ 7.61-7.57 (m, 1H), 7.14-7.11 (m, 1H), 6.99-6.94 (m, 1H), 4.00-3.95 (m, 4zH), 2.63-2.57 (m, 1H), 2.42-2.36 (m, 2H), 2.17-2.10 (m, 2H), 2.05-2.01 (m, 2H), 1.72-1.69 (m, 2H).

### (b) 4-(2-Chloro-4-fluorophenyl)cyclohex-3-enone (I-223.2)

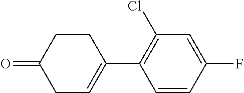

To a solution of 8-(2-chloro-4-fluorophenyl)-1,4-dioxaspiro[4.5]decan-8-ol (10.7 g, 37.3 mmol) in CH₂Cl₂ (150 mL) was added H₂SO₄/water (25 mL, v/v=1:1). The reaction mixture was stirred at 30 V for 7 h. Saturated NaHCO₃ solution was added carefully until the pH was approximately 7. The phases were separated, and the aqueous phase was re-extracted with CH₂Cl₂ (3×50 mL). The combined organic phases were washed with brine (50 mL) and dried over Na₂SO₄. After concentration in vacuo, the crude material was purified by column chromatography (petroleum ether: ethyl acetate=10:1) on silica gel to give the desired product as a yellow oil (6.40 g, 76%). ¹H NMR (400 MHz, CDCl₃): δ 7.20-7.12 (m, 2H), 6.99-6.95 (m, 1H), 5.78-5.76 (m, 1H), 3.06-3.04 (m, 2H), 2.78-2.75 (m, 2H), 2.66-2.63 (m, 2H).

### (c) 4-(2-Chloro-4-fluorophenyl)cyclohexanol (I-223.3)

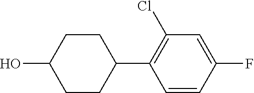

To a solution of 4-(2-chloro-4-fluorophenyl)cyclohex-3-enone (6.40 g, 28.5 mmol) in CH₂Cl₂ (100 mL) was added PtO₂ (0.50 g). The mixture was stirred under H₂ atmosphere at room temperature for 12 h, filtered and concentrated in vacuo to give the crude product (6.27 g, 96%) as a yellow oil. The crude product was used directly for the next step. MS (ESI): m/z 211 [M−17]⁺.

### (d) 4-(2-Chloro-4-fluorophenyl)cyclohexanone (I-223.4)

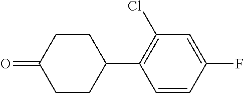

To a solution of 4-(2-chloro-4-fluorophenyl)cyclohexanol (6.0 g, 26.2 mmol) in CH₂Cl₂ (60 mL) was added DMP (32.1 g, 78.7 mmol). The reaction mixture was stirred at 20 V for 1 h. Sodium hydroxide (0.5 N) was added to quench the reaction. The two phases were separated; the organic phase was washed with water (100 mL) and brine (100 mL), and dried over Na₂SO₄. After concentration in vacuo, the crude material was purified by column chromatography (petroleum ether:ethyl acetate=15:1) on silica gel to give a white solid (3.3 g, 56%). MS (ESI): m/z 227 (M+H)⁺.

### (e) (4-(2-Chloro-4-fluorophenyl)cyclohex-1-enyloxy)trimethylsilane (I-223.5)

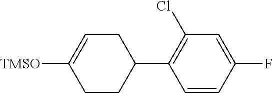

To a solution of 4-(2-chloro-4-fluorophenyl)cyclohexanone (2.9 g, 12.9 mmol) and triethylamine (3.9 g, 38.3 mmol) in dichloromethane (50 mL) was added trimethylsilyl trifluoromethanesulfonate (5.7 g, 25.8 mmol) dropwise at 0° C. After stirring the reaction for 15 min, the organic layer was washed with water (2×50 mL) and brine (25 mL) and dried over Na₂SO₄. The solvent was filtered and concentrated to give product (3.6 g, 95%), which was used directly for the next step. MS (ESI): m/z 299 (M+H)⁺.

### (f) 2-Bromo-4-(2-chloro-4-fluorophenyl)cyclohexanone (I-223.6)

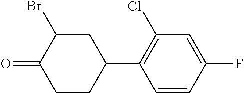

To a solution of N-bromosuccinimide (2.5 g, 14.0 mmol) and sodium acetate (105 mg, 1.3 mmol) in THF/water (50 mL, v/v=1:1) was added (4-(2-chloro-4-fluorophenyl)cyclohex-1-enyloxy)trimethylsilane (3.8 g, 12.7 mmol). The reaction mixture was stirred at room temperature for 3.5 h. The reaction was quenched with Na₂SO₃ solution (10%) until colorless, then extracted with diethyl ether (2×60 mL). The combined organic phases were washed NaHCO₃ solution (50 mL), water (50 mL) and brine (50 mL), then dried over MgSO₄. After concentration in vacuo, the crude material was purified by column chromatography (petroleum ether:ethyl acetate=3:1) on silica gel to give the product as a white solid (2.8 g, 71%).

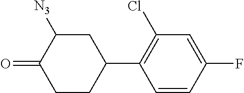

To a solution of 2-bromo-4-(2-chloro-4-fluorophenyl)cyclohexanone (2 g, 6.5 mmol) in dimethylformamide (15 mL) was added NaN₃ (855 mg, 13 mmol). The reaction was stirred at room temperature for 2 h. Water (45 mL) was added, and the mixture was extracted with EtOAc (3×10 mL). The combined organic phases were washed with water (2×20 mL) and brine (2×20 mL), dried over MgSO₄ and concentrated to give the crude product (1.6 g) as an oil, which was used directly for the next step. MS (ESI): m/z 239 (M−28)⁺.

### (h) 2-Amino-4-(2-chloro-4-fluorophenyl)cyclohexanol (I-223.8)

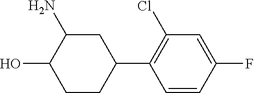

To a solution of 2-azido-4-(2-chloro-4-fluorophenyl)cyclohexanone (1.6 g, 6 mmol) in THF (30 mL) was added LiAlH₄ (450 mg, 12 mmol) at 0° C. After 20 min at rt, Na₂SO₄.10H₂O (1 g) was added into the reaction mixture slowly, followed by water (10 mL). The mixture was filtered and concentrated to give crude the product as yellow oil, which was used directly for the next step. MS (ESI): m/z 244 (M+H)⁺.

### (i) N-(5-(2-Chloro-4-fluorophenyl)-2-hydroxycyclohexyl)picolinamide (I-223.9)

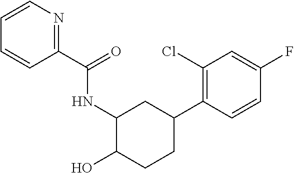

2-Amino-4-(2-chloro-4-fluorophenyl)cyclohexanol (2 g, 8 mmol), picolinic acid (1.1 g, 9 mmol), 1-ethyl-3-(3-dimethylaminopropyl)carbodiimide (3.1 g, 16 mmol), hydroxybenzotriazole (2.5 g, 16 mmol), and triethylamine (1.2 g, 12 mmol) were combined in DCM (80 mL) and stirred for 1 h at room temperature. Water (100 mL) was added and the organic layer was separated, washed with water (80 mL) and brine (80 mL) and dried with Na₂SO₄. The organic layer was filtered and concentrated to give the crude product, which was purified by silica chromatography (petroleum ether:ethyl acetate=5:1) to give the target product (800 mg, 35%) as a white solid; MS (ESI): m/z 349 (M+H)⁺.

### (j) N-(5-(2-Chloro-4-fluorophenyl)-2-oxocyclohexyl)picolinamide (I-223.10)

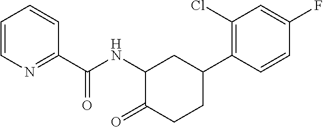

N-(5-(2-Chloro-4-fluorophenyl)-2-hydroxycyclohexyl)picolinamide (800 mg, 2.3 mmol) and Dess-Martin periodinane (2.94 g, 6.9 mmol) were combined in dichloromethane (30 mL) and stirred at rt for 15 min. The reaction was quenched with NaOH (0.5N). The organic layer was washed with brine, dried over Na₂SO₄, filtered and concentrated to give crude product. The crude product was purified by silica chromatography (petroleum ether:ethyl acetate=5:1) to give the desired product as a white solid (500 mg, 63%). MS (ESI): m/z 349 (M+H)⁺.

### (k) Compound 223: 5-(2-Chloro-4-fluorophenyl)-2-(pyridin-2-yl)-4,5,6,7-tetrahydrobenzo[d]oxazole

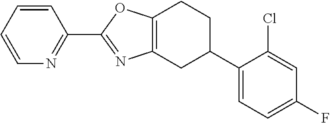

N-(5-(2-Chloro-4-fluorophenyl)-2-oxocyclohexyl)picolinamide (500 mg, 1.4 mmol) and Burgess reagent (1 g, 4.3 mmol) were added in dry tetrahydrofuran (10 mL) in a 30 mL microwave vial. The container was flushed with nitrogen and heated to 150° C. for 40 min. via microwave. The solvent was removed and the residue partitioned between water (20 mL and dichloromethane (20 ml). The aqueous layer was extracted with dichloromethane (2×20 mL). The combined organic layers were washed with brine, dried over Na₂SO₄, filtered and concentrated to give crude product. The crude product was purified by chromatography (petroleum ether:ethyl acetate=5:1) on silica gel to give the desired product as a white solid (300 mg, 63%). ¹H NMR (400 MHz, CDCl₃): δ 8.72 (d, 1H), 8.10 (d, 1H), 7.83-7.78 (m, 1H), 7.36-7.34 (m, 1H), 7.28-7.25 (m, 1H), 7.17-7.14 (m, 1H), 7.00-6.96 (m, 1H), 3.60-3.53 (m, 1H), 3.02-2.92 (m, 1H), 2.85-2.84 (m, 2H), 2.70-2.60 (m, 1H), 2.18-2.03 (m, 2H). MS (ESI): m/z 329 (M+H)⁺.

### 224. Compound 224: (R)-5-(2-chloro-4-fluorophenyl)-2-(pyridin-2-yl)-4,5,6,7-tetrahydrobenzo[d]oxazole

### 225. Compound 225: (S)-5-(2-chloro-4-fluorophenyl)-2-(pyridin-2-yl)-4,5,6,7-tetrahydrobenzo[d]oxazole

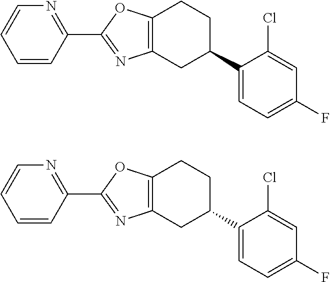

Racemic 5-(2-chloro-4-fluorophenyl)-2-(pyridin-2-yl)-4,5,6,7-tetrahydrobenzo[d]oxazole (200 mg) was dissolved in isopropanol and loaded to a RegisPak (Regis technologies)(3 cm×25 cm) chiral HPLC column. The mobile phase consisted of supercritical CO₂ (150 bar) with 0.5% isopropylamine in isopropanol as cosolvent. (R)-5-(2-Chloro-4-fluorophenyl)-2-(pyridin-2-yl)-4,5,6,7-tetrahydrobenzo[d]oxazole (78 mg) eluted as Peak 1 (t_{R}=1.7 min), and (S)-5-(2-chloro-4-fluorophenyl)-2-(pyridin-2-yl)-4,5,6,7-tetrahydrobenzo[d]oxazole (66 mg) eluted as Peak 2 (t_{R}=2.8 min). ¹H NMR (400 MHz, CDCl₃): δ 8.72 (d, 1H), 8.10 (d, 1H), 7.83-7.78 (m, 1H), 7.36-7.34 (m, 1H), 7.28-7.25 (m, 1H), 7.17-7.14 (m, 1H), 7.00-6.96 (m, 1H), 3.60-3.53 (m, 1H), 3.02-2.92 (m, 1H), 2.85-2.84 (m, 2H), 2.70-2.60 (m, 1H), 2.18-2.03 (m, 2H). MS (ESI): m/z 329 (M+H)⁺.

### 226. Synthesis of Compounds 226-391

The following compounds are prepared using the schemes and procedures described above, from suitable starting materials.

Compound 226: 3-(2-(pyridin-2-yl)-4,5,6,7-tetrahydrobenzo[d]oxazole-6-carbonyl)benzonitrile

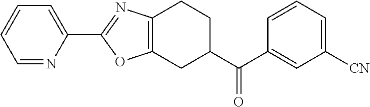

### Compound 227: 3-((2-(pyridin-2-yl)-4,5,6,7-tetrahydrobenzo[d]oxazol-6-yl)methyl)benzonitrile

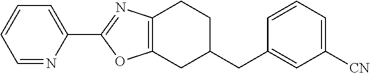

### Compound 228: 3-((2-(pyridin-2-yl)-4,5,6,7-tetrahydrobenzo[d]oxazol-5-yl)methyl)benzonitrile

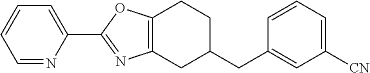

### Compound 229: 3-fluoro-5-((2-(pyridin-2-yl)-4,5,6,7-tetrahydrobenzo[d]oxazol-5-yl)methyl)benzonitrile

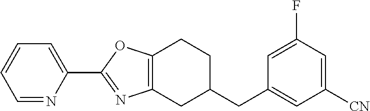

### Compound 230: 2-(3-chlorophenyl)-5-(pyridin-2-ylmethyl)-4,5,6,7-tetrahydrobenzo[d]oxazole

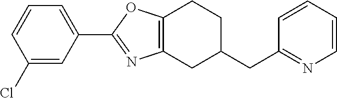

### Compound 231: (4-fluorophenyl)(2-(4-fluorophenyl)-4,5,6,7-tetrahydrobenzo[d]oxazol-5-yl)methanone

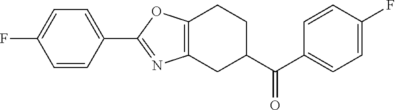

### Compound 232: 3-(2-benzyl-4,5,6,7-tetrahydrobenzo[d]oxazol-5-yl)-5-fluorobenzonitrile

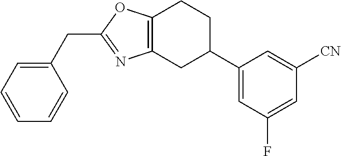

### Compound 233: 3-fluoro-5-(2-(pyridin-2-yl)-4,5,6,7-tetrahydrobenzo[d]thiazol-6-yl)benzonitrile

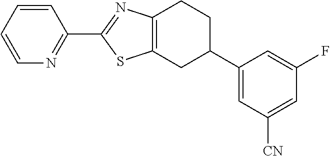

### Compound 234: 3-fluoro-5-(2-(pyridin-2-yl)-4,5,6,7-tetrahydro-1H-benzo[d]imidazol-6-yl)benzonitrile

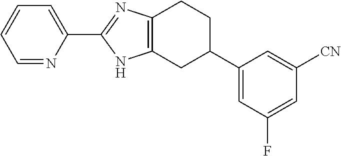

### Compound 235: 2-(3-chlorophenyl)-7-(pyridin-3-yl)-5,6,7,8-tetrahydroimidazo[1,2-a]pyridine

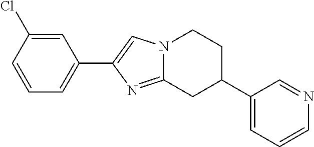

### Compound 236: 2-(3-chlorophenyl)-7-(pyridin-2-yl)-5,6,7,8-tetrahydroimidazo[1,2-a]pyridine

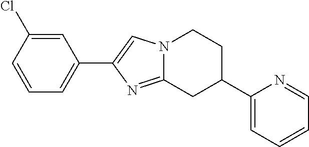

### Compound 237: 2-(2-(3-chlorophenyl)-5,6,7,8-tetrahydroimidazo[1,2-a]pyridin-7-yl)nicotinonitrile

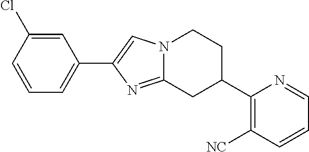

### Compound 238: 3-fluoro-5-(2-(pyridin-2-yl)-5,6,7,8-tetrahydroimidazo[1,2-a]pyridin-7-yl)benzonitrile

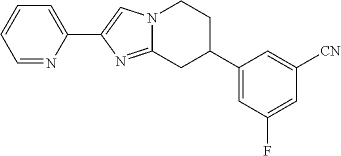

### Compound 239: 3-(2-(pyridin-2-yl)-5,6,7,8-tetrahydroimidazo[1,2-a]pyridin-7-yl)benzonitrile

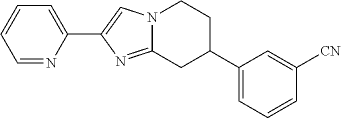

### Compound 240: 3-fluoro-5-(2-(pyridin-2-yl)-5,6,7,8-tetrahydro-[1,2,4]triazolo[1,5-a]pyridin-7-yl)benzonitrile

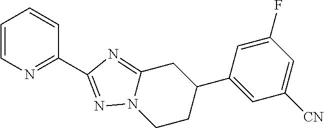

### Compound 241: 7-(3-chlorophenyl)-2-(pyridin-2-yl)-5,6,7,8-tetrahydro-[1,2,4]triazolo[1,5-a]pyridine

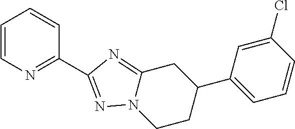

### Compound 242: 7-(2-chlorophenyl)-2-(pyridin-2-yl)-5,6,7,8-tetrahydro-[1,2,4]triazolo[1,5-a]pyridine

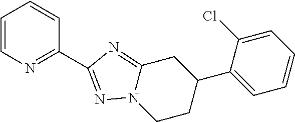

### Compound 243: 7-(3-chloropyridin-2-yl)-2-(pyridin-2-yl)-5,6,7,8-tetrahydro-[1,2,4]triazolo[1,5-a]pyridine

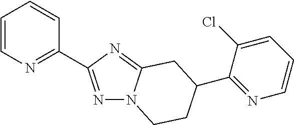

### Compound 244: 7-(2-chloro-4-fluorophenyl)-2-(pyridin-2-yl)-5,6,7,8-tetrahydro-[1,2,4]triazolo[1,5-a]pyridine

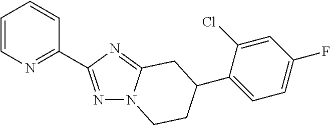

### Compound 245: 7-(3-fluorophenyl)-2-(pyridin-2-yl)-5,6,7,8-tetrahydro-[1,2,4]triazolo[1,5-a]pyridine

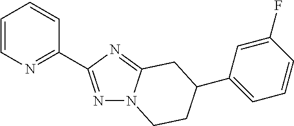

### Compound 246: 3-(2-(pyridin-2-yl)-4,5,6,7-tetrahydrobenzo[d]oxazol-6-yl)benzonitrile

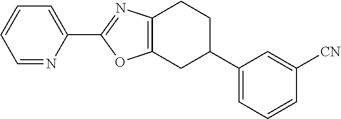

### Compound 247: 2,6-di(pyridin-2-yl)-4,5,6,7-tetrahydrobenzo[d]oxazole

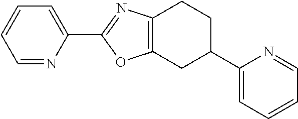

### Compound 248: 2-(2-(pyridin-2-yl)-4,5,6,7-tetrahydrobenzo[d]oxazol-6-yl)nicotinonitrile

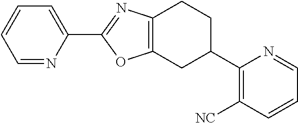

### Compound 249: 3-fluoro-5-(2-(pyridin-2-yl)-4,5,6,7-tetrahydrobenzo[d]oxazol-6-yl)benzonitrile

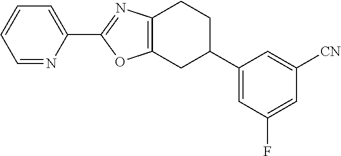

### Compound 250: 2-(pyridin-2-yl)-6-(pyridin-3-yl)-4,5,6,7-tetrahydrobenzo[d]oxazole

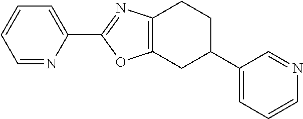

### Compound 251: 6-(2-(pyridin-2-yl)-4,5,6,7-tetrahydrobenzo[d]oxazol-6-yl)picolinonitrile

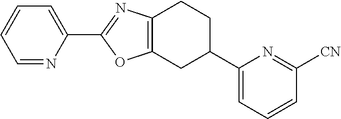

### Compound 252: 2-(2-(pyridin-2-yl)-4,5,6,7-tetrahydrobenzo[d]oxazol-6-yl)isonicotinonitrile

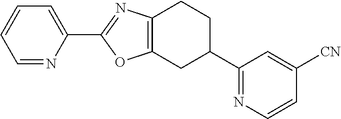

### Compound 253: 5-(2-(pyridin-2-yl)-4,5,6,7-tetrahydrobenzo[d]oxazol-6-yl)nicotinonitrile

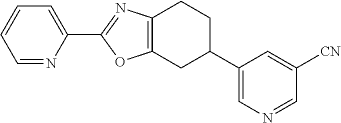

### Compound 254: 4-(2-(pyridin-2-yl)-4,5,6,7-tetrahydrobenzo[d]oxazol-6-yl)picolinonitrile

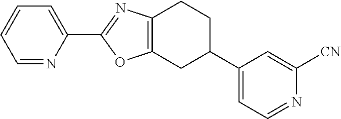

### Compound 255: 3-bromo-5-(2-(pyridin-2-yl)-4,5,6,7-tetrahydrobenzo[d]oxazol-6-yl)benzonitrile

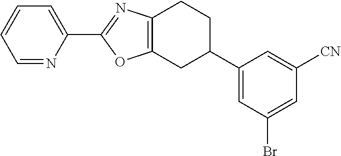

### Compound 256: 3-morpholino-5-(2-(pyridin-2-yl)-4,5,6,7-tetrahydrobenzo[d]oxazol-6-yl)benzonitrile

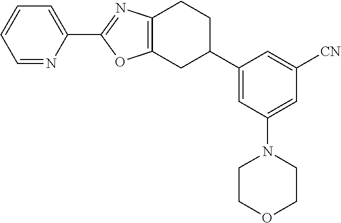

### Compound 257: 6-(3-fluoro-5-(pyridin-4-yl)phenyl)-2-(pyridin-2-yl)-4,5,6,7-tetrahydrobenzo[d]oxazole

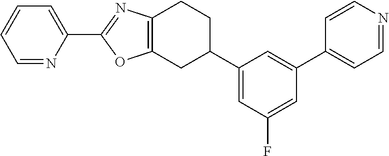

### Compound 258: 2-(6-(3-cyanophenyl)-4,5,6,7-tetrahydrobenzo[d]oxazol-2-yl)pyridine 1-oxide

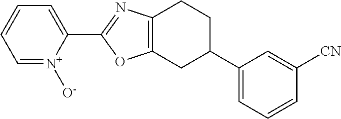

### Compound 259: 5-(2-(pyridin-2-yl)-4,5,6,7-tetrahydrobenzo[d]oxazol-6-yl)isophthalonitrile

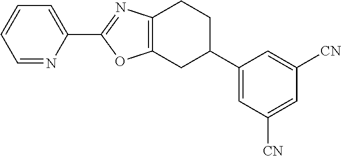

### Compound 260: 6-(3,5-difluorophenyl)-2-(pyridin-2-yl)-4,5,6,7-tetrahydrobenzo[d]oxazole

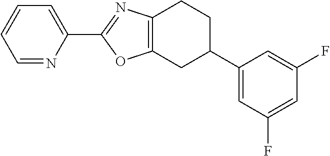

### Compound 261: 2-phenyl-6-(pyrazin-2-yl)-4,5,6,7-tetrahydrobenzo[d]oxazole

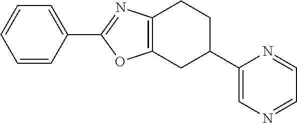

### Compound 262: 2-(2-phenyl-4,5,6,7-tetrahydrobenzo[d]oxazol-6-yl)nicotinonitrile

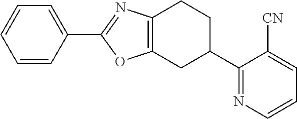

### Compound 263: 2-phenyl-6-(pyridin-2-yl)-4,5,6,7-tetrahydrobenzo[d]oxazole

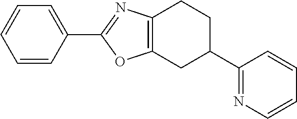

### Compound 264: 3-fluoro-5-(2-phenyl-4,5,6,7-tetrahydrobenzo[d]oxazol-6-yl)benzonitrile

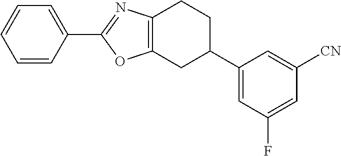

### Compound 265: 2-(pyridin-2-yl)-6-(thiazol-2-yl)-4,5,6,7-tetrahydrobenzo[d]oxazole

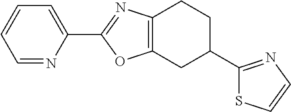

### Compound 266: 6-(pyridin-2-yl)-2-m-tolyl-4,5,6,7-tetrahydrobenzo[d]oxazole

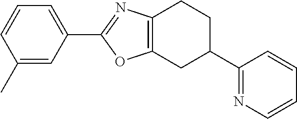

### Compound 267: 2-(2-m-tolyl-4,5,6,7-tetrahydrobenzo[d]oxazol-6-yl)nicotinonitrile

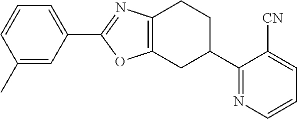

### Compound 268: 3-(6-(pyridin-2-yl)-4,5,6,7-tetrahydrobenzo[d]oxazol-2-yl)benzonitrile

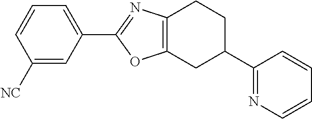

### Compound 269: 2-(2-(3-cyanophenyl)-4,5,6,7-tetrahydrobenzo[d]oxazol-6-yl)nicotinonitrile

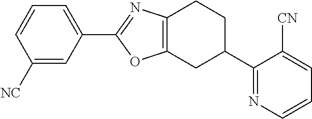

### Compound 270: 3,3′-(4,5,6,7-tetrahydrobenzo[d]oxazole-2,6-diyl)dibenzonitrile

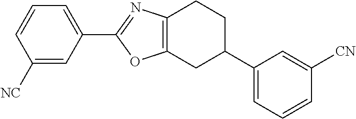

### Compound 271: 2-(2-chlorophenyl)-6-(pyridin-2-yl)-4,5,6,7-tetrahydrobenzo[d]oxazole

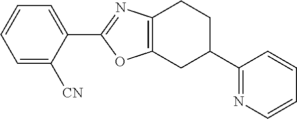

### Compound 272: 2-(2-(2-chlorophenyl)-4,5,6,7-tetrahydrobenzo[d]oxazol-6-yl)nicotinonitrile

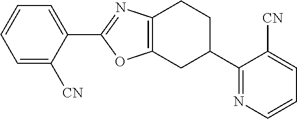

### Compound 273: 2-(4-chlorophenyl)-6-(pyridin-2-yl)-4,5,6,7-tetrahydrobenzo[d]oxazole

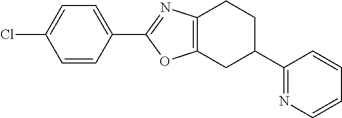

### Compound 274: 2-(3-methoxyphenyl)-6-(pyridin-2-yl)-4,5,6,7-tetrahydrobenzo[d]oxazole

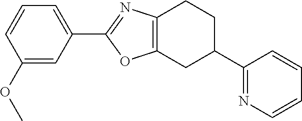

### Compound 275: 2-(2-(3-methoxyphenyl)-4,5,6,7-tetrahydrobenzo[d]oxazol-6-yl)nicotinonitrile

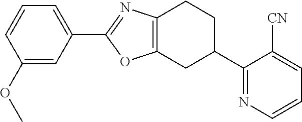

### Compound 276: 3-(2-(5-methylpyridin-2-yl)-4,5,6,7-tetrahydrobenzo[d]oxazol-6-yl)benzonitrile

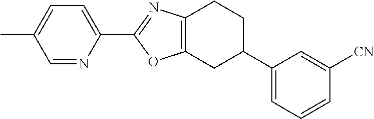

### Compound 277: 3-(2-(pyridin-4-yl)-4,5,6,7-tetrahydrobenzo[d]oxazol-6-yl)benzonitrile

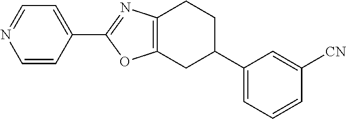

### Compound 278: 3-(2-(4-methylpyridin-2-yl)-4,5,6,7-tetrahydrobenzo[d]oxazol-6-yl)benzonitrile

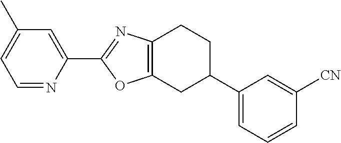

### Compound 279: 3-(2-(pyridin-3-yl)-4,5,6,7-tetrahydrobenzo[d]oxazol-6-yl)benzonitrile

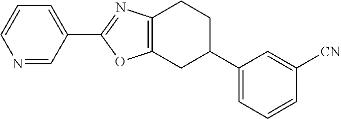

### Compound 280: 3-fluoro-5-(4-methyl-2-(pyridin-2-yl)-4,5,6,7-tetrahydrobenzo[d]oxazol-6-yl)benzonitrile

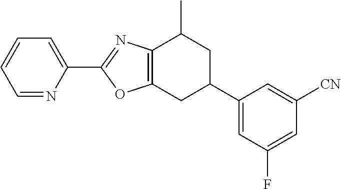

### Compound 281: 3-fluoro-5-(5-methyl-2-(pyridin-2-yl)-4,5,6,7-tetrahydrobenzo[d]oxazol-6-yl)benzonitrile

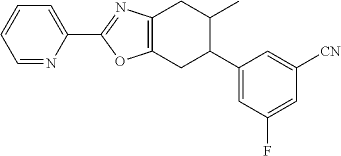

### Compound 282: 6-(2-chlorophenyl)-2-(pyridin-2-yl)-4,5,6,7-tetrahydrobenzo[d]oxazole

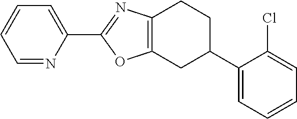

### Compound 283: 6-(3-chloropyridin-2-yl)-2-(pyridin-2-yl)-4,5,6,7-tetrahydrobenzo[d]oxazole

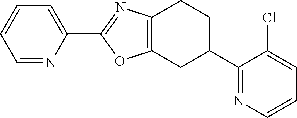

### Compound 284: 6-(2-chloro-4-fluorophenyl)-2-(pyridin-2-yl)-4,5,6,7-tetrahydrobenzo[d]oxazole

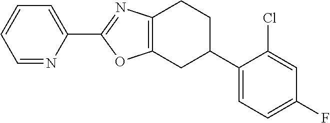

### Compound 285: 3-(2-(pyridin-2-yl)-4,5,6,7-tetrahydrobenzo[d]oxazol-5-yl)benzonitrile

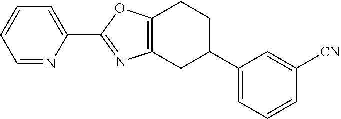

### Compound 286: 2-(2-(pyridin-2-yl)-4,5,6,7-tetrahydrobenzo[d]oxazol-5-yl)isonicotinonitrile

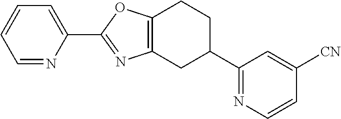

### Compound 287: 5-(2-(pyridin-2-yl)-4,5,6,7-tetrahydrobenzo[d]oxazol-5-yl)nicotinonitrile

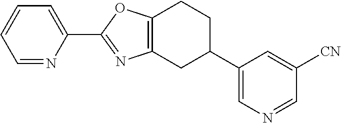

### Compound 288: 4-(2-(pyridin-2-yl)-4,5,6,7-tetrahydrobenzo[d]oxazol-5-yl)picolinonitrile

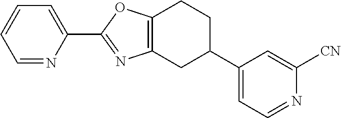

### Compound 289: 2,5-di(pyridin-2-yl)-4,5,6,7-tetrahydrobenzo[d]oxazole

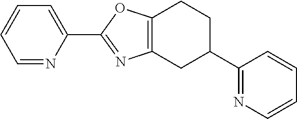

### Compound 290: 2-(2-(pyridin-2-yl)-4,5,6,7-tetrahydrobenzo[d]oxazol-5-yl)nicotinonitrile

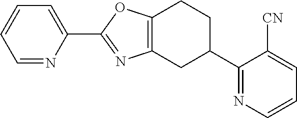

### Compound 291: 6-(2-(pyridin-2-yl)-4,5,6,7-tetrahydrobenzo[d]oxazol-5-yl)picolinonitrile

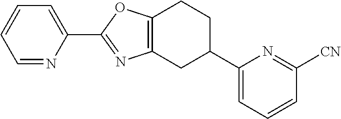

### Compound 292: 5-(3,5-difluorophenyl)-2-(pyridin-2-yl)-4,5,6,7-tetrahydrobenzo[d]oxazole

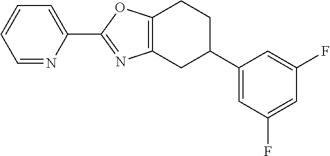

### Compound 293: 3-(2-(pyridin-2-yl)-4,5,6,7-tetrahydrobenzo[d]oxazol-5-yl)-5-(trifluoromethyl)benzonitrile

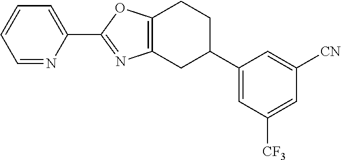

### Compound 294: 3-fluoro-5-(2-(5-fluoropyridin-2-yl)-4,5,6,7-tetrahydrobenzo[d]oxazol-5-yl)benzonitrile

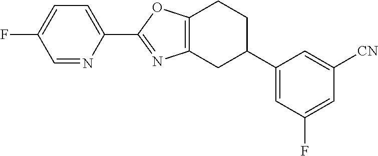

### Compound 295: 2-fluoro-4-(2-(pyridin-2-yl)-4,5,6,7-tetrahydrobenzo[d]oxazol-5-yl)benzonitrile

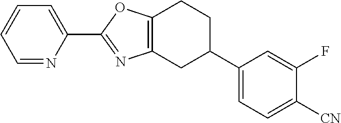

### Compound 296: 2-(2-phenyl-4,5,6,7-tetrahydrobenzo[d]oxazol-5-yl)nicotinonitrile

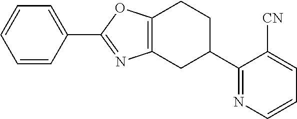

### Compound 297: 2-phenyl-5-(pyridin-2-yl)-4,5,6,7-tetrahydrobenzo[d]oxazole

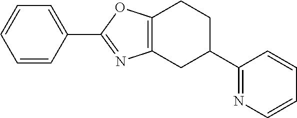

### Compound 298: 2-(2-(3-chlorophenyl)-4,5,6,7-tetrahydrobenzo[d]oxazol-5-yl)nicotinonitrile

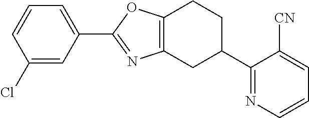

### Compound 299: 2-(3-chlorophenyl)-5-(pyridin-2-yl)-4,5,6,7-tetrahydrobenzo[d]oxazole

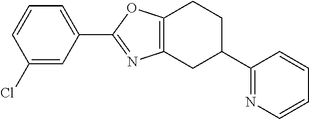

### Compound 300: 2-(3-chlorophenyl)-5-(pyrazin-2-yl)-4,5,6,7-tetrahydrobenzo[d]oxazole

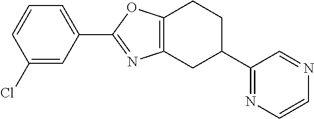

### Compound 301: 2-(4-methoxyphenyl)-5-(pyrazin-2-yl)-4,5,6,7-tetrahydrobenzo[d]oxazole

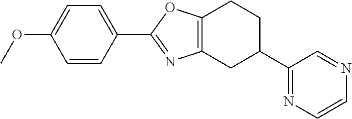

### Compound 302: 2-(2-(4-methoxyphenyl)-4,5,6,7-tetrahydrobenzo[d]oxazol-5-yl)nicotinonitrile

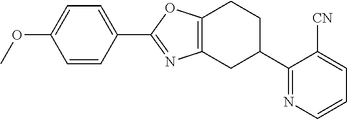

### Compound 303: 2-(2-(4-(trifluoromethyl)phenyl)-4,5,6,7-tetrahydrobenzo[d]oxazol-5-yl)nicotinonitrile

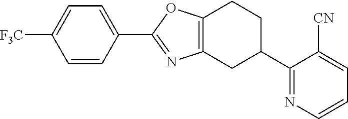

### Compound 304: 5-(pyrazin-2-yl)-2-(4-(trifluoromethyl)phenyl)-4,5,6,7-tetrahydrobenzo[d]oxazole

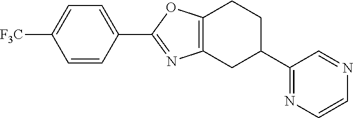

### Compound 305: 2-(2-(4-fluorophenyl)-4,5,6,7-tetrahydrobenzo[d]oxazol-5-yl)nicotinonitrile

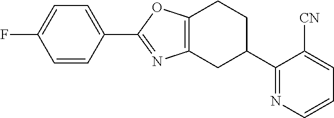

### Compound 306: 2-(4-fluorophenyl)-5-(pyrazin-2-yl)-4,5,6,7-tetrahydrobenzo[d]oxazole

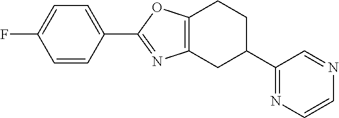

### Compound 307: 2-(4-fluorophenyl)-5-(pyridin-2-yl)-4,5,6,7-tetrahydrobenzo[d]oxazole

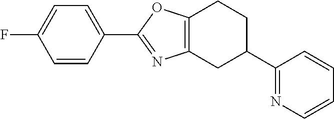

### Compound 308: 2-phenyl-5-(pyrazin-2-yl)-4,5,6,7-tetrahydrobenzo[d]oxazole

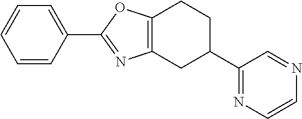

### Compound 309: 2-(3-chlorophenyl)-5-(pyridin-3-yl)-4,5,6,7-tetrahydrobenzo[d]oxazole

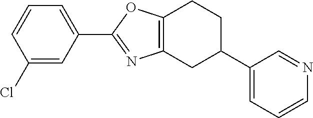

### Compound 310: 3-(2-phenyl-4,5,6,7-tetrahydrobenzo[d]oxazol-5-yl)pyrazine-2-carbonitrile

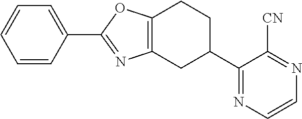

### Compound 311: 3-(2-(3-chlorophenyl)-4,5,6,7-tetrahydrobenzo[d]oxazol-5-yl)pyrazine-2-carbonitrile

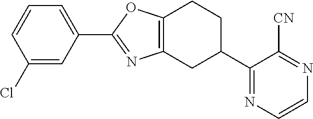

### Compound 312: 2-(pyridin-2-yl)-5-(thiazol-2-yl)-4,5,6,7-tetrahydrobenzo[d]oxazole

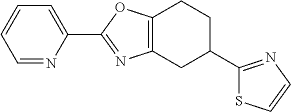

### Compound 313: 5-(3-fluoro-5-methoxyphenyl)-2-(pyridin-2-yl)-4,5,6,7-tetrahydrobenzo[d]oxazole

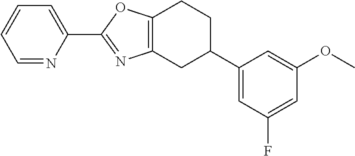

### Compound 314: 2-fluoro-5-(2-(pyridin-2-yl)-4,5,6,7-tetrahydrobenzo[d]oxazol-5-yl)benzonitrile

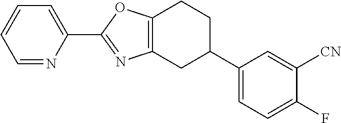

### Compound 315: 5-(pyridin-2-yl)-2-m-tolyl-4,5,6,7-tetrahydrobenzo[d]oxazole

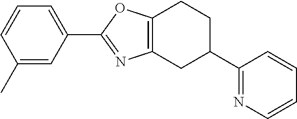

### Compound 316: 2-(2-m-tolyl-4,5,6,7-tetrahydrobenzo[d]oxazol-5-yl)nicotinonitrile

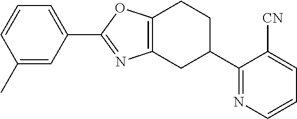

### Compound 317: 3-(5-(pyridin-2-yl)-4,5,6,7-tetrahydrobenzo[d]oxazol-2-yl)benzonitrile

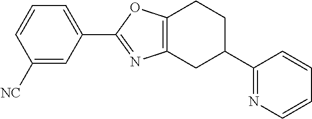

### Compound 318: 2-(2-(3-cyanophenyl)-4,5,6,7-tetrahydrobenzo[d]oxazol-5-yl)nicotinonitrile

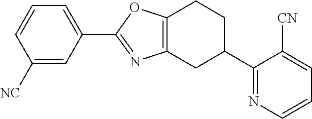

### Compound 319: 3,3′-(4,5,6,7-tetrahydrobenzo[d]oxazole-2,5-diyl)dibenzonitrile

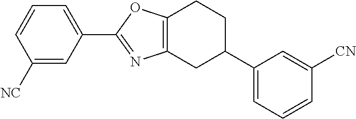

### Compound 320: 2-(2-(3-methoxyphenyl)-4,5,6,7-tetrahydrobenzo[d]oxazol-5-yl)nicotinonitrile

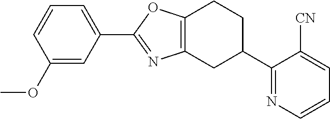

### Compound 321: 3-(2-(pyridin-4-yl)-4,5,6,7-tetrahydrobenzo[d]oxazol-5-yl)benzonitrile

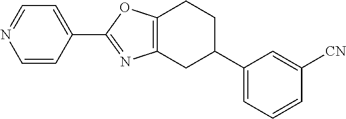

### Compound 322: 2-(3-methylisoxazol-5-yl)-5-(pyridin-2-yl)-4,5,6,7-tetrahydrobenzo[d]oxazole

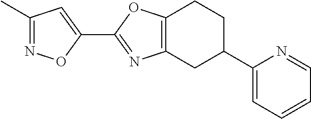

### Compound 323: 3-fluoro-5-(2-(oxazol-2-yl)-4,5,6,7-tetrahydrobenzo[d]oxazol-5-yl)benzonitrile

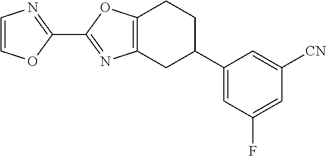

### Compound 324: 3-fluoro-5-(7-methyl-2-(pyridin-2-yl)-4,5,6,7-tetrahydrobenzo[d]oxazol-5-yl)benzonitrile

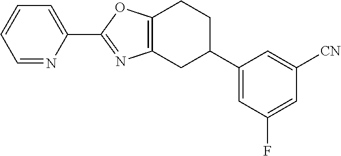

### Compound 325: 5-(3-chlorophenyl)-2-(pyridin-2-yl)-4,5,6,7-tetrahydrobenzo[d]oxazole

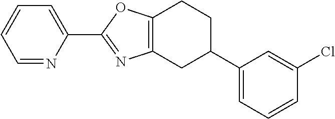

### Compound 326: 5-(3-fluorophenyl)-2-(pyridin-2-yl)-4,5,6,7-tetrahydrobenzo[d]oxazole

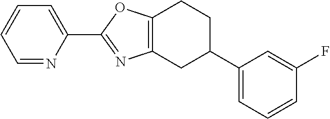

### Compound 327: 3-fluoro-5-(2-(pyrimidin-4-yl)-4,5,6,7-tetrahydrobenzo[d]oxazol-5-yl)benzonitrile

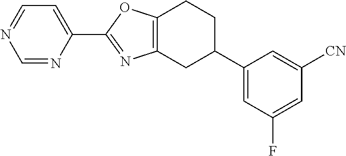

### Compound 328: 5-(3-fluorophenyl)-2-(pyrimidin-4-yl)-4,5,6,7-tetrahydrobenzo[d]oxazole

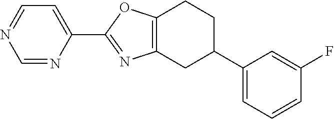

### Compound 329: 5-(3-chlorophenyl)-2-(pyrimidin-4-yl)-4,5,6,7-tetrahydrobenzo[d]oxazole

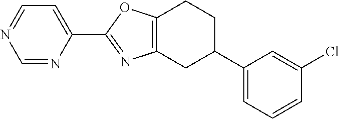

### Compound 330: 3-fluoro-5-(2-(3-methylpyridin-2-yl)-4,5,6,7-tetrahydrobenzo[d]oxazol-5-yl)benzonitrile

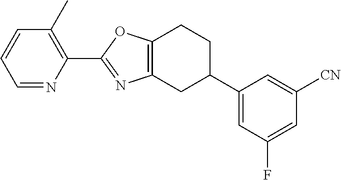

### Compound 331: 5-(3-fluorophenyl)-2-(3-methylpyridin-2-yl)-4,5,6,7-tetrahydrobenzo[d]oxazole

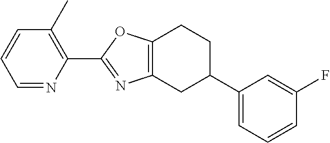

### Compound 332: 5-(3-chlorophenyl)-2-(3-methylpyridin-2-yl)-4,5,6,7-tetrahydrobenzo[d]oxazole

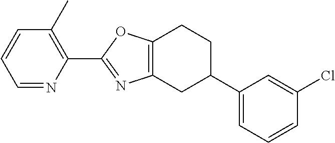

### Compound 333: 5-(3-fluorophenyl)-2-(5-fluoropyridin-2-yl)-4,5,6,7-tetrahydrobenzo[d]oxazole

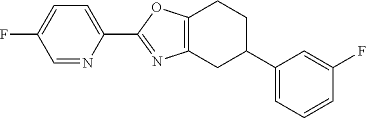

### Compound 334: 5-(3-chlorophenyl)-2-(5-fluoropyridin-2-yl)-4,5,6,7-tetrahydrobenzo[d]oxazole

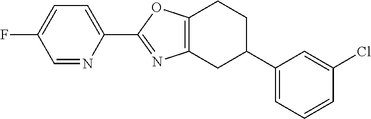

### Compound 335: 5-phenyl-2-(pyridin-2-yl)-4,5,6,7-tetrahydrobenzo[d]oxazole

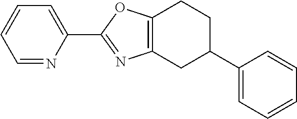

### Compound 336: 5-(3-methoxyphenyl)-2-(pyridin-2-yl)-4,5,6,7-tetrahydrobenzo[d]oxazole

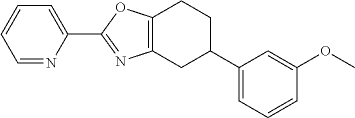

### Compound 337: 5-(3-fluorophenyl)-2-(pyrazin-2-yl)-4,5,6,7-tetrahydrobenzo[d]oxazole

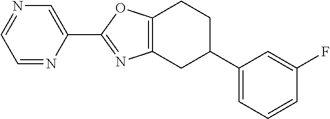

### Compound 338: 5-(3-chlorophenyl)-2-(pyrazin-2-yl)-4,5,6,7-tetrahydrobenzo[d]oxazole

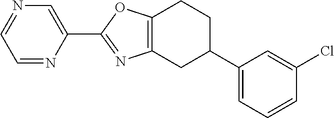

### Compound 339: 3-fluoro-5-(2-(pyrazin-2-yl)-4,5,6,7-tetrahydrobenzo[d]oxazol-5-yl)benzonitrile

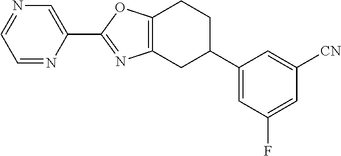

### Compound 340: 2-(pyridin-2-yl)-5-(pyrimidin-5-yl)-4,5,6,7-tetrahydrobenzo[d]oxazole

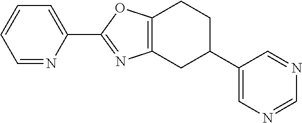

### Compound 341: 2-(pyridin-2-yl)-5-(pyrimidin-2-yl)-4,5,6,7-tetrahydrobenzo[d]oxazole

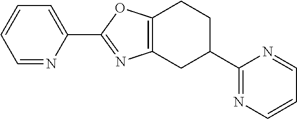

### Compound 342: 5-(3-chloro-4-fluorophenyl)-2-(pyridin-2-yl)-4,5,6,7-tetrahydrobenzo[d]oxazole

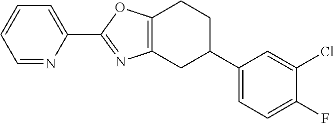

### Compound 343: 3-fluoro-5-(2-(2-methylthiazol-4-yl)-4,5,6,7-tetrahydrobenzo[d]oxazol-5-yl)benzonitrile

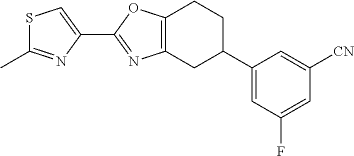

### Compound 344: 5-(3-chloropyridin-2-yl)-2-(pyridin-2-yl)-4,5,6,7-tetrahydrobenzo[d]oxazole

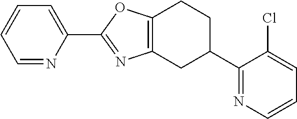

### Compound 345: 3-(2-(pyridin-2-yl)-5,6-dihydro-4H-cyclopenta[d]oxazol-5-yl)benzonitrile

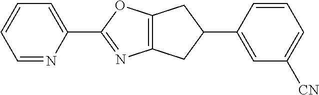

### Compound 346: 2-(2-(pyridin-2-yl)-5,6-dihydro-4H-cyclopenta[d]oxazol-5-yl)nicotinonitrile

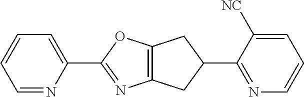

### Compound 347: 3-(5-(pyridin-2-yl)-5,6-dihydro-4H-cyclopenta[d]oxazol-2-yl)benzonitrile

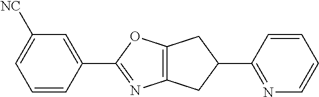

### Compound 348: 3-fluoro-5-(2-(pyridin-2-yl)-5,6-dihydro-4H-cyclopenta[d]oxazol-5-yl)benzonitrile

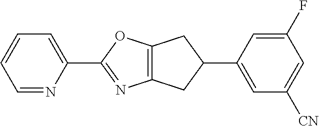

### Compound 349: 5-(2-chlorophenyl)-2-(pyridin-2-yl)-5,6-dihydro-4H-cyclopenta[d]oxazole

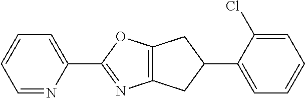

### Compound 350: 5-(3-chloropyridin-2-yl)-2-(pyridin-2-yl)-5,6-dihydro-4H-cyclopenta[d]oxazole

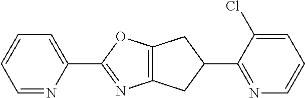

### Compound 351: 5-(2-chloro-4-fluorophenyl)-2-(pyridin-2-yl)-5,6-dihydro-4H-cyclopenta[d]oxazole

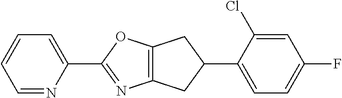

### Compound 352: 2-(2-(3-cyanophenyl)-5,6-dihydro-4H-cyclopenta[d]oxazol-5-yl)nicotinonitrile

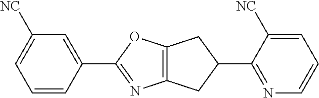

### Compound 353: 3-(2-(pyridin-2-yl)-5,6,7,8-tetrahydro-4H-cyclohepta[d]oxazol-6-yl)benzonitrile

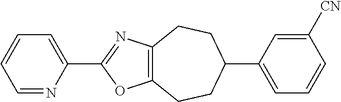

### Compound 354: 3-fluoro-5-(2-(pyridin-2-yl)-5,6,7,8-tetrahydro-4H-cyclohepta[d]oxazol-6-yl)benzonitrile

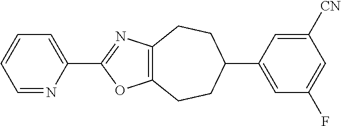

### Compound 355: 6-(2-chlorophenyl)-2-(pyridin-2-yl)-5,6,7,8-tetrahydro-4H-cyclohepta[d]oxazole

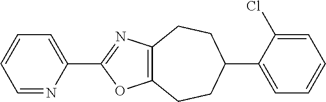

### Compound 356: 6-(3-chloropyridin-2-yl)-2-(pyridin-2-yl)-5,6,7,8-tetrahydro-4H-cyclohepta[d]oxazole

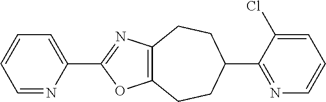

### Compound 357: 6-(2-chloro-4-fluorophenyl)-2-(pyridin-2-yl)-5,6,7,8-tetrahydro-4H-cyclohepta[d]oxazole

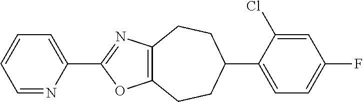

### Compound 358: 3-fluoro-5-(2-(pyridin-2-yl)-5,6,7,8-tetrahydro-4H-cyclohepta[d]oxazol-7-yl)benzonitrile

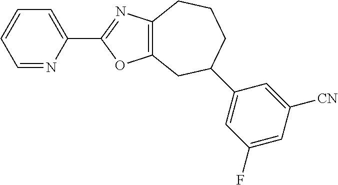

### Compound 359: 3-methoxy-5-(2-(pyridin-2-yl)-5,6,7,8-tetrahydro-4H-cyclohepta[d]oxazol-7-yl)benzonitrile

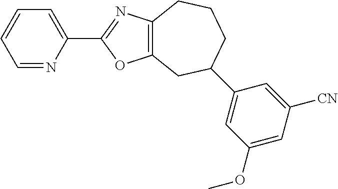

### Compound 360: 3-(2-(pyrimidin-2-yl)-5,6,7,8-tetrahydro-4H-cyclohepta[d]oxazol-7-yl)benzonitrile

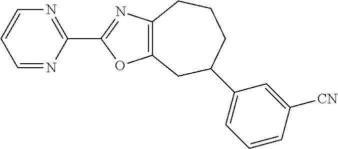

### Compound 361: 3-(2-(pyridin-2-yl)-5,6,7,8-tetrahydro-4H-cyclohepta[d]oxazol-7-yl)benzonitrile

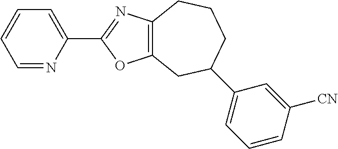

### Compound 362: 3-(2-(pyridin-2-yl)-5,6,7,8-tetrahydro-4H-cyclohepta[d]oxazol-7-yl)-5-(trifluoromethyl)benzonitrile

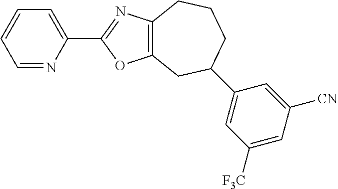

### Compound 363: 2-(pyridin-2-yl)-7-(3-(trifluoromethyl)phenyl)-5,6,7,8-tetrahydro-4H-cyclohepta[d]oxazole

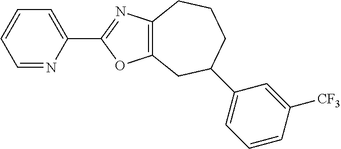

### Compound 364: 3-fluoro-5-(2-(5-fluoropyridin-2-yl)-5,6,7,8-tetrahydro-4H-cyclohepta[d]oxazol-7-yl)benzonitrile

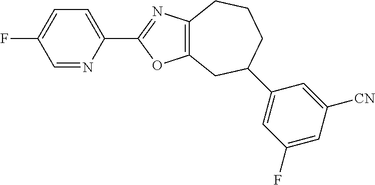

### Compound 365: 3-(2-(5-fluoropyridin-2-yl)-5,6,7,8-tetrahydro-4H-cyclohepta[d]oxazol-7-yl)benzonitrile

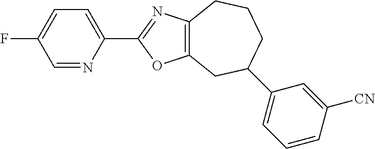

### Compound 366: 7-(2-chlorophenyl)-2-(pyridin-2-yl)-5,6,7,8-tetrahydro-4H-cyclohepta[d]oxazole

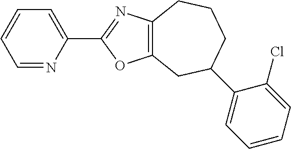

### Compound 367: 7-(3-chloropyridin-2-yl)-2-(pyridin-2-yl)-5,6,7,8-tetrahydro-4H-cyclohepta[d]oxazole

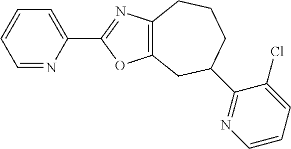

### Compound 368: 7-(2-chloro-4-fluorophenyl)-2-(pyridin-2-yl)-5,6,7,8-tetrahydro-4H-cyclohepta[d]oxazole

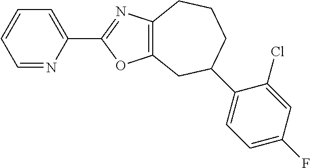

### Compound 369: 3-fluoro-5-(2-(pyridin-2-yl)-5,6,7,8-tetrahydro-4H-cyclohepta[d]oxazol-5-yl)benzonitrile

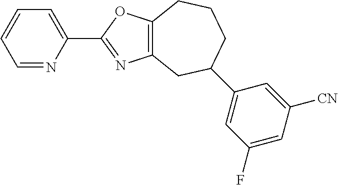

### Compound 370: 3-(2-(pyridin-2-yl)-5,6,7,8-tetrahydro-4H-cyclohepta[d]oxazol-5-yl)benzonitrile

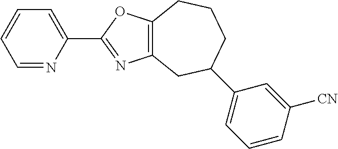

### Compound 371: 5-(3-fluorophenyl)-2-(pyridin-2-yl)-5,6,7,8-tetrahydro-4H-cyclohepta[d]oxazole

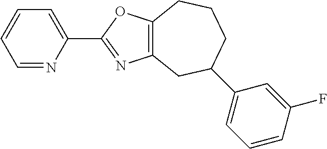

### Compound 372: 5-(3,5-difluorophenyl)-2-(pyridin-2-yl)-5,6,7,8-tetrahydro-4H-cyclohepta[d]oxazole

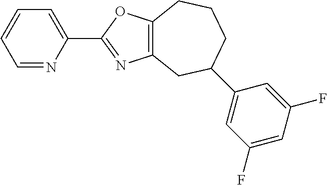

### Compound 373: 5-(3-chlorophenyl)-2-(pyridin-2-yl)-5,6,7,8-tetrahydro-4H-cyclohepta[d]oxazole

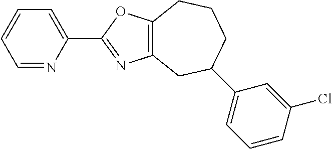

### Compound 374: 5-(6-methoxypyridin-2-yl)-2-(pyridin-2-yl)-5,6,7,8-tetrahydro-4H-cyclohepta[d]oxazole

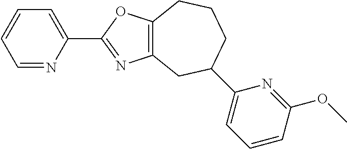

### Compound 375: 5-(5-fluoropyridin-3-yl)-2-(pyridin-2-yl)-5,6,7,8-tetrahydro-4H-cyclohepta[d]oxazole

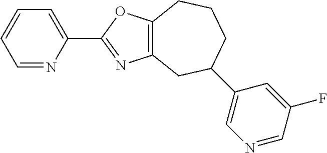

### Compound 376: 5-(3-fluorophenyl)-2-(5-fluoropyridin-2-yl)-5,6,7,8-tetrahydro-4H-cyclohepta[d]oxazole

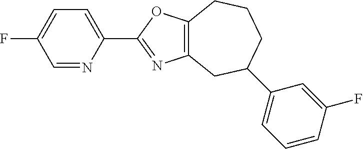

### Compound 377: 5-(3-chlorophenyl)-2-(5-fluoropyridin-2-yl)-5,6,7,8-tetrahydro-4H-cyclohepta[d]oxazole

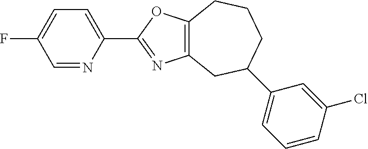

### Compound 378: 3-fluoro-5-(2-(5-fluoropyridin-2-yl)-5,6,7,8-tetrahydro-4H-cyclohepta[d]oxazol-5-yl)benzonitrile

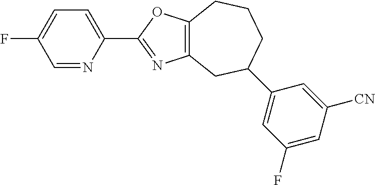

### Compound 379: 5-(3-fluorophenyl)-2-(pyrimidin-4-yl)-5,6,7,8-tetrahydro-4H-cyclohepta[d]oxazole

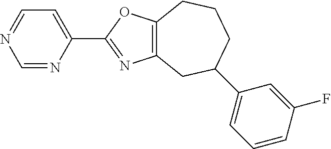

### Compound 380: 5-(3-chlorophenyl)-2-(pyrimidin-4-yl)-5,6,7,8-tetrahydro-4H-cyclohepta[d]oxazole

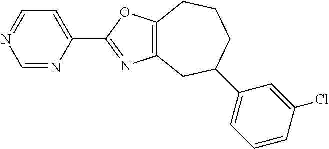

### Compound 381: 3-fluoro-5-(2-(pyrimidin-4-yl)-5,6,7,8-tetrahydro-4H-cyclohepta[d]oxazol-5-yl)benzonitrile

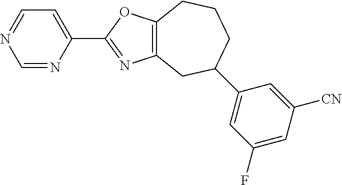

### Compound 382: 5-(3-fluorophenyl)-2-(pyrazin-2-yl)-5,6,7,8-tetrahydro-4H-cyclohepta[d]oxazole

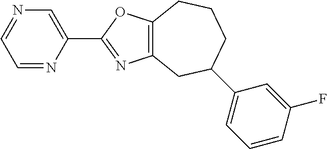

### Compound 383: 5-(3-chlorophenyl)-2-(pyrazin-2-yl)-5,6,7,8-tetrahydro-4H-cyclohepta[d]oxazole

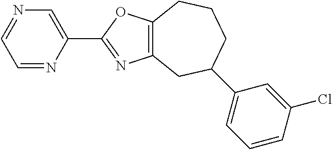

### Compound 384: 3-fluoro-5-(2-(pyrazin-2-yl)-5,6,7,8-tetrahydro-4H-cyclohepta[d]oxazol-5-yl)benzonitrile

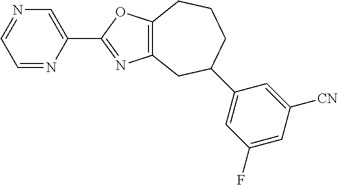

### Compound 385: 2-(pyridin-2-yl)-5-(pyrimidin-5-yl)-5,6,7,8-tetrahydro-4H-cyclohepta[d]oxazole

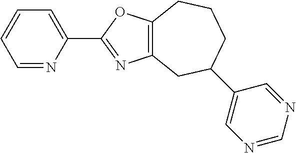

### Compound 386: 2-(pyridin-2-yl)-5-(pyrimidin-2-yl)-5,6,7,8-tetrahydro-4H-cyclohepta[d]oxazole

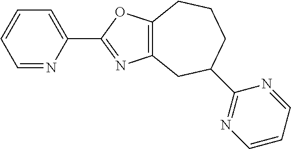

### Compound 387: 5-(3-chloro-4-fluorophenyl)-2-(pyridin-2-yl)-5,6,7,8-tetrahydro-4H-cyclohepta[d]oxazole

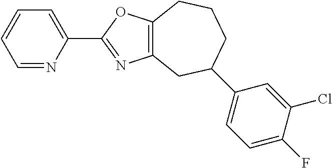

### Compound 388: 3-fluoro-5-(2-(2-methylthiazol-4-yl)-5,6,7,8-tetrahydro-4H-cyclohepta[d]oxazol-5-yl)benzonitrile

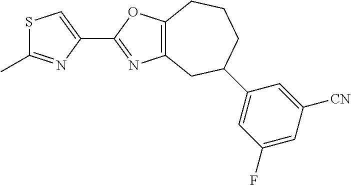

### Compound 389: 5-(2-chlorophenyl)-2-(pyridin-2-yl)-5,6,7,8-tetrahydro-4H-cyclohepta[d]oxazole

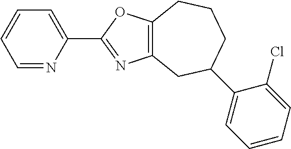

### Compound 390: 5-(3-chloropyridin-2-yl)-2-(pyridin-2-yl)-5,6,7,8-tetrahydro-4H-cyclohepta[d]oxazole

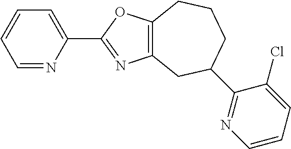

Compound 391: 5-(2-chloro-4-fluorophenyl)-2-(pyridin-2-yl)-5,6,7,8-tetrahydro-4H-cyclohepta[d]oxazole

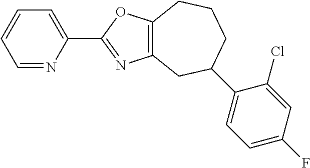

B. mGluR5 In Vitro Functional Assay

The functional assay utilized an aequorin cell line expressing human recombinant mGluR5 receptor, an inducible cell line that expresses the human receptor under the control of a promoter induced by doxycycline.

mGluR5 cells in mid-log phase, grown 18 hours prior to the test in antibiotic-free media supplemented with doxycycline (600 ng/mL), were detached by gentle flushing with PBS-EDTA (5 nM EDTA). The cells were recovered by centrifugation and resuspended in assay buffer (HBSS, 2.1 nM CaCl₂, 3 ug/mL glutamate-pyruvate transaminase, 4 nM MEM sodium pyruvate, 0.1% BSA protease-free). Cells were incubated at room temperature for at least 4 hours with Coelenterazine h (Molecular Probes). The cell suspension (60 uL) containing the test compound was mixed with a solution of reference agonist (30 uL) at its EC₈₀, following an incubation of 3 min. after the first injection. The resulting emission of light was recorded using a Hamamatsu Functional Drug Screening System 6000 (FDSS 6000). To standardize the emission of recorded light (determination of the “100% signal’) across plates and across different experiments, some of the wells contained 100 uM digitonin or a saturating concentration (20 mM) of ATP. Plates also contained the reference agonist (glutamate) at a concentration equivalent to the EC₈₀ obtained during assay validation. Percentages of inhibition were calculated on the basis of the activation induced by the reference agonist at a concentration equal to the EC₈₀. Dose-response data were analyzed with XLFit (IDBS) software using nonlinear regression applied to a sigmoidal dose-response model.

In one embodiment, representative IC₅₀ values are obtained for the examples.

In one embodiment, IC₅₀ values were obtained for selected examples. The tables below summarize IC₅₀ values obtained in duplicate for the selected examples.

The following abbreviations are used: IC₅₀<1 uM: +++; IC₅₀<10 uM: ++; IC₅₀>10 uM: +.

TABLE 1

| Compound | |
|---|---|
| No. | IC₅₀ |
| 1 | +++ |
| 2 | ++ |
| 3 | +++ |
| 4 | +++ |
| 5 | + |
| 6 | ++ |
| 7 | ++ |
| 8 | ++ |
| 9 | ++ |
| 10 | +++ |
| 11 | + |
| 12 | ++ |
| 13 | ++ |
| 14 | ++ |
| 15 | +++ |
| 16 | ++ |
| 17 | +++ |
| 18 | ++ |
| 19 | ++ |
| 20 | + |
| 21 | +++ |
| 22 | ++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | ++ |
| 36 | + |
| 37 | ++ |
| 38 | ++ |
| 39 | +++ |
| 40 | +++ |
| 41 | +++ |
| 42 | + |
| 43 | + |
| 44 | + |
| 45 | + |
| 46 | + |
| 47 | ++ |
| 48 | ++ |
| 49 | ++ |
| 50 | ++ |
| 51 | +++ |
| 52 | ++ |
| 53 | +++ |
| 54 | ++ |
| 55 | +++ |
| 56 | +++ |
| 57 | ++ |
| 58 | +++ |
| 59 | +++ |
| 60 | +++ |
| 61 | +++ |
| 62 | + |
| 63 | ++ |
| 64 | +++ |
| 65 | + |
| 66 | ++ |
| 67 | +++ |
| 68 | ++ |
| 69 | ++ |
| 70 | +++ |
| 71 | ++ |
| 72 | +++ |
| 73 | + |
| 74 | ++ |
| 75 | ++ |
| 76 | ++ |
| 77 | + |
| 78 | + |
| 79 | + |
| 80 | ++ |
| 81 | +++ |
| 82 | +++ |
| 83 | +++ |
| 84 | +++ |
| 85 | +++ |
| 86 | + |
| 87 | +++ |
| 88 | +++ |
| 89 | +++ |
| 90 | +++ |
| 91 | +++ |
| 92 | +++ |
| 93 | +++ |
| 94 | +++ |
| 95 | ++ |
| 96 | +++ |
| 97 | +++ |
| 98 | +++ |
| 99 | +++ |
| 100 | +++ |
| 101 | +++ |
| 102 | +++ |
| 103 | +++ |
| 104 | +++ |
| 105 | ++ |
| 106 | ++ |
| 107 | ++ |
| 108 | ++ |
| 109 | +++ |
| 110 | +++ |
| 111 | + |
| 112 | +++ |
| 113 | +++ |
| 114 | +++ |

TABLE 2

| Compound | |
|---|---|
| No. | IC₅₀ |
| 148 | ++ |
| 149 | ++ |
| 150 | + |
| 151 | + |
| 115 | +++ |
| 116 | +++ |
| 118 | +++ |
| 119 | ++ |
| 120 | ++ |
| 121 | ++ |
| 122 | + |
| 123 | + |
| 124 | +++ |
| 125 | +++ |
| 126 | +++ |
| 146 | +++ |
| 152 | +++ |
| 127 | +++ |
| 153 | +++ |
| 154 | +++ |
| 132 | ++ |
| 131 | ++ |
| 155 | + |
| 156 | +++ |
| 157 | +++ |
| 158 | +++ |
| 159 | +++ |
| 160 | + |
| 161 | +++ |
| 162 | +++ |
| 163 | +++ |
| 164 | ++ |
| 165 | +++ |
| 166 | +++ |
| 167 | +++ |
| 168 | ++ |
| 169 | +++ |
| 170 | +++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | ++ |
| 185 | +++ |
| 186 | +++ |
| 187 | +++ |
| 188 | +++ |
| 189 | +++ |
| 190 | ++ |
| 191 | + |
| 192 | + |
| 193 | +++ |
| 194 | +++ |
| 195 | + |
| 196 | + |
| 144 | +++ |
| 133 | +++ |
| 134 | +++ |
| 135 | +++ |
| 136 | +++ |
| 137 | +++ |
| 138 | +++ |
| 139 | ++ |
| 140 | ++ |
| 141 | +++ |
| 197 | +++ |
| 198 | +++ |
| 199 | +++ |
| 200 | + |
| 201 | ++ |
| 202 | +++ |
| 203 | +++ |
| 204 | + |
| 145 | +++ |
| 205 | +++ |
| 206 | + |
| 207 | ++ |
| 208 | + |
| 209 | +++ |
| 210 | +++ |
| 211 | +++ |
| 212 | +++ |
| 213 | +++ |
| 214 | +++ |
| 215 | +++ |
| 216 | +++ |
| 217 | +++ |
| 218 | ++ |
| 219 | +++ |
| 220 | +++ |
| 221 | +++ |
| 222 | +++ |
| 223 | +++ |
| 224 | +++ |
| 225 | +++ |

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

All of the patents, patent applications and publications referred to herein are incorporated herein by reference in their entireties. Citation or identification of any reference in this application is not an admission that such reference is available as prior art to this application. The full scope of the disclosure is better understood with reference to the appended claims.

1. A compound of formula (I): 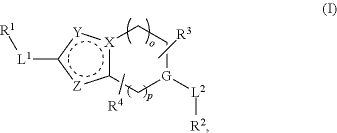
or a pharmaceutically acceptable salt, solvate, or stereoisomer thereof, wherein
R¹ is cycloalkyl, heterocyclyl, aryl, or heteroaryl, each of which is optionally substituted;
R² is cycloalkyl, heterocyclyl, aryl, or heteroaryl, each of which is optionally substituted;
R³ and R⁴ are each independently hydrogen, halogen, or lower alkyl; or
when R³ and R⁴ are attached to the same carbon atom, CR³R⁴ is C═O, or R³ and R⁴ may be combined with the carbon atom to which they are attached to form a 3- to 7-membered Spiro cycloalkyl; or
when R³ and R⁴ are attached to different carbon atoms, R³ and R⁴ may be combined with the carbon atoms to which they are attached to form a 3- to 7-membered bridged or fused cycloalkyl;
L¹ is a bond, —S—, —SO—, —SO₂—, —O—, —NR⁹—, —CR⁵R⁶—, —CR⁵R⁶—CR⁷R⁸—, optionally substituted cycloalkyl, optionally substituted heterocyclyl; optionally substituted aryl, or optionally substituted heteroaryl;
L² is a bond, —O—, —NR⁹—, —CR⁵R⁶— or —CR⁵R⁶—CR⁷R⁸—;
X is C or N;
Y is O, S, N, NR¹⁰, or CR¹⁰;
Z is O, S, N, NR¹⁰, or CR¹⁰; wherein Y and Z are not both O or both S;
R⁵ and R⁶ are each independently hydrogen, halogen, or lower alkyl, or CR⁵R⁶ is C═O; or R⁵ and R⁶ may be combined with the carbon atom to which they are attached to form a 3- to 7-membered cycloalkyl;
R⁷ and R⁸ are each independently hydrogen, halogen, or lower alkyl, or CR⁷R⁸ is C═O; or R⁷ and R⁸ may be combined with the carbon atom to which they are attached to form a 3- to 7-membered cycloalkyl;
R⁹ and R¹⁰ are each independently hydrogen or lower alkyl;
G is N or CH;
o is 0, 1, or 2; and
p is 1 or 2.

2-71. (canceled)
